(19) United States
(12) Patent Application Publication
(10) Pub. No.: US 2025/0213571 A1
(43) Pub. Date: Jul. 3, 2025

(54) TYK2 INHIBITORS AND USES THEREOF

(71) Applicant: Alumis Inc., South San Francisco, CA (US)

(72) Inventors: Ken Brameld, South San Francisco, CA (US); Dean Dragoli, South San Francisco, CA (US); Robert Zamboni, Beaconsfield (CA)

(21) Appl. No.: 18/847,389

(22) PCT Filed: Mar. 16, 2023
(86) PCT No.: PCT/US2023/064520

§ 371 Date: Sep. 16, 2024

Related U.S. Application Data
(60) Provisional application No. 63/320,318, filed on Mar. 16, 2022.

(51) Int. Cl. A61K 31/519; C07D 471/22; C07D 487/22; C07D 498/22

(52) U.S. Cl. CPC A61K 31/519; C07D 471/22; C07D 487/22; C07D 498/22

(57) ABSTRACT

Described herein are compounds that are useful in treating a TYK2-mediated disorder. In some embodiments, the TYK2-mediated disorder is an autoimmune disorder, an inflammatory disorder, a proliferative disorder, an endocrine disorder, a neurological disorder, or a disorder associated with transplantation.

### CROSS REFERENCE TO RELATED APPLICATIONS

This application claims the benefit of, and priority to, U.S. Provisional Patent Application No. 63/320,318 filed on Mar. 16, 2022; the content of which is hereby incorporated by reference herein in its entirety.

### BACKGROUND

TYK2 is a non-receptor tyrosine kinase member of the Janus kinase (JAKs) family of protein kinases. The mammalian JAK family consists of four members, TYK2, JAK, JAK2, and JAK3. JAK proteins, including TYK2, are integral to cytokine signaling. TYK2 associates with the cytoplasmic domain of type I and type II cytokine receptors, as well as interferon types I and III receptors, and is activated by those receptors upon cytokine binding. Cytokines implicated in TYK2 activation include interferons (e.g., IFN-α, IFN-β, IFN-κ, IFN-δ, IFN-ε, IFN-τ, IFN-ω, and IFN-ζ (also known as limitin), and interleukins (e.g., IL-4, IL-6, IL-10, IL-11, IL-12, IL-13, L-22, IL-23, IL-27, IL-31, oncostatin M, ciliary neurotrophic factor, cardiotrophin 1, cardiotrophin-like cytokine, and LIF). The activated TYK2 then goes on to phosphorylate further signaling proteins such as members of the STAT family, including STAT1, STAT2, STAT4, and STAT6.

TYK2 activation by IL-23, has been linked to inflammatory bowel disease (IBD), Crohn's disease, and ulcerative colitis. A genome-wide association study of 2,622 individuals with psoriasis identified associations between disease susceptibility and TYK2. Knockout or tyrphostin inhibition of TYK2 significantly reduces both IL-23 and IL-22-induced dermatitis.

TYK2 also plays a role in respiratory diseases such as asthma, chronic obstructive pulmonary disease (COPD), lung cancer, and cystic fibrosis. Goblet cell hyperplasia (GCH) and mucous hypersecretion is mediated by IL-13-induced activation of TYK2, which in turn activates STAT6.

Decreased TYK2 activity leads to protection of joints from collagen antibody-induced arthritis, a model of human rheumatoid arthritis. Mechanistically, decreased TYK2 activity reduced the production of Th1/Th17-related cytokines and matrix metalloproteases, and other key markers of inflammation.

TYK2 knockout mice showed complete resistance in experimental autoimmune encephalomyelitis (EAE, an animal model of multiple sclerosis (MS)), with no infiltration of CD4 T cells in the spinal cord, as compared to controls, suggesting that TYK2 is essential to pathogenic CD4-mediated disease development in MS. This corroborates earlier studies linking increased TYK2 expression with MS susceptibility. Loss of function mutation in TYK2, leads to decreased demyelination and increased remyelination of neurons, further suggesting a role for TYK2 inhibitors in the treatment of MS and other CNS demyelination disorders.

TYK2 is the sole signaling messenger common to both IL-12 and IL-23. TYK2 knockout reduced methylated BSA injection-induced footpad thickness, imiquimod-induced psoriasis-like skin inflammation, and dextran sulfate sodium or 2,4,6-trinitrobenzene sulfonic acid-induced colitis in mice.

Joint linkage and association studies of various type I IFN signaling genes with systemic lupus erythematosus (SLE, an autoimmune disorder), showed a strong, and significant correlation between loss of function mutations to TYK2 and decreased prevalence of SLE in families with affected members. Genome-wide association studies of individuals with SLE versus an unaffected cohort showed highly significant correlation between the TYK2 locus and SLE.

TYK2 has been shown to play an important role in maintaining tumor surveillance and TYK2 knockout mice showed compromised cytotoxic T cell response, and accelerated tumor development. However, these effects were linked to the efficient suppression of natural killer (NK) and cytotoxic T lymphocytes, suggesting that TYK2 inhibitors would be highly suitable for the treatment of autoimmune disorders or transplant rejection. Although other JAK family members such as JAK3 have similar roles in the immune system, TYK2 has been suggested as a superior target because of its involvement in fewer and more closely related signaling pathways, leading to fewer off-target effects.

Studies in T-cell acute lymphoblastic leukemia (T-ALL) indicate that T-ALL is highly dependent on IL-10 via TYK2 via STAT1-mediated signal transduction to maintain cancer cell survival through upregulation of anti-apoptotic protein BCL2. Knockdown of TYK2, but not other JAK family members, reduced cell growth. Specific activating mutations to TYK2 that promote cancer cell survival include those to the FERM domain (G36D, S47N, and R425H), the JH2 domain (V731I), and the kinase domain (E957D and R1027H). However, it was also identified that the kinase function of TYK2 is required for increased cancer cell survival, as TYK2 enzymes featuring kinase-dead mutations (M978Y or M978F) in addition to an activating mutation (E957D) resulted in failure to transform.

Thus, selective inhibition of TYK2 has been suggested as a suitable target for patients with IL-10 and/or BCL2-addicted tumors, such as 70% of adult T-cell leukemia cases. TYK2 mediated STAT3 signaling has also been shown to mediate neuronal cell death caused by amyloid-β (Aβ) peptide. Decreased TYK2 phosphorylation of STAT3 following Aβ administration lead to decreased neuronal cell death, and increased phosphorylation of STAT3 has been observed in postmortem brains of Alzheimer's patients.

Inhibition of JAK-STAT signaling pathways is also implicated in hair growth, and the reversal of the hair loss associated with alopecia areata.

Accordingly, compounds that inhibit the activity of TYK2 are beneficial, especially those with selectivity over JAK2. Such compounds should deliver a pharmacological response that favorably treats one or more of the conditions described herein without the side-effects associated with the inhibition of JAK2.

Accordingly, there is a need to provide novel inhibitors having more effective or advantageous pharmaceutically relevant properties, like selectivity over other JAK kinases (especially JAK2).

### SUMMARY

Described herein are compounds that are useful in treating a TYK2-mediated disorder. In some embodiments, the TYK2-mediated disorder is an autoimmune disorder, an inflammatory disorder, a proliferative disorder, an endocrine disorder, a neurological disorder, or a disorder associated with transplantation. In some embodiments, the TYK2-mediated disorder is cancer.

For example, disclosed herein is a compound represented by Formula I.

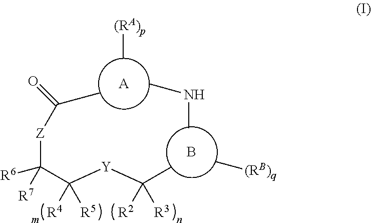

or a pharmaceutically acceptable salt and/or a stereoisomer thereof, wherein
Ring A is a 6,5 or 6,6 fused bicyclic heteroaryl containing two, three, or four ring nitrogens;
R^{A} is independently selected for each occurrence from the group consisting of hydrogen, halogen, —NR^{a}R^{b}, C₁-C₆alkyl, and C₁-C₆alkoxy; wherein C₁-C₆alkyl and C₁-C₆alkoxy may optionally be substituted by one or more halogens or deuteriums;
Ring B is a 6,5 or 6,6 fused bicyclic heteroaryl containing one, two or three ring nitrogens;
R^{B} is independently selected for each occurrence from the group consisting of hydrogen, halogen, —NR^{a}R^{b}, C₁-C₆alkyl, and C₁-C₆alkoxy; wherein C₁-C₆alkyl and C₁-C₆alkoxy may optionally be substituted by one or more halogens or deuteriums;
Y is selected from the group consisting of O, N(R^{a}), S(O)_{w}, CH₂ and a bond;
Z is selected from the group consisting of N(R^{a}) and O;
R² and R³ are each independently selected from the group consisting of hydrogen, deuterium, and C₁-C₆alkyl optionally substituted by one or more substituents each independently selected from R^{P}; or
R² and R³, together with the carbon to which they are attached, may be joined together to form a C₃-C₆cycloalkyl optionally substituted by one or more substituents each independently selected from R^{P};
R⁴ and R⁵ are each independently selected for each occurrence from the group consisting of hydrogen, deuterium, and C₁-C₆alkyl optionally substituted by one or more substituents each independently selected from R; or
R⁴ and R⁵, together with the carbon to which they are attached, may be joined together to form a C₃-C₆cycloalkyl optionally substituted by one or more substituents each independently selected from R^{P};
R⁶ and R⁷ are independently selected from the group consisting of hydrogen, deuterium, halogen, hydroxyl, —NR^{a}R^{b}, cyano, oxo, C₁-C₆alkyl, C₂-C₆alkenyl, C₂-C₆alkynyl, C₁-C₆heteroalkyl, and C₁-C₆alkoxy; wherein R⁴ may be substituted on an available carbon by hydroxyl or one or more substituents each independently selected from R; or
R⁶ and R⁷, together with the carbon to which they are attached, may be joined together to form a C₃-C₆cycloalkyl optionally substituted by one or more substituents each independently selected from R^{P};
R is independently selected for each occurrence from the group consisting of deuterium, halogen, hydroxyl, —NR^{a}R^{b}, cyano, oxo, C₁-C₆alkyl, and C₁-C₆alkoxy;
R^{a} and R^{b} are independently selected for each occurrence from the group consisting of hydrogen and C₁-C₆alkyl, wherein C₁-C₆alkyl may optionally substituted by one or more halogens or deuteriums;
m is 1 or 2;
n is 0 or 1;
p is 0, 1, or 2;
q is 0, 1 or 2; and
w is 0, 1 or 2.

Also disclosed herein is a compound represented by:

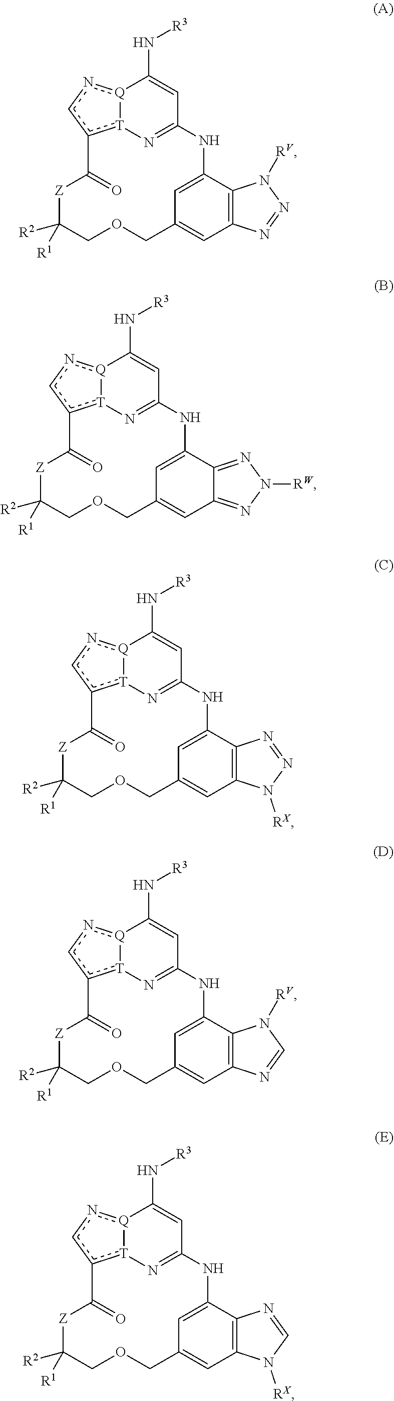

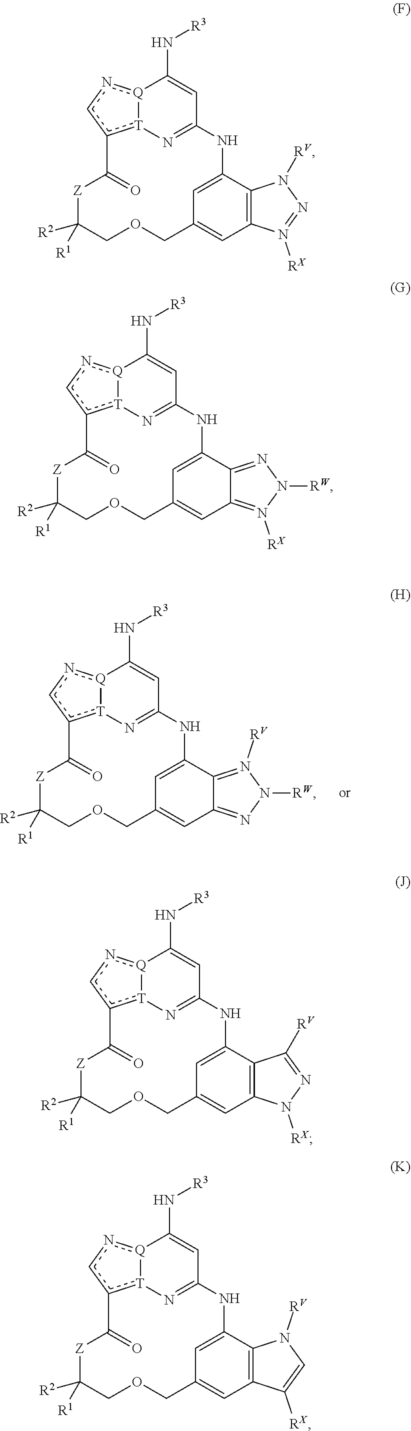

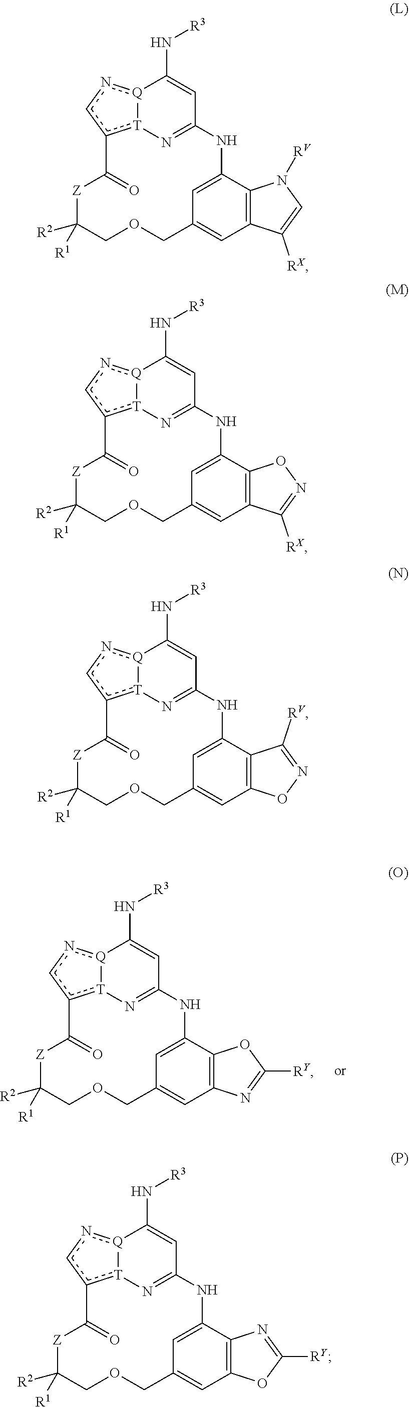

or a pharmaceutically acceptable salt and/or a stereoisomer thereof, wherein:
Q is N and T is C; or
Q is C and T is N;
   wherein Q and T are each members of a heteroaromatic ring system;
Z is NH or O;
R^{V}, R^{W} and R^{X} are each independently selected from the group consisting of hydrogen and C₁-C₄alkyl optionally substituted by one or more deuteriums;
R¹ and R² are independently selected from the group consisting of hydrogen, deuterium, and C₁-C₄alkyl, wherein C₁-C₄alkyl may be substituted on an available carbon by hydroxyl; or
R¹ and R², together with the carbon to which they are attached, may be joined together to form cyclopropyl; and
R³ is hydrogen or C₁-C₄alkyl optionally substituted by one or more deuteriums.

Also disclosed herein is a pharmaceutical composition comprising a therapeutically effective amount of the compound disclosed herein, or a pharmaceutically acceptable salt, stereoisomer, or solvate thereof, and a pharmaceutically acceptable excipient.

Also disclosed herein is a method of inhibiting a TYK2 enzyme in a patient or biological sample comprising contacting said patient or biological sample with a compound disclosed herein, or a pharmaceutically acceptable salt, stereoisomer, or solvate thereof.

Also disclosed herein is a method of treating a TYK2-mediated disorder comprising administering to a patient in need thereof a compound disclosed herein, or a pharmaceutically acceptable salt, stereoisomer, or solvate thereof. In some embodiments, the TYK2-mediated disorder is an autoimmune disorder, an inflammatory disorder, a proliferative disorder, an endocrine disorder, a neurological disorder, or a disorder associated with transplantation. In some embodiments, the disorder is associated with type I interferon, IL-10, IL-12, or IL-23 signaling.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Oxo” refers to ═O.

“Alkyl” refers to an optionally substituted straight-chain, or optionally substituted branched-chain saturated hydrocarbon monoradical having from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s) and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₅ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe. —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₅ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula -Oalkyl wherein alkyl is as defined above. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Aryl” refers to a radical derived from a hydrocarbon ring system comprising hydrogen, 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbomyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuterium atoms. In some embodiments, the alkyl is substituted with one deuterium atom. In some embodiments, the alkyl is substituted with one, two, or three deuterium atoms. In some embodiments, the alkyl is substituted with one, two, three, four, five, or six deuterium atoms. Deuteroalkyl includes, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogen atoms. In some embodiments, the alkyl is substituted with one, two, or three halogen atoms. In some embodiments, the alkyl is substituted with one, two, three, four, five, or six halogen halogens. Haloalkyl includes, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro or iodo. In some embodiments. halogen is fluoro or chloro. In some embodiments, halogen is fluoro.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous and sulfur. In some embodiments, the heterocycloalkyl comprises 1 or 2 heteroatoms selected from nitrogen and oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the cycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-TH-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein, “treatment,” “prevention,” “amelioration,” or “inhibition” encompass delaying the onset of the disorder, or a symptom or condition thereof.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

As used herein, the term “TYK2-mediated” disorders, diseases, and/or conditions as used herein means any disease or other deleterious condition in which TYK2 or a mutant thereof is known to play a role. Accordingly, another embodiment relates to treating or lessening the severity of one or more diseases in which TYK2, or a mutant thereof, is known to play a role. Such TYK2-mediated disorders include but are not limited to autoimmune disorders, inflammatory disorders, proliferative disorders, endocrine disorders, neurological disorders, and disorders associated with transplantation.

### Compounds

Described herein are compounds that are useful in treating a TYK2-mediated disorder. In some embodiments, the TYK2-mediated disorder is an autoimmune disorder, an inflammatory disorder, a proliferative disorder, an endocrine disorder, a neurological disorder, or a disorder associated with transplantation. In some embodiments, the TYK2-mediated disorder is cancer.

For example, disclosed herein is a compound represented by Formula I.

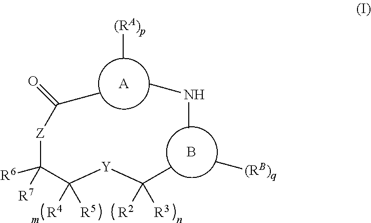

or a pharmaceutically acceptable salt and/or a stereoisomer thereof; wherein
Ring A is a 6,5 or 6,6 fused bicyclic heteroaryl containing two, three, or four ring nitrogens;
R^{A} is independently selected for each occurrence from the group consisting of hydrogen, halogen, —NR^{a}R^{b}, C₁-C₆alkyl, and C₁-C₆alkoxy; wherein C₁-C₆alkyl and C₁-C₆alkoxy may optionally be substituted by one or more halogens or deuteriums;
Ring B is a 6,5 or 6,6 fused bicyclic heteroaryl containing one, two or three ring nitrogens;
R^{B} is independently selected for each occurrence from the group consisting of hydrogen, halogen, —NR^{a}R^{b}, C₁-C₆alkyl, and C₁-C₆alkoxy; wherein C₁-C₆alkyl and C₁-C₆alkoxy may optionally be substituted by one or more halogens or deuteriums;
Y is selected from the group consisting of O, N(R^{a}), S(O)_{w}, CH₂ and a bond;
Z is selected from the group consisting of N(R^{a}) and O;
R² and R³ are each independently selected from the group consisting of hydrogen, deuterium, and C₁-C₆alkyl optionally substituted by one or more substituents each independently selected from R^{P}; or
R² and R³, together with the carbon to which they are attached, may be joined together to form a C₃-C₆cycloalkyl optionally substituted by one or more substituents each independently selected from R^{P};
R⁴ and R⁵ are each independently selected for each occurrence from the group consisting of hydrogen, deuterium, and C₁-C₆alkyl optionally substituted by one or more substituents each independently selected from R; or
R⁴ and R⁵, together with the carbon to which they are attached, may be joined together to form a C₃-C₆cycloalkyl optionally substituted by one or more substituents each independently selected from R^{P};
R⁶ and R⁷ are independently selected from the group consisting of hydrogen, deuterium, halogen, hydroxyl, —NR^{a}R^{b}, cyano, oxo, C₁-C₆alkyl, C₂-C₆alkenyl, C₂-C₆alkynyl, C₁-C₆heteroalkyl, and C₁-C₆alkoxy; wherein R⁴ may be substituted on an available carbon by hydroxyl or one or more substituents each independently selected from R; or
R⁶ and R⁷, together with the carbon to which they are attached, may be joined together to form a C₃-C₆cycloalkyl optionally substituted by one or more substituents each independently selected from R^{P};
R^{P} is independently selected for each occurrence from the group consisting of deuterium, halogen, hydroxyl, —NR^{a}R^{b}, cyano, oxo, C₁-C₆alkyl, and C₁-C₆alkoxy;
R^{a} and R^{b} are independently selected for each occurrence from the group consisting of hydrogen and C₁-C₆alkyl, wherein C₁-C₆alkyl may optionally substituted by one or more halogens or deuteriums;
m is 1 or 2;
n is 0 or 1;
p is 0, 1, or 2;
q is 0, 1 or 2; and
w is 0, 1 or 2.

In some embodiments, ring A is represented by:

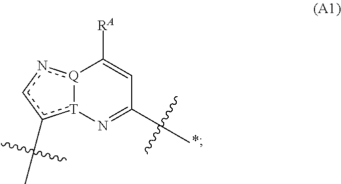

wherein:
Q is N and T is C; or
Q is C and T is N; and
R^{A} is selected from the group consisting of —N(H)CH₃, —NH₂, hydrogen, and —OCH₃;
   wherein * represents the point of attachment to the —NH— group in Formula I.

For example, in certain embodiments ring A is selected from the group consisting of:

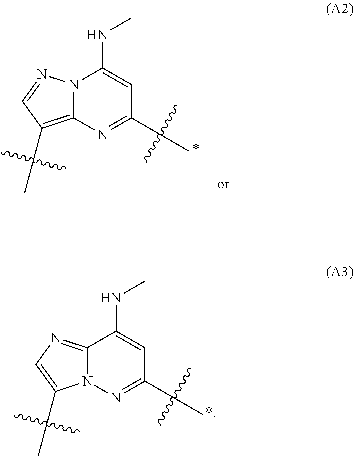 [SMILES: *c1cc(NC)n2ncc(*)c2n1 ; *c1cc(NC)c2ncc(*)n2n1]

In other embodiments, ring A is represented by:

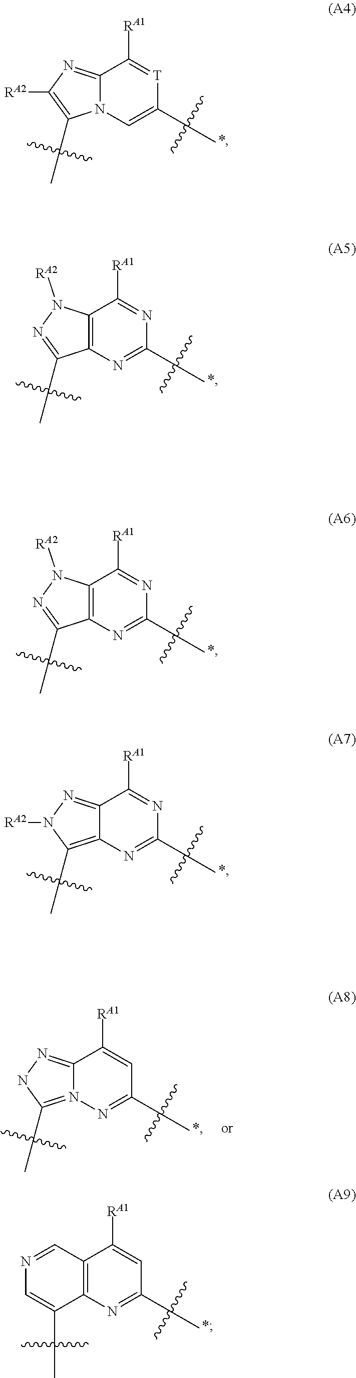

wherein:
T is N or CH;
R^{A1} is selected from the group consisting of —N(H)CH₃, —NH₂, hydrogen, and —OCH₃; and
R^{A2} is selected from the group consisting of hydrogen and —CH₃;
   wherein * represents the point of attachment to the —NH— group in Formula I.

For example, in certain embodiments ring A is selected from the group consisting of:

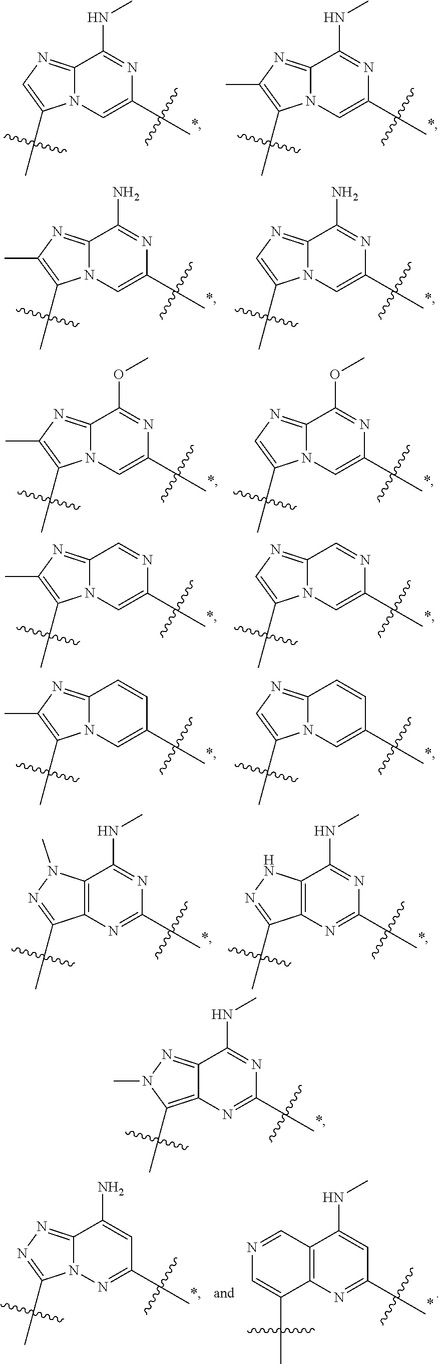

In further embodiments, ring B is represented by:

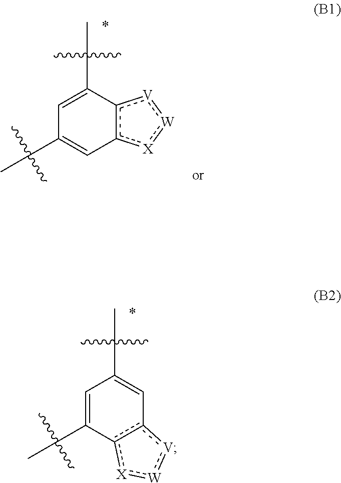

wherein:
V is N(R^{V}), W is N, and X is N; or
V is N, W is N(R^{V}), and X is N; or
V is N, W is N, and X is N(R^{X}); or
V is N, W is C(R^{W}), and X is N(R^{X}); or
V is N(R^{V}), W is C(R^{W}), and X is N; or
V is N(R^{V}), W is N, and X is C(R^{X}); or
V is N, W is N(R^{W}), and C is C(R^{X}); or
V is C(R^{V}), W is N(R^{W}), and X is N; or
V is C(R^{V}), W is N, and X is N(R^{X}); or
V is N, W is C(R^{W}), and X is C(R^{X}); or
V is C(R^{V}), W is C(R^{W}), and X is N; or
V is O, W is N, and X is N(R^{X}); or
V is C(R^{V}), W is N, and X is O; or
V is O, W is C(R^{W}), and X is N; or
V is N, W is C(R^{W}), and X is O; and
R^{V}, R^{W} and R^{X} are each independently selected from the group consisting of hydrogen and C₁-C₆alkyl; wherein C₁-C₆alkyl may optionally be substituted by one or more substituents each independently selected from the group consisting of deuterium, halogen, and hydroxyl;
   wherein * represents the point of attachment to the —NH— group in Formula I.

For example, in certain embodiments ring B is represented by:

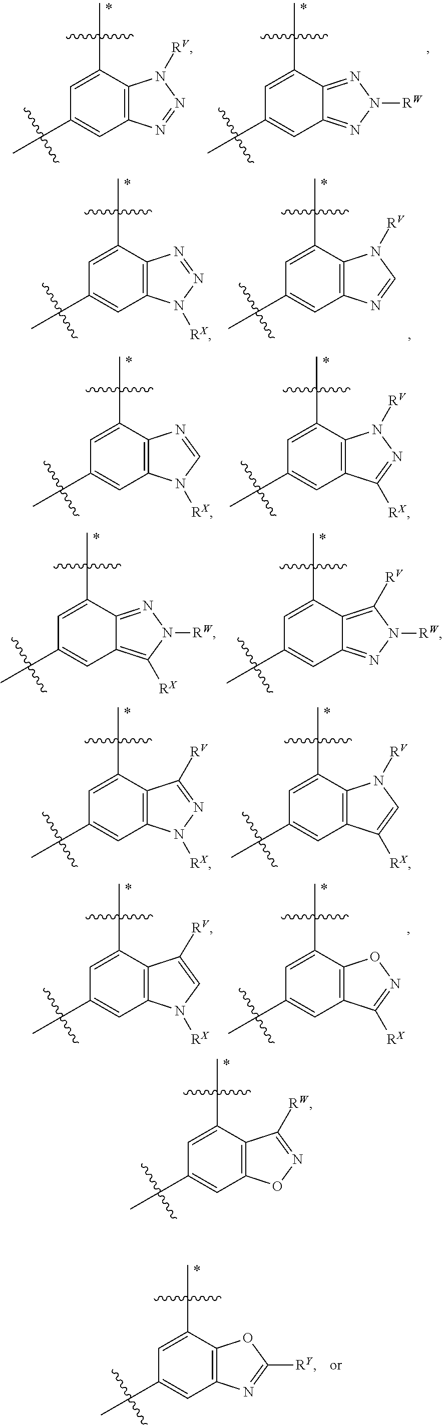

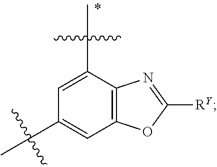

wherein R^{V}, R^{W} and R^{X} are each independently selected from the group consisting of hydrogen, —CH₃, and —CD₃.

In other embodiments, B is represented by:

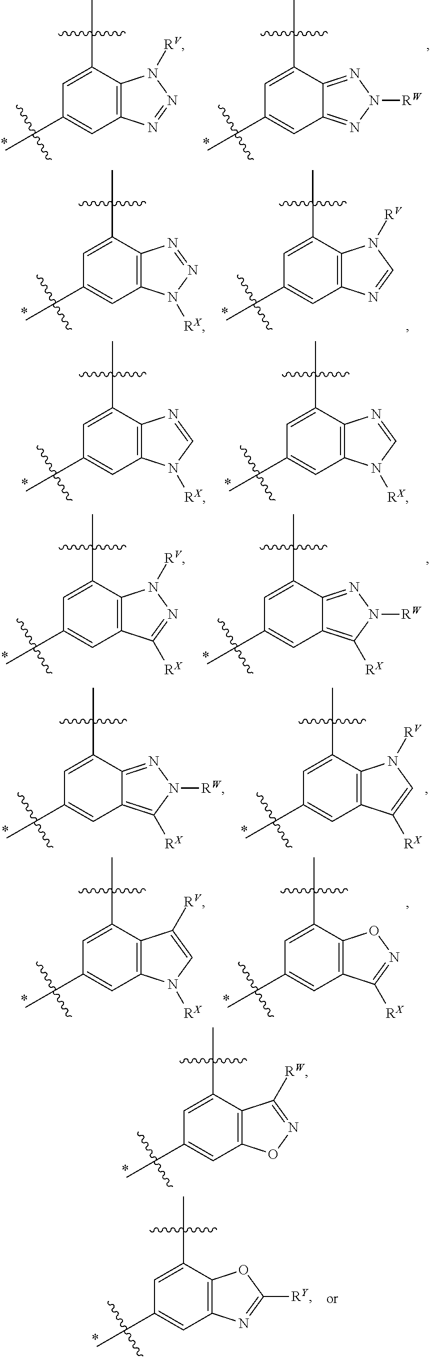

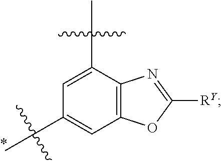

wherein R_{V}, R^{W} and R^{X} are each independently selected from the group consisting of hydrogen, —CH₃, and —CD₃.

In still further embodiments, ring B is represented by:

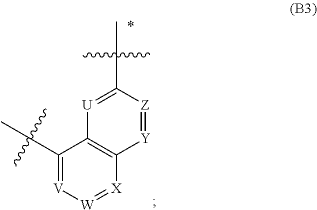

wherein:
U, V, W, X, Y, and Z are each independently selected from N and C(Re); wherein one or two of U, V, W, X, Y, and Z are N; and
R^{B} is independently selected for each occurrence from the group consisting of hydrogen, halogen, C₁-C₃alkyl, and C₁-C₃alkoxy;
   wherein * represents the point of attachment to the —NH— group in Formula 1.

For example, in certain embodiments ring B is selected from the group consisting of:

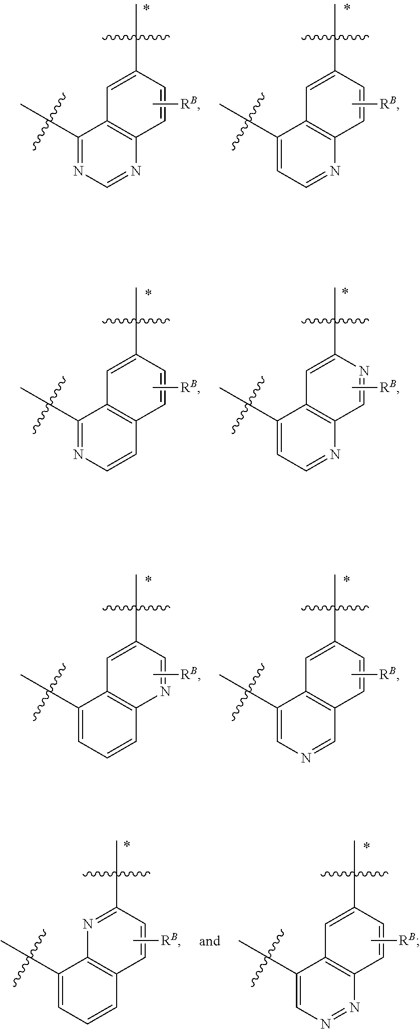

wherein R^{B} is selected from the group consisting of hydrogen, —CH₃, and —OCH₃.

In other embodiments, ring B is selected from the group consisting of:

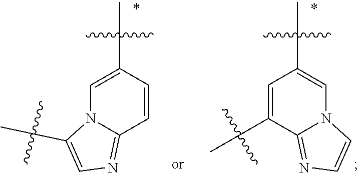 [SMILES: *c1ccc2ncc(*)n2c1 ; *c1cc(*)c2nccn2c1]

wherein * represents the point of attachment to the —NH— group in Formula I.

In some embodiments, a compound of Formula I may be represented by Formula II:

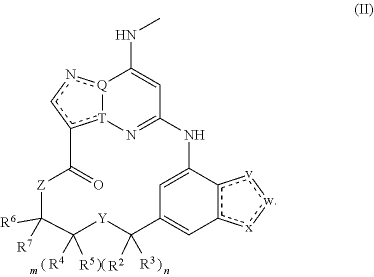

In some embodiments, a compound disclosed herein is represented by:

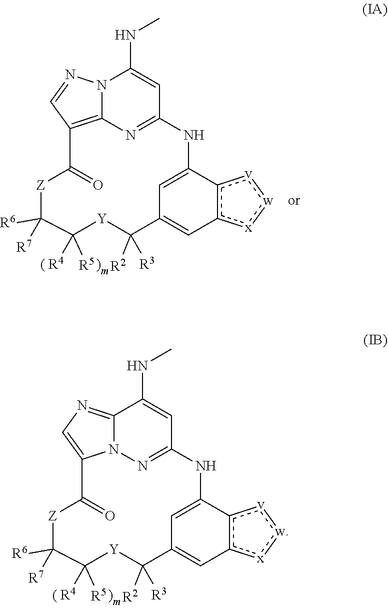

In other embodiments, a compound disclosed herein is represented by:

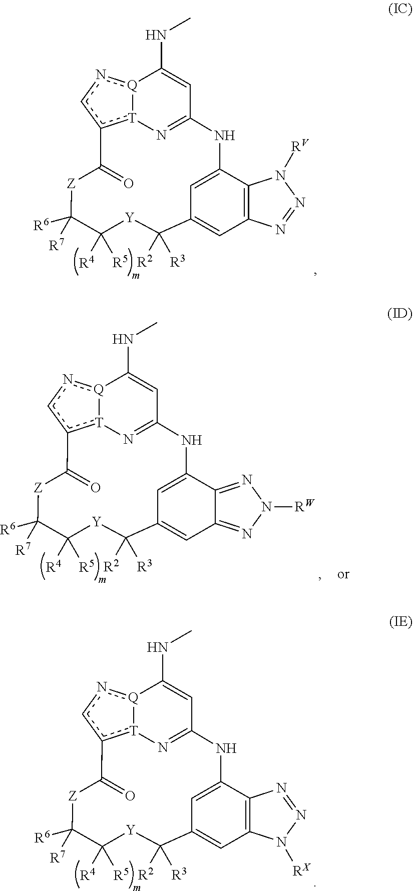

In still other embodiments, a compound disclosed herein is represented by:

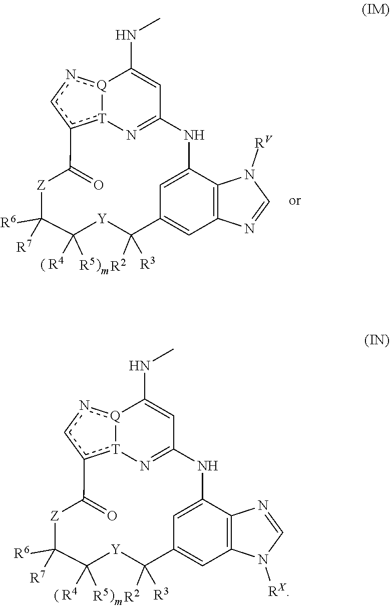

In further embodiments, a compound disclosed herein is represented by:

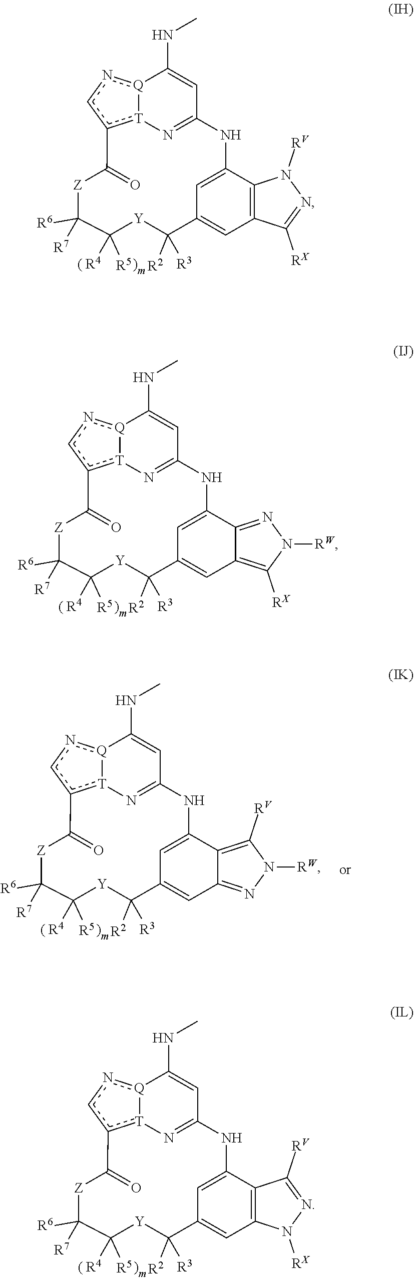

In some embodiments, a compound disclosed herein is represented by:

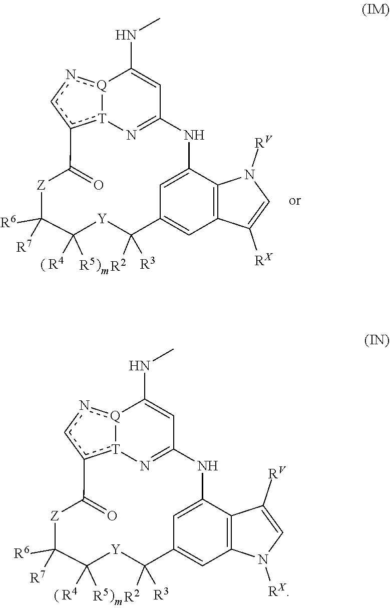

In some embodiments, a compound disclosed herein is represented by.:

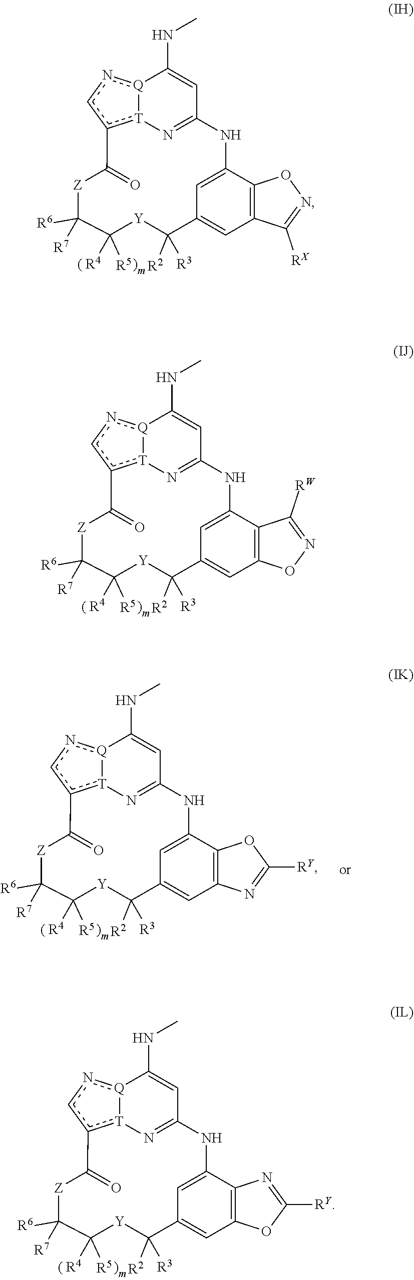

In some embodiments, R^{X}, R^{W} and R^{V} are each C₁-C₆alkyl optionally substituted by one or more deuteriums. For example, in certain embodiments R^{X}, R^{W} and R^{V} are each independently selected from —CH₃ and —CD₃.

In other embodiments, Y is selected from the group consisting of O, NH, NCH₃, S, S(O), and S(O)₂. For example, in certain embodiments Y is O.

In some embodiments, Z is selected from O, NH, and NCH₃. For example, in certain embodiments Z in NH.

In still other embodiments, R² and R³ are each independently selected from the group consisting of hydrogen, deuterium, —CH₃, and —CF₃; or R² and R³, together with the carbon to which they are attached, join to form cyclopropyl.

In some embodiments, m is 1. In other embodiments, m is 2. In still other embodiments, n is 0. In certain embodiments, n is 1.

In further embodiments, R⁴ and R⁵ are independently selected for each occurrence from the group consisting of hydrogen, deuterium, —CH₃, and —CF₃; or R4 and R⁵, together with the carbon to which they are attached, join to form cyclopropyl.

In other embodiments, R⁶ and R⁷ are each independently selected from the group consisting of hydrogen, deuterium, and C₁-C₆alkyl, wherein C₁-C₆alkyl may optionally be substituted by one or more halogen or hydroxyl groups. For example, in certain embodiments R⁶ and R⁷ are each independently selected from the group consisting of hydrogen, deuterium, —CH₃, —CF₃, —CH₂OH, —CH(CH₃)₂, and —C(CH₃)₂OH. In still other embodiments, R⁶ and R⁷, together with the carbon to which they are attached, join to form cyclopropyl.

Also disclosed herein is a compound represented by:

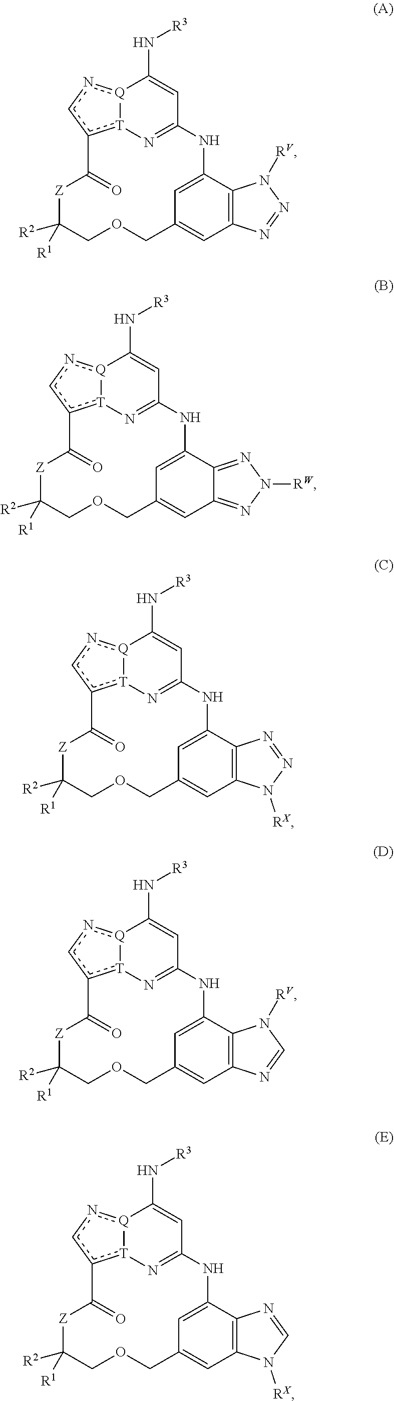

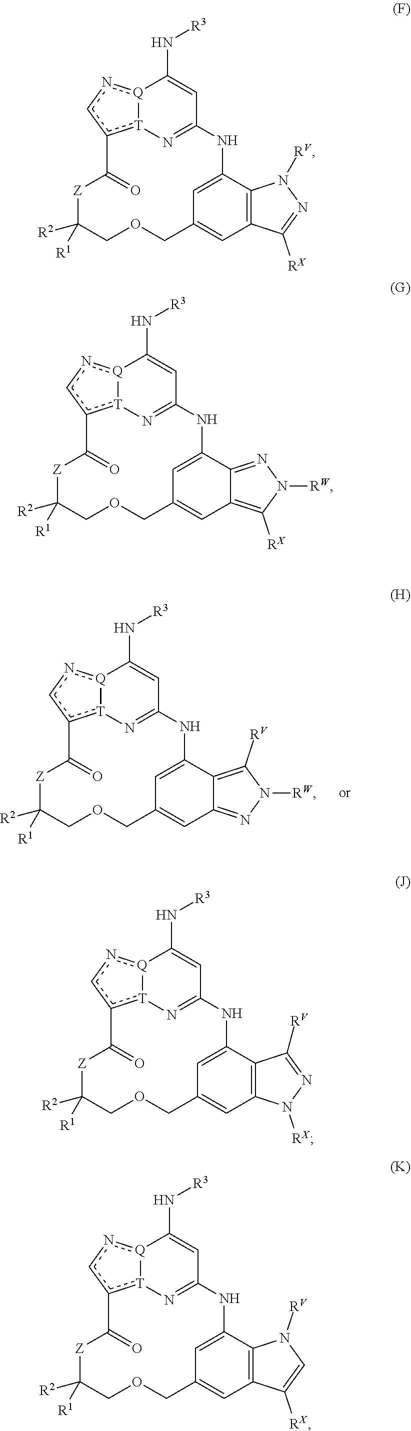

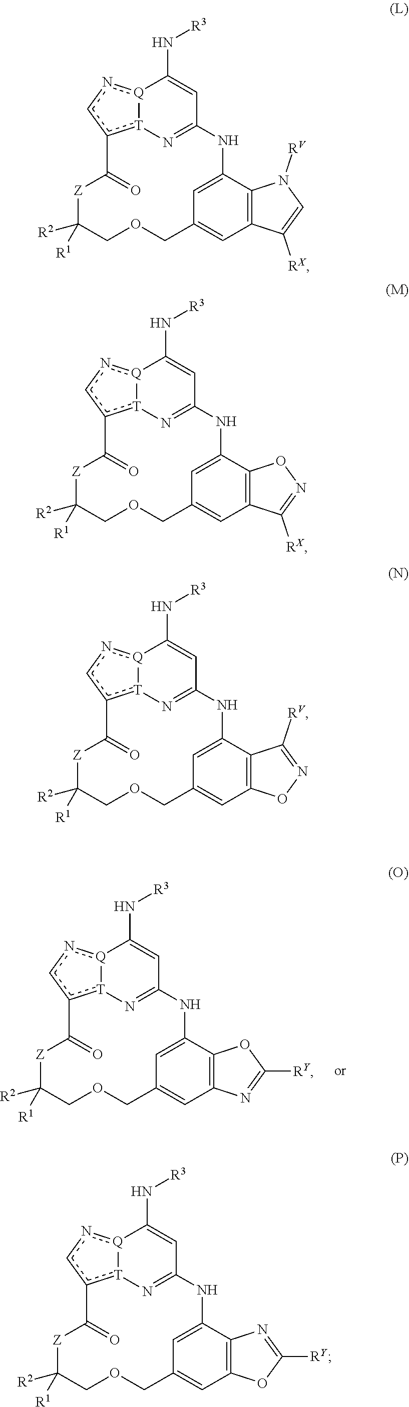

or a pharmaceutically acceptable salt and/or a stereoisomer thereof, wherein:
Q is N and T is C; or
Q is C and T is N;
   wherein Q and T are each members of a heteroaromatic ring system;
Z is NH or O;
R^{V}, R^{W} and R^{X} are each independently selected from the group consisting of hydrogen and C₁-C₄alkyl optionally substituted by one or more deuteriums;
R¹ and R² are independently selected from the group consisting of hydrogen, deuterium, and C₁-C₄alkyl, wherein C₁-C₄alkyl may be substituted on an available carbon by one or more halogen or hydroxyl; or
R¹ and R², together with the carbon to which they are attached, may be joined together to form cyclopropyl; and
R³ is hydrogen or C₁-C₄alkyl optionally substituted by one or more deuteriums.

In some embodiments, R^{X}, R^{W} and R^{V} are each selected from the group consisting of hydrogen, —CH₃ and —CD₃. In other embodiments, Z is NH.

In certain embodiments, R¹ and R² are each independently selected from the group consisting of hydrogen, deuterium, —CH₃, —CF₃, —CH₂OH, —CH(CH₃)₂, and —C(CH₃)₂OH. In other embodiments, R¹ and R², together with the carbon to which they are attached, join to form cyclopropyl.

In further embodiments, R³ is —CH₃. In some embodiments, Q is N and T is C. In other embodiments, Q is C and T is N.

Also disclosed herein is a compound selected from any of the compounds shown in Table 1, or a pharmaceutically acceptable salt and/or a stereoisomer thereof.

TABLE 1

| Compound | Structure |
|---|---|
| 1 | 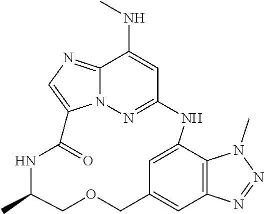 [SMILES: CNc1cc2nn3c(cnc13)C(=O)N[C@H](C)COCc1cc(c3c(c1)nnn3C)N2] |
| 2 | 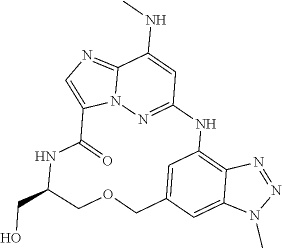 [SMILES: CNc1cc2nn3c(cnc13)C(=O)N[C@H](CO)COCc1cc(c3nnn(C)c3c1)N2] |
| 3 | 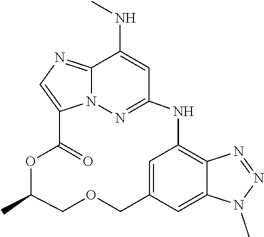 [SMILES: CNc1cc2nn3c(cnc13)C(=O)O[C@H](C)COCc1cc(c3nnn(C)c3c1)N2] |
| 4 | 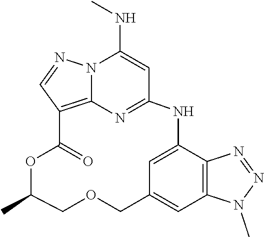 [SMILES: CNc1cc2nc3c(cnn13)C(=O)O[C@H](C)COCc1cc(c3nnn(C)c3c1)N2] |
| 5 | 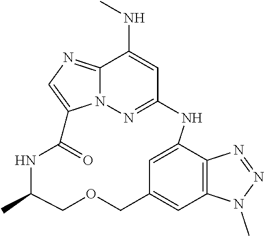 [SMILES: CNc1cc2nn3c(cnc13)C(=O)N[C@H](C)COCc1cc(c3nnn(C)c3c1)N2] |
| 6 | 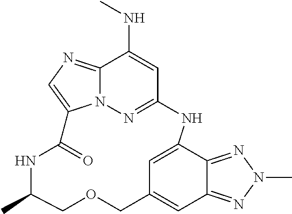 |
| 7 | 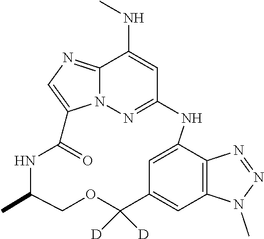 [SMILES: [2H]C1([2H])OC[C@@H](C)NC(=O)c2cnc3c(NC)cc(nn23)Nc2cc1cc1c2nnn1C] |
| 8 | 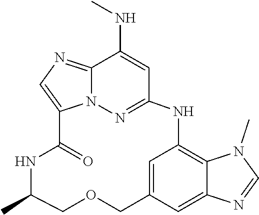 [SMILES: CNc1cc2nn3c(cnc13)C(=O)N[C@H](C)COCc1cc(c3c(c1)ncn3C)N2] |
| 9 | 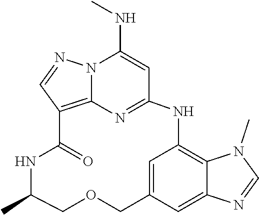 [SMILES: CNc1cc2nc3c(cnn13)C(=O)N[C@H](C)COCc1cc(c3c(c1)ncn3C)N2] |
| 10 | 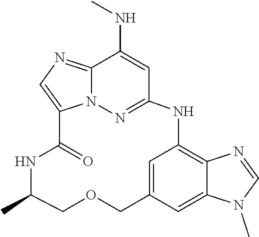 [SMILES: CNc1cc2nn3c(cnc13)C(=O)N[C@H](C)COCc1cc(c3ncn(C)c3c1)N2] |
| 11 | 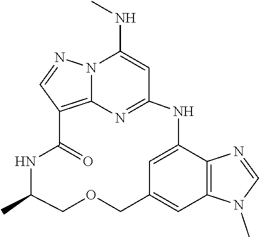 [SMILES: CNc1cc2nc3c(cnn13)C(=O)N[C@H](C)COCc1cc(c3ncn(C)c3c1)N2] |
| 12 | 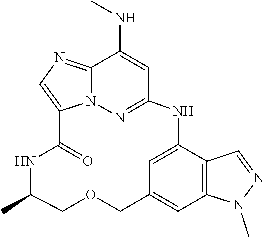 [SMILES: CNc1cc2nn3c(cnc13)C(=O)N[C@H](C)COCc1cc(c3cnn(C)c3c1)N2] |
| 13 | 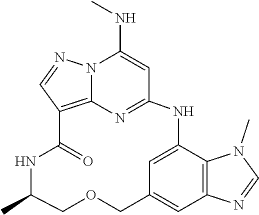 [SMILES: CNc1cc2nc3c(cnn13)C(=O)N[C@H](C)COCc1cc(c3c(c1)ncn3C)N2] |
| 14 | 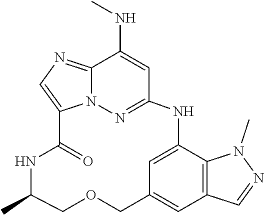 [SMILES: CNc1cc2nn3c(cnc13)C(=O)N[C@H](C)COCc1cc(c3c(cnn3C)c1)N2] |
| 15 | 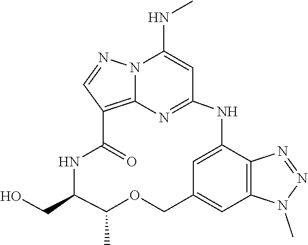 [SMILES: CNc1cc2nc3c(cnn13)C(=O)N[C@H](CO)[C@@H](C)OCc1cc(c3nnn(C)c3c1)N2] |
| 16 | 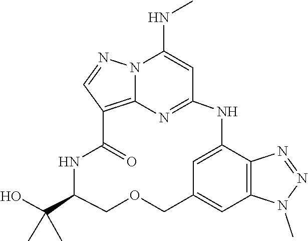 [SMILES: CNc1cc2nc3c(cnn13)C(=O)N[C@H](C(C)(C)O)COCc1cc(c3nnn(C)c3c1)N2] |
| 17 | 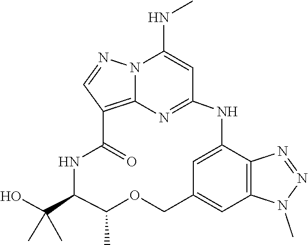 [SMILES: CNc1cc2nc3c(cnn13)C(=O)N[C@H](C(C)(C)O)[C@@H](C)OCc1cc(c3nnn(C)c3c1)N2] |
| 18 | 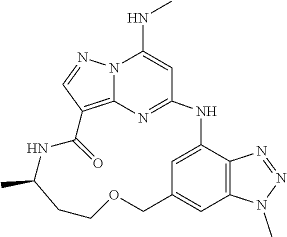 [SMILES: CNc1cc2nc3c(cnn13)C(=O)N[C@H](C)CCOCc1cc(c3nnn(C)c3c1)N2] |
| 19 | 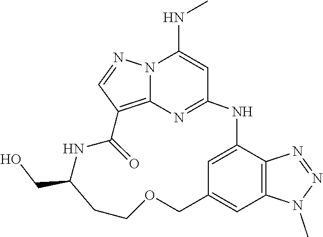 [SMILES: CNc1cc2nc3c(cnn13)C(=O)N[C@H](CO)CCOCc1cc(c3nnn(C)c3c1)N2] |
| 20 | 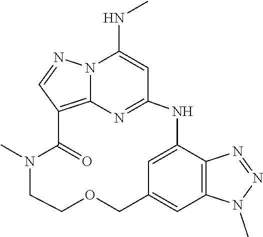 [SMILES: CNc1cc2nc3c(cnn13)C(=O)N(C)CCOCc1cc(c3nnn(C)c3c1)N2] |
| 21 | 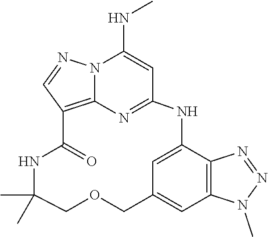 [SMILES: CNc1cc2nc3c(cnn13)C(=O)NC(C)(C)COCc1cc(c3nnn(C)c3c1)N2] |
| 22 | 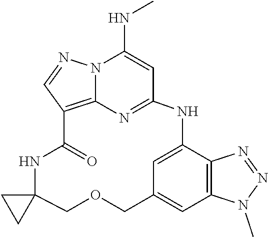 [SMILES: CNc1cc2nc3c(cnn13)C(=O)NC1(CC1)COCc1cc(c3nnn(C)c3c1)N2] |
| 23 | 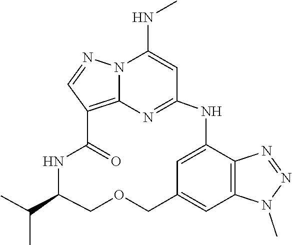 [SMILES: CNc1cc2nc3c(cnn13)C(=O)N[C@H](C(C)C)COCc1cc(c3nnn(C)c3c1)N2] |
| 24 | 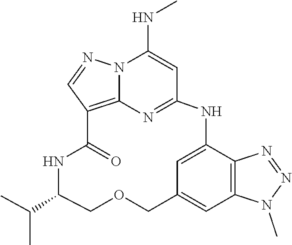 [SMILES: CNc1cc2nc3c(cnn13)C(=O)N[C@@H](C(C)C)COCc1cc(c3nnn(C)c3c1)N2] |
| 25 | 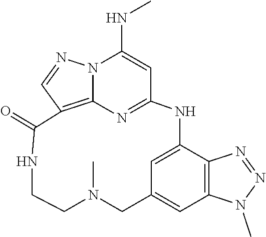 [SMILES: CNc1cc2nc3c(cnn13)C(=O)NCCN(C)Cc1cc(c3nnn(C)c3c1)N2] |
| 26 | 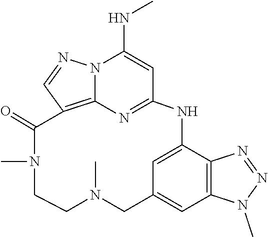 [SMILES: CNc1cc2nc3c(cnn13)C(=O)N(C)CCN(C)Cc1cc(c3nnn(C)c3c1)N2] |
| 27 | 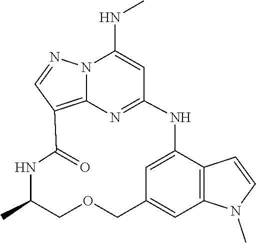 [SMILES: CNc1cc2nc3c(cnn13)C(=O)N[C@H](C)COCc1cc(c3ccn(C)c3c1)N2] |
| 28 | 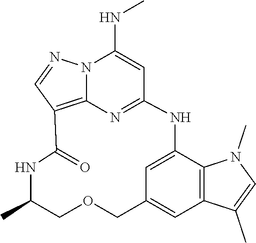 [SMILES: CNc1cc2nc3c(cnn13)C(=O)N[C@H](C)COCc1cc(c3c(c1)c(C)cn3C)N2] |
| 29 | 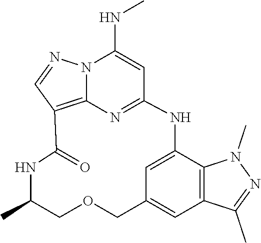 [SMILES: CNc1cc2nc3c(cnn13)C(=O)N[C@H](C)COCc1cc(c3c(c1)c(C)nn3C)N2] |
| 30 | 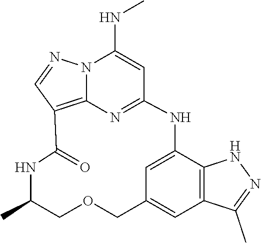 [SMILES: CNc1cc2nc3c(cnn13)C(=O)N[C@H](C)COCc1cc(c3[nH]nc(C)c3c1)N2] |
| 31 | 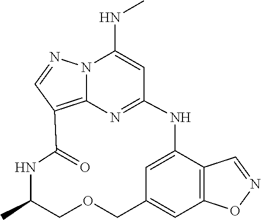 [SMILES: CNc1cc2nc3c(cnn13)C(=O)N[C@H](C)COCc1cc(c3cnoc3c1)N2] |
| 32 | 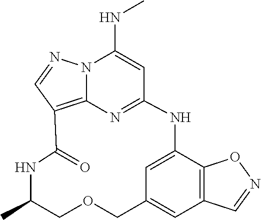 [SMILES: CNc1cc2nc3c(cnn13)C(=O)N[C@H](C)COCc1cc(c3oncc3c1)N2] |
| 33 | 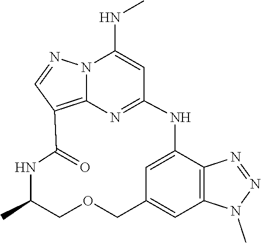 [SMILES: CNc1cc2nc3c(cnn13)C(=O)N[C@H](C)COCc1cc(c3nnn(C)c3c1)N2] |
| 34 | 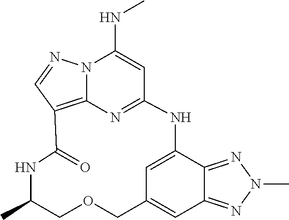 |
| 35 | 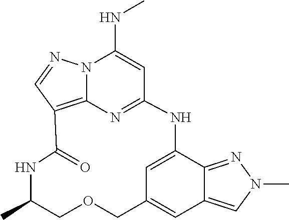 |
| 36 | 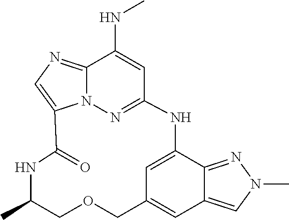 |
| 37 | 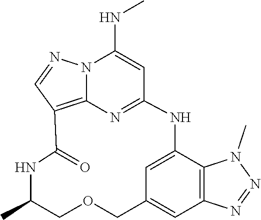 [SMILES: CNc1cc2nc3c(cnn13)C(=O)N[C@H](C)COCc1cc(c3c(c1)nnn3C)N2] |
| 38 | 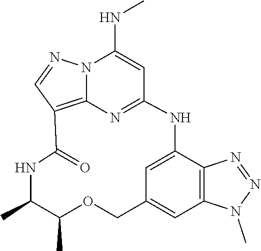 [SMILES: CNc1cc2nc3c(cnn13)C(=O)N[C@H](C)[C@H](C)OCc1cc(c3nnn(C)c3c1)N2] |
| 39 | 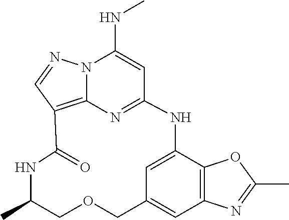 |
| 40 | 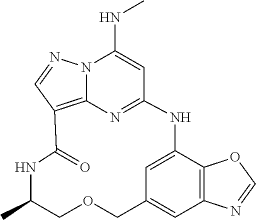 [SMILES: CNc1cc2nc3c(cnn13)C(=O)N[C@H](C)COCc1cc(c3ocnc3c1)N2] |
| 41 | 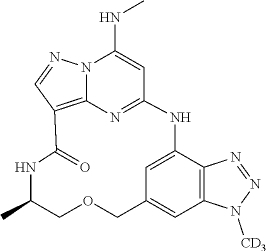 [SMILES: [2H]C([2H])([2H])n1nnc2c3cc(cc21)COC[C@@H](C)NC(=O)c1cnn2c(NC)cc(nc12)N3] |
| 42 | 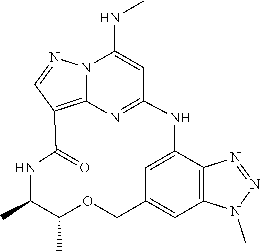 [SMILES: CNc1cc2nc3c(cnn13)C(=O)N[C@H](C)[C@@H](C)OCc1cc(c3nnn(C)c3c1)N2] |
| 43 | 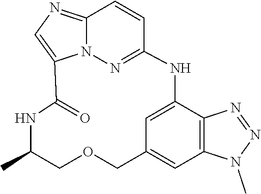 [SMILES: C[C@@H]1COCc2cc(c3nnn(C)c3c2)Nc2ccc3ncc(n3n2)C(=O)N1] |
| 44 | 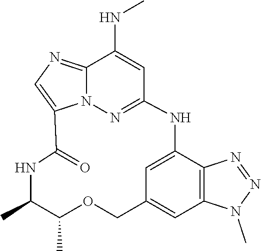 [SMILES: CNc1cc2nn3c(cnc13)C(=O)N[C@H](C)[C@@H](C)OCc1cc(c3nnn(C)c3c1)N2] |
| 45 | 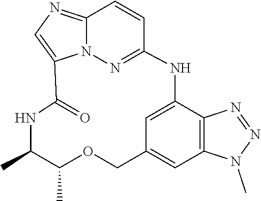 [SMILES: C[C@H]1NC(=O)c2cnc3ccc(nn23)Nc2cc(cc3c2nnn3C)CO[C@@H]1C] |
| 46 | 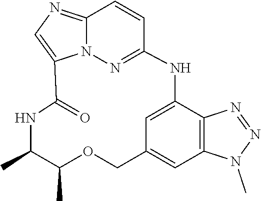 [SMILES: C[C@@H]1OCc2cc(c3nnn(C)c3c2)Nc2ccc3ncc(n3n2)C(=O)N[C@@H]1C] |
| 46 | 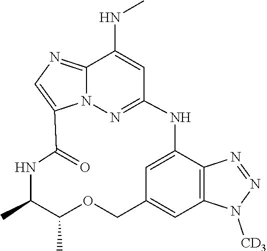 [SMILES: [2H]C([2H])([2H])n1nnc2c3cc(cc21)CO[C@H](C)[C@@H](C)NC(=O)c1cnc2c(NC)cc(nn12)N3] |
| 47 | 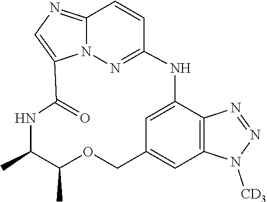 [SMILES: [2H]C([2H])([2H])n1nnc2c3cc(cc21)CO[C@@H](C)[C@@H](C)NC(=O)c1cnc2ccc(nn12)N3] |
| 48 | 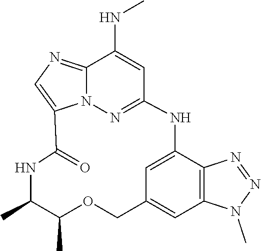 [SMILES: CNc1cc2nn3c(cnc13)C(=O)N[C@H](C)[C@H](C)OCc1cc(c3nnn(C)c3c1)N2] |
| 49 | 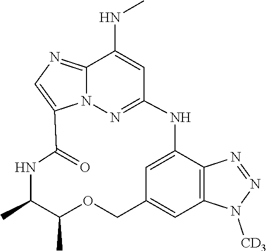 [SMILES: [2H]C([2H])([2H])n1nnc2c3cc(cc21)CO[C@@H](C)[C@@H](C)NC(=O)c1cnc2c(NC)cc(nn12)N3] |
| 50 | 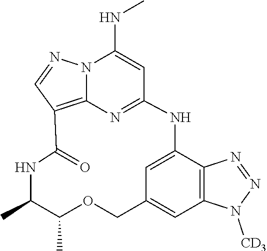 [SMILES: [2H]C([2H])([2H])n1nnc2c3cc(cc21)CO[C@H](C)[C@@H](C)NC(=O)c1cnn2c(NC)cc(nc12)N3] |
| 51 | 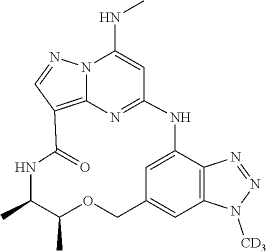 [SMILES: [2H]C([2H])([2H])n1nnc2c3cc(cc21)CO[C@@H](C)[C@@H](C)NC(=O)c1cnn2c(NC)cc(nc12)N3] |
| 52 | 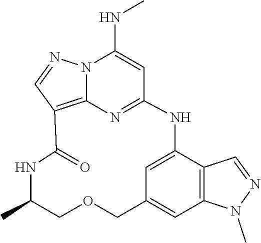 [SMILES: CNc1cc2nc3c(cnn13)C(=O)N[C@H](C)COCc1cc(c3cnn(C)c3c1)N2] |
| 53 | 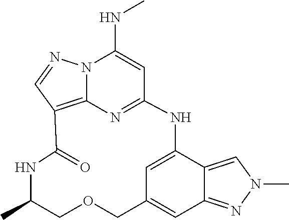 |
| 54 | 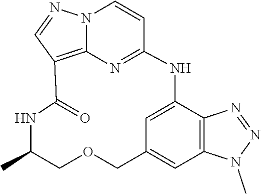 [SMILES: C[C@@H]1COCc2cc(c3nnn(C)c3c2)Nc2ccn3ncc(c3n2)C(=O)N1] |
| 55 | 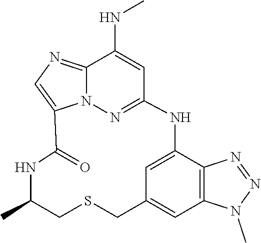 [SMILES: CNc1cc2nn3c(cnc13)C(=O)N[C@H](C)CSCc1cc(c3nnn(C)c3c1)N2] |
| 56 | 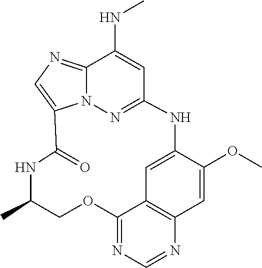 |
| 57 | 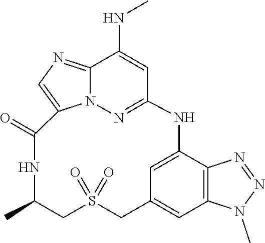 [SMILES: CNc1cc2nn3c(cnc13)C(=O)N[C@H](C)CS(=O)(=O)Cc1cc(c3nnn(C)c3c1)N2] |
| 58 | 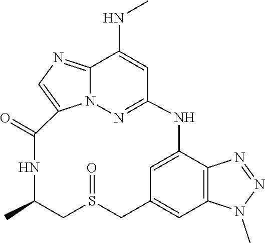 [SMILES: CNc1cc2nn3c(cnc13)C(=O)N[C@H](C)CS(=O)Cc1cc(c3nnn(C)c3c1)N2] |
| 59 | 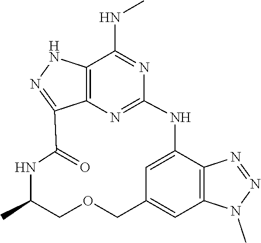 [SMILES: CNc1nc2nc3c(n[nH]c13)C(=O)N[C@H](C)COCc1cc(c3nnn(C)c3c1)N2] |
| 60 | 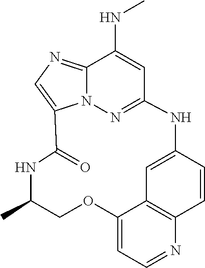 [SMILES: CNc1cc2nn3c(cnc13)C(=O)N[C@H](C)COc1ccnc3ccc(cc13)N2] |
| 61 | 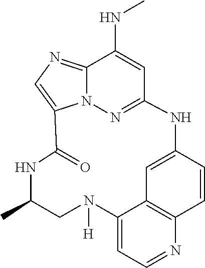 [SMILES: CNc1cc2nn3c(cnc13)C(=O)N[C@H](C)CNc1ccnc3ccc(cc13)N2] |
| 62 | 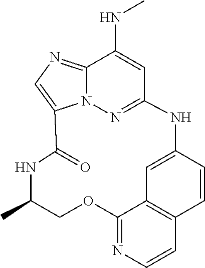 [SMILES: CNc1cc2nn3c(cnc13)C(=O)N[C@H](C)COc1nccc3ccc(cc13)N2] |
| 63 | 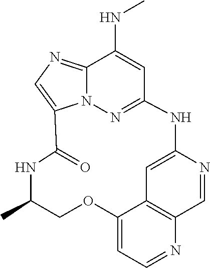 [SMILES: CNc1cc2nn3c(cnc13)C(=O)N[C@H](C)COc1ccnc3cnc(cc13)N2] |
| 64 | 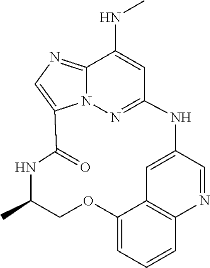 [SMILES: CNc1cc2nn3c(cnc13)C(=O)N[C@H](C)COc1cccc3ncc(cc13)N2] |
| 65 | 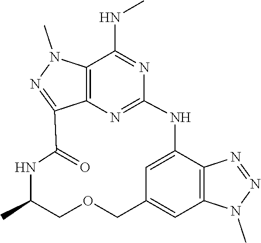 [SMILES: CNc1nc2nc3c(nn(C)c13)C(=O)N[C@H](C)COCc1cc(c3nnn(C)c3c1)N2] |
| 66 | 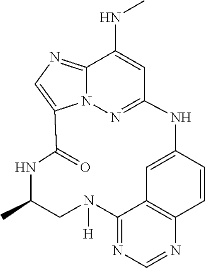 [SMILES: CNc1cc2nn3c(cnc13)C(=O)N[C@H](C)CNc1ncnc3ccc(cc13)N2] |
| 67 | 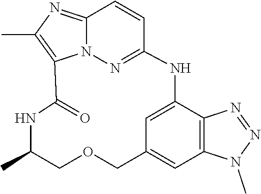 [SMILES: Cc1nc2ccc3nn2c1C(=O)N[C@H](C)COCc1cc(c2nnn(C)c2c1)N3] |
| 68 | 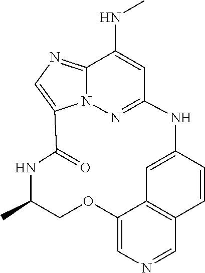 [SMILES: CNc1cc2nn3c(cnc13)C(=O)N[C@H](C)COc1cncc3ccc(cc13)N2] |
| 69 | 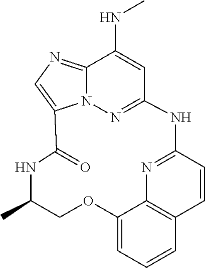 [SMILES: CNc1cc2nn3c(cnc13)C(=O)N[C@H](C)COc1cccc3ccc(nc13)N2] |
| 70 | 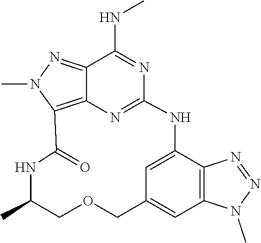 [SMILES: CNc1nc2nc3c(n(C)nc13)C(=O)N[C@H](C)COCc1cc(c3nnn(C)c3c1)N2] |
| 71 | 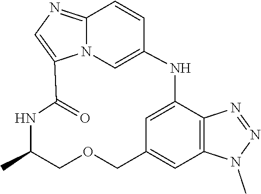 [SMILES: C[C@@H]1COCc2cc(c3nnn(C)c3c2)Nc2ccc3ncc(n3c2)C(=O)N1] |
| 72 | 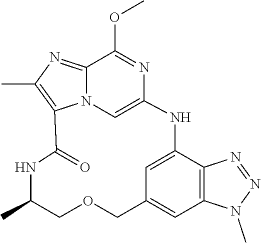 [SMILES: COc1nc2cn3c(c(C)nc13)C(=O)N[C@H](C)COCc1cc(c3nnn(C)c3c1)N2] |
| 73 | 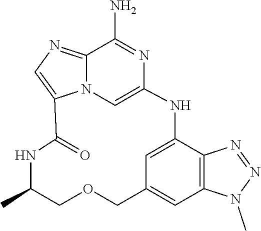 [SMILES: C[C@@H]1COCc2cc(c3nnn(C)c3c2)Nc2cn3c(cnc3c(N)n2)C(=O)N1] |
| 74 | 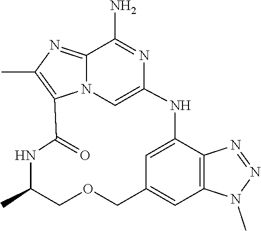 [SMILES: Cc1nc2c(N)nc3cn2c1C(=O)N[C@H](C)COCc1cc(c2nnn(C)c2c1)N3] |
| 75 | 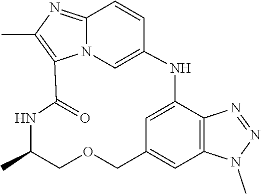 [SMILES: Cc1nc2ccc3cn2c1C(=O)N[C@H](C)COCc1cc(c2nnn(C)c2c1)N3] |
| 76 | 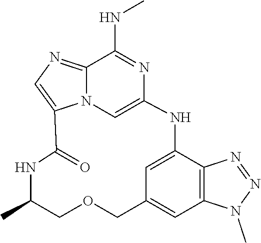 [SMILES: CNc1nc2cn3c(cnc13)C(=O)N[C@H](C)COCc1cc(c3nnn(C)c3c1)N2] |
| 77 | 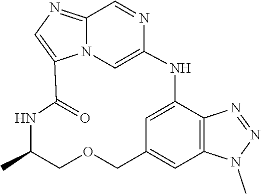 [SMILES: C[C@@H]1COCc2cc(c3nnn(C)c3c2)Nc2cn3c(cnc3cn2)C(=O)N1] |
| 78 | 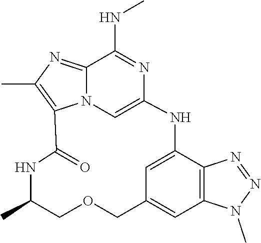 [SMILES: CNc1nc2cn3c(c(C)nc13)C(=O)N[C@H](C)COCc1cc(c3nnn(C)c3c1)N2] |
| 79 | 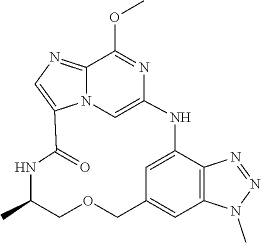 [SMILES: COc1nc2cn3c(cnc13)C(=O)N[C@H](C)COCc1cc(c3nnn(C)c3c1)N2] |
| 80 | 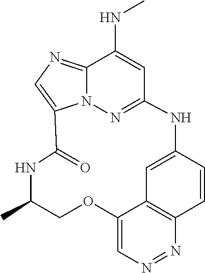 [SMILES: CNc1cc2nn3c(cnc13)C(=O)N[C@H](C)COc1cnnc3ccc(cc13)N2] |
| 81 | 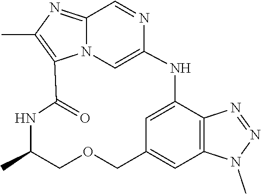 [SMILES: Cc1nc2cnc3cn2c1C(=O)N[C@H](C)COCc1cc(c2nnn(C)c2c1)N3] |
| 82 | 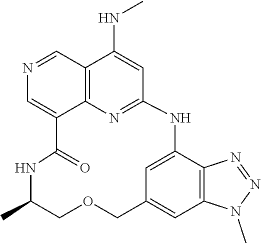 [SMILES: CNc1cc2nc3c(cncc13)C(=O)N[C@H](C)COCc1cc(c3nnn(C)c3c1)N2] |
| 83 | 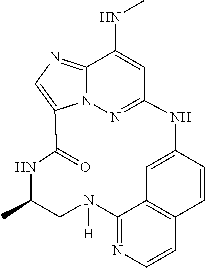 [SMILES: CNc1cc2nn3c(cnc13)C(=O)N[C@H](C)CNc1nccc3ccc(cc13)N2] |
| 84 | 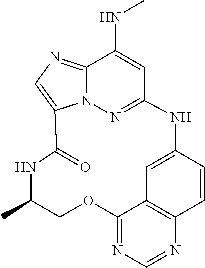 [SMILES: CNc1cc2nn3c(cnc13)C(=O)N[C@H](C)COc1ncnc3ccc(cc13)N2] |
| 85 | 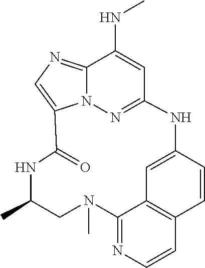 [SMILES: CNc1cc2nn3c(cnc13)C(=O)N[C@H](C)CN(C)c1nccc3ccc(cc13)N2] |
| 86 | 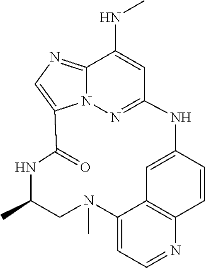 [SMILES: CNc1cc2nn3c(cnc13)C(=O)N[C@H](C)CN(C)c1ccnc3ccc(cc13)N2] |
| 87 | 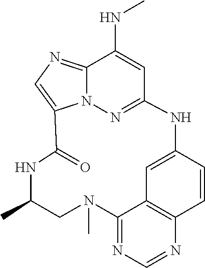 [SMILES: CNc1cc2nn3c(cnc13)C(=O)N[C@H](C)CN(C)c1ncnc3ccc(cc13)N2] |
| 88 | 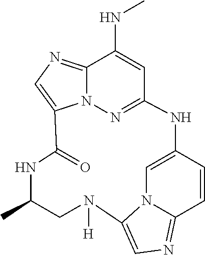 [SMILES: CNc1cc2nn3c(cnc13)C(=O)N[C@H](C)CNc1cnc3ccc(cn13)N2] |
| 89 | 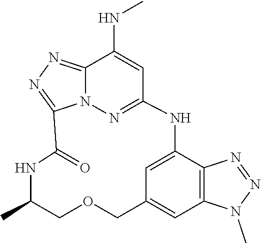 [SMILES: CNc1cc2nn3c(nnc13)C(=O)N[C@H](C)COCc1cc(c3nnn(C)c3c1)N2] |
| 90 | 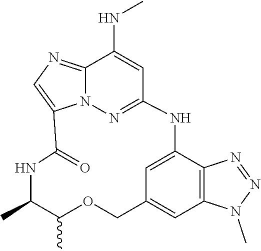 [SMILES: CNc1cc2nn3c(cnc13)C(=O)N[C@H](C)C(C)OCc1cc(c3nnn(C)c3c1)N2] |
| 91 | 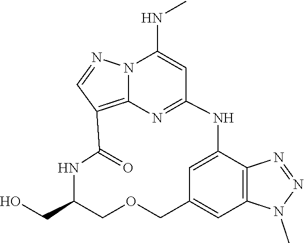 [SMILES: CNc1cc2nc3c(cnn13)C(=O)N[C@H](CO)COCc1cc(c3nnn(C)c3c1)N2] |
| 92 | 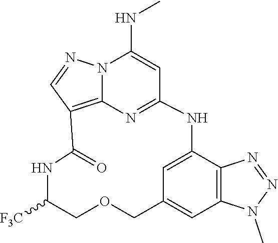 [SMILES: CNc1cc2nc3c(cnn13)C(=O)NC(C(F)(F)F)COCc1cc(c3nnn(C)c3c1)N2] |
| 93 | 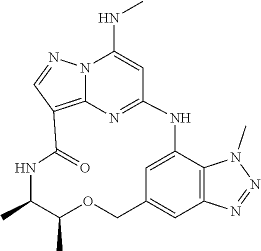 [SMILES: CNc1cc2nc3c(cnn13)C(=O)N[C@H](C)[C@H](C)OCc1cc(c3c(c1)nnn3C)N2] |
| 94 | 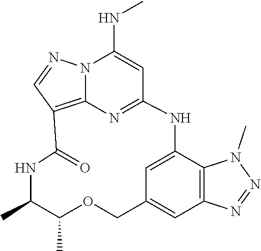 [SMILES: CNc1cc2nc3c(cnn13)C(=O)N[C@H](C)[C@@H](C)OCc1cc(c3c(c1)nnn3C)N2] |
| 95 | 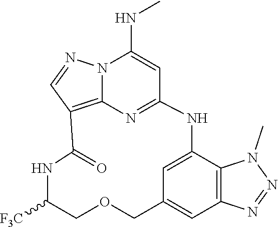 [SMILES: CNc1cc2nc3c(cnn13)C(=O)NC(C(F)(F)F)COCc1cc(c3c(c1)nnn3C)N2] |
| 96 | 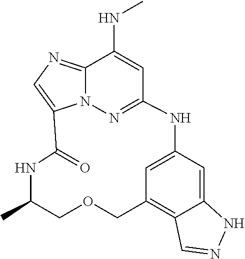 [SMILES: CNc1cc2nn3c(cnc13)C(=O)N[C@H](C)COCc1cc(cc3[nH]ncc13)N2] |
| 97 | 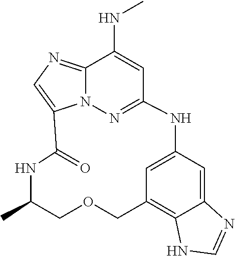 [SMILES: CNc1cc2nn3c(cnc13)C(=O)N[C@H](C)COCc1cc(cc3nc[nH]c13)N2] |
| 98 | 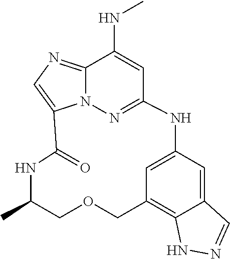 [SMILES: CNc1cc2nn3c(cnc13)C(=O)N[C@H](C)COCc1cc(cc3cn[nH]c13)N2] |
| 99 | 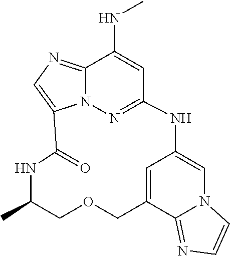 [SMILES: CNc1cc2nn3c(cnc13)C(=O)N[C@H](C)COCc1cc(cn3ccnc13)N2] |
| 100 | 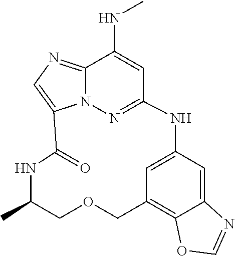 [SMILES: CNc1cc2nn3c(cnc13)C(=O)N[C@H](C)COCc1cc(cc3ncoc13)N2] |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Labeled Compounds

In some embodiments, the compounds described herein exist in their isotopically labeled forms. In some embodiments, the methods disclosed herein include methods of treating diseases by administering such isotopically labeled compounds. In some embodiments, the methods disclosed herein include methods of treating diseases by administering such isotopically labeled compounds as pharmaceutical compositions. Thus, in some embodiments, the compounds disclosed herein include isotopically labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid. 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal, and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In some embodiments, the present method involves the administration of about 0.1 μg to about 50 mg of at least one compound described herein per kg body weight of the subject. For a 70 kg patient, dosages of from about 10 μg to about 200 mg of the compound disclosed herein would be more commonly used, depending on a subject's physiological response.

By way of example only, the dose of the compound described herein for methods of treating a disease as described herein is about 0.001 to about 1 mg/kg body weight of the subject per day, for example, about 0.001 mg, about 0.002 mg, about 0.005 mg, about 0.010 mg, 0.015 mg, about 0.020 mg, about 0.025 mg, about 0.050 mg, about 0.075 mg, about 0.1 mg, about 0.15 mg, about 0.2 mg, about 0.25 mg, about 0.5 mg, about 0.75 mg, or about 1 mg/kg body weight per day. In some embodiments, the dose of compound described herein for the described methods is about 1 to about 1000 mg/kg body weight of the subject being treated per day, for example, about 1 mg, about 2 mg, about 5 mg, about 10 mg, about 15 mg, about 20 mg, about 25 mg, about 50 mg, about 75 mg, about 100 mg, about 150 mg, about 200 mg, about 250 mg, about 500 mg, about 750 mg, or about 1000 mg per day.

### Methods of Treatment

The compounds disclosed herein, or pharmaceutically acceptable salts, solvates, or stereoisomers thereof, are useful for the inhibition of kinase activity of one or more enzymes. In some embodiments the kinase inhibited by the compounds and methods is TYK2.

Provided herein are compounds that are inhibitors of TYK2 and are therefore useful for treating one or more disorders associated with activity of TYK2 or mutants thereof.

Provided herein are methods for treating a disease or disorder, wherein the disease or disorder is an autoimmune disorders, inflammatory disorders, proliferative disorders, endocrine disorders, neurological disorders, or disorders associated with transplantation, said method comprising administering to a patient in need thereof. a pharmaceutical composition comprising an effective amount of a compound described herein, or a pharmaceutically acceptable salt, solvate, or stereoisomer thereof.

In some embodiments, the disease or disorder is an autoimmune disorder. In some embodiments the disease or disorder is selected from type 1 diabetes, systemic lupus erythematosus, multiple sclerosis, psoriasis, Behget's disease, POEMS syndrome, Crohn's disease, ulcerative colitis, and inflammatory bowel disease.

In some embodiments, the disease or disorder is an inflammatory disorder. In some embodiments, the inflammatory disorder is rheumatoid arthritis, asthma, chronic obstructive pulmonary disease, psoriasis, hepatomegaly, Crohn's disease, ulcerative colitis, inflammatory bowel disease.

In some embodiments, the disease or disorder is a proliferative disorder. In some embodiments, the proliferative disorder is cancer. In some embodiments, the disease or disorder is a proliferative disorder. In some embodiments, the proliferative disorder is a hematological cancer. In some embodiments the proliferative disorder is a leukemia. In some embodiments, the leukemia is a T-cell leukemia. In some embodiments the T-cell leukemia is T-cell acute lymphoblastic leukemia (T-ALL). In some embodiments the proliferative disorder is polycythemia vera, myelofibrosis, essential or thrombocytosis.

In some embodiments, the disease or disorder is an endocrine disorder. In some embodiments, the endocrine disorder is polycystic ovary syndrome, Crouzon's syndrome, or type 1 diabetes.

In some embodiments, the disease or disorder is a neurological disorder. In some embodiments, the neurological disorder is Alzheimer's disease.

In some embodiments the proliferative disorder is associated with one or more activating mutations in TYK2. In some embodiments, the activating mutation in TYK2 is a mutation to the FERM domain, the JH2 domain, or the kinase domain. In some embodiments the activating mutation in TYK2 is selected from G36D, S47N, R425H, V731I, E957D, and R1027H.

In some embodiments, the disease or disorder is associated with transplantation. In some embodiments the disease or disorder associated with transplantation is transplant rejection, or graft versus host disease.

In some embodiments the disease or disorder is associated with type I interferon, IL-10, IL-12, or IL-23 signaling. In some embodiments the disease or disorder is associated with type I interferon signaling. In some embodiments the disease or disorder is associated with IL-10 signaling. In some embodiments the disorder is associated with IL-12 signaling. In some embodiments the disease or disorder is associated with IL-23 signaling.

Provided herein are methods for treating an inflammatory or allergic condition of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Provided herein are methods for treating other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, muscle wasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis. nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease is acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), or osteoarthritis.

In some embodiments the inflammatory disease is a Th1 or Th17 mediated disease. In some embodiments the Th17 mediated disease is selected from Systemic lupus erythematosus, Multiple sclerosis, and inflammatory bowel disease (including Crohn's disease or ulcerative colitis).

In some embodiments the inflammatory disease is Sjogren's syndrome, allergic disorders, osteoarthritis, conditions of the eye such as ocular allergy, conjunctivitis, keratoconjunctivitis sicca, vernal conjunctivitis, or diseases affecting the nose such as allergic rhinitis.

For example, disclosed herein is a method of inhibiting a TYK2 enzyme in a patient or biological sample, comprising contacting said patient or biological sample with a therapeutically effective amount of a compound disclosed herein, or the pharmaceutical composition disclosed herein.

Also disclosed herein is method of inhibiting TYK2 activity in a patient in need thereof, comprising administering to the patient a therapeutically effective amount of a compound disclosed, or the pharmaceutical composition disclosed herein. In some embodiments, inhibiting TYK2 activity is associated with treating a disease or disorder selected from the group consisting of, e.g., Crohn's disease, rheumatoid arthritis, psoriasis, systemic lupus erythematosus, ulcerative colitis, psoriatic arthritis, and systemic sclerosis.

Further disclosed herein is a TYK2-mediated disorder in a patient in need thereof, comprising administering to the patient a therapeutically effective amount of a disclosed compound or pharmaceutical composition. In some embodiments, a contemplated TYK2-mediated disorder may be, for example, an autoimmune disorder, an inflammatory disorder, a proliferative disorder, an endocrine disorder, a neurological disorder, or a disorder associated with transplantation. In other embodiments, a contemplated disorder is associated with type I interferon, IL-10, IL-12, or IL-23 signalling.

For example, provided herein is a method of treating one or more of. Crohn's disease, rheumatoid arthritis, psoriasis, systemic lupus erythematosus, ulcerative colitis, psoriatic arthritis, and systemic sclerosis in a patient in need thereof, comprising administering to the patient an effective amount of any one of the compounds disclosed herein, or a disclosed pharmaceutical composition.

### Combination Therapy

In certain instances, the compound described herein, or a pharmaceutically acceptable salt, solvate, or stereoisomer thereof, is administered in combination with a second therapeutic agent.

In some embodiments, the benefit experienced by a patient is increased by administering one of the compounds described herein with a second therapeutic agent (which also includes a therapeutic regimen) that also has therapeutic benefit.

In one specific embodiment, a compound described herein, or a pharmaceutically acceptable salt, solvate, or stereoisomer thereof, is co-administered with a second therapeutic agent, wherein the compound described herein, or a pharmaceutically acceptable salt, solvate, or stereoisomer thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply additive of the two therapeutic agents or the patient experiences a synergistic benefit.

In certain embodiments, different therapeutically effective dosages of the compounds disclosed herein will be utilized in formulating a pharmaceutical composition and/or in treatment regimens when the compounds disclosed herein are administered in combination with a second therapeutic agent. Therapeutically effective dosages of drugs and other agents for use in combination treatment regimens are optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt, solvate, or stereoisomer thereof, is initiated prior to, during, or after treatment with a second agent described herein, and continues until any time during treatment with the second agent or after termination of treatment with the second agent. It also includes treatments in which a compound described herein, or a pharmaceutically acceptable salt, solvate, or stereoisomer thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g., the disease, disorder, or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated, and so forth. In additional embodiments, when co-administered with a second therapeutic agent, the compound provided herein is administered either simultaneously with the second therapeutic agent, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt, solvate, or stereoisomer thereof, as well as combination therapies, are administered before, during, or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, the compound of described herein, or a pharmaceutically acceptable salt, solvate, or stereoisomer thereof, is administered in combination with an adjuvant. In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced).

### EXAMPLES

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. At least some of the compounds identified as “Intermediates” herein are contemplated as compounds of the disclosure.

### Example 1: Synthesis of (7R,E)-3¹,7-Dimethyl-1⁸-(methylamino)-3¹H-5-oxa-2,8-diaza-1(6,3),3(4,6)-diimidazo[1,2-b]pyridazinacyclononaphan-9-one (Compound 1)

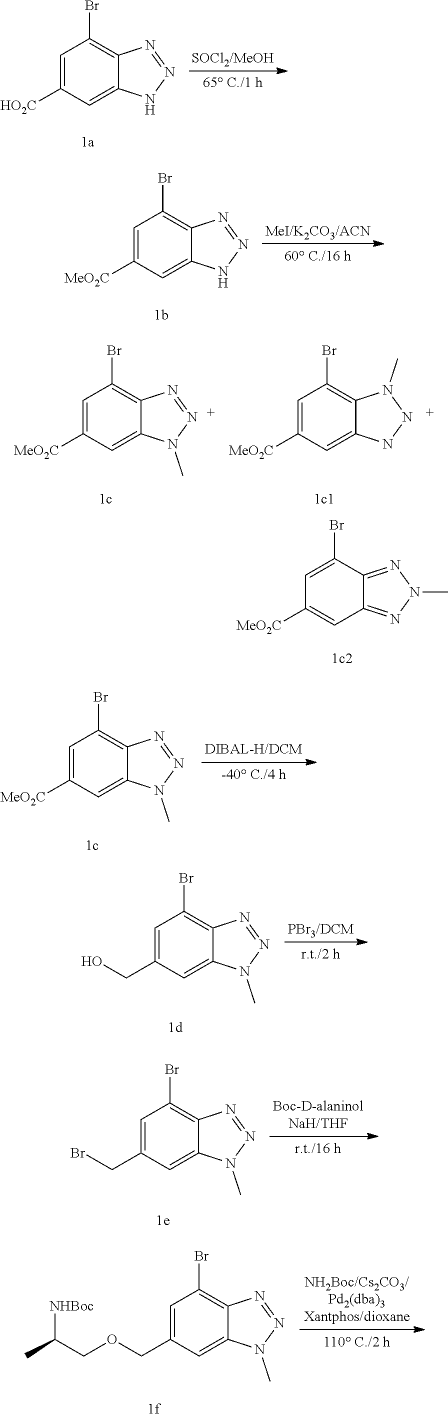

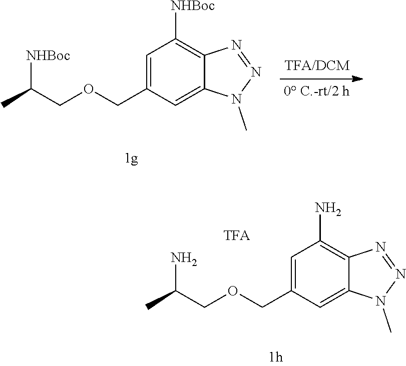 [SMILES: C[C@H](COCc1cc(NC(=O)OC(C)(C)C)c2nnn(C)c2c1)NC(=O)OC(C)(C)C ; C[C@@H](N)COCc1cc(N)c2nnn(C)c2c1]

### Methyl 4-bromo-1H-benzo[d][1,2,3]triazole-6-carboxylate (1b)

To a stirred solution of 1a (500 mg, 2.06 mmol, 1.00 eq) in dry methanol (10 mL, 4A MS) at 0° C., thionyl chloride (0.45 mL, 6.2 mmol, 3.0 eq) was added dropwise over 1 min. The reaction mixture was heated at reflux for 1 hour. The mixture was slowly poured into a saturated aqueous NaHCO₃ solution (30 mL) and extracted with DCM (3×30 mL). The combined organics were washed with brine, dried over MgSO₄, filtered, and concentrated under reduced pressure to provide the product 1b (350 mg, 1.37 mmol, 66%) as a white solid. R_{f}=0.25, Hexanes/EtOAc, 70:30. ¹H NMR (300 MHz, Acetone-d₆) δ 8.59 (s, 1H), 8.22 (s, 1H), 3.97 (s, 3H).

### Methyl 4-bromo-1-methyl-1H-1,2,3-benzotriazole-6-carboxylate (1c)

To a stirred solution of 1b (100 mg, 0.39 mmol, 1.00 eq) in ACN (2.0 mL) was added K₂CO₃ (108 mg, 0.781 mmol, 2.00 eq) and Mel (73 μL, 1.2 mmol, 3.0 eq) at room temperature. The resulting mixture was stirred at 60° C. for 16 hours. After cooled to room temperature, the solid was filtered out and filtrate was concentrated. Purification by silica gel flash column chromatography provided the product 1c (30 mg, 28%) as a white solid and a mixture of 1c1/1c2 (60 mg, 57%, 1:1) as a white solid. 1c: (R_{f}=0.30, Hexanes/EtOAc, 70:30). ¹H NMR (300 MHz, Acetone-d₆) d 8.48 (d, J=1.2 Hz, 1H), 8.15 (d, J=1.2 Hz, 1H), 4.49 (s, 3H), 3.97 (s, 3H). 1c1/1c2: (R_{f}=0.60, Hexanes/EtOAc, 70:30). ¹H NMR (300 MHz, CDCl₃) δ 8.71 (s, 1H), 8.58 (s, 1H), 8.33 (s, 1H), 8.24 (s, 1H), 4.60 (d, J=1.1 Hz, 6H), 3.98 (s, 6H).

### 4-Bromo-1-methyl-1H-1,2,3-benzotriazol-6-yl)methanol (1d)

To a stirred solution of 1c (400 mg, 1.48 mmol, 1.00 eq) in DCM (15 mL) was added DIBAL-H (4.4 mL, 4.4 mmol, 3.0 eq, 1M in hexanes) dropwise at −40° C. The resulting mixture was stirred at −40° C. for 4 hours. Methanol (0.2 mL) was added at −40° C. and the reaction was stirred for 10 minutes followed by addition of DCM (10 mL) and a solution of 1N HCl (5 mL). The aqueous phase was extracted with DCM (3×10 mL). The combined organics were washed with water, dried over MgSO₄, filtered, and concentrated under reduced pressure. Purification by silica gel flash column chromatography (R_{f}=0.25, Hexanes/EtOAc, 30:70) provided the product 1d (330 mg, 92%) as a white solid. ¹H NMR (300 MHz, Acetone-d₆) δ 7.74 (s, 1H), 7.60 (s, 1H), 4.83 (d, J=5.6 Hz, 2H), 4.61 (t, J=5.7 Hz, 1H), 4.35 (s, 3H).

### 4-Bromo-6-(bromomethyl)-1-methyl-1H-1,2,3-benzotriazole (1e)

To a stirred solution of 1d (370 mg, 1.53 mmol, 1.00 eq) in DCM (7.6 mL) at 0° C., PBr₃ (2.3 mL, 2.3 mmol, 1.5 eq) was added dropwise. The resulting mixture was stirred at 0° C. for 30 minutes and then room temperature for 2 hours. Water (0.5 mL) was added slowly to quench the reaction. Then the mixture was diluted with DCM (20 mL) and water (20 mL). The aqueous phase was extracted with DCM (3×20 mL). The combined organics were washed with brine, dried over MgSO₄, filtered, and concentrated under reduced pressure. Purification by silica gel flash column chromatography (R_{f}=0.60, Hexanes/EtOAc, 50:50) provided the product 1e (336 mg, 72%) as a white solid. ¹H NMR (300 MHz, CDCl₃) δ 7.61 (s, 1H), 7.49 (s, 1H), 4.60 (s, 2H), 4.31 (s, 3H).

### tert-Butyl N-[(2R)-1-[(4-bromo-1-methyl-1H-1,2,3-benzotriazol-6-yl)methoxy]propan-2-yl]carbamate (1f)

To a stirred solution of Boc-D-alaninol (172 mg, 0.98 mmol, 1.5 eq) in THF (6.6 mL, 0.1 M) was added NaH (34 mg, 0.85 mmol, 1.3 eq) at 0° C. The resulting mixture was stirred for 30 minutes at 0° C., then 1e (200 mg, 0.66 mmol, 1.0 eq) in THF (1.0 mL) was added dropwise. The reaction mixture was stirred at room temperature for 16 hours. The mixture was poured into a saturated aqueous solution of NH₄Cl (5 mL), which was extracted with EtOAc (3×5 mL). The combined organics were washed with brine, dried over MgSO₄, filtered, and concentrated under reduced pressure. The crude product was purified by silica gel flash column chromatography (R_{f}=0.40, Hexanes/EtOAc, 50:50) to afford the product 1f (202 mg, 77%,) as a white solid. ¹H NMR (300 MHz, CDCl₃) δ 7.52 (s, 1H), 7.45 (s, 1H), 4.74-4.57 (m, 3H), 4.30 (s, 3H), 3.92 (s, 1H), 3.50 (d, J=4.8 Hz, 2H), 1.54 (s, 9H), 1.44 (s, 9H), 1.22 (d, J=6.7 Hz, 3H).

### tert-Butyl (R)-(6-((2-((tert-butoxycarbonyl)amino)propoxy)methyl)-1-methyl-1H-benzo[d][1,2,3]triazol-4-yl)carbamate (1g)

To a solution of 1f (50.0 mg, 0.125 mmol, 1.00 eq) in dioxane (2.5 mL) were added Cs₂CO₃ (82 mg, 0.25 mmol, 2.0 eq), NH₂Boc (29 mg, 0.25 mmol, 2.0 eq), Xantphos (15 mg, 0.025 mmol, 0.20 eq) and Pd₂(dba)₃-CHCl₃ (13 mg, 0.013 mmol, 0.10 eq). The resulting mixture was stirred for 2 hours at 110° C. under inert atmosphere. After cooling to room temperature, the mixture was concentrated under reduced pressure. Purification by silica gel flash column chromatography (R_{f}=0.50, Hexanes/EtOAc, 50:50) provided 1g (49 mg, 90%) as a yellow solid. ¹H NMR (300 MHz, CDCl₃) δ 7.89 (s, 1H), 7.87 (s, 1H), 7.14 (s, 1H), 4.66 (d, J=2.5 Hz, 3H), 4.27 (s, 3H), 3.90 (s, 1H), 3.49 (d, J=4.5 Hz, 2H), 1.56 (s, 9H), 1.43 (s, 9H), 1.22 (d, J=6.7 Hz, 3H).

### (R)-6-((2-Aminopropoxy)methyl)-1-methyl-1H-benzo[d][1,2,3]triazol-4-amine (1h)

To a solution of 1g (0.13 g, 0.30 mmol, 1.0 eq) in DCM (1.5 mL, 0.2 M) was added TFA (0.45 mL, 6.0 mmol, 20 eq) at 0° C. The resulting mixture was stirred for 2 hours at r.t. The solution was concentrated under reduced pressure to provide 1h (70 mg, quant.) as a brown oil.

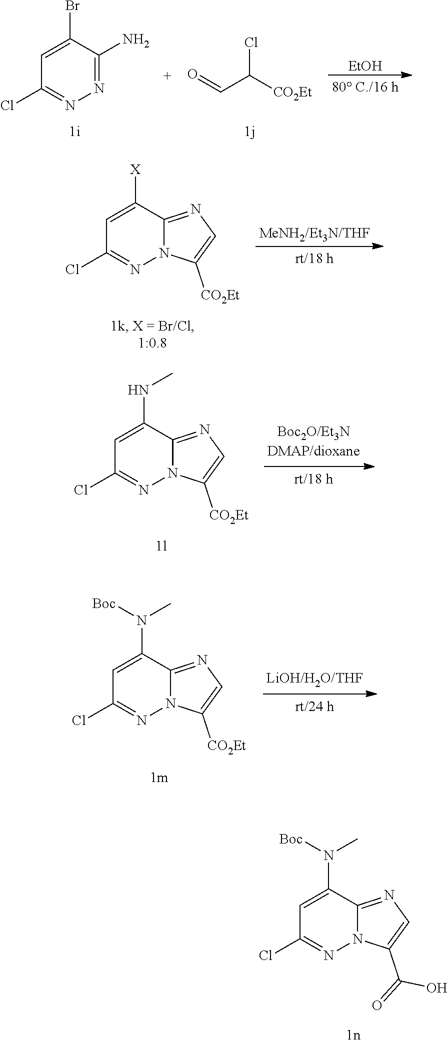

### Ethyl 8-bromo-6-chloroimidazo[1,2-b]pyridazine-3-carboxylate (1k1) and Ethyl 6,8-dichloro imidazo[1,2-b]pyridazine-3-carboxylate (1k2)

To a 25 mL round-bottomed flask were added 9 (1.0 g, 4.8 mmol, 1.0 eq) and 10 (0.87 g, 5.8 mmol, 1.2 eq) in anhydrous EtOH (10 mL, 0.5 M). The solution was heated to 80° C. for 16 hours. The solution was quenched with ethyl acetate and washed with water. The organic layer was dried over MgSO₄ and concentrated under reduced pressure. Purification by silica gel flash column chromatography provided a mixture of 1k1/2 (1.0 g, 73%, 1:0.8) as a yellow solid. TLC: R_{f}=0.60 (DCM/EtOAc, 90:10). ¹H NMR (300 MHz, CDCl₃) 1k1: δ 8.38 (s, 1H), 7.57 (s, 1H), 4.47 (q, J=7.2 Hz, 2H), 1.44 (t, J=7.1 Hz, 3H). 1k2: δ 8.37 (s, 1H), 7.38 (s, 1H), 4.47 (q, J=7.2 Hz, 2H), 1.44 (t, J=7.1 Hz, 3H).

### Ethyl 6-chloro-8-(methylamino)imidazo[1,2-b]pyridazine-3-carboxylate (11)

To a solution of 1k1/2 (1.0 g, 3.8 mmol, 1.0 eq) in anhydrous THF (38 mL, 0.1 M) under inert atmosphere were added Et₃N (2.1 mL, 15 mmol, 4.0 eq) and MeNH₂ (1.7 mL, 15 mmol, 4.0 eq, 33 wt. % in absolute EtOH) at 0° C. The reaction mixture was stirred at room temperature for 18 hours. The mixture was diluted with NH₄Cl solution (30 mL) and extracted with EtOAc (3×30 mL). The combined organics were washed with brine, dried over MgSO₄, filtered, and concentrated under reduced pressure. Purification by silica gel flash column chromatography provided 1l (0.88 g, 90%,) as a yellow solid. TLC: R_{f}=0.50 (DCM/EtOAc, 90:10). ¹H NMR (300 MHz, CDCl₃) δ 8.07 (s, 1H), 6.14 (s, 1H), 5.97 (br s, 1H), 4.43 (q, J=7.2 Hz, 2H), 3.06 (d, J=5.3 Hz, 3H), 1.41 (t, J=7.1 Hz, 3H).

### Ethyl 8-{[(tert-butoxy)carbonyl](methyl)amino}-6-chloroimidazo[1,2-b]pyridazine-3-carboxylate (1m)

To a solution of 1l (0.88 g, 3.5 mmol, 1.0 eq) in dioxane (35 mL, 0.1 M) under inert atmosphere were added Boc₂O (2.7 g, 4.1 mmol, 1.2 eq), Et₃N (0.72 mL, 5.2 mmol, 1.5 eq) and DMAP (21 mg, 0.17 mmol, 0.050 eq). The reaction mixture was stirred at room temperature for 18 hours. The solvent was removed under reduced pressure. Purification by silica gel flash column chromatography provided 1m (1.07 g, 87%) as a yellow oil. TLC: R_{f}=0.45 (DCM/EtOAc, 95:5). ¹H NMR (300 MHz, CDCl₃) δ 8.27 (s, 1H), 7.33 (s, 1H), 4.45 (q, J=7.1 Hz, 2H), 3.58 (s, 3H), 1.50 (s, 9H), 1.43 (t, J=7.1 Hz, 3H).

### 8-((tert-Butoxycarbonyl)(methyl)amino)-6-chloroimidazo[1,2-b]pyridazine-3-carboxylic acid (in)

To a solution of 1m (0.5 g, 1.4 mmol, 1.0 eq) in THF (7.0 mL, 0.2 M) were added LiOH·H₂O (0.18 g, 4.2 mmol, 3.0 eq), the resulting mixture was stirred for 30 minutes, and then 5 drops of water were added. The reaction mixture was stirred for 20 hours at r.t., and quenched by addition of 1 M HCl solution to pH 2-3. The aqueous layer was extracted with DCM (3×10 mL). The combined organics were washed with brine, dried over Na₂SO₄, filtered, and concentrated to provide in (0.4 g, 86%) as a white solid. ¹H NMR (300 MHz, DMSO-d₆) δ 13.29 (br s, 1H), 8.32 (s, 1H), 7.61 (s, 1H), 3.41 (s, 3H), 1.39 (s, 9H).

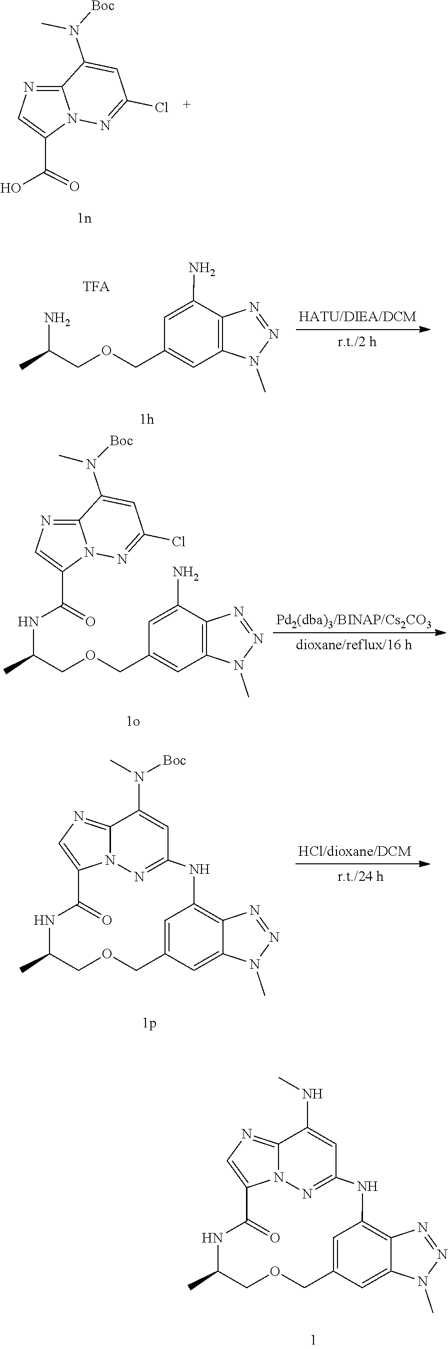 [SMILES: CN(C(=O)OC(C)(C)C)c1cc(Cl)nn2c(C(=O)O)cnc12 ; C[C@@H](N)COCc1cc(N)c2nnn(C)c2c1 ; C[C@H](COCc1cc(N)c2nnn(C)c2c1)NC(=O)c1cnc2c(N(C)C(=O)OC(C)(C)C)cc(Cl)nn12 ; C[C@@H]1COCc2cc(c3nnn(C)c3c2)Nc2cc(N(C)C(=O)OC(C)(C)C)c3ncc(n3n2)C(=O)N1 ; CNc1cc2nn3c(cnc13)C(=O)N[C@H](C)COCc1cc(c3nnn(C)c3c1)N2]

### tert-Butyl (R)-(3-((1-((4-amino-1-methyl-1H-benzo[d][1,2,3]triazol-6-yl)methoxy)propan-2-yl)carbamoyl)-6-chloroimidazo[1,2-b]pyridazin-8-yl)(methyl)carbamate (to)

To a solution of 1n (97 mg, 0.30 mmol, 1.0 eq) in DCM (4.3 mL, 0.1 M) were added HATU (0.16 g, 0.43 mmol, 1.4 eq) and DIEA (0.60 mL, 3.4 mmol, 11 eq). The resulting mixture was stirred for 20 minutes at r.t., and then 1h (100 mg, 0.43 mmol, 1.4 eq) was added. The reaction was stirred for 2 hours at r.t. The solution was concentrated under reduced pressure. Purification by silica gel flash column chromatography (R_{f}=0.45, DCM/MeOH, 95:5) provided to (90 mg, 56%) as a yellow solid. ¹H NMR (300 MHz, CDCl₃) δ 8.55 (d, J=7.9 Hz, 1H), 8.38 (s, 1H), 7.34 (s, 1H), 6.80 (s, 1H), 6.50 (s, 1H), 4.66 -4.58 (m, 2H), 4.50-4.49 (m, 1H), 4.18 (s, 3H), 3.74-3.69 (m, 1H), 3.63 (s, 3H), 3.22-3.15 (m, 1H), 1.52 (s, 9H), 1.39 (d, J=7.3 Hz, 3H).

### tert-Butyl ((7R,E)-3¹,7-dimethyl-9-oxo-3¹H-5-oxa-2,8-diaza-1(6,3),3(4,6)-diimidazo[1,2-b]pyridazinacyclononaphane-1⁸-yl)(methyl)carbamate (1p)

To a solution of 1o (90 mg, 0.17 mmol, 1.0 eq) in anhydrous dioxane (3.3 mL, 0.05 M)) were added Cs₂CO₃ (108 mg, 0.33 mmol, 2.0 eq), Pd₂(dba)₃ (15 mg, 0.017 mmol, 0.1 eq) and BINAP (5.2 mg, 0.0080 mmol, 0.050 eq). The resulting mixture was stirred for 16 hours at reflux under N₂. The mixture was concentrated under reduced pressure and purified by silica gel flash column chromatography (R_{f}=0.42, DCM/MeOH, 95:5) to provide 1p (42 mg, 50%) as a yellow solid. ¹H NMR (300 MHz, CDCl₃) δ 8.72 (d, J=5.9 Hz, 1H), 8.51 (s, 1H), 8.37 (s, 1H), 8.10 (s, 1H), 7.18 (s, 1H), 6.95 (s, 1H), 4.96 (d, J=14.2 Hz, 1H), 4.58 (d, J=14.2 Hz, 1H), 4.40-4.33 (m, 1H), 4.29 (s, 3H), 3.63 (dd, J=9.5, 2.7 Hz, 1H), 3.56 (s, 3H), 3.43 (t, J=9.3 Hz, 1H), 1.54 (s, 9H), 1.22 (d, J=6.5 Hz, 4H).

### (7R,E)-3¹,7-Dimethyl-1⁸-(methylamino)-3¹H-5-oxa-2,8-diaza-1(6,3),3(4,6)-diimidazo[1,2-b]pyridazinacyclononaphan-9-one

To a solution of 1p (42 mg, 0.080 mmol, 1.0 eq) in DCM (3.2 mL, 0.025 M) was added HCl (3.0 mL, 4M in dioxane) dropwise at 0° C. The reaction mixture was stirred for 24 hours at r.t. The reaction mixture was concentrated. The crude product was dissolved in MeOH (2 mL), and Na₂CO₃ was added to the solution. The mixture was stirred for 10 min at r.t., filtered, and concentrated under reduced pressure. Purification by reverse C18 column chromatography (R_{f}=0.40, DCM/MeOH, 95:5) to provide the title compound (14.8 mg, 47%) as an off-white solid. Formula: C₁₉H₂₁N₉O₂. MW: 407.44. LCMS [M+H]⁺=408.10. ¹H NMR (300 MHz, CDCl₃) δ 8.89 (d, J=5.6 Hz, 1H), 8.39 (s, 1H), 8.10 (s, 1H), 7.72 (s, 1H), 6.94 (s, 1H), 5.99 (q, J=5.0 Hz, 1H), 5.75 (s, 1H), 4.92 (d, J=14.2 Hz, 1H), 4.61 (d, J=14.2 Hz, 1H), 4.42-4.31 (m, 1H), 4.29 (s, 3H), 3.62 (dd, J=9.5, 2.7 Hz, 1H), 3.47 (t, J=9.1 Hz, 1H), 3.07 (d, J=5.2 Hz, 3H), 1.27 (d, J=6.5 Hz, 3H).

### Example 2: Synthesis of (7R,E)-7-(Hydroxymethyl)-3¹-methyl-1⁸-(methylamino)-3¹H-5-oxa-2,8-diaza-1(6,3),3(4,6)-diimidazo[1,2-b]pyridazinacyclononaphan-9-one (Compound 2)

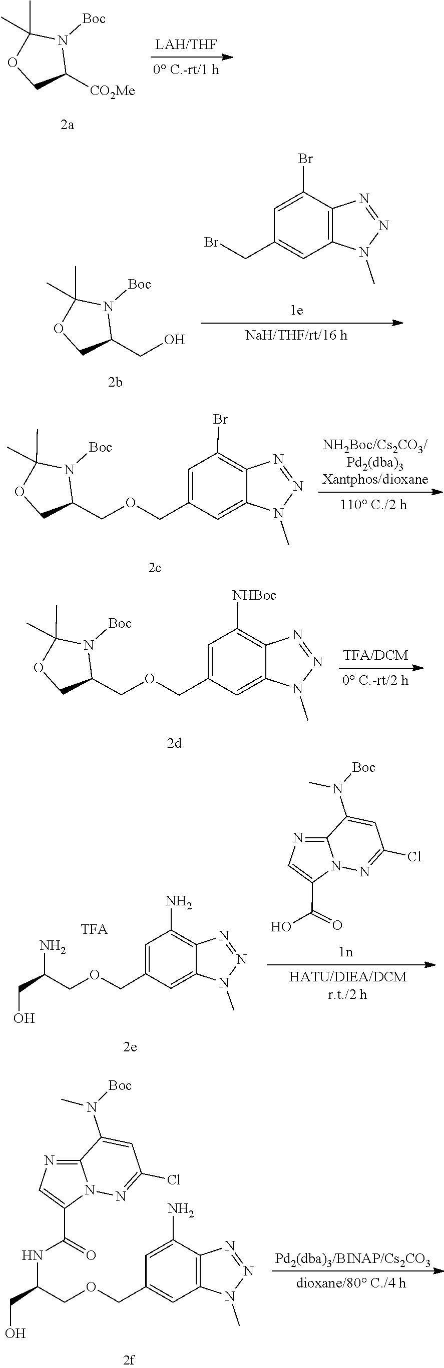

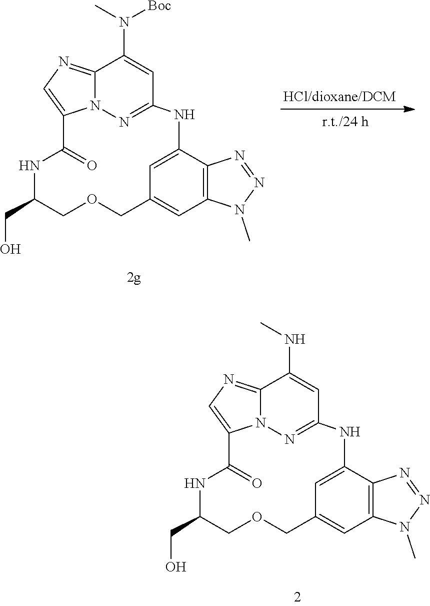 [SMILES: CN(C(=O)OC(C)(C)C)c1cc2nn3c(cnc13)C(=O)N[C@H](CO)COCc1cc(c3nnn(C)c3c1)N2 ; CNc1cc2nn3c(cnc13)C(=O)N[C@H](CO)COCc1cc(c3nnn(C)c3c1)N2]

### tert-Butyl (S)-4-(hydroxymethyl)-2,2-dimethyloxazolidine-3-carboxylate (2b)

To a solution of 2a (2.0 g, 7.7 mmol, 1.0 eq) in anhydrous THF (15 mL) was added LiAlH4 (5.8 mL, 11 mmol, 1.5 eq, 2.0 M in THF) at 0° C. The resulting mixture was stirred at room temperature for 1 hour. The reaction was quenched with a solution of 2.0 M NaOH (15 mL). After extraction with EtOAc (3×15 mL), the mixture was dried over Na₂SO₄, filtered, and concentrated in vacuo. Purification by silica gel flash chromatography (R_{f}=0.5, hexanes/EtOAc, 50:50) provided 2b (1.6 g, 90%) as a colorless oil. ¹H NMR (300 MHz, Acetone-d₆) δ 4.06-3.75 (m, 4H), 3.70 (dt, J=9.4, 4.5 Hz, 1H), 3.42-3.32 (m, 1H), 1.52-1.41 (m, 15H).

### tert-Butyl (S)-4-(((4-bromo-1-methyl-1H-benzo[d][1,2,3]triazol-6-yl)methoxy)methyl)-2,2-dimethyloxazolidine-3-carboxylate (2c)

To a solution of 2b (0.68 g, 2.9 mmol, 1.2 eq) in anhydrous THF (25 mL) was added NaH (0.13 g, 3.2 mmol, 1.3 eq) in portions at 0° C. The mixture was stirred for 30 minutes at 0° C., and 1e (0.75 g, 2.5 mmol, 1.0 eq) was added. The resulting mixture was stirred at room temperature for 16 hours. The mixture was poured into a saturated aqueous solution of NH₄Cl (5 mL), which was extracted with EtOAc (3×5 mL). The combined organics were washed with brine, dried over Na₂SO₄, filtered, and concentrated under reduced pressure. The crude product was purified by silica gel flash column chromatography (R_{f}=0.30, Hexanes/EtOAc, 60:40) to afford 2c (1.1 g, 98%) as a white solid. LCMS: [M+H]⁺=456.78. ¹H NMR (300 MHz, CDCl₃) δ 7.51 (s, 1H), 7.47 (s, 1H), 4.69 (s, 2H), 4.30 (s, 3H), 4.15-4.10 (m, 1H), 4.06-3.95 (m, 1H), 3.75-3.73 (m, 1H), 3.64-3.59 (m, 1H), 3.54-3.47 (m, 1H), 1.67-1.34 (m, 15H).

### tert-Butyl (S)-4-(((4-((tert-butoxycarbonyl)amino)-1-methyl-1H-benzo[d][1,2,3]triazol-6-yl) methoxy)methyl)-2,2-dimethyloxazolidine-3-carboxylate (2d)

To a solution of 2c (0.56 g, 1.2 mmol, 1.0 eq) in dioxane (12 mL) were added Cs₂CO₃ (0.80 g, 2.5 mmol, 2.0 eq), NH₂Boc (0.57 g, 4.9 mmol, 4.0 eq), Xantphos (35 mg, 0.061 mmol, 0.050 eq) and Pd₂(dba)₃ (112 mg, 0.123 mmol, 0.100 eq). The resulting mixture was stirred for 16 hours at reflux under inert atmosphere. After cooling to room temperature, the mixture was concentrated under reduced pressure. Purification by silica gel flash column chromatography (R_{f}=0.50, hexanes/EtOAc, 50:50) provided 2d (0.31 g, 51%) as a colorless oil. LCMS: [M+H]⁺=492.00. ¹H NMR (300 MHz, CDCl₃) δ 7.91 (s, 1H), 7.87 (s, 1H), 7.15 (d, J=6.3 Hz, 1H), 4.68 (s, 2H), 4.26 (s, 3H), 4.14-4.04 (m, 1H), 3.99 (td, J=9.3, 8.7, 4.3 Hz, 1H), 3.82-3.69 (m, 1H), 3.69-3.56 (m, 1H), 3.47 (q, J=7.8, 7.2 Hz, 1H), 1.56 (s, 9H), 1.48 (s, 9H), 1.40 (s, 6H).

### (R)-2-Amino-3-((4-amino-1-methyl-1H-benzo[d][1,2,3]triazol-6-yl)methoxy)propan-1-ol (2e)

To a solution of 2d (0.31 g, 0.62 mmol, 1.0 eq) in DCM (3.1 mL) was added TFA (0.95 mL, 12 mmol, 20 eq) at 0° C. The resulting mixture was stirred for 2 hours at room temperature, The mixture was concentrated under reduced pressure to provide 2e (0.14 g, quant.) as a brown oil. LCMS: [M+H]⁺=252.31.

### tert-Butyl (R)-(3-((1-((4-amino-1-methyl-1H-benzo[d][1,2,3]triazol-6-yl)methoxy)-3-hydroxy propan-2-yl)carbamoyl)-6-chloroimidazo[1,2-b]pyridazin-8-yl)(methyl)carbamate (2f)

To a solution of 1n (0.14 g, 0.44 mmol, 1.0 eq) in anhydrous DMF (6.3 mL) were added HATU (0.17 g, 0.44 mmol, 1.0 eq) and DIEA (0.61 mL, 3.5 mmol, 8.0 eq). The resulting mixture was stirred at room temperature for 20 min, and then 2e (0.14 g, 0.53 mmol, 1.2 eq) was added. The reaction was stirred at room temperature for 2 hours. The solution was concentrated under reduced pressure. Purification by silica gel flash column chromatography (R_{f}=0.40, DCM/MeOH, 95:5) provided 2f (0.13 g, 53%) as a white foam. LCMS: [M+H]⁺=560.23. ¹H NMR (300 MHz, CDCl₃) δ 8.99 (d, J=7.4 Hz, 1H), 8.40 (s, 1H), 7.37 (s, 1H), 6.78 (s, 1H), 6.48 (s, 1H), 4.66 (s, 2H), 4.63 (s, 2H), 4.40 (dq, J=9.0, 4.6 Hz, 1H), 4.18 (s, 3H), 4.06-3.94 (m, 1H), 3.84 (qd, J=9.8, 4.5 Hz, 3H), 3.65 (s, 3H), 2.99-2.85 (m, 1H), 1.52 (s, 9H).

### tert-Butyl ((7R,E)-7-(hydroxymethyl)-3¹-methyl-9-oxo-3¹H-5-oxa-2,8-diaza-1(6,3),3(4,6)-diimidazo[1,2-b]pyridazinacyclononaphane-1⁸-yl)(methyl)carbamate (2g)

To a solution of 2f (0.13 g, 0.23 mmol, 1.0 eq) in anhydrous dioxane (23 mL) were added Cs₂CO₃ (0.15 g, 0.46 mmol, 2.0 eq), Pd₂(dba)₃ (21 mg, 0.023 mmol, 0.10 eq) and BINAP (7.2 mg, 0.012 mmol, 0.050 eq). The reaction mixture was stirred for 16 hours at reflux under N₂. The reaction mixture was filtered over a celite pad, concentrated, and purified by silica gel flash column chromatography (R_{f}=0.38, DCM/MeOH, 95:5) to provide 2g (30 mg, 25%) as a white solid. LCMS: [M+H]⁺=524.18. ¹H NMR (300 MHz, CDCl₃) δ 8.82 (d, J=5.4 Hz, 1H), 8.33 (s, 1H), 8.21 (s, 1H), 8.18 (s, 1H), 7.11 (s, 1H), 6.90 (s, 1H), 4.82 (d, J=14.2 Hz, 1H), 4.70 (d, J=14.2 Hz, 1H), 4.45-4.31 (m, 1H), 4.20 (s, 3H), 4.01-3.75 (m, 5H), 3.56 (s, 3H), 1.55 (s, 9H).

### (7R,E)-7-(Hydroxymethyl)-3¹-methyl-1⁸-(methylamino)-3¹H-5-oxa-2,8-diaza-1(6,3),3(4,6)-diimidazo[1,2-b]pyridazinacyclononaphan-9-one

To a solution of 2g (30 mg, 0.057 mmol, 1.0 eq) in DCM (2.3 mL) was added HCl (1.4 mL, 4.0 M in dioxane) dropwise at 0° C. The reaction mixture was stirred for 24 hours at r.t. The reaction mixture was concentrated. The crude product was dissolved in MeOH (2 mL), and Na₂CO₃ was added to the solution. The mixture was stirred for 10 min at r.t., filtered, and concentrated under reduced pressure. Purification by silica gel flash chromatography (R_{f}=0.25, DCM/MeOH, 95:5) provided the title compound (14 mg, 58%) as a white solid. Formula: C₁₉H₂₁N₉O₃. MW: 423.44. LCMS: [M+H]′=424.15. ¹H NMR (300 MHz, DMSO-d₆) δ 9.99 (s, 1H), 8.80 (d, J=5.9 Hz, 1H), 8.23 (s, 1H), 7.87 (s, 1H), 7.58 (d, J=5.0 Hz, 1H), 7.28 (s, 1H), 6.39 (s, 1H), 4.88 (d, J=14.2 Hz, 1H), 4.74 (t, J=5.5 Hz, 1H), 4.56 (d, J=14.2 Hz, 1H), 4.28 (s, 3H), 4.08-3.96 (m, 1H), 3.75-3.64 (m, 1H), 3.61-3.38 (m, 3H), 2.91 (d, J=4.8 Hz, 3H).

### Example 3: Synthesis of (7R,E)-3′,7-Dimethyl-1⁸-(methylamino)-3¹H-5,8-dioxa-2-aza-1(6,3),3(4,6)-diimidazo[1,2-b]pyridazinacyclononaphan-9-one (Compound 3)

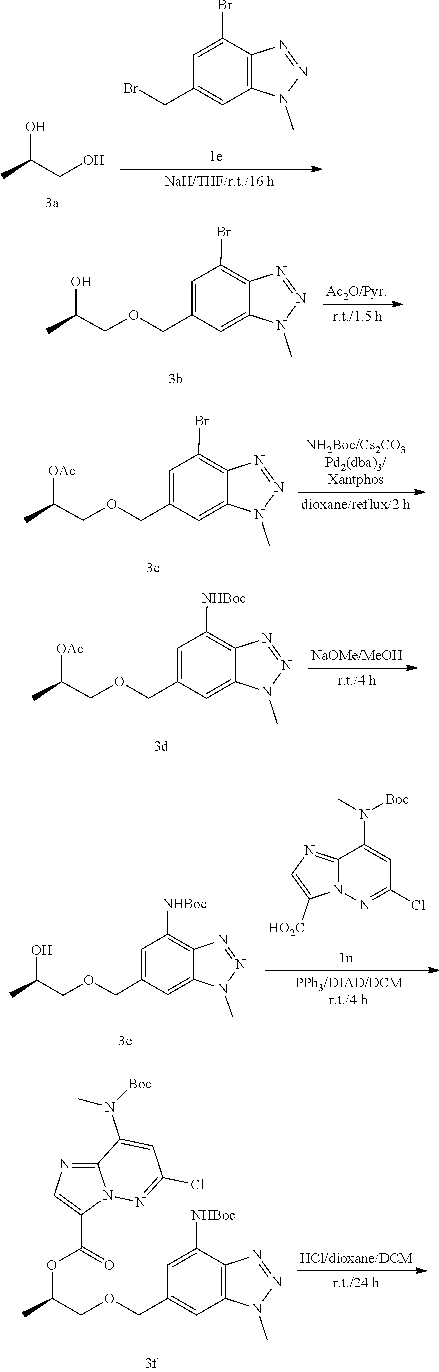

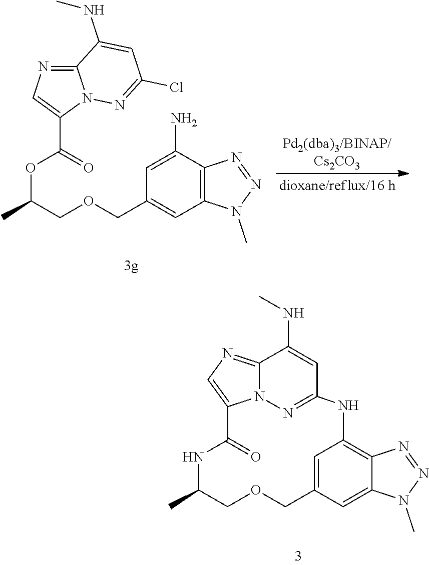 [SMILES: CNc1cc(Cl)nn2c(C(=O)O[C@H](C)COCc3cc(N)c4nnn(C)c4c3)cnc12 ; CNc1cc2nn3c(cnc13)C(=O)N[C@H](C)COCc1cc(c3nnn(C)c3c1)N2]

### (R)-1-((4-Bromo-1-methyl-1H-benzo[d][1,2,3]triazol-6-yl)methoxy)propan-2-ol (3b)

To a solution of 3a (0.22 g, 2.9 mmol, 1.5 eq) in anhydrous THF (19 mL) was added NaH (0.1 g, 2.5 mmol, 1.3 eq) in portions at 0° C. The mixture was stirred for 30 minutes at 0° C., and 1e (0.59 g, 1.9 mmol, 1.0 eq) was added. The resulting mixture was stirred at room temperature for 16 hours. The mixture was poured into a saturated aqueous solution of NH₄Cl (5 mL) and extracted with EtOAc (3×5 mL). The combined organics were washed with brine, dried over Na₂SO₄, filtered, and concentrated under reduced pressure. Purification by silica gel flash chromatography (R_{f}=0.35, 100% EtOAc) provided 3b (0.43 g, 73%) as a yellowish solid. LCMS: [M+H]⁺=301.96. ¹H NMR (300 MHz, CDCl₃) δ 7.52 (s, 1H), 7.46 (s, 1H), 4.70 (s, 2H), 4.30 (s, 3H), 4.15-3.96 (m, 1H), 3.54 (dd, J=9.4, 3.1 Hz, 1H), 3.37 (dd, J=9.4, 7.9 Hz, 1H), 2.31 (d, J=3.2 Hz, 1H), 1.19 (d, J=6.4 Hz, 3H).

### (R)-1-((4-Bromo-1-methyl-1H-benzo[d][1,2,3]triazol-6-yl)methoxy)propan-2-yl acetate (3c)

To a solution of 3b (0.43 g, 1.4 mmol, 1.0 eq) in pyridine (2.8 mL) was added Ac₂O (4.0 mL, 42 mmol, 30 eq) at 0° C. The resulting mixture was stirred for 1 hour at r.t. The solution was concentrated under reduced pressure. Purification by silica gel flash chromatography (R_{f}=0.35, hexanes/EtOAc, 50:50) provided 3c (0.41 g, 84%) as a colorless oil. LCMS: [M+H]=343.99. ¹H NMR (300 MHz, CDCl₃) δ 7.51 (d, J 0.7 Hz, 1H), 7.44 (d, J=1.1 Hz, 1H), 5.16 (pd, J=6.4, 4.3 Hz, 1H), 4.78-4.58 (m, 2H), 4.29 (s, 3H), 3.64-3.48 (m, 2H), 2.07 (s, 3H), 1.27 (d, J=6.5 Hz, 3H).

### (R)-1-((4-((tert-Butoxycarbonyl)amino)-1-methyl-1H-benzo[d][1,2,3]triazol-6-yl)methoxy) propan-2-yl acetate (3d)

To a solution of 3c (0.20 g, 0.58 mmol, 1.0 eq) in dioxane (5.8 mL) were added Cs₂CO₃ (0.38 g, 1.2 mmol, 2.0 eq), NH₂Boc (0.27 g, 2.3 mmol, 4.0 eq), Xantphos (17 mg, 0.029 mmol, 0.050 eq) and Pd₂(dba)₃ (53 mg, 0.058 mmol, 0.10 eq). The resulting mixture was stirred for 16 hours at reflux under inert atmosphere. After cooling to room temperature, the mixture was concentrated under reduced pressure. Purification by flash chromatography on silica gel (R_{f}=0.60, hexanes/EtOAc, 50:50) provided 3d (0.2 g, 90%) as a colorless oil.

¹H NMR (300 MHz, CDCl₃) δ 7.88 (s, 1H), 7.86 (s, 1H), 7.14 (s, 1H), 5.14 (pd, J=6.4, 4.3 Hz, 1H), 4.77-4.57 (m, 2H), 4.25 (s, 3H), 3.66-3.46 (m, 2H), 2.06 (s, 3H), 1.54 (s, 9H), 1.26 (d, J=6.4 Hz, 3H).

### tert-Butyl (R)-(6-((2-hydroxypropoxy)methyl)-1-methyl-1H-benzo[d][1,2,3]triazol-4-yl) carbamate (3e)

To a solution of 3d (0.18 g, 0.47 mmol, 1.0 eq) in MeOH (4.8 mL) was added NaOMe (0.22 mL, 0.95 mmol, 2.0 eq). The resulting mixture was stirred for 2 hours at r.t. under inert atmosphere. Then it was quenched with 1.0 M HCl (˜1 mL), filtered, and evaporated under reduced pressure. Purification by flash chromatography on silica gel (R_{f}=0.20, hexanes/EtOAc, 50:50) provided 3e (0.15 g, 94%) as a yellowish foam. LCMS: [M+H]⁺=337.06. ¹H NMR (300 MHz, CDCl₃) δ 7.92 (s, 1H), 7.88 (s, 1H), 7.15 (s, 1H), 4.70 (s, 2H), 4.27 (s, 3H), 4.12-3.98 (m, 1H), 3.54 (dd, J=9.4, 3.0 Hz, 1H), 3.36 (dd, J=9.4, 8.1 Hz, 1H), 2.38 (s, 1H), 1.56 (s, 9H), 1.18 (d, J=6.4 Hz, 3H).

### (R)-1-((4-((tert-Butoxycarbonyl)amino)-1-methyl-1H-benzo[d][1,2,3]triazol-6-yl)methoxy) propan-2-yl 8-((tert-butoxycarbonyl)(methyl)amino)-6-chloroimidazo[1,2-b]pyridazine-3-carboxylate (3f)

To a solution of 1n (74 mg, 0.22 mmol, 1.0 eq) in DCM (4.5 mL) were added DIAD (68 mg, 0.34 mmol, 1.5 eq), Ph₃P (89 mg, 0.34 mmol, 1.5 eq) at 0° C. The resulting mixture was stirred for 5 minutes, and 3e (80 mg, 0.24 mmol, 1.1 eq) in DCM (1.4 mL) was added. The resulting mixture was stirred for 2 hours at r.t and then filtered over a celite pad. Purification by flash chromatography on silica gel (R_{f}=0.50, hexanes/EtOAc, 50:50) provided 3f (0.12 g, 84%) as a white foam. LCMS: [M+H]⁺=645.05.

### (R)-1-((4-Amino-1-methyl-1H-benzo[d][1,2,3]triazol-6-yl)methoxy)propan-2-yl 6-chloro-8-(methylamino)imidazo[1,2-b]pyridazine-3-carboxylate (3g)

To a solution of 3f (0.12 g, 0.19 mmol, 1.0 eq) in DCM (2.0 mL) was added HCl (0.30 mL, 4.0 M in dioxane) at 0° C. The resulting mixture was stirred for 24 hours at room temperature, The mixture was concentrated under reduced pressure and purified by flash chromatography on silica gel (R_{f}=0.45, DCM/MeOH, 95:5) provided 3g (60 mg, 71%) as a white solid. LCMS: [M+H]⁺=445.06. ¹H NMR (300 MHz, CDCl₃) δ 8.06 (s, 1H), 6.78 (s, 1H), 6.46 (s, 1H), 6.14 (s, 1H), 6.08 (d, J=5.3 Hz, 1H), 5.52-5.38 (m, 1H), 4.79-4.56 (m, 4H), 4.18 (s, 3H), 3.84-3.59 (m, 2H), 3.06 (d, J=5.5 Hz, 3H), 1.41 (d, J=6.4 Hz, 3H).

### (7R,E)-3¹,7-Dimethyl-1⁸-(methylamino)-3¹H-5,8-dioxa-2-aza-1(6,3),3(4,6)-diimidazo[1,2-b]pyridazinacyclononaphan-9-one

To a solution of 3g (50 mg, 0.11 mmol, 1.0 eq) in anhydrous dioxane (4.0 mL) were added Cs₂CO₃ (73 mg, 0.22 mmol, 2.0 eq), Pd₂(dba)₃ (10 mg, 0.011 mmol, 0.10 eq) and BINAP (3.5 mg, 0.0060 mmol, 0.050 eq). The resulting mixture was stirred for 16 hours at reflux under N₂. The reaction mixture was filtered over a celite pad, concentrated under reduced pressure and purified by silica gel flash column chromatography (R_{f}=0.45, DCM/MeOH, 95:5) to provide the title compound (15 mg, 33%) as a white solid. LCMS: [M+H]⁺=409.12. 1H NMR (300 MHz, CDCl₃) δ 9.16 (s, 1H), 8.15 (s, 1H), 7.73 (s, 1H), 6.82 (s, 1H), 5.78 (s, 2H), 5.31 (t, J=6.4 Hz, 1H), 4.87 (q, J=15.4 Hz, 2H), 4.26 (s, 3H), 3.95-3.77 (m, 2H), 3.06 (d, J=5.2 Hz, 3H), 1.49 (d, J=6.4 Hz, 3H).

### Example 4: Synthesis of (R,1³E,1⁴E)-3¹,7-dimethyl-1⁷-(methylamino)-3¹H-5,8-dioxa-2-aza-1(5,3),3(4,6)-dipyrazolo [1,5-a]pyrimidinacyclononaphan-9-one (Compound 4)

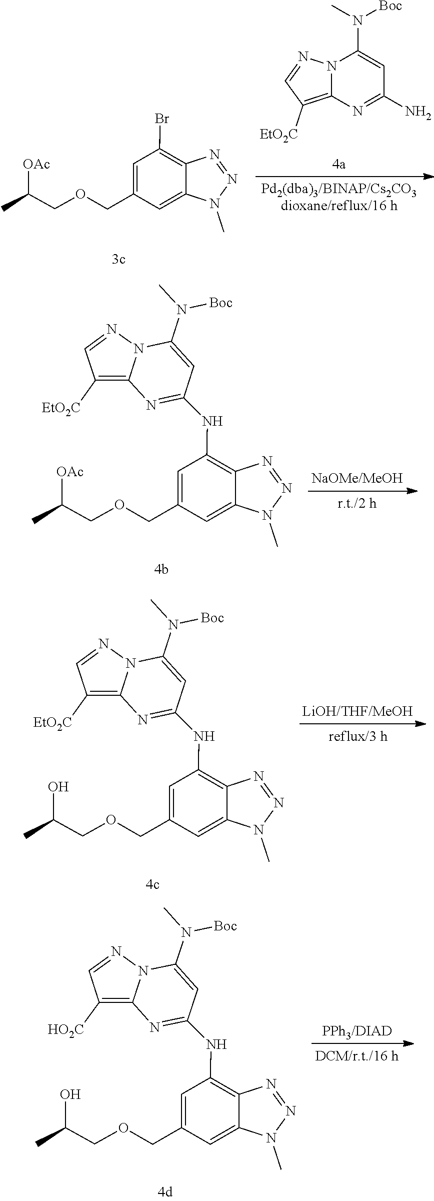 [SMILES: CC(=O)O[C@H](C)COCc1cc(Br)c2nnn(C)c2c1 ; CCOC(=O)c1cnn2c(N(C)C(=O)OC(C)(C)C)cc(N)nc12 ; CCOC(=O)c1cnn2c(N(C)C(=O)OC(C)(C)C)cc(Nc3cc(COC[C@@H](C)OC(C)=O)cc4c3nnn4C)nc12 ; CCOC(=O)c1cnn2c(N(C)C(=O)OC(C)(C)C)cc(Nc3cc(COC[C@@H](C)O)cc4c3nnn4C)nc12 ; C[C@@H](O)COCc1cc(Nc2cc(N(C)C(=O)OC(C)(C)C)n3ncc(C(=O)O)c3n2)c2nnn(C)c2c1]

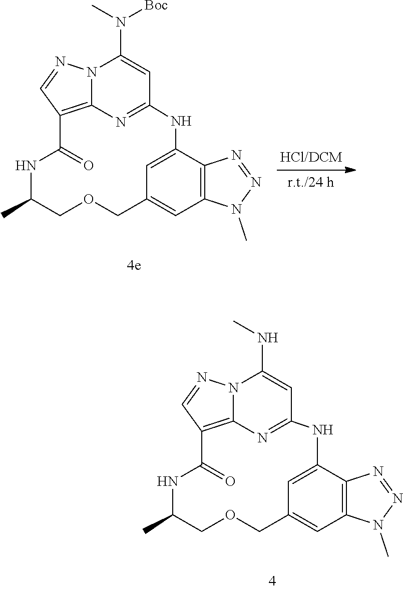 [SMILES: C[C@@H]1COCc2cc(c3nnn(C)c3c2)Nc2cc(N(C)C(=O)OC(C)(C)C)n3ncc(c3n2)C(=O)N1 ; CNc1cc2nc3c(cnn13)C(=O)N[C@H](C)COCc1cc(c3nnn(C)c3c1)N2]

### Ethyl (R)-5-((6-((2-acetoxypropoxy)methyl)-1-methyl-1H-benzo[d][1,2,3]triazol-4-yl)amino) 7-((tert-butoxycarbonyl)(methyl)amino)pyrazolo[1,5-a]pyrimidine-3-carboxylate (4b)

To a solution of 3c (0.20 g, 0.59 mmol, 1.0 eq) in anhydrous dioxane (5.9 mL) were added 4a (0.22 g, 0.65 mmol, 1.1 eq), Cs₂CO₃ (0.39 g, 1.2 mmol, 2.0 eq), BINAP (68 mg, 0.12 mmol, 0.20 eq) and Pd₂(dba)₃ (54 mg, 0.059 mmol, 0.10 eq). The resulting mixture was stirred for 16 hours at reflux under inert atmosphere. After cooling to room temperature, the mixture was concentrated under reduced pressure. Purification by silica gel flash column chromatography (R_{f}=0.60, DCM/MeOH, 95:5) provided 4b (0.26 g, 74%) as a yellow solid. LCMS [M+H]⁺=597.15.

### Ethyl (R)-7-((tert-butoxycarbonyl)(methyl)amino)-5-((6-((2-hydroxypropoxy)methyl)-1-methyl-1H-benzo[d][1,2,3]triazol-4-yl)amino)pyrazolo[1,5-a]pyrimidine-3-carboxylate (4c)

To a solution of 4b (0.38 g, 0.63 mmol, 1.0 eq) in MeOH (13 mL) was added NaOMe (0.29 mL, 1.3 mmol, 2.0 eq) at 0° C. The resulting mixture was stirred for 2 hours at r.t. under inert atmosphere. Then it was quenched with 1.0 M HCl (˜1 mL), filtered and evaporated under reduced pressure. Purification by flash chromatography on silica gel (R_{f}=0.20, DCM/MeOH, 95:5) provided 4c (0.29 g, 83%) as a yellowish foam. LCMS: [M+H]⁺=555.13.

### (R)-7-((tert-Butoxycarbonyl)(methyl)amino)-5-((6-((2-hydroxypropoxy)methyl)-1-methyl-1H-benzo[d][1,2,3]triazol-4-yl)amino)pyrazolo[1,5-a]pyrimidine-3-carboxylic acid (4d)

To a solution of 4c (0.14 g, 0.24 mmol, 1.0 eq) in a mixture of THF/MeOH/water (1:1:1, 3.6 mL) was added LiOH monohydrate (0.20 g, 4.8 mmol, 20 eq). The reaction mixture was stirred at reflux for 3 hours. The mixture was concentrated, dissolved in DCM (3.0 mL) and treated with 1.0 M HCl to a pH of 3˜4. It was extracted with DCM (3×5 mL), dried over Na₂SO₄, and concentrated to provided 4d (90 mg, 70%) as a white solid. LCMS [M+H]⁺=527.09.

### tert-Butyl ((R,1³E,1⁴E)-3′,7-dimethyl-9-oxo-3¹H-5,8-dioxa-2-aza-1(5,3),3(4,6)-dipyrazolo [1,5-a]pyrimidinacyclononaphane-1⁷-yl)(methyl)carbamate (4e)

To a solution of 4d (90 mg, 0.17 mmol, 1.0 eq) in DCM (8.5 mL) were added DIAD (69 mg, 0.34 mmol, 2.0 eq) and Ph₃P (90 mg, 0.34 mmol, 2.0 eq). The resulting mixture was stirred for 16 hours at r.t., filtered over a celite pad and concentrated under reduced pressure. Purification by silica gel flash column chromatography (R_{f}=0.45, DCM/MeOH, 95:5) provided 4e (45 mg, 52%) as a white solid. LCMS: [M+H]⁺=509.09.

### (R,1³E,1⁴E)-3¹,7-dimethyl-1⁷-(methylamino)-3¹H-5,8-dioxa-2-aza-1(5,3),3(4,6)-dipyrazolo [1,5-a]pyrimidinacyclononaphan-9-one

To a solution of 4e (45 mg, 0.088 mmol, 1.0 eq) in DCM (3.5 mL) was added HCl (2.2 mL, 4.0 M in dioxane) at 0° C. The resulting mixture was stirred for 24 hours at r.t., concentrated and dissolved in MeOH (2 mL). The mixture was added Na₂CO₃, filtered and concentrated under reduced pressure. Purification by silica gel chromatography (R_{f}=0.45, DCM/MeOH, 95:5) provided the title compound (20 mg, 55%) as a white solid. Formula: C₁₉H₂₀N₈O₃. MW: 408.42. LCMS: [M+H]⁺=409.06. ¹H NMR (300 MHz, CDCl₃) δ 9.66 (s, 1H), 8.35 (s, 1H), 8.06 (s, 1H), 6.87 (s, 1H), 6.21 (q, J=5.4 Hz, 1H), 5.56 (s, 1H), 5.21-5.08 (m, 1H), 4.93 (d, J=15.3 Hz, 1H), 4.82 (d, J=15.2 Hz, 1H), 4.27 (s, 3H), 3.90 (d, J=4.7 Hz, 2H), 3.11 (d, J=5.2 Hz, 3H), 1.54 (d, J=6.4 Hz, 3H).

### Example 5: (7R,E)-3¹,7-Dimethyl-1⁸-(methylamino)-3¹H-5-oxa-2,8-diaza-1(6,3),3(7,5)-diimidazo[1,2-b]pyridazinacyclononaphan-9-one (Compound 5)

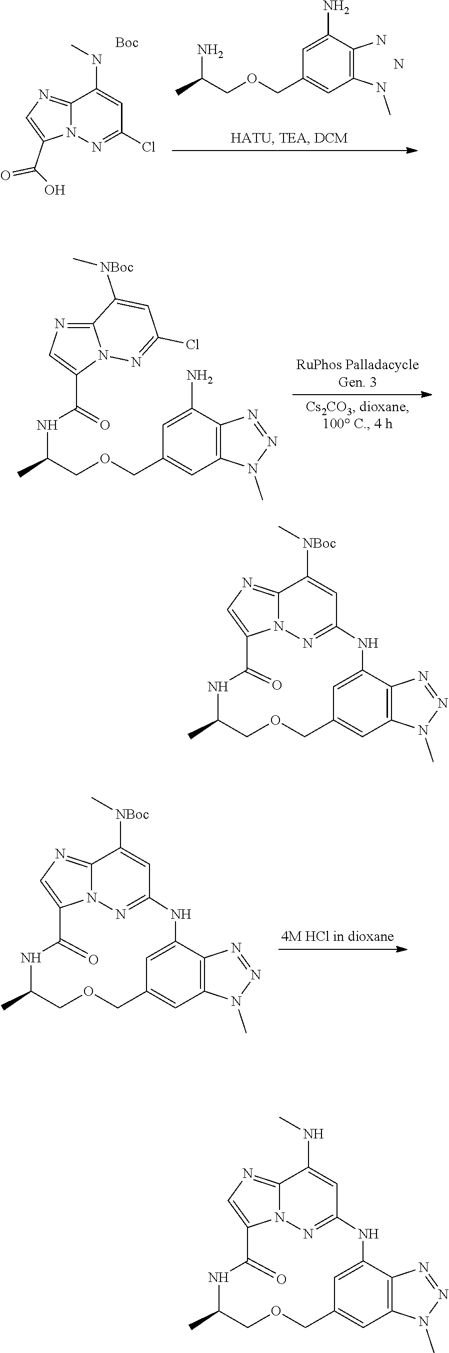

To a stirred solution of 8-[(tert-butoxycarbonyl)(methyl)amino]-6-chloroimidazo[1,2-b]pyridazine-3-carboxylic acid (750 mg, 2.3 mmol, 1 equiv) and 6-([(2R)-2-aminopropoxy]methyl-1-methyl-1,2,3-benzotriazol-4-amine (702 mg, 3 mmol, 1.3 equiv) in DMF (10 mL) were added DIEA (1480 mg, 11 mmol, 5 equiv) and HATU (1750 mg, 4.6 mmol, 2 equiv) in portions at room temperature under nitrogen atmosphere. The resulting mixture was stirred for 3 h at room temperature under nitrogen atmosphere. The desired product could be detected by LCMS. The residue was purified by reversed-phase flash chromatography with the following conditions: column, C18 silica gel; mobile phase, MeCN in water, 40% to 50% gradient in 10 min; detector, UV 254 nm. This resulted in tert-butyl N-[(14R)-7,14-dimethyl-16-oxo-12-oxa-2,5,6,7,15,19,23,24-octaazapentacyclo[15.5.2.1\hat{ }(3,10.0\hat{ }(4,8.0\hat{ }(20,24]pentacosa-1(23),3(25),4(8),5,9,17,19,21-octaen-21-yl]-N-methylcarbamate (830 mg, 71%) as a yellow solid.

To a stirred solution of tert-butyl N-(3-([(2R)-1-[(7-amino-3-methyl-1,2,3-benzotriazol-5-yl)methoxy]propan-2-yl]carbamoyl-6-chloroimidazo[1,2-b]pyridazin-8-yl)-N-methylcarbamate (810 mg, 1.5 mmol, 1 equiv) and RuPhos Palladacycle Gen.3 (124 mg, 0.15 mmol, 0.1 equiv) in dioxane (5 mL) was added Cs₂CO₃ (970 mg, 3 mmol, 2 equiv) in portions at room temperature under nitrogen atmosphere. The resulting mixture was stirred for 4 h at 100° C. under nitrogen atmosphere. The resulting mixture was concentrated under vacuum. The residue was purified by reversed-phase flash chromatography with the following conditions: column, C18 silica gel; mobile phase, MeCN in water, 50% to 60% gradient in 10 min; detector, UV 254 nm. This resulted in tert-butyl N-[(14R)-7,14-dimethyl-16-oxo-12-oxa-2,5,6,7,15,19,23,24-octaazapentacyclo[15.5.2.1\hat{ }(3,10.0\hat{ }(4,8.0\hat{ }(20,24]pentacosa-1(23),3(25),4(8),5,9,17,19,21-octaen-21-yl]-N-methylcarbamate (540 mg, 71%) as a yellow solid.

To a stirred solution of tert-butyl N-[(14R)-7,14-dimethyl-16-oxo-12-oxa-2,5,6,7,15,19,23,24-octaazapentacyclo[15.5.2.1\hat{ }(3,10.0\hat{ }(4,8.0\hat{ }(20,24]pentacosa-1(23),3(25),4(8),5,9,17,19,21-octaen-2l-yl]-N-methylcarbamate (490 mg, 1 mmol, 1 equiv) in dioxane was added 4 N HCl(gas)in 1,4-dioxane (5 mL) dropwise at room temperature under nitrogen atmosphere. The desired product could be detected by LCMS. The resulting mixture was concentrated under reduced pressure. The precipitated solids were collected by filtration and washed with diethyl ether (5×10 mL). This resulted in (14R)-7,14-dimethyl-21-(methylamino)-12-oxa-2,5,6,7,15,19,23,24-octaazapentacyclo[15.5.2.1\hat{ }(3,10.0\hat{ }(4,8.0\hat{ }(20,24]pentacosa-1(23),3(25),4(8),5,9,17,19,21-octaen-16-one (396 mg, 98%) as a yellow solid. LCMS: [M+H]⁺=408.05. NMR: 1H NMR (400 MHz, DMSO-d₆) δ 10.15 (s, 1H), 8.85 (d, J=5.7 Hz, 1H), 8.21-8.13 (m, 2H), 7.97 (s, 1H), 7.38-7.30 (m, 1H), 6.53 (s, 1H), 4.85 (d, J=14.1 Hz, 1H), 4.58 (d, J=14.2 Hz, 1H), 4.29 (s, 3H), 4.08 (ddt, J=8.7, 5.9, 3.1 Hz, 1H), 3.56 (dd, J=9.8, 2.7 Hz, 1H), 3.32 (t, J=9.5 Hz, 1H), 2.94 (s, 3H), 1.11 (d, J=6.5 Hz, 3H).

### Example 6: (1⁵E,3⁴E,7R)-3²,7-dimethyl-1⁸-(methylamino)-3²H-5-oxa-2,8-diaza-1(6,3),3(4,6)-diimidazo [1,2-b]pyridazinacyclononaphan-9-one (Compound 6)

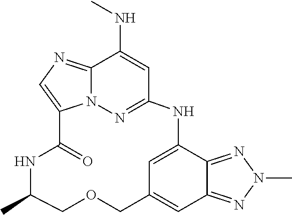

White solid. R_{f}=0.40 (DCM/MeOH, 95:5). Formula: C₁₉H₂₁N₉O₂. MW: 407.44. LCMS: [M+H]⁺=408.17. ¹H NMR (300 MHz, CDCl₃) δ 8.88 (d, J=5.6 Hz, 1H), 8.30 (s, 1H), 8.09 (s, 1H), 7.30 (s, 1H), 7.11 (s, 1H), 6.06 (d, J=5.4 Hz, 1H), 5.69 (s, 1H), 4.86 (d, J=13.8 Hz, 1H), 4.58 (d, J=13.9 Hz, 1H), 4.50 (s, 3H), 4.39-4.24 (m, 1H), 3.59 (dd, J=9.7, 2.7 Hz, 1H), 3.45 (t, J=9.1 Hz, 1H), 3.06 (d, J=5.1 Hz, 3H), 1.24 (s, 3H).

### Example 7: (7R,E)-3′,7-Dimethyl-1⁸-(methylamino)-3¹H-5-oxa-2,8-diaza-1(6,3),3(4,6)-diimidazo[1,2-b]pyridazinacyclononaphan-9-one-4,4-d₂ (Compound 7)

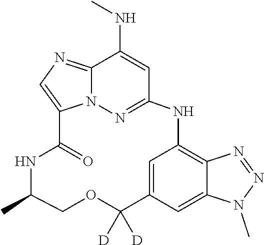 [SMILES: [2H]C1([2H])OC[C@@H](C)NC(=O)c2cnc3c(NC)cc(nn23)Nc2cc1cc1c2nnn1C]

White solid. R_{f}=0.40 (DCM/MeOH, 95:5). Formula: C₁₉H₁₉D₂N₉O₂. MW: 409.45. LCMS: [M+H]⁺=410.23. ¹H NMR (300 MHz, CDCl₃) δ 8.89 (d, J=5.8 Hz, 1H), 8.39 (s, 1H), 8.10 (s, 1H), 7.86 (s, 1H), 6.92 (s, 1H), 6.12 (q, J=4.7 Hz, 1H), 5.74 (s, 1H), 4.43-4.19 (m, 4H), 3.62 (dd, J=9.5, 2.7 Hz, 1H), 3.46 (t, J=9.2 Hz, 1H), 3.04 (d, J=5.1 Hz, 3H), 1.26 (d, J=6.5 Hz, 3H).

### Example 8: (7R,E)-3′,7-Dimethyl-1⁸-(methylamino)-3¹H-5-oxa-2,8-diaza-1(6,3)-imidazo[1,2-b] pyridazina-3(7,5)-benzo[d]imidazolacyclononaphan-9-one (Compound 8)

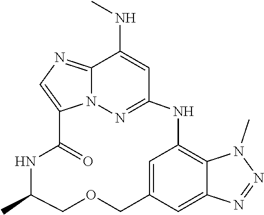 [SMILES: CNc1cc2nn3c(cnc13)C(=O)N[C@H](C)COCc1cc(c3c(c1)nnn3C)N2]

White solid. R_{f}=0.25 (DCM/MeOH, 95:5). Formula: C₂₀H₂₂N₈O₂. MW: 406.45. LCMS: [M+H]⁺=407.05. ¹H NMR (300 MHz, DMSO-d₆) δ 8.88 (s, 1H), 8.63 (d, J=5.1 Hz, 1H), 8.09 (s, 1H), 7.82 (s, 1H), 7.78 (s, 1H), 7.49 (q, J=4.6 Hz, 1H), 7.36 (s, 1H), 5.92 (s, 1H), 4.72 (d, J=12.7 Hz, 1H), 4.47 (d, J=12.9 Hz, 1H), 4.10 (s, 3H), 3.97-3.81 (m, 1H), 3.17 (d, J=5.2 Hz, 2H), 2.91 (d, J=4.8 Hz, 3H), 1.08 (d, J=6.4 Hz, 3H).

### Example 9: (R,1³E,1⁴E)-3¹,7-Dimethyl-1⁷-(methylamino)-3¹H-5-oxa-2,8-diaza-1(5,3)-pyrazolo[1,5-a]pyrimidina-3(7,5)-benzo[d]imidazolacyclononaphan-9-one (Compound 9)

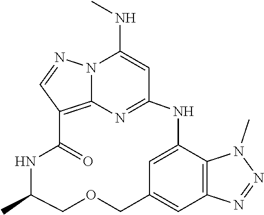 [SMILES: CNc1cc2nc3c(cnn13)C(=O)N[C@H](C)COCc1cc(c3c(c1)nnn3C)N2]

White solid. R_{f}=0.30 (DCM/MeOH, 95:5). Formula: C₂₀H₂₂N₈O₂. MW: 406.45. LCMS: [M+H]⁺=408.09. ¹H NMR (300 MHz, DMSO-d₆) δ 9.35 (s, 1H), 8.09 (s, 3H), 7.94-7.84 (m, 2H), 7.37 (d, J=1.4 Hz, 1H), 5.67 (s, 1H), 4.71 (d, J=12.7 Hz, 1H), 4.46 (d, J=12.7 Hz, 1H), 4.16-4.04 (m, 3H), 3.80 (s, 1H), 3.17 (d, J=5.3 Hz, 2H), 2.95 (d, J=4.8 Hz, 3H), 1.07 (d, J=6.4 Hz, 3H).

### Example 10: (7R,E)-3′,7-Dimethyl-1⁸-(methylamino)-3¹H-5-oxa-2,8-diaza-1(6,3)-imidazo[1,2-b]pyrida-zina-3(4,6)-benzo[d]imidazolacyclononaphan-9-one (Compound 10)

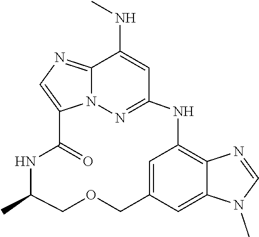 [SMILES: CNc1cc2nn3c(cnc13)C(=O)N[C@H](C)COCc1cc(c3ncn(C)c3c1)N2]

White solid. R_{f}=0.25 (DCM/MeOH, 95:5). Formula: C₂₀H₂₂N₈O₂. MW: 406.45. LCMS: [M+H]⁺=407.12. ¹H NMR (300 MHz, CDCl₃) δ 8.97 (d, J=5.5 Hz, 1H), 8.31 (d, J =1.2 Hz, 1H), 8.07 (s, 1H), 7.84 (s, 1H), 7.64 (s, 1H), 7.26 (s, 1H), 6.90 (s, 1H), 6.05 (t, J=5.4 Hz, 1H), 5.72 (s, 1H), 5.01-4.85 (m, 1H), 4.59 (d, J=13.8 Hz, 1H), 4.30 (q, J=6.2 Hz, 1H), 3.87 (d, J=3.3 Hz, 3H), 3.59 (dd, J=9.6, 2.7 Hz, 1H), 3.53-3.40 (m, 1H), 3.02 (d, J=5.1 Hz, 3H), 1.27 (s, 2H).

### Example 11: (R,1³E,1⁴E)-3′,7-Dimethyl-17-(methylamino)-3¹H-5-oxa-2,8-diaza-1(5,3)-pyrazolo[1,5-a]pyrimidina-3(4,6)-benzo[d]imidazolacyclononaphan-9-one (Compound 11)

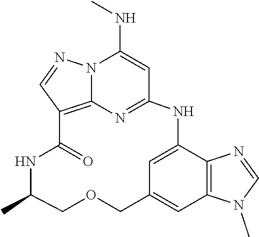 [SMILES: CNc1cc2nc3c(cnn13)C(=O)N[C@H](C)COCc1cc(c3ncn(C)c3c1)N2]

White solid. R_{f}=0.35 (DCM/MeOH, 95:5). Formula: C₂₀H₂₂N₈O₂. MW: 406.45. LCMS: [M+H]⁺=406.86. ¹H NMR (300 MHz, CDCl₃) δ 8.66 (s, 1H), 8.50 (d, J=5.7 Hz, 1H), 8.32 (s, 1H), 8.04 (s, 1H), 7.81 (s, 1H), 6.93 (s, 1H), 6.19 (d, J=5.7 Hz, 1H), 5.50 (s, 1H), 4.82 (d, J=13.5 Hz, 1H), 4.70 (d, J=13.6 Hz, 1H), 4.22 (dd, J=13.1, 6.7 Hz, 1H), 3.86 (s, 3H), 3.61 (dt, J=16.1, 8.9 Hz, 2H), 3.07 (d, J=5.2 Hz, 3H), 1.33 (d, J=6.5 Hz, 3H).

### Example 12: (7R,E)-3¹,7-Dimethyl-1⁸-(methylamino)-3¹H-5-oxa-2,8-diaza-1(6,3)-imidazo[1,2-b]pyridazina-3(4,6)-indazolacyclononaphan-9-one (Compound 12)

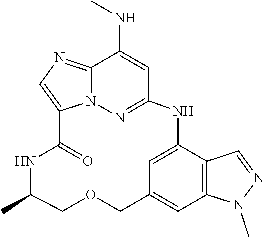 [SMILES: CNc1cc2nn3c(cnc13)C(=O)N[C@H](C)COCc1cc(c3cnn(C)c3c1)N2]

White solid. R_{f}=0.30 (DCM/MeOH, 95:5). Formula: C₂₀H₂₂N₈O₂. MW: 406.45. LCMS: [M+H]⁺=406.92. ¹H NMR (300 MHz, CDCl₃) δ 8.93 (d, J=5.5 Hz, 1H), 8.12 (s, 1H), 8.07 (s, 2H), 6.92 (s, 1H), 6.87 (s, 1H), 6.06 (s, 1H), 5.70 (s, 1H), 4.90 (d, J=13.7 Hz, 1H), 4.56 (d, J=13.8 Hz, 1H), 4.29 (dd, J=11.2, 7.0 Hz. 1H), 4.08 (s, 3H), 3.57 (dd, J=9.6, 2.7 Hz, 1H), 3.40 (t, J=9.3 Hz, 1H), 3.03 (d, J=5.1 Hz, 3H), 1.25 (d, J=6.5 Hz, 3H).

### Example 13: (R,1³E,1⁴E)-3′,7-Dimethyl-1⁷-(methylamino)-3¹H-5-oxa-2,8-diaza-1(5,3)-pyrazolo[1,5-a]pyrimidina-3(7,5)-indazolacyclononaphan-9-one (Compound 13)

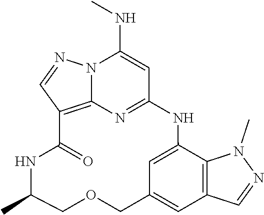 [SMILES: CNc1cc2nc3c(cnn13)C(=O)N[C@H](C)COCc1cc(c3c(cnn3C)c1)N2]

White solid. R_{f}=0.30 (DCM/MeOH, 95:5). Formula: C₂₀H₂₂N₈O₂. MW: 406.45. LCMS: [M+H]⁺=407.05. ¹H NMR (300 MHz, CDCl₃) δ 8.21 (d, J=4.8 Hz, 1H), 8.17 (s, 1H), 8.15 (s, 1H), 8.03 (s, 1H), 7.91 (s, 1H), 7.34 (s, 1H), 6.25 (q, J=5.1 Hz, 1H), 5.56 (s, 1H), 4.75 (d, J=12.7 Hz, 1H), 4.47 (d, J=12.9 Hz, 1H), 4.41 (s, 3H), 4.14-3.94 (m, 1H), 3.40 (dd, J=9.4, 2.7 Hz, 1H), 3.21 (t, J=8.9 Hz, 1H), 3.00 (d, J=5.2 Hz, 3H), 1.19 (d, J=6.4 Hz, 3H).

### Example 14: (7R,E)-3′,7-Dimethyl-1⁸-(methylamino)-3¹H-5-oxa-2,8-diaza-1(6,3)-imidazo[1,2-b]pyridazina-3(7,5)-indazolacyclononaphan-9-one (Compound 14)

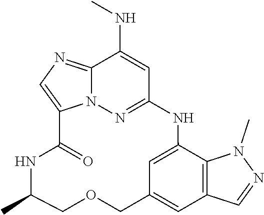 [SMILES: CNc1cc2nn3c(cnc13)C(=O)N[C@H](C)COCc1cc(c3c(cnn3C)c1)N2]

White solid. R_{f}=0.35 (DCM/MeOH, 95:5). Formula: C₂₀H₂₂N₈O₂. MW: 406.45. LCMS: [M+H]⁺=407.12. ¹H NMR 300 MHz, CDCl₃) δ 8.59 (d, J=5.1 Hz, 1H), 8.12 (s, 1H), 8.03 (s, 1H), 7.93 (s, 1H), 7.36 (s, 1H), 6.63 (s, 1H), 6.05 (s, 1H), 5.65 (s, 1H), 4.79 (d, J=12.9 Hz, 1H), 4.49 (d, J=12.9 Hz, 1H), 4.42 (s, 3H), 4.22-4.06 (m, 1H), 3.42 (dd, J=9.6, 2.7 Hz, 1H), 3.21 (t, J=9.1 Hz, 1H), 3.05 (d, J=4.3 Hz, 3H), 1.22 (d, J=6.5 Hz, 3H).

### Example 15: Synthesis of Compound 33

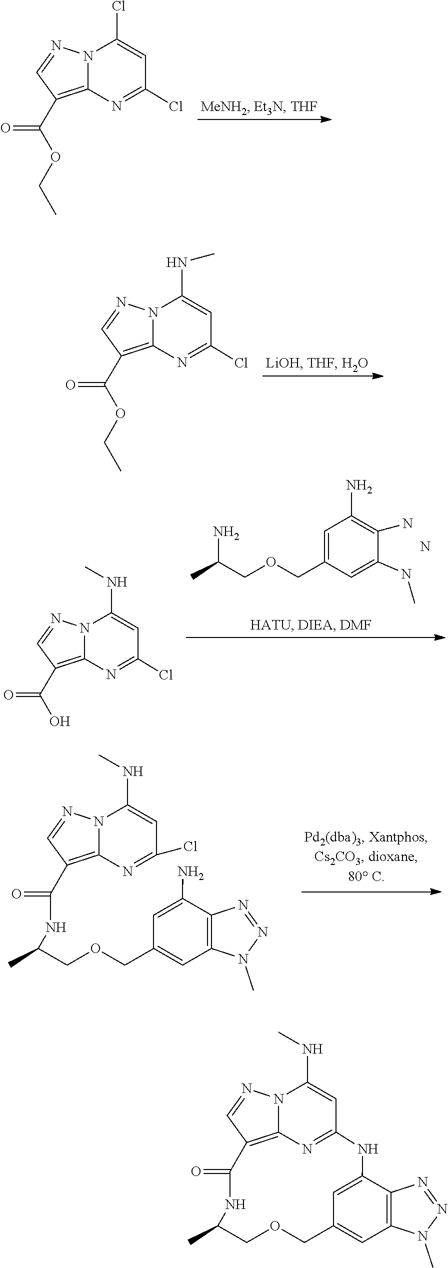

To a stirred mixture of ethyl 5,7-dichloropyrazolo[1,5-a]pyrimidine-3-carboxylate (3.5 g, 13.5 mmol, 1 equiv) and Et₃N (10.9 g, 108 mmol, 8 equiv) in THF (40 mL) was added MeNH₂(1677 mg, 54 mmol, 4 equiv) in portions at 0° C. The resulting mixture was stirred for overnight at room temperature under nitrogen atmosphere. The desired product could be detected by LCMS. The resulting mixture was concentrated under vacuum. The residue was purified by silica gel column chromatography, eluted with PE/EA (1:1) to afford ethyl 5-chloro-7-(methylamino)pyrazolo[1,5-a]pyrimidine-3-carboxylate (2.8 g, 82%) as a white solid.

To a stirred mixture of ethyl 5-chloro-7-(methylamino)pyrazolo[1,5-a]pyrimidine-3-carboxylate (3.4 g, 13.4 mmol, 1 equiv) in THF (35 mL) was added LiOH (639 mg, 26.7 mmol, 2 equiv) in H₂O (35 mL) at room temperature. The resulting mixture was stirred for overnight at 50° C. under nitrogen atmosphere. The residue was acidified to pH 5 with conc. HCl. The precipitated solids were collected by filtration and washed with water. The crude product mixture was used in the next step directly without further purification.

To a stirred mixture of 5-chloro-7-(methylamino)pyrazolo[1,5-a]pyrimidine-3-carboxylic acid (1 g, 4.4 mmol, 1 equiv), 6-([(2R)-2-aminopropoxy]methyl-1-methyl-1,2,3-benzotriazol-4-amine (1.3 g, 5.3 mmol, 1.2 equiv) in DMF (10 mL) was added HATU (2.5 g, 6.6 mmol, 1.5 equiv) and DIEA (4.56 g, 35.3 mmol, 8 equiv) at room temperature. The resulting mixture was stirred for 2 h at room temperature. The residue was purified by reversed-phase flash chromatography with the following conditions: column, C18 silica gel; mobile phase, MeCN in water, 10% to 95% gradient in 40 min; detector, UV 254 nm. This resulted in N-[(2R)-1-[(7-amino-3-methyl-1,2,3-benzotriazol-5-yl)methoxy]propan-2-yl]-5-chloro-7-(methylamino)pyrazolo[1,5-a]pyrimidine-3-carboxamide (1.1 g, 56%) as a light brown solid.

To a stirred mixture of N-[(2R)-1-[(7-amino-3-methyl-1,2,3-benzotriazol-5-yl)methoxy]propan-2-yl]-5-chloro-7-(methylamino)pyrazolo[1,5-a]pyrimidine-3-carboxamide (2 g, 4.5 mmol, 1 equiv) in dioxane (20 mL) was added Brettphos Pd G3 (408 mg, 0.45 mmol, 0.1 equiv), Brettphos (242 mg, 0.45 mmol, 0.1 equiv) and t-BuOK (1 g, 9 mmol, 2 equiv) at room temperature. The resulting mixture was stirred for overnight at 90° C. under nitrogen atmosphere. The desired product could be detected by LCMS. The residue was purified by silica gel column chromatography, eluted with PE/EA to afford (14R)-7,14-dimethyl-21-(methylamino)-12-oxa-2,5,6,7,15,19,20,23-octaazapentacyclo[15.5.2.1\hat{ }(3,10.0(4,8.0\hat{ }(20,24]pentacosa-1(23),3(25),4(8),5,9,17(24),18,21-octaen-16-one (2.2 g) as a white solid. The crude product (2.2 g) was purified by Chiral-HPLC with the following conditions (Column: CHIRAL ART Cellulose-SB, 2*25 cm, 5 m; Mobile Phase A: MtBE (10 mM NH3-MeOH), Mobile Phase B: EtOH—HPLC; Flow rate: 20 mL/min; Gradient: 30% B to 30% B in 15 min; Wave Length: 218/312 nm; RT1 (min): 7.2; RT2(min): 12.42; Sample Solvent: ETOH: DCM=1: 1; Injection Volume: 0.5 mL; Number Of Runs: 6) to afford (14R)-7,14-dimethyl-21-(methylamino)-12-oxa-2,5,6,7,15,19,20,23-octaazapentacyclo[15.5.2.1\hat{ }(3,10.0\hat{ }(4,8.0\hat{ }(20,24]pentacosa-1(23),3(25),4(8),5,9,17(24),18,21-octaen-16-one (469 mg, 25%) as a white solid. LCMS: [M+H]⁺=408.18. NMR: 1H NMR (400 MHz, DMSO-d6) δ 10.33 (s, 1H), 8.53 (d, J=1.2 Hz, 1H), 8.37 (d, J=5.4 Hz, 1H), 8.18 (s, 1H), 7.99 (q, J=4.8 Hz, 1H), 7.32 (d, J=1.1 Hz, 1H), 6.11 (s, 1H), 4.78 (d, J=13.9 Hz, 1H), 4.65 (d, J=13.9 Hz, 1H), 4.29 (s, 3H), 3.97 (q, J=6.3 Hz, 1H), 3.58 (dd, J=9.5, 2.6 Hz, 1H), 3.42 (dd, J=9.5, 7.3 Hz, 1H), 2.95 (d, J=4.8 Hz, 3H), 1.15 (d, J=6.5 Hz, 3H).

### Example 16: Synthesis of Compound 54

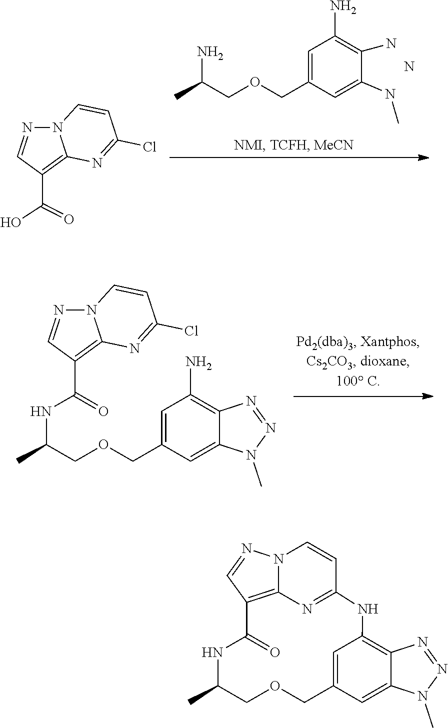

To a stirred mixture of 5-chloropyrazolo[1,5-a]pyrimidine-3-carboxylic acid (500 mg, 2.5 mmol, 1 equiv) and 6-([(2R)-2-aminopropoxy]methyl-1-methyl-1,2,3-benzotriazol-4-amine (893 mg, 3.8 mmol, 1.5 equiv) in MeCN (10 ml) were added NMI (644 mg, 7.8 mmol, 3.1 equiv) and TCFH (781 mg, 2.8 mmol, 1.1 equiv) in portions at room temperature under argon atmosphere. The resulting mixture was stirred for 3 h at room temperature under argon atmosphere. The desired product could be detected by LCMS. The resulting mixture was concentrated under reduced pressure. The residue was purified by reversed-phase flash chromatography with the following conditions: column, C18 silica gel; mobile phase, MeCN in water, 10% to 50% gradient in 10 min; detector, UV 254 nm. to afford N-[(2R)-1-[(7-amino-3-methyl-1,2,3-benzotriazol-5-yl)methoxy]propan-2-yl]-5-chloropyrazolo[1,5-a]pyrimidine-3-carboxamide (200 mg, 19%) as a yellow solid.

To a stirred mixture of N-[(2R)-1-[(7-amino-3-methyl-1,2,3-benzotriazol-5-yl)methoxy]propan-2-yl]-5-chloropyrazolo[1,5-a]pyrimidine-3-carboxamide (210 mg, 0.5 mmol, 1 equiv) and Cs₂CO₃ (330 mg, 1 mmol, 2 equiv) in 1,4-dioxane (4 ml) were added Pd₂(dba)₃ (46 mg, 0.05 mmol, 0.1 equiv) and Xantphos (58 mg, 0.1 mmol, 0.2 equiv) in portions at room temperature under air atmosphere. The resulting mixture was stirred for 2 h at 100° C. under nitrogen atmosphere. The desired product could be detected by LCMS. The resulting mixture was concentrated under reduced pressure. The crude product (110 mg) was purified by Prep-HPLC with the following conditions (Column: Xselect CSH C18 OBD Column 30*150 mm 5 m, n; Mobile Phase A: water (0.1% FA), Mobile Phase B: ACN; Flow rate: 60 mL/min mL/min; Gradient: 14% B to 30% B in 8 min; Wave Length: 254 nm/220 nm; RT1(min): 9.35) to afford (14R)-7,14-dimethyl-12-oxa-2,5,6,7,15,19,20,23-octaazapentacyclo[15.5.2.1\hat{ }(3,10.0\hat{ }(4,8.0\hat{ }(20,24]pentacosa-1(23),3,5,8,10(25),17(24),18,21-octaen-16-one (60.1 mg, 31%) was obtained as a white solid. LCMS: [M+H]⁺=379.00. NMR: ¹H NMR (400 MHz, DMSO-d₆) δ 11.01 (s, 1H), 8.90 (dd, J=7.6, 0.9 Hz, 1H), 8.62 (s, 1H), 8.24 (s, 1H), 8.17 (d, J=5.6 Hz, 1H), 7.45 (s, 1H), 7.08 (d, J=7.6 Hz, 1H), 4.79 (d, J=13.8 Hz, 1H), 4.70 (d, J=13.8 Hz, 1H), 4.31 (s, 3H), 4.00 (q, J=7.5, 6.8 Hz, 1H), 3.62 (dd, J=9.5, 2.6 Hz, 1H), 3.46 (dd, J=9.4, 6.9 Hz, 1H), 1.17 (d, J=6.5 Hz, 3H).

### Example 17: Synthesis of Compound 55

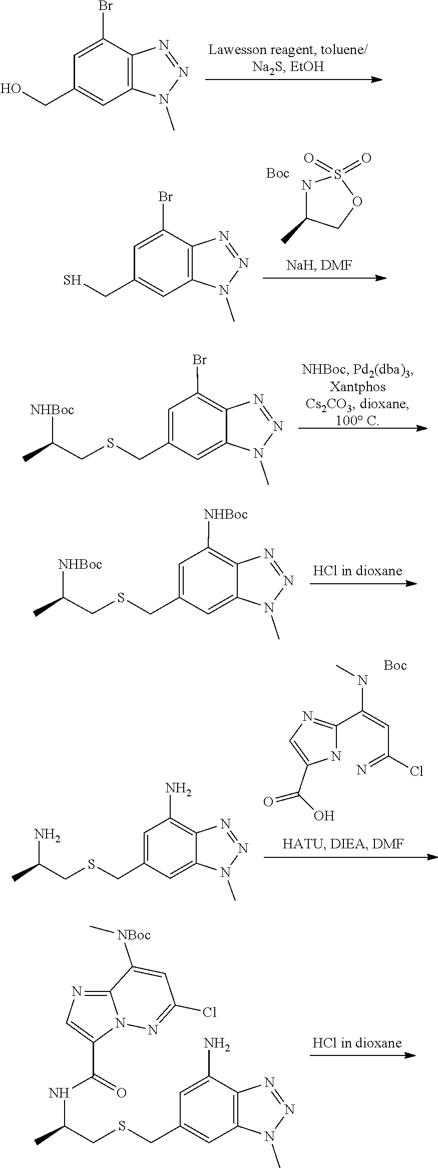

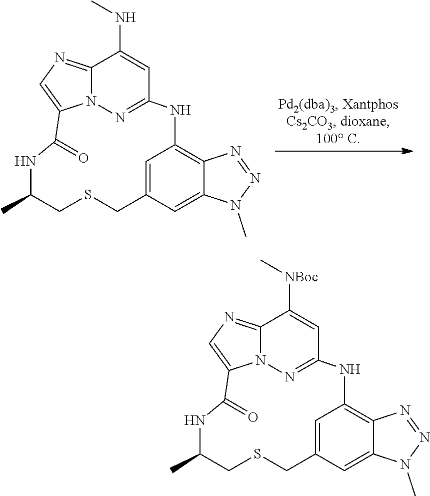

A solution of (7-bromo-3-methyl-1,2,3-benzotriazol-5-yl)methanol (2 g, 8.3 mmol, 1 equiv) and Lawesson reagent (6.7 g, 16.5 mmol, 2 equiv) in toluene (20 mL) was stirred for overnight at 100° C. under argon atmosphere. The desired product could be detected by LCMS. The resulting mixture was concentrated under vacuum. The residue was purified by silica gel column chromatography, eluted with PE/EA (1:1) to afford (7-bromo-3-methyl-1,2,3-benzotriazol-5-yl)methanethiol (940 mg, 44%) as a white solid.

A solution of (7-bromo-3-methyl-1,2,3-benzotriazol-5-yl)methanethiol (710 mg, 2.8 mmol, 1 equiv) and NaH (110 mg, 2.8 mmol, 1 equiv, 60% in oil) in DMF (5 mL) was stirred for 5 min at room temperature under argon atmosphere. To the above mixture was added tert-butyl (4R)-4-methyl-2,2-dioxo-1,2lambda6,3-oxathiazolidine-3-carboxylate (45.9 mg, 0.19 mmol, 1 equiv) in DMF (2 mL) dropwise at room temperature. The resulting mixture was stirred for additional 30 min at room temperature. The above mixture was dropped into the mixture of H₂SO₄ (20% in H₂O) (8 mL) and DCM (8 mL). The resulting mixture was stirred for 30 min at room temperature. The desired product could be detected by LCMS. The resulting mixture was concentrated under vacuum. The residue was purified by reversed-phase flash chromatography with the following conditions: column, C18 silica gel; mobile phase, MeCN in water, 40% to 95% gradient in 30 min; detector, UV 254 nm. This resulted in tert-butyl N-[(2R)-1-([(7-bromo-3-methyl-1,2,3-benzotriazol-5-yl)methyl]sulfanylpropan-2-yl]carbamate (410 mg, 35%) as an off-white solid.

To a stirred solution of tert-butyl N-[(2R)-1-([(7-bromo-3-methyl-1,2,3-benzotriazol-5-yl)methyl]sulfanylpropan-2-yl]carbamate (760 mg, 1.8 mmol, 1 equiv), tert-butyl carbamate (321 mg, 2.7 mmol, 1.5 equiv) and tert-butyl carbamate (321 mg, 2.7 mmol, 1.5 equiv), Cs₂CO₃ (1788 mg, 5.5 mmol, 3 equiv) in 1,4-dioxane (8 mL) were added Pd₂(dba)₃ (167 mg, 0.18 mmol, 0.1 equiv) and Xantphos (211 mg, 0.37 mmol, 0.2 equiv) in portions at room temperature under argon atmosphere. The resulting mixture was stirred for 2 h at 100° C. under argon atmosphere. The desired product could be detected by LCMS. The residue was purified by silica gel column chromatography, eluted with PE/EA (5:1) to afford tert-butyl N-[6-(([(2R)-2-[(tert-butoxycarbonyl)amino]propyl]sulfanylmethyl)-1-methyl-1,2,3-benzotriazol-4-yl]carbamate (414 mg, 50%) as an orange oil.

A solution of tert-butyl N-[6-(([(2R)-2-[(tert-butoxycarbonyl)amino]propyl]sulfanylmethyl)-1-methyl-1,2,3-benzotriazol-4-yl]carbamate (400 mg, 0.89 mmol, 1 equiv) and 4M HCl(gas) in 1,4-dioxane (10 mL) was stirred for overnight at room temperature under argon atmosphere. The desired product could be detected by LCMS. The resulting mixture was concentrated under vacuum. The crude product was used in the next step directly without further purification.

To a stirred solution of 6-(([(2R)-2-aminopropyl]sulfanylmethyl)-1-methyl-1,2,3-benzotriazol-4-amine (270 mg, 1.1 mmol, 1 equiv) and 8-[(tert-butoxycarbonyl)(methyl)amino]-6-chloroimidazo[1,2-b]pyridazine-3-carboxylic acid (350 mg, 1.1 mmol, 1 equiv) in DMF (5 mL) were added DIEA (1110 mg, 8.6 mmol, 8 equiv) and HATU (612 mg, 1.6 mmol, 1.5 equiv) in portions at room temperature under argon atmosphere. The resulting mixture was stirred for 2 h at room temperature under argon atmosphere. The desired product could be detected by LCMS. The residue was purified by reversed-phase flash chromatography with the following conditions: column, C18 silica gel; mobile phase, MeCN in water, 30% to 80% gradient in 20 min; detector, UV 254 nm. This resulted in tert-butyl N-(3-([(2R)-1-([(7-amino-3-methyl-1,2,3-benzotriazol-5-yl)methyl]sulfanylpropan-2-yl]carbamoyl-6-chloroimidazo[1,2-b]pyridazin-8-vl)-N-methylcarbamate (350 mg, 58%) as an off-white solid.

A solution of tert-butyl N-(3-([(2R)-1-([(7-amino-3-methyl-1,2,3-benzotriazol-5-yl)methyl]sulfanylpropan-2-yl]carbamoyl-6-chloroimidazo[1,2-b]pyridazin-8-yl)-N-methylcarbamate (330 mg, 0.59 mmol, 1 equiv), RuPhos Palladacycle Gen.3 (98.6 mg, 0.12 mmol, 0.2 equiv) and Cs₂CO₃ (383 mg, 1.2 mmol, 2 equiv) in 1,4-dioxane (10 mL) was stirred for 4 h at 100° C. under argon atmosphere. The desired product could be detected by LCMS. The residue was purified by reversed-phase flash chromatography with the following conditions: column, C18 silica gel; mobile phase, MeCN in water, 30% to 80% gradient in 30 min; detector, UV 254 nm. This resulted in tert-butyl N-[(14R)-7,14-dimethyl-16-oxo-12-thia-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaen-21-yl]-N-methylcarbamate (202 mg, 65%) as an off-white solid.

A solution of tert-butyl N-[(14R)-7,14-dimethyl-16-oxo-12-thia-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaen-21-yl]-N-methylcarbamate (40 mg, 0.08 mmol, 1 equiv) and 4M HCl(gas) in 1,4-dioxane (2.0 mL) was stirred for 2 h at room temperature under argon atmosphere. The desired product could be detected by LCMS. The resulting mixture was concentrated under vacuum. The crude product (60 mg) was purified by Prep-HPLC with the following conditions (Column: YMC-Actus Triart C18 ExRS 30*150 mm, 5 μm; Mobile Phase A: water (10 mmol/L NH4HCO3), Mobile Phase B: ACN; Flow rate: 60 mL/min mL/min; Gradient: 21% B to 38% B in 10 min; Wave Length: 254 nm/220 nm; RT1(min): 11.4) to afford (14R)-7,14-dimethyl-21-(methylamino)-12-thia-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaen-16-one (22.7 mg, 69%) as a white solid. LCMS:[M+H]′=424.10. NMR:1H NMR (400 MHz, DMSO-d6) δ 9.86 (s, 1H), 8.76 (d, J=6.5 Hz, 1H), 8.29 (d, J=1.3 Hz, 1H), 7.86 (s, 1H), 7.59 (q, J=4.6 Hz, 1H), 7.30 (s, 1H), 6.32 (s, 1H), 4.27 (s, 3H), 4.06 (d, J=15.9 Hz, 1H), 3.94 (d, J=15.6 Hz, 1H), 3.89 (s, 1H), 2.95 (dd, J=12.2, 2.4 Hz, 1H), 2.91 (d, J=4.8 Hz, 3H), 2.10 (t, J=12.2 Hz, 1H), 1.11 (d, J=6.3 Hz, 3H).

### Example 18: Synthesis of Compound 56

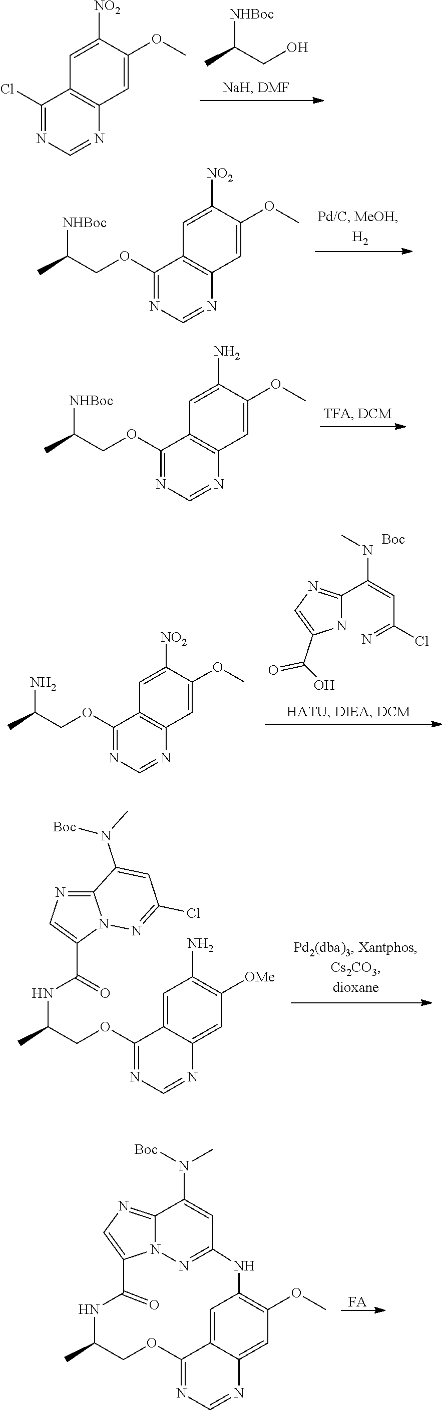

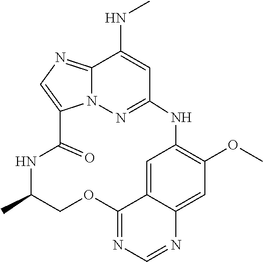

A solution of 4-chloro-7-methoxy-6-nitroquinazoline (1.4 g, 5.8 mmol, 1 equiv) in DMF (10 mL) was treated with tert-butyl N-[(2R)-1-hydroxypropan-2-yl]carbamate (1.02 g, 5.8 mmol, 1 equiv) followed by the addition of NaH (11 mg, 0.5 mmol, 1.1 equiv) for 2 h at room temperature. The desired product could be detected by LCMS. The reaction was quenched with water at room temperature. The resulting mixture was extracted with EtOAc (3×50 mL) dried over anhydrous Na₂SO₄. After filtration, the filtrate was concentrated under reduced pressure. The residue was purified by silica gel column chromatography, eluted with PE/EA (3:7) to afford tert-butyl N-[(2R)-1-[(7-methoxy-6-nitroquinazolin-4-yl) oxy]propan-2-yl]carbamate (1.5 g, 68%) as a yellow solid.

A solution of tert-butyl N-[(2R)-1-[(7-methoxy-6-nitroquinazolin-4-yl) oxy]propan-2-yl]carbamate (1.6 g, 4.2 mmol, 1 equiv) and Palladium (319 mg, 3 mmol, 0.7 equiv) in MeOH (20 mL) was stirred for overnight at room temperature under hydrogen atmosphere. The desired product could be detected by LCMS. The resulting mixture was filtered, the filter cake was washed with MeOH (3×50 mL). The filtrate was concentrated under reduced pressure. This resulted in tert-butyl N-[(2R)-1-[(6-amino-7-methoxyquinazolin-4-yl) oxy]propan-2-yl]carbamate (900 mg, 61%) as a light yellow solid. The crude product was used in the next step directly without further purification.

A solution of tert-butyl N-[(2R)-1-[(6-amino-7-methoxyquinazolin-4-yl) oxy]propan-2-yl]carbamate (450 mg, 1.3 mmol, 1 equiv) and TFA (2.5 mL) in DCM (5 mL) was stirred for 2 h at room temperature under air atmosphere. The desired product could be detected by LCMS. The resulting mixture was concentrated under vacuum. The crude product mixture was used in the next step directly without further purification.

A solution of 4-[(2S)-2-aminopropoxy]-7-methoxyquinazolin-6-amine (500 mg, 2.0 mmol, 1 equiv) in DCM (20 mL) was treated with DIEA (1.6 g, 12.1 mmol, 6 equiv) followed by the addition of HATU (1.2 g, 3 mmol, 1.5 equiv) and 8-[(tert-butoxycarbonyl) (methyl)amino]-6-chloroimidazo[1,2-b]pyridazine-3-carboxylic acid (724 mg, 2.2 mmol, 1.1 equiv). The resulting mixture was stirred for 2 h. The desired product could be detected by LCMS. The resulting mixture was concentrated under vacuum. The residue was dissolved in acetonitrile (5 mL). The residue was purified by reversed-phase flash chromatography with the following conditions: column, C18 silica gel; mobile phase, MeCN in water, 40% to 60% gradient in 10 min; detector, UV 254 nm. This resulted in tert-butyl N-(3-([(2R)-1-[(6-amino-7-methoxyquinazolin-4-yl) oxy]propan-2-yl]carbamoyl-6-chloroimidazo[1,2-b]pyridazin-8-yl)-N-methylcarbamate (400 mg, 36%) as a light yellow solid.

A solution of tert-butyl N-(3-([(2R)-1-[(6-amino-7-methoxyquinazolin-4-yl) oxy]propan-2-yl]carbamoyl-6-chloroimidazo[1,2-b]pyridazin-8-yl)-N-methylcarbamate (400 mg, 0.7 mmol, 1 equiv) in 1,4-dioxane (10 mL) was treated with Cs2CO₃ (701 mg, 2.1 mmol, 3 equiv) followed by the addition of Xantphos (416 mg. 0.7 mmol, 1 equiv) and Pd₂(dba)₃ (329 mg, 0.4 mmol, 0.5 equiv) at room temperature. The resulting mixture was stirred at 100° C. for overnight. The desired product could be detected by LCMS. The resulting mixture was filtered, the filter cake was washed with acetonitrile (3×20 mL). The filtrate was concentrated under reduced pressure. The residue was purified by reversed-phase flash chromatography with the following conditions: column, C18 silica gel; mobile phase, MeCN in water, 50% to 60% gradient in 10 min; detector, UV 254 nm. This resulted in tert-butyl N-[(12R)-21-methoxy-12-methyl-10-oxo-14-oxa-2,4,5,7,11,16,18-heptaazapentacvclo [13.6.2.2\hat{ }(3,6.0\hat{ }(5,9.0\hat{ }(19,23]pentacosa-1(21),3,6,8,15,17,19,22,24-nonaen-24-yl]-N-methylcarbamate (160 mg, 43%) as a light yellow solid.

A solution of tert-butyl N-[(12R)-21-methoxy-12-methyl-10-oxo-14-oxa-2,4,5,7,11,16,18-heptaazapentacyclo [13.6.2.2\hat{ }(3,6.0\hat{ }(5,9.0\hat{ }(19,23]pentacosa-1(21),3,6,8,15,17,19,22,24-nonaen-24-yl]-N-methylcarbamate (50 mg, 0.1 mmol, 1 equiv) in formic acid (3 mL) for 1 h at 40° C. under air atmosphere. The mixture was neutralized to pH 6 with saturated NaHCO₃. The desired product could be detected by LCMS. The crude product was purified by Prep-HPLC with the following conditions (Column: XBridge Shield RP18 OBD Column 30*150 mm, 5m; Mobile Phase A: water (10 mmol/L NH4HCO3), Mobile Phase B: ACN; Flow rate: 60 mL/min mL/min; Gradient: isocratic 17% B to 35% B in 10 min; Wave Length: 254 nm/220 nm; RT1(min): 10.8) to afford (12R)-21-methoxy-12-methyl-24-(methylamino)-14-oxa-2,4,5,7,11,16,18-heptaazapentacyclo[13.6.2.2\hat{ }(3,6.0\hat{ }(5,9.0\hat{ }(19,23]pentacosa-1(21),3,6,8,15,17,19,22,24-nonaen-10-one (2 mg, 5%) as a white solid. LCMS: [M+H]⁺=421.20. NMR: ¹H NMR (400 MHz, DMSO-d₆) δ 9.25 (s, 1H), 9.06 (s, 1H), 8.75 (s, 1H), 8.69 (d, J=8.6 Hz, 1H), 7.89 (s, 1H), 7.54 (d, J=5.0 Hz, 1H), 7.41 (s, 1H), 6.39 (s, 1H), 4.82 (dd, J=10.8, 2.4 Hz, 1H), 4.53 (s, 1H), 4.33 (dd. J=10.8, 3.5 Hz, 1H), 4.11 (s, 3H), 2.90 (d, J=4.8 Hz, 3H), 0.99 (d, J=6.8 Hz, 3H).

### Example 19: Synthesis of Compound 57

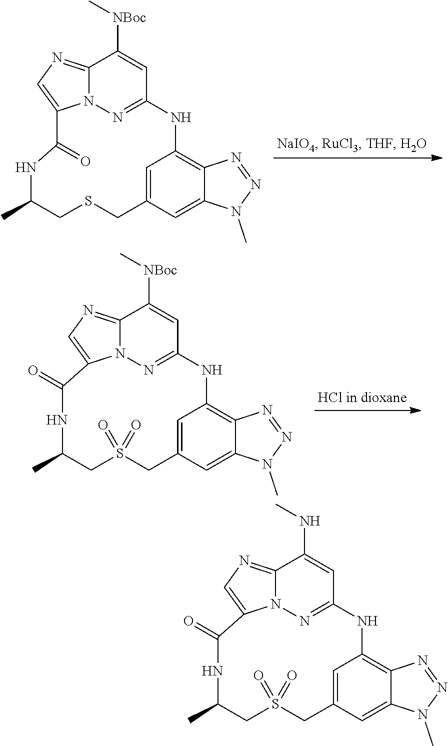

A solution of tert-butyl N-[(14R)-7,14-dimethyl-16-oxo-12-thia-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaen-2l-yl]-N-methylcarbamate (50 mg, 0.1 mmol, 1 equiv), NaIO4 (61.3 mg, 0.29 mmol, 3 equiv) and RuCl₃·H₂O (4.3 mg, 0.02 mmol, 0.2 equiv) in THF/H₂O (3 mL/3 mL) was stirred for 2 h at room temperature under argon atmosphere. The desired product could be detected by LCMS. The resulting mixture was concentrated under vacuum. The residue was purified by reversed-phase flash chromatography with the following conditions: column, C18 silica gel; mobile phase, MeCN in water, 10% to 50% gradient in 30 min; detector, UV 254 nm. This resulted in tert-butyl N-[(14R)-7,14-dimethyl-12,12,16-trioxo-12lambda6-thia-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaen-21-yl]-N-methylcarbamate (42 mg, 79%) as an off-white solid.

A solution of tert-butyl N-[(14R)-7,14-dimethyl-12,12,16-trioxo-12lambda6-thia-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaen-21-yl]-N-methylcarbamate (42 mg, 0.08 mmol, 1 equiv) and 4M HCl in 1,4-dioxane (2.1 mL) was stirred for 2 h at room temperature under argon atmosphere. The desired product could be detected by LCMS. The resulting mixture was concentrated under vacuum. The crude product (30 mg) was purified by Prep-HPLC with the following conditions (Column: XBridge Shield RP18 OBD Column 30*150 mm, 5 m; Mobile Phase A: water (10 mmol/L NH4HCO3), Mobile Phase B: ACN; Flow rate: 60 mL/min m/min; Gradient: 14% B to 34% B in 8 min; Wave Length: 254 nm/220 nm; RT1(min): 8.22) to afford (14R)-7,14-dimethyl-21-(methylamino)-12lambda6-thia-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaene-12,12,16-trione (16.4 mg, 47%) as a white solid. LCMS:[M+H]⁺=456.05. NMR: 1H NMR (400 MHz, DMSO-d₆) δ 9.98 (s, 1H), 8.74 (d, J=5.7 Hz, 1H), 8.32 (d, J=1.2 Hz, 1H), 7.83 (s, 1H), 7.59 (q, J=4.8 Hz, 1H), 7.39 (d, J=1.3 Hz, 1H), 6.30 (s, 1H), 4.80 (d, J=15.1 Hz, 1H), 4.64 (d, J=15.0 Hz, 1H), 4.31 (s, 3H), 3.06-2.97 (m, 1H), 2.91 (d, J=4.8 Hz, 3H), 1.26 (d, J=6.5 Hz, 3H).

### Example 20: Synthesis of Compound 58

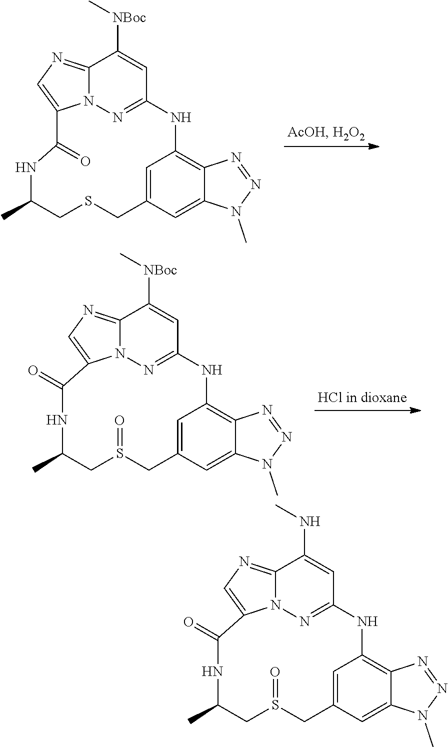

To a stirred solution of tert-butyl N-[(14R)-7,14-dimethyl-16-oxo-12-thia-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaen-2l-yl]-N-methylcarbamate (50 mg, 0.10 mmol, 1 equiv) and AcOH (2.5 mL, 43.6 mmol) was added H₂O₂(30%) (1.0 mL) dropwise at room temperature under argon atmosphere. The resulting mixture was stirred for 5 h at room temperature under argon atmosphere. The desired product could be detected by LCMS. The residue was purified by reversed-phase flash chromatography with the following conditions: column, C18 silica gel; mobile phase, MeCN in water, 10% to 50% gradient in 30 min; detector, UV 254 nm. This resulted in tert-butyl N-[(14R)-7,14-dimethyl-12,16-dioxo-12lambda4-thia-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaen-21-yl]-N-methylcarbamate (40 mg, 77%) as an off-white solid.

A solution of tert-butyl N-[(14R)-7,14-dimethyl-12,16-dioxo-12lambda4-thia-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaen-2l-yl]-N-methylcarbamate (40 mg, 0.07 mmol, 1 equiv) and 4M HCl(gas) in 1,4-dioxane (2.0 mL) was stirred for 2 h at room temperature under argon atmosphere. The desired product could be detected by LCMS. The resulting mixture was concentrated under vacuum. The crude product (40 mg) was purified by Prep-HPLC with the following conditions (Column: XBridge Prep Phenyl OBD Column 19*250 mm, 5 m; Mobile Phase A: water (10 mmol/L NH4HCO3+0.1% NH3·H2O), Mobile Phase B: ACN; Flow rate: 60 mL/min mL/min; Gradient: 10% B to 26% B in 8 min; Wave Length: 254 nm/220 nm; RT1(min): 18.61) to afford (14R)-7,14-dimethyl-21-(methylamino)-12lambda4-thia-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaene-12,16-dione (8 mg, 24%) as a white solid. LCMS :[M+H]⁺=440.10. NMR: 1H NMR (400 MHz, DMSO-d6) δ 9.90 (s, 1H), 8.54 (d, J=7.3 Hz, 1H), 7.87 (s, 1H), 7.62 (q, J=4.8 Hz, 1H), 7.52 (d, J=1.2 Hz, 1H), 7.39 (s, 1H), 6.25 (s, 1H), 4.74 (d, J=13.8 Hz, 1H), 4.48-4.39 (m, 1H), 4.30 (s, 3H), 4.19 (d, J=14.0 Hz, 1H), 2.91 (d, J=4.9 Hz, 3H), 2.83 (d, J=13.3 Hz, 1H), 2.69 (dd, J=14.1, 2.5 Hz, 1H), 1.16 (d, J=6.4 Hz, 3H).

### Example 21: Synthesis of Compound 59

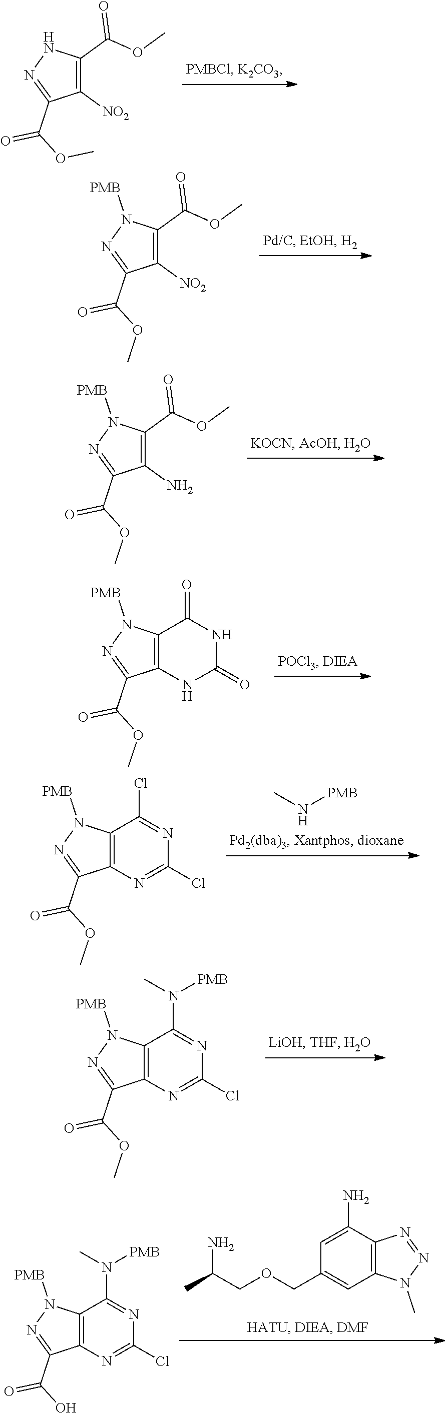

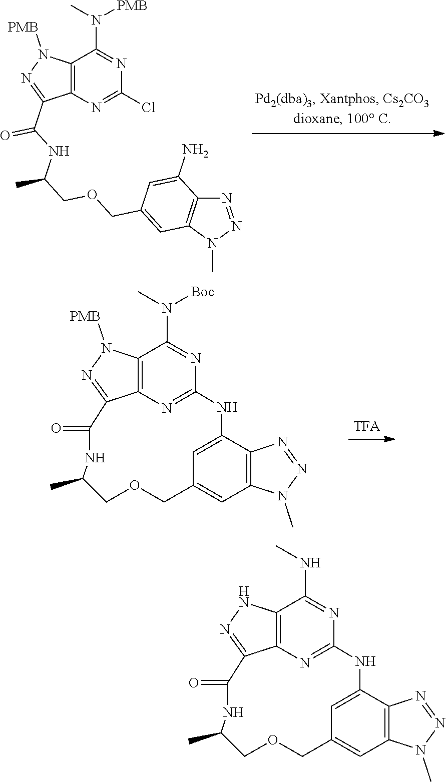

A solution of 3,5-dimethyl 4-nitro-TH-pyrazole-3,5-dicarboxylate (5.4 g, 23.6 mmol, 1 equiv) in DMF (50 mL) was treated with K₂CO₃ (7.8 g, 56.6 mmol, 2.4 equiv) at room temperature under air atmosphere followed by the addition of PMBCl (4.43 g, 28.3 mmol, 1.2 equiv) dropwise at room temperature. The resulting mixture was stirred for 4 h at 50′C under nitrogen atmosphere. The desired product could be detected by LCMS. The reaction was quenched with water at room temperature. The aqueous layer was extracted with EtOAc (3×20 mL). The organic layers were combined, dried and concentrated under vacuum. The residue was purified by reversed-phase flash chromatography with the following conditions: column, C18 silica gel; mobile phase, MeCN in water, 10% to 50% gradient in 10 min; detector, UV 254 nm. to afford 3,5-dimethyl 1-[(4-methoxyphenyl)methyl]-4-nitropyrazole-3,5-dicarboxylate (5 g, 61%) as a white liquid.

To a stirred mixture of 3,5-dimethyl 1-[(4-methoxyphenyl)methyl]-4-nitropyrazole-3,5-dicarboxylate (5 g, 14.3 mmol, 1 equiv) in EtOH (50 mL) was added Pd/C (10%, 5.00 g,) in portions at room temperature. The resulting mixture was stirred for 1 h at room temperature under hydrogen atmosphere. The desired product could be detected by LCMS. The resulting mixture was filtered. The filtrate was concentrated under reduced pressure. The resulting mixture was concentrated under reduced pressure. The residue was purified by reversed-phase flash chromatography with the following conditions: column, C18 silica gel; mobile phase, MeCN in water, 10% to 50% gradient in 10 min; detector, UV 254 nm. to afford 3,5-dimethyl 4-amino-1-[(4-methoxyphenyl)methyl]pyrazole-3,5-dicarboxylate (4 g, 88%) as a white solid.

To a stirred solution of 3,5-dimethyl 4-amino-1-[(4-methoxyphenyl)methyl]pyrazole-3,5-dicarboxylate (3 g, 9.4 mmol, 1 equiv) in AcOH (50 mL) was added potassium cyanate (3.05 g, 37.6 mmol, 4 equiv) in H₂O (1 ml) dropwise at room temperature under nitrogen atmosphere. The resulting mixture was stirred for overnight at room temperature under nitrogen atmosphere. The desired intermediate could be detected by LCMS. To the stirred mixture was added H₂O (6 mL) at room temperature under air atmosphere. The precipitated solids were collected by filtration and washed with H₂O (3×100 mL). This resulted in intermediates (2.5 g) as a white solid. To the stirred intermediate (2.5 g) in MeOH (50 mL) was added NaOH (0.45 g, 11.274 mmol, 1.2 equiv) at room temperature under nitrogen atmosphere. The resulting mixture was stirred for 20 h at room temperature under nitrogen atmosphere. The desired product could be detected by LCMS. The mixture was acidified to pH 3 with HCl (aq.) (2M). The precipitated solids were collected by filtration and washed with water (3×20 mL). The residue was purified by reversed-phase flash chromatography with the following conditions: column, C18 silica gel; mobile phase, MeOH in water, 20% to 100% gradient in 50 min; detector, UV 254 nm. This resulted in methyl 1-[(4-methoxyphenyl)methyl]-5,7-dioxo-4H,6H-pyrazolo[4,3-d]pyrimidine-3-carboxylate (2 g, 64%) as a light brown solid.

To a stirred mixture of methyl 1-[(4-methoxyphenyl)methyl]-5,7-dioxo-4H,6H-pyrazolo[4,3-d]pyrimidine-3-carboxylate (2.4 g, 7.266 mmol, 1 equiv) in POCl3 (20 mL) was added DIEA (7.6 mL, 43.6 mmol, 6.00 equiv) dropwise at room temperature under air atmosphere. The resulting mixture was stirred for overnight at 100° C. under nitrogen atmosphere. The solvent was removed under reduced pressure. The reaction was quenched with water at 0° C. The resulting mixture was concentrated under vacuum. The residue was purified by reversed-phase flash chromatography with the following conditions: column, C18 silica gel; mobile phase, MeCN in water, 10% to 50% gradient in 10 min; detector, UV 254 nm. to afford methyl 5,7-dichloro-1-[(4-methoxyphenyl)methyl]pyrazolo[4,3-d]pyrimidine-3-carboxylate (550 mg, 21%) as a yellow solid.

To a stirred mixture of methyl 5,7-dichloro-1-[(4-methoxyphenyl)methyl]pyrazolo[4,3-d]pyrimidine-3-carboxylate (600 mg, 1.634 mmol, 1 equiv) and [(4-methoxyphenyl)methyl](methyl)amine (370.62 mg, 2.451 mmol, 1.5 equiv) in 1,4-dioxane (20 mL) were added Cs₂CO₃ (1065 mg, 3.3 mmol, 2 equiv), Xantphos (189.10 mg, 0.33 mmol, 0.2 equiv) and Pd₂(dba)₃ (150 mg, 0.16 mmol, 0.1 equiv) in portions at room temperature under air atmosphere. The resulting mixture was stirred for 2 h at 100° C. under nitrogen atmosphere. The desired product could be detected by LCMS. The resulting mixture was concentrated under reduced pressure. The residue was purified by reversed-phase flash chromatography with the following conditions: column, C18 silica gel; mobile phase, MeCN in water, 10% to 50% gradient in 10 min; detector, UV 254 nm. to afford methyl 5-chloro-1-[(4-methoxyphenyl)methyl]-7-([(4-methoxyphenyl)methyl](methyl)aminopyrazolo[4,3-d]pyrimidine-3-carboxylate (600 mg, 76%) as a white solid.

To a stirred mixture of methyl 5-chloro-1-[(4-methoxyphenyl)methyl]-7-([(4-methoxyphenyl)methyl](methyl)aminopyrazolo[4,3-d]pyrimidine-3-carboxylate (550 mg, 1.14 mmol, 1 equiv) in THF (10 mL) and H₂O (3 mL) was added LiOH (137 mg, 5.7 mmol, 5 equiv) in portions at room temperature under air atmosphere. The resulting mixture was stirred for 2 h at room temperature under nitrogen atmosphere. The desired product could be detected by LCMS. The resulting mixture was concentrated under reduced pressure. The residue was purified by silica gel column chromatography, eluted with CH2Cl₂/MeOH (5:1) to afford 5-chloro-1-[(4-methoxyphenyl)methyl]-7-([(4-methoxyphenyl)methyl](methyl)aminopyrazolo[4,3-d]pyrimidine-3-carboxylic acid (500 mg, 94%) as a white solid.

To a stirred mixture of 5-chloro-1-[(4-methoxyphenyl)methyl]-7-([(4-methoxyphenyl)methyl](methyl)aminopyrazolo[4,3-d]pyrimidine-3-carboxylic acid (350 mg, 0.75 mmol, 1 equiv) and 6-([(2R)-2-aminopropoxy]methyl-1-methyl-1,2,3-benzotriazol-4-amine (211 mg, 0.9 mmol, 1.2 equiv) in DMF were added HATU (569 mg, 1.5 mmol, 2 equiv) and DIEA (580 mg. 4.5 mmol, 6 equiv) in portions at room temperature under air atmosphere. The resulting mixture was stirred for 2 h at room temperature under nitrogen atmosphere. The desired product could be detected by LCMS. The residue was purified by reversed-phase flash chromatography with the following conditions: column, C18 silica gel; mobile phase, MeCN in water, 10% to 50% gradient in 10 min; detector, UV 254 nm. to afford N-[(2R)-1-[(7-amino-3-methyl-1,2,3-benzotriazol-5-yl)methoxy]propan-2-yl]-5-chloro-1-[(4-methoxyphenyl)methyl]-7-([(4-methoxyphenyl)methyl](methyl)aminopyrazolo[4,3-d]pyrimidine-3-carboxamide (400 mg, 78%) as a white solid.

To a stirred mixture of N-[(2R)-1-[(7-amino-3-methyl-1,2,3-benzotriazol-5-yl)methoxy]propan-2-yl]-5-chloro-1-[(4-methoxyphenyl)methyl]-7-([(4-methoxyphenyl)methyl](methyl)aminopyrazolo[4,3-d]pyrimidine-3-carboxamide (500 mg, 0.73 mmol, 1 equiv) and Cs₂CO₃ (475 mg, 1.5 mmol, 2 equiv) in 1,4-dioxane (20 mL) were added Pd₂(dba)₃ (67 mg, 0.073 mmol, 0.1 equiv) and Xantphos (84 mg, 0.15 mmol, 0.2 equiv) in portions at room temperature under air atmosphere. The resulting mixture was stirred for overnight at 100° C. under nitrogen atmosphere. The desired product could be detected by LCMS. The resulting mixture was concentrated under reduced pressure. The residue was purified by reversed-phase flash chromatography with the following conditions: column, C18 silica gel; mobile phase, MeCN in water, 10% to 50% gradient in 10 min; detector, UV 254 nm. to afford (14R)-19-[(4-methoxyphenyl)methyl]-21-([(4-methoxyphenyl)methyl](methyl)amino-7,14-dimethyl-12-oxa-2,5,6,7,15,18,19,22,23-nonaazapentacyclo[15.5.2.1\hat{ }(3,10.0\hat{ }(4,8.0\hat{ }(20,24]pentacosa-1(23),3(25),4(8),5,9,17,20(24),21-octaen-16-one (350 mg, 74%) as a yellow solid.

A mixture of (14R)-19-[(4-methoxyphenyl)methyl]-21-([(4-methoxyphenyl)methyl](methyl)amino-7,14-dimethyl-12-oxa-2,5,6,7,15,18,19,22,23-nonaazapentacyclo[15.5.2.1\hat{ }(3,10.0\hat{ }(4,8.0\hat{ }(20,24]pentacosa-1(23),3(25),4(8),5,9,17,20(24),21-octaen-16-one (35 mg, 0.054 mmol, 1 equiv) and TFA (3.5 mL,) was stirred for overnight at 500° C. under nitrogen atmosphere. To the above mixture was added H2SO₄ (30 uL) dropwise at room temperature. The resulting mixture was stirred for additional 2 h at room temperature. The desired product could be detected by LCMS. The mixture was neutralized to pH 7 with saturated NaHCO₃ (a_{q}.). The aqueous layer was extracted with EtOAc (3×100 mL). The organic layers were combined, dried and concentrated under vacuum. The crude product was purified by Prep-HPLC with the following conditions (Column: YMC-Actus Triart C18 ExRS 30*150 mm, 5 μm; Mobile Phase A: water (10 mmol/L NH₄HCO₃+0.05% NH₃H₂O), Mobile Phase B: ACN; Flow rate: 60 mL/min mL/min; Gradient: 12% B to 30% B in 10 mm; Wave Length: 254 nm/220 nm; RT1(min): 14.43) to afford (14R)-7,14-dimethyl-21-(methylamino)-12-oxa-2,5,6,7,15,18,19,22,23-nonaazapentacyclo[15.5.2.1\hat{ }(3,10.0\hat{ }(4,8.0\hat{ }(20,24]pentacosa-1(23),3(25),4(8),5,9,17,20(24),21-octaen-16-one) (6.8 mg, 30%) as a white solid. LCMS: [M+H]⁺=409.00. NMR: ¹H NMR (400 MHz, DMSO-d₆) δ 14.58 (s, 1H), 8.92 (d, J=16.4 Hz, 2H), 8.72 (s, 1H), 8.40 (s, 1H), 7.34-7.12 (m, 1H), 4.74 (s, 2H), 4.27 (s, 3H), 4.13-4.01 (m, 1H), 3.77-3.55 (m, 2H), 3.08 (d, J=4.5 Hz, 3H), 1.24 (d, J=6.8 Hz, 3H).

### Example 22: Synthesis of Compound 60

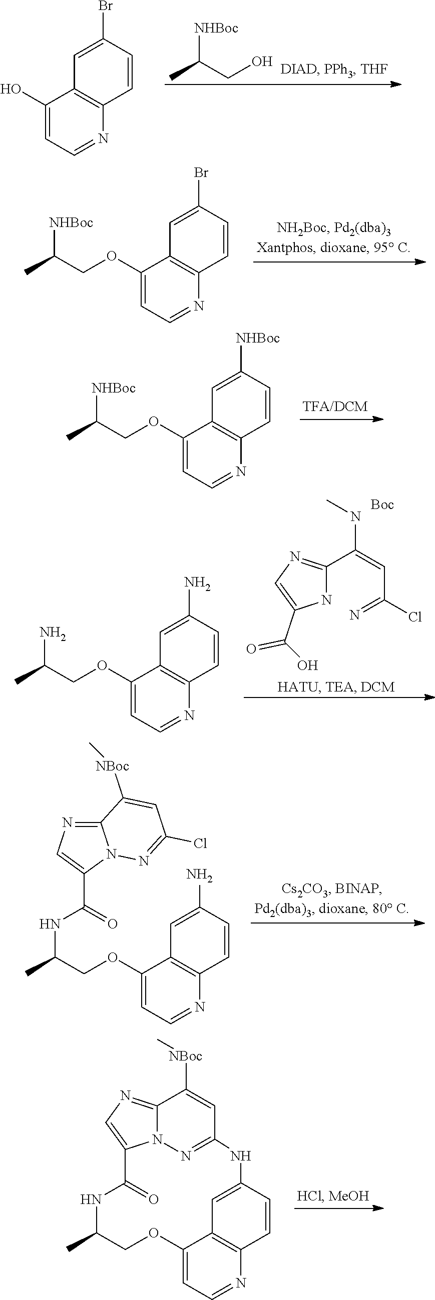

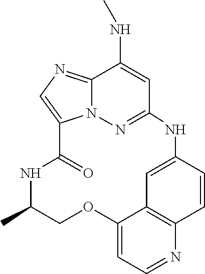 [SMILES: CNc1cc2nn3c(cnc13)C(=O)N[C@H](C)COc1ccnc3ccc(cc13)N2]

To a stirred solution/mixture of DIAD (2.71 g, 13.4 mmol, 1.5 equiv) and PPh₃ (7.02 g, 26.8 mmol, 3 equiv) in THF (20 mL) was added 6-bromoquinolin-4-ol (2 g, 8.93 mmol, 1 equiv) and (tert-butoxycarbonyl)[(2R)-1-hydroxypropan-2-yl]aminyl (2.33 g, 13.4 mmol, 1.5 equiv) in portions at 0° C. under nitrogen atmosphere. The resulting mixture was stirred for 4 h at room temperature under nitrogen atmosphere. The reaction was monitored by LCMS. The desired product could be detected by LCMS. The resulting mixture was concentrated under vacuum. The residue was purified by silica gel column chromatography, eluted with PE/EA (1:1) to afford [(2R)-1-[(6-bromoquinolin-4-yl)oxy]propan-2-yl](tert-butoxycarbonyl)aminyl (2.0 g, 59%) as a colorless oil.

A solution of tert-butyl N-[(2R)-1-[(6-bromoquinolin-4-yl)oxy]propan-2-yl]carbamate (2 g, 5.25 mmol, 1 equiv), tert-butyl carbamate (0.04 g, 0.31 mmol, 1.2 equiv), Pd₂(dba)₃ (480.36 mg, 0.53 mmol, 0.1 equiv), Xantphos (30.27 mg, 0.05 mmol, 0.2 equiv) and Cs₂CO₃ (3418.25 mg, 10.49 mmol, 2 equiv) in 1,4-dioxane (20 mL) was stirred for 1 overnight at 110° C. under argon atmosphere. Desired product could be detected by LCMS. The resulting mixture was concentrated under vacuum. The desired product could be detected by LCMS. The precipitated solids were collected by filtration and washed with MeOH (5×3 mL). The residue was purified by reversed-phase flash chromatography with the following conditions: column, C18 silica gel; mobile phase, MeCN in Water (0.1% FA), 10% to 80% gradient in 10 min; detector, UV 254 nm. to afford tert-butyl N-[(2R)-1-((6-[(tert-butoxycarbonyl)amino]quinolin-4-yloxy)propan-2-yl]carbamate (1.80 g, 82%) as a colorless oil.

To a stirred solution/mixture of tert-butyl N-[(2R)-1-((6-[(tert-butoxycarbonyl)amino]quinolin-4-yloxy)propan-2-yl]carbamate (100 mg, 0.24 mmol, 1 equiv) in DCM (2 mL) was added TFA (0.25 mL) dropwise at 0° C. under air atmosphere. The resulting mixture was stirred for 1 h at room temperature under air atmosphere. The reaction was monitored by LCMS. The desired product could be detected by LCMS. The resulting mixture was concentrated under vacuum. The crude product was used in the next step directly without further purification.

To a stirred mixture of 8-[(tert-butoxycarbonyl)(methyl)amino]-6-chloroimidazo[1,2-b]pyridazine-3-carboxylic acid (1353.45 mg, 4.14 mmol, 1 equiv) and HATU (2362.56 mg, 6.21 mmol, 1.5 equiv) and DIEA (1606.13 mg, 12.43 mmol, 3 equiv) in DCM (20 mL) was added 4-[(2R)-2-aminopropoxy]quinolin-6-amine (0.9 g, 4.14 mmol, 1 equiv) in portions at room temperature under air atmosphere. The resulting mixture was stirred for 2 h at room temperature under nitrogen atmosphere. The reaction was monitored by LCMS. The desired product could be detected by LCMS. The precipitated solids were collected by filtration and washed with 1,4-dioxane (3×3 mL). The resulting mixture was concentrated under vacuum. The residue was purified by reversed-phase flash chromatography with the following conditions: column, C18 silica gel; mobile phase, MeCN in Water (0.1% FA), 10% to 70% gradient in 10 min; detector, UV 254 nm. to afford tert-butyl N-(3-([(2R)-1-[(6-aminoquinolin-4-yl)oxy]propan-2-yl]carbamoyl-6-chloroimidazo[1,2-b]pyridazin-8-yl)-N-methylcarbamate (500 mg, 23%) as a yellow crude oil.

To a stirred solution/mixture of tert-butyl N-(3-([(2R)-1-[(6-aminoquinolin-4-yl)oxy]propan-2-yl]carbamoyl-6-chloroimidazo[1,2-b]pyridazin-8-yl)-N-methylcarbamate (290 mg, 0.55 mmol, 1 equiv) and Cs₂CO₃ (359.27 mg, 1.10 mmol, 2 equiv) in 1,4-dioxane were added dioxane (13 mL) and Pd₂(dba)₃ (50.49 mg, 0.06 mmol, 0.1 equiv) in portions at room temperature under air atmosphere. The resulting mixture was stirred for overnight at 80° C. under argon atmosphere. The reaction was monitored by LCMS. Desired product could be detected by LCMS. The precipitated solids were collected by filtration and washed with MeOH (5×3 mL). The desired product could be detected by LCMS. The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. to afford tert-butyl N-methyl-N-[(12R)-12-methyl-10-oxo-14-oxa-2,4,5,7,11,18-hexaazapentacyclo[13.6.2.2\hat{ }(3,6.0\hat{ }(5,9.0\hat{ }(19,23]pentacosa-1(22),3,6,8,15,17,19(23),20,24-nonaen-24-yl]carbamate (180 mg, 67%) as a yellow syrup.

A solution/mixture of tert-butyl N-methyl-N-[(12R)-12-methyl-10-oxo-14-oxa-2,4,5,7,11,18-hexaazapentacyclo[13.6.2.2\hat{ }(3,6.0\hat{ }(5,9.0\hat{ }(19,23]pentacosa-1(22),3,6,8,15,17,19(23),20,24-nonaen-24-yl]carbamate (170 mg, 0.35 mmol, 1 equiv) in DCM (4 mL) was stirred for 5 min at 0° C. under air atmosphere. To the above mixture was added HCl_{(gas)} in 1,4-dioxane (2 mL, 4N) dropwise over 1 min at 0° C. The resulting mixture was stirred for additional 2 h at room temperature. The reaction was monitored by LCMS. The desired product could be detected by LCMS. The resulting mixture was concentrated under vacuum. The crude product (120 mg) was purified by Prep-HPLC with the following conditions (Column: XBridge Prep Phenyl OBD Column, 19*150 mm, 5 μm; Mobile Phase A: Water (0.1% FA), Mobile Phase B: ACN; Flow rate: 25 mL/min; Gradient: 6% B to 21% B in 8 min, 21% B; Wave Length: 254/220 nm; RT1(min): 9.68; Number Of Runs: 0) to afford (12R)-12-methyl-24-(methylamino)-14-oxa-2,4,5,7,11,18-hexaazapentacyclo[13.6.2.2\hat{ }(3,6.0\hat{ }(5,9.0\hat{ }(19,23]pentacosa-1(22),3,6,8,15,17,19(23),20,24-nonaen-10-one (56 mg, 40%) as a white solid. LCMS:(ES,m/z): [M+H]⁺=390.00. NMR: ¹H NMR (400 MHz, DMSO-d6) δ 9.79 (s, 1H), 9.23 (d, J=2.6 Hz, 1H), 8.76 (dd, J=65.5, 5.8 Hz, 2H), 8.08-7.77 (m, 2H), 7.56 (dt, J=9.2, 3.5 Hz, 2H), 7.30 (d, J=4.9 Hz, 1H), 5.87 (s, 11H), 4.42 (d, J=8.3 Hz, 2H), 4.15 (d, J=8.0 Hz, 1H), 2.91 (d, J=4.8 Hz, 3H), 1.29 (d, J=6.1 Hz, 3H).

### Example 23: Synthesis of Compound 61

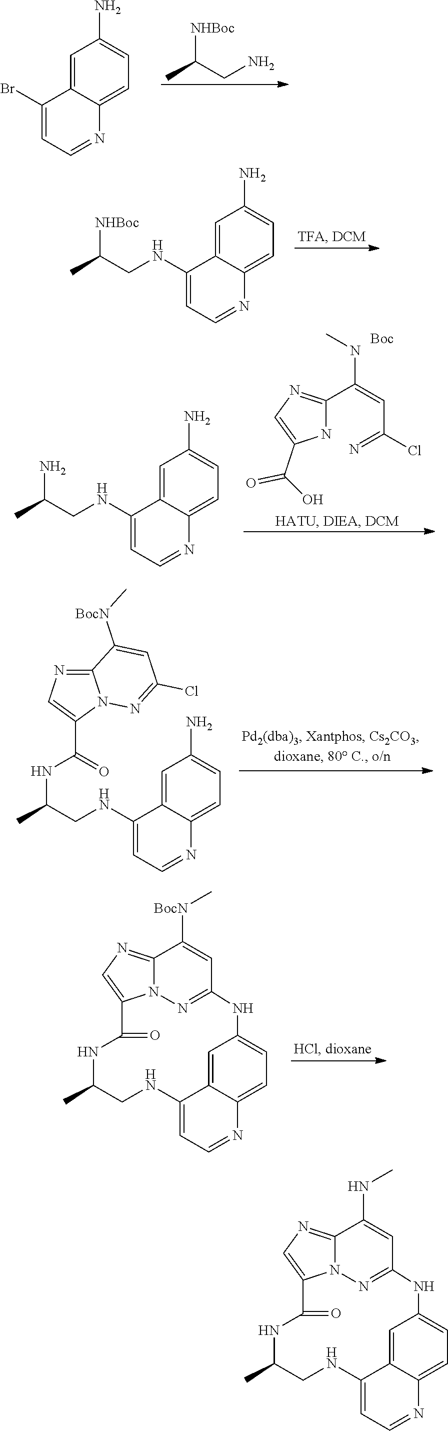

To a stirred solution/mixture of 4-bromoquinolin-6-amine (1 g, 4.483 mmol, 1 equiv) and tert-butyl N-[(2R)-1-aminopropan-2-yl]carbamate (937.33 mg, 5.38 mmol, 1.2 equiv) in 1,4-dioxane (15 mL) were added t-BuBrettPhos PD G3 (406.37 mg, 0.45 mmol, 0.1 equiv) and BrettPhos (240.63 mg, 0.45 mmol, 0.1 equiv) and CS₂CO₃ (2921.19 mg, 8.97 mmol, 2 equiv) in portions at room temperature. The resulting mixture was stirred for overnight at 90° C. under argon atmosphere. The reaction was monitored by LCMS. The desired product could be detected by LCMS. The resulting mixture was concentrated under vacuum. The residue was purified by reversed-phase flash chromatography with the following conditions: column, C18 silica gel; mobile phase, MeCN in Water (0.1% NH₃·H₂O), 10% to 90% gradient in 15 min; detector, UV 254 nm. to afford tert-butyl N-[(2R)-1-[(6-aminoquinolin-4-yl)amino]propan-2-yl]carbamate (158 mg, 11%) as a light yellow solid.

A solution/mixture of tert-butyl N-[(2R)-1-[(6-aminoquinolin-4-yl)amino]propan-2-yl]carbamate (170 mg, 0.54 mmol, 1 equiv) in DCM (10 mL) was stirred for 3 mm at 0° C. under nitrogen atmosphere. To the above mixture was added TFA (1 mL) dropwise over 1 min at 0° C. The resulting mixture was stirred for additional 1 h at room temperature. The reaction was monitored by LCMS. The desired product could be detected by LCMS. The resulting mixture was concentrated under vacuum. The crude product was used in the next step directly without further purification.

To a stirred solution/mixture of N4-[(2R)-2-aminopropyl]quinoline-4,6-diamine (93 mg, 0.430 mmol, 1 equiv) and 8-[(tert-butoxycarbonyl)(methyl)amino]-6-chloroimidazo[1,2-b]pyridazine-3-carboxylic acid (70.25 mg, 0.22 mmol, 0.5 equiv) in DCM (2 mL) were added HATU (245.24 mg, 0.65 mmol, 1.5 equiv) and DIEA (166.72 mg, 1.29 mmol, 3.0 equiv) in portions at room temperature. The resulting mixture was stirred for 2 h at room temperature under nitrogen atmosphere. The reaction was monitored by LCMS. The resulting mixture was concentrated under vacuum. The resulting mixture was concentrated under vacuum. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. to afford tert-butyl N-(3-([(2R)-1-[(6-aminoquinolin-4-yl)amino]propan-2-yl]carbamoyl-6-chloroimidazo[1,2-b]pyridazin-8-yl)-N-methylcarbamate (110 mg, 49%) as a light yellow solid.

To a stirred solution/mixture of tert-butyl N-(3-([(2R)-1-[(6-aminoquinolin-4-yl)amino]propan-2-yl]carbamoyl-6-chloroimidazo[1,2-b]pyridazin-8-yl)-N-methylcarbamate (90 mg, 0.17 mmol, 1 equiv) and Cs₂CO₃ (111.71 mg, 0.34 mmol, 2 equiv) in 1,4-dioxane (2 mL) were added XantPhos (19.84 mg, 0.034 mmol, 0.2 equiv) and Pd₂(dba)₃ (15.70 mg, 0.017 mmol, 0.1 equiv) in portions at room temperature. The resulting mixture was stirred for overnight at 90° C. under argon atmosphere. The reaction was monitored by LCMS. The resulting mixture was concentrated under vacuum. The resulting mixture was concentrated under vacuum. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. to afford tert-butyl N-methyl-N-[(12R)-12-methyl-10-oxo-2,4,5,7,11,14,18-heptaazapentacyclo[13.6.2.2\hat{ }(3,6.0\hat{ }(5,9.0\hat{ }(19,23]pentacosa-1(21),3,6,8,15,17,19,22,24-nonaen-24-yl]carbamate (52 mg, 62%) as alight yellow solid.

A mixture of tert-butyl N-methyl-N-[(12R)-12-methyl-10-oxo-2,4,5,7,11,14,18-heptaazapentacyclo[13.6.2.2\hat{ }(3,6.0\hat{ }(5,9.0\hat{ }(19,23]pentacosa-1(21),3,6,8,15,17,19,22,24-nonaen-24-yl]carbamate (37 mg, 0.08 mmol, 1 equiv) and HCl(gas) in 1,4-dioxane (2 mL, 4N) in THF was stirred for 2 h at room temperature under nitrogen atmosphere. The reaction was monitored by LCMS. The resulting mixture was concentrated under vacuum. The resulting mixture was concentrated under vacuum. The crude product (30 mg) was purified by Prep-HPLC with the following conditions (Column: YMC-Actus Triart C18 ExRS 30*150 mm, 5 μm; Mobile Phase A: Water (10 mmol/L NH4HCO3+0.05% NH3H2O), Mobile Phase B: ACN; Flow rate: 60 mL/min mL/min; Gradient: 49% B to 66% B in 10 min; Wave Length: 254 nm/220 nm; RT1(min): 4.83) to afford (12R)-12-methyl-24-(methylamino)-2,4,5,7,11,14,18-heptaazapentacyclo[13.6.2.2\hat{ }{3,6}0.\hat{ }{5,9}0.\hat{ }{19,23}]pentacosa-1(21),3,6,8,15,17,19,22,24-nonaen-10-one (16.7 mg, 55%) as a white solid. LCMS:(ES, m/z): [M+H]⁺=389.00. NMR: 1H NMR (400 MHz, Methanol-d₄) δ 9.21 (d, J=2.5 Hz, 1H), 8.39 (d, J=5.0 Hz, 1H), 7.94 (s, 1H), 7.84 (d, J=9.1 Hz, 1H), 7.43 (dd, J=9.0, 2.6 Hz, 1H), 7.02 (d, J=5.0 Hz, 1H), 5.87 (s, 1H), 4.32 (td, J=6.5, 2.8 Hz, 1H), 3.45 (qd, J=14.2, 4.5 Hz, 2H), 3.02 (s, 3H), 1.12 (d, J=6.7 Hz, 3H).

### Example 24: Synthesis of Compound 62

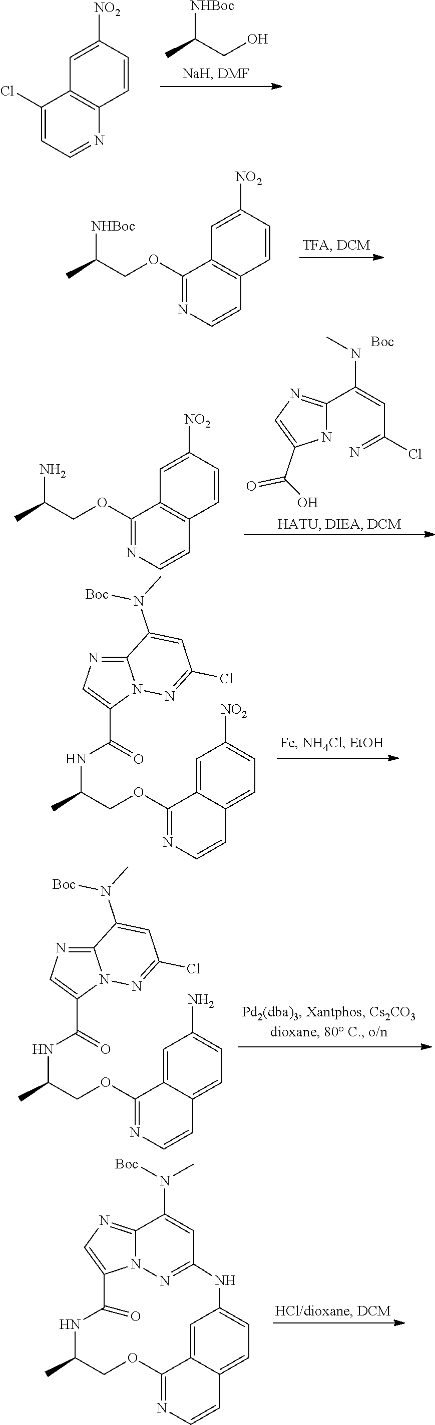

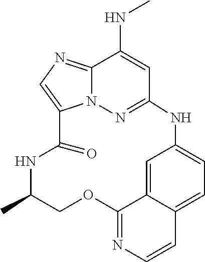 [SMILES: CNc1cc2nn3c(cnc13)C(=O)N[C@H](C)COc1nccc3ccc(cc13)N2]

To a stirred mixture of t-chloro-7-nitroisoquinoline (1 g, 4.794 mmol, 1 equiv) and tert-butyl N-[(2R)-1-hydroxypropan-2-yl]carbamate (0.84 g, 4.8 mmol, 1 equiv) in DMF (10 mL) was added NaH (0.13 g, 5.3 mmol, 1.1 equiv) in portions at 0° C. under nitrogen atmosphere. The resulting mixture was stirred for 2 h at room temperature under nitrogen atmosphere. The desired product could be detected by LCMS. The reaction was quenched with water at room temperature. The resulting mixture was concentrated under vacuum. The residue was purified by reversed-phase flash chromatography with the following conditions: column, C18 silica gel; mobile phase, MeCN in water, 100% to 50% c gradient in 10 min; detector, UV 254 nm. to afford tert-butyl N-[(2R)-1-[(7-nitroisoquinolin-1-yl)oxy]propan-2-yl]carbamate (300 mg, 18%) as a yellow solid.

To a stirred solution of tert-butyl N-[(2R)-1-[(7-nitroisoquinolin-1-yl)oxy]propan-2-yl]carbamate (250 mg, 0.72 mmol, 1 equiv) in DCM (5 mL) was added TFA (3 mL) dropwise at 0° C. under nitrogen atmosphere. The resulting mixture was stirred for 2 h at room temperature under nitrogen atmosphere. The desired product could be detected by LCMS. The resulting mixture was concentrated under reduced pressure. The resulting mixture was used in the next step directly without further purification.

To a stirred mixture of (2R)-1-[(7-nitroisoquinolin-1-yl)oxy]propan-2-amine (200 mg, 0.81 mmol, 1 equiv) and 8-[(tert-butoxycarbonyl)(methyl)amino]-6-chloroimidazo[1,2-b]pyridazine-3-carboxylic acid (291 mg, 0.9 mmol, 1.1 equiv) in DCM (10 mL) were added DIEA (0.70 mL, 4 mmol, 5 equiv) and HATU (461 mg, 1.2 mmol, 1.5 equiv) in portions at room temperature under air atmosphere. The resulting mixture was stirred for 2 h at room temperature under nitrogen atmosphere. The desired product could be detected by LCMS. The reaction was quenched with water at room temperature. The resulting mixture was concentrated under reduced pressure. The residue was purified by reversed-phase flash chromatography with the following conditions: column, C18 silica gel; mobile phase, MeCN in water, 10% to 50% gradient in 10 min; detector, UV 254 nm. to afford tert-butyl N-(6-chloro-3-([(2R)-1-[(7-nitroisoquinolin-1-yl)oxy]propan-2-yl]carbamoylimidazo[1,2-b]pyridazin-8-yl)-N-methylcarbamate (250 mg, 56%) as a yellow solid.

To a stirred mixture of tert-butyl N-(6-chloro-3-([(2R)-1-[(7-nitroisoquinolin-1-yl)oxy]propan-2-yl]carbamoylimidazo[1,2-b]pyridazin-8-yl)-N-methylcarbamate (370 mg, 0.67 mmol, 1 equiv) in EtOH (10 mL) were added Fe (186 mg, 3.3 mmol, 5 equiv) and NH₄Cl (178 mg, 3.3 mmol, 5 equiv) in portions at room temperature under air atmosphere. The resulting mixture was stirred for 1 h at 80° C. under nitrogen atmosphere. The desired product could be detected by LCMS. The resulting mixture was concentrated under reduced pressure. The residue was purified by reversed-phase flash chromatography with the following conditions: column, C18 silica gel; mobile phase, MeCN in water, 10% to 50% gradient in 10 min; detector, UV 254 nm. to afford tert-butyl N-(3-([(2R)-1-[(7-aminoisoquinolin-1-yl)oxy]propan-2-yl]carbamoyl-6-chloroimidazo[1,2-b]pyridazin-8-yl)-N-methylcarbamate (300 mg, 86%) as a white solid.

To a stirred mixture of tert-butyl N-(3-([(2R)-1-[(7-aminoisoquinolin-1-yl)oxy]propan-2-yl]carbamoyl-6-chloroimidazo[1,2-b]pyridazin-8-yl)-N-methylcarbamate (270 mg, 0.5 mmol, 1 equiv) and Cs₂CO₃ (669 mg, 2.1 mmol, 4 equiv) in 1,4-dioxane (10 mL) were added Pd₂(dba)₃ (94 mg, 0.10 mmol, 0.2 equiv) and Xantphos (119 mg, 0.21 mmol, 0.4 equiv in portions at room temperature under air atmosphere. The resulting mixture was stirred for overnight at 80° C. under nitrogen atmosphere. The desired product could be detected by LCMS. The resulting mixture was concentrated under reduced pressure. The crude product (180 mg) was purified by Prep-HPLC with the following conditions (Column: XBridge Shield RP18 OBD Column 30*150 mm, 5 m; Mobile Phase A: water (10 mmol/L NH₄HCO₃+0.1% NH₃·H₂O), Mobile Phase B: ACN; Flow rate: 60 mL/min mL/min; Gradient: 33% B to 50% B in 8 min; Wave Length: 254 nm/220 nm; RT1(min): 8.37) to afford tert-butyl N-methyl-N-[(12R)-12-methyl-10-oxo-14-oxa-2,4,5,7,11,16-hexaazapentacyclo[13.6.2.2\hat{ }(3,6.0\hat{ }(5,9.0\hat{ }(19,23]pentacosa-1(21),3,6,8,15,17,19,22,24-nonaen-24-yl]carbamate (150 mg, 60%) as a white solid.

A solution of tert-butyl N-methyl-N-[(12R)-12-methyl-10-oxo-14-oxa-2,4,5,7,11,16-hexaazapentacyclo[13.6.2.2\hat{ }(3,6.0\hat{ }(5,9.0\hat{ }(19,23]pentacosa-1(21),3,6,8,15,17,19,22,24-nonaen-24-yl]carbamate (50 mg, 0.1 mmol, 1 equiv) in DCM (5 mL) was treated with 4M HCl(gas) in 1,4-dioxane (2 mL) at 0° C. under nitrogen atmosphere. The resulting mixture was stirred for 1 h at room temperature under nitrogen atmosphere. The desired product could be detected by LCMS. The mixture was neutralized to pH 7 with saturated NaHCO_{3(aq.)}. The aqueous layer was extracted with EtOAc (3×50 mL). The resulting mixture was concentrated under vacuum. The crude product (50 mg) was purified by Prep-HPLC with the following conditions (Column: XBridge Shield RP18 OBD Column 30*150 mm, 5 m; Mobile Phase A: water (10 mmol/L NH₄HCO3), Mobile Phase B: ACN; Flow rate: 60 mL/min mL/min; Gradient: 27% B to 47% B in 8 min; Wave Length: 254 nm/220 nm; RT1(min): 6.2) to afford (12R)-12-methyl-24-(methylamino)-14-oxa-2,4,5,7,11,16-hexaazapentacyclo[13.6.2.2\hat{ }(3,6.0\hat{ }(5,9.0\hat{ }(19,23]pentacosa-1(21),3,6,8,15,17,19,22,24-nonaen-10-one (37 mg, 90%) as a white solid. LCMS: [M+H]⁺=390.00. NMR: ¹H NMR (400 MHz, DMSO-d₆) δ 9.75 (s, 1H), 9.28 (d, 0.1=2.3 Hz, 1H), 8.84 (d, J=8.0 Hz, 1H), 7.98 (d, J=5.8 Hz, 1H), 7.89 (d, J=8.0 Hz, 2H), 7.56 (td, J=9.0, 8.5, 3.3 Hz, 2H), 7.48 (d, J=5.8 Hz, 1H), 5.84 (s, 1H), 4.67 (dd, J=10.6, 3.2 Hz, 1H), 4.47 (d, J=7.7 Hz, 1H), 4.21 (dd, J=10.5, 3.1 Hz, 1H), 2.92 (d, J=4.9 Hz, 3H), 1.14 (d, J=6.7 Hz, 3H).

### Example 25: Synthesis of Compound 63

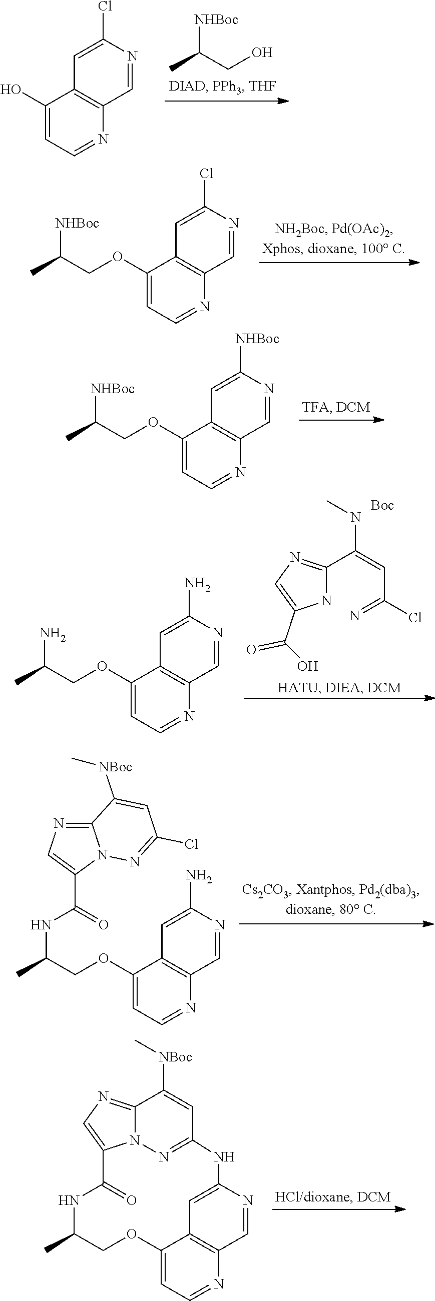

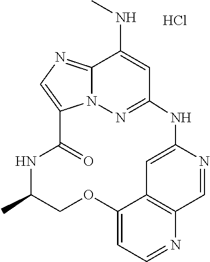 [SMILES: CNc1cc2nn3c(cnc13)C(=O)N[C@H](C)COc1ccnc3cnc(cc13)N2 ; Cl]

To a stirred mixture of 6-chloro-1,7-naphthyridin-4-ol (2 g, 11.1 mmol, 1 equiv) and (tert-butoxycarbonyl)[(2R)-1-hydroxypropan-2-yl]aminyl (4.8 g, 27.7 mmol, 2.5 equiv) in THF (50 mL) were added DIAD (6.7 g, 33.2 mmol, 3 equiv) and PPh₃ (8.7 g, 33.2 mmol, 3 equiv) in portions at 0° C. under nitrogen atmosphere. The resulting mixture was stirred for overnight at room temperature under nitrogen atmosphere. The desired product could be detected by LCMS. The reaction was quenched with water at room temperature. The resulting mixture was concentrated under reduced pressure. The residue was purified by silica gel column chromatography, eluted with PE/EA (1:1) to afford (tert-butoxycarbonyl)[(2R)-1-[(6-chloro-1,7-naphthyridin-4-yl)oxy]propan-2-yl]aminyl (1.3 g, 36%) as a white solid.

To a stirred mixture of tert-butyl N-[(2R)-1-[(6-chloro-1,7-naphthyridin-4-yl)oxy]propan-2-yl]carbamate (1.7 g, 5.03 mmol, 1 equiv) and tert-butyl carbamate (1.2 g, 10.1 mmol, 2 equiv) in 1,4-dioxane (30 mL) were added Pd(OAc)₂ (113 mg, 0.5 mmol, 0.1 equiv), Xphos (6 mg, 0.012 mmol, 0.2 equiv) and Cs₂CO₃ (3.28 g, 10.1 mmol, 2 equiv) in portions at room temperature under nitrogen atmosphere. The resulting mixture was stirred for overnight at 100° C. under nitrogen atmosphere. The desired product could be detected by LCMS. The reaction was quenched with water at room temperature. The aqueous layer was extracted with EtOAc (3×50 mL). The organic layers were combined, dried and concentrated under vacuum. The residue was purified by reversed-phase flash chromatography with the following conditions: column, C18 silica gel; mobile phase, MeCN in water, 10% to 50% gradient in 10 min; detector, UV 254 nm. to afford tert-butyl N-[(2R)-1-((6-[(tert-butoxycarbonyl)amino]-1,7-naphthyridin-4-yloxy)propan-2-yl]carbamate (1 g, 47%) as a yellow solid.

To a stirred solution of tert-butyl N-[(2R)-1-((6-[(tert-butoxycarbonyl)amino]-1,7-naphthyridin-4-yloxy)propan-2-yl]carbamate (600 mg, 1.4 mmol, 1 equiv) in DCM (12 mL) was added TFA (3.00 mL) dropwise at 0° C. under nitrogen atmosphere. The resulting mixture was stirred for 2 h at room temperature under nitrogen atmosphere. The desired product could be detected by LCMS. The resulting mixture was concentrated under reduced pressure. The crude product was used in the next step directly without further purification.

To a stirred mixture of 4-[(2R)-2-aminopropoxy]-1,7-naphthyridin-6-amine (300 mg, 1.38 mmol, 1 equiv) and 8-[(tert-butoxycarbonyl)(methyl)amino]-6-chloroimidazo[1,2-b]pyridazine-3-carboxylic acid (449 mg, 1.4 mmol, 1 equiv) in DMF (20 mL) were added HATU (1.6 g, 4.1 mmol, 3 equiv) and DIEA (1.07 g, 8.25 mmol, 6 equiv) in portions at room temperature under nitrogen atmosphere. The resulting mixture was stirred for 2 h at room temperature under nitrogen atmosphere. The desired product could be detected by LCMS. The reaction was quenched with water at room temperature. The resulting mixture was concentrated under reduced pressure. The residue was purified by reversed-phase flash chromatography with the following conditions: column, C18 silica gel; mobile phase, MeCN in water, 10% to 50% gradient in 10 min; detector, UV 254 nm. to afford tert-butyl N-(3-([(2R)-1-[(6-amino-1,7-naphthyridin-4-yl)oxy]propan-2-yl]carbamoyl-6-chloroimidazo[1,2-b]pyridazin-8-yl)-N-methylcarbamate (350 mg, 48%) as a white solid.

To a stirred mixture of tert-butyl N-(3-{[(2R)-1-[(6-amino-1,7-naphthyridin-4-yl)oxy]propan-2-yl]carbamoyl}-6-chloroimidazo[1,2-b]pyridazin-8-yl)-N-methylcarbamate (300 mg, 0.57 mmol, 1 equiv) and Cs₂CO₃ (371 mg, 1.14 mmol, 2 equiv) in 1,4-dioxane (10 mL) were added XantPhos (66 mg, 0.11 mmol, 0.2 equiv) and Pd₂(dba)₃ (52 mg, 0.057 mmol, 0.1 equiv) in portions at room temperature under nitrogen atmosphere. The resulting mixture was stirred for overnight at 80° C. under nitrogen atmosphere. The desired product could be detected by LCMS. The reaction was quenched with water at room temperature. The aqueous layer was extracted with EtOAc (3×100 mL). The organic layers were combined, dried and concentrated under vacuum. The residue was purified by reversed-phase flash chromatography with the following conditions: column, C18 silica gel; mobile phase, MeCN in water, 10% to 50% gradient in 10 min; detector, UV 254 nm. to afford tert-butyl N-methyl-N-[(12R)-12-methyl-10-oxo-14-oxa-2,4,5,7,11,18,21-heptaazapentacyclo[13.6.2.2\hat{ }{3,6}0.\hat{ }{5,9}0.\hat{ }{19,23}]pentacosa-1(22),3,6,8,15,17,19(23),20,24-nonaen-24-yl]carbamate (150 mg, 54%) as a yellow solid.

To a stirred solution of tert-butyl N-methyl-N-[(12R)-12-methyl-10-oxo-14-oxa-2,4,5,7,11,18,21-heptaazapentacyclo[13.6.2.2\hat{ }(3,6.0\hat{ }(5,9.0\hat{ }(19,23]pentacosa-1(22),3,6,8,15,17,19(23),20,24-nonaen-24-yl]carbamate (50 mg, 0.1 mmol, 1 equiv) in DCM (1 mL) was added 4 N HCl(gas) in 1,4-dioxane (4 mL) dropwise at 0° C. under nitrogen atmosphere. The resulting mixture was stirred for 2 h at room temperature under nitrogen atmosphere. The desired product could be detected by LCMS. The resulting mixture was concentrated under reduced pressure to afford (12R)-12-methyl-24-(methylamino)-14-oxa-2,4,5,7,11,18,21-heptaazapentacyclo[13.6.2.2\hat{ }(3,6.0\hat{ }(5,9.0\hat{ }(19,23]pentacosa-1(22),3,6,8,15,17,19(23),20,24-nonaen-10-one hydrochloride (19.7 mg, 43%) as a yellow solid. LCMS: [M+H]⁺=391.00. NMR: ¹H NMR (400 MHz, DMSO-d₆) δ 10.33 (s, 1H), 9.24 (s, 1H), 9.05 (s, 1H), 8.85 (d, J=4.9 Hz, 1H), 8.72 (d, J=7.0 Hz, 1H), 7.99 (s, 1H), 7.70 (s, 1H), 7.54 (d, J=4.9 Hz, 1H), 6.19 (s, 1H), 4.51-4.42 (m, 2H), 4.25 (d, J=8.4 Hz, 1H), 2.91 (s, 3H), 1.29 (d, J=6.1 Hz, 3H).

### Example 26: Synthesis of Compound 64

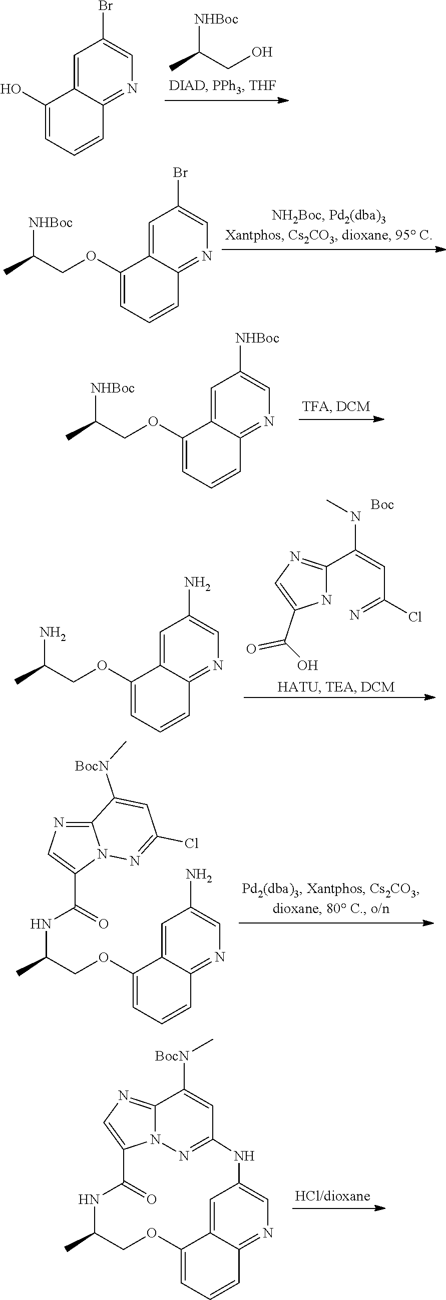

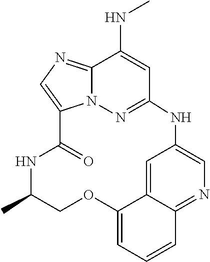 [SMILES: CNc1cc2nn3c(cnc13)C(=O)N[C@H](C)COc1cccc3ncc(cc13)N2]

A solution of 3-bromoquinolin-5-ol (2 g, 9.0 mmol, 1 equiv) in DMF (10 mL) was treated with NaH (214 mg, 9.0 mmol, 1 equiv) for 5 min at room temperature under argon atmosphere followed by the addition of tert-butyl (4R)-4-methyl-2,2-dioxo-1,2lambda6,3-oxathiazolidine-3-carboxylate (106 mg, 0.45 mmol, 1 equiv) dropwise at room temperature. The resulting mixture was stirred for 2 h at room temperature under argon atmosphere. To the above mixture was added H₂SO₄ (4 mL) dropwise at room temperature. The resulting mixture was stirred for additional 1 h at room temperature. The reaction was monitored by LCMS. The desired product could be detected by LCMS. The reaction was quenched by the addition of water/ice (100 mL) at 0° C. The resulting mixture was extracted with CH₂Cl₂ (3×100 mL). The combined organic layers were washed with saturated salt solution (3×100 mL), dried over anhydrous Na₂SO₄. After filtration, the filtrate was concentrated under reduced pressure. This resulted in tert-butyl N-[(2R)-1-[(3-bromoquinolin-5-yl) oxy]propan-2-yl]carbamate (3.3 g, 96%) as a black solid. The crude product was used in the next step directly without further purification.

To a stirred mixture of tert-butyl N-[(2R)-1-[(3-bromoquinolin-5-yl) oxy]propan-2-yl] carbamate (3.3 g, 8.5 mmol, 1 equiv) and dicaesium (1+) carbonate (5.6 g, 17.0 mmol, 2 equiv) in 1,4-dioxane (30 mL) was added tert-butyl carbamate (2.0 g, 17.0 mmol, 2 equiv) and Pd₂(dba)₃ (0.9 g, 0.85 mmol, 0.1 equiv), Xantphos (1 g, 1.7 mmol, 0.2 equiv) in portions at room temperature under argon atmosphere. The resulting mixture was stirred for 6 h at 95° C. under argon atmosphere. The reaction was monitored by LCMS. The desired product could be detected by LCMS. The resulting mixture was concentrated under vacuum. The residue was purified by silica gel column chromatography, eluted with PE/EA (7:1) to afford tert-butyl N-[(2R)-1-((3-[(tert-butoxycarbonyl) amino]quinolin-5-yloxy) propan-2-yl]carbamate (3.4 g, 96%) as a black solid.

To a stirred mixture of tert-butyl N-[(2R)-1-((3-[(tert-butoxycarbonyl) amino]quinolin-5-yloxy) propan-2-yl]carbamate (3.3 g, 7.9 mmol, 1 equiv) in DCM (24 mL) was added TFA (12 mL) in portions at room temperature under argon atmosphere. The resulting mixture was stirred for 1 h at room temperature under argon atmosphere. The reaction was monitored by LCMS. The desired product could be detected by LCMS. The resulting mixture was concentrated under vacuum. The crude resulting mixture was used in the next step directly without further purification.

To a stirred mixture of 5-[(2R)-2-aminopropoxy]quinolin-3-amine (100 mg, 0.5 mmol, 1 equiv) and 8-[(tert-butoxycarbonyl) (methyl)amino]-6-chloroimidazo[1,2-b]pyridazine-3-carboxylic acid (150 mg, 0.5 mmol, 1 equiv), HATU (263 mg, 0.7 mmol, 1.5 equiv) in DCM (10 mL) was added TEA (140 mg, 1.4 mmol, 3 equiv) dropwise at room temperature under argon atmosphere. The resulting mixture was stirred for 2 h at room temperature under argon atmosphere. The reaction was monitored by LCMS. The desired product could be detected by LCMS. The residue was purified by silica gel column chromatography, eluted with CH₂Cl₂/MeOH (9:1) to afford tert-butyl N-(3-([(2R)-1-[(3-aminoquinolin-5-yl) oxy]propan-2-yl]carbamoyl-6-chloroimidazo[1,2-b]pyridazin-8-yl)-N-methylcarbamate (100 mg. 41%) as a yellow green oil.

To a stirred mixture of tert-butyl N-(3-([(2R)-1-[(3-aminoquinolin-5-yl) oxy]propan-2-yl]carbamoyl-6-chloroimidazo[1,2-b]pyridazin-8-yl)-N-methylcarbamate (90 mg, 0.17 mmol, 1 equiv) and Xantphos (19.8 mg, 0.03 mmol, 0.2 equiv) in 1,4-dioxane (5 mL) was added Pd₂(dba)₃ (15.7 mg, 0.02 mmol, 0.1 equiv) in portions at room temperature under argon atmosphere. The resulting mixture was stirred for overnight at 80° C. under argon atmosphere. The reaction was monitored by LCMS. The desired product could be detected by LCMS. The crude product was purified by reverse phase flash to afford tert-butyl N-methy 1-N-[(12R)-12-methyl-10-oxo-14-oxa-2,4,5,7,11,20-hexaazapentacyclo [13.6.2.2\hat{ }{3,6}0.\hat{ }{5,9}0.\hat{ }{19,23}]pentacosa 1(21),3,6,8,15,17,19,22,24-nonaen-24-yl]carbamate (55 mg, 66%) as a white solid.

To a stirred mixture of tert-butyl N-methyl-N-[(12R)-12-methyl-10-oxo-14-oxa-2,4,5,7,11,20-hexaazapentacyclo [13.6.2.2\hat{ }(3,6.0\hat{ }(5,9.0\hat{ }(19,23]pentacosa-1(21),3,6,8,15,17,19,22,24-nonaen-24-yl]carbamate (70 mg, 0.14 mmol, 1 equiv) in THF (5 mL) was added conc·HCl (3 mL) dropwise at room temperature under argon atmosphere. The resulting mixture was stirred for 3 h at room temperature under argon atmosphere. The reaction was monitored by LCMS. The desired product could be detected by LCMS. The resulting mixture was concentrated under reduced pressure. The crude product (50 mg) was purified by Prep-HPLC with the following conditions (Column: XBridge Prep OBD C18 Column 30*150 mm, 5 m; Mobile Phase A: water (10 mmol/L NH₄HCO3+0.1% NH3·H2O), Mobile Phase B: ACN; Flow rate: 60 mL/min mL/min; Gradient: 14% B to 32% B in 10 min; Wave Length: 254 nm/220 nm; RT1(min): 11.25) to afford (12R)-12-methyl-24-(methylamino)-14-oxa-2,4,5,7,11,20-hexaazapentacyclo [13.6.2.2\hat{ }(3,6.0\hat{ }(5,9.0\hat{ }(19,23]pentacosa-1(21),3,6,8,15,17,19,22,24-nonaen-10-one (21 mg, 38%) as a white solid. LCMS:[M+H]f=390.15. NMR: ¹H NMR (300 MHz, DMSO-d6) δ 10.01 (s, 1H), 9.59-9.52 (m, 1H), 8.82-8.68 (m, 2H), 7.89 (s, 1H), 7.72 (d, J=8.4 Hz, 1H), 7.63-7.47 (m, 2H), 7.37 (dd, J=7.5, 1.0 Hz, 1H), 5.87 (s, 1H), 4.37 (s, 1H), 4.21 (dd, J=10.1, 6.2 Hz, 1H), 4.06 (dd, J=10.0, 2.2 Hz, 1H), 2.92 (d, J=4.8 Hz, 3H), 1.32 (d, J=6.6 Hz, 3H).

### Example 27: Synthesis of Compound 65

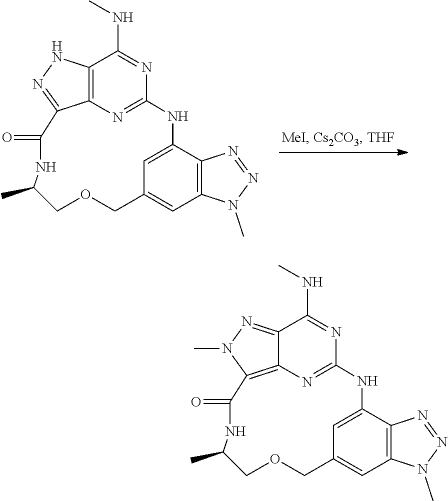

To a stirred mixture of (14R)-7,14-dimethyl-21-(methylamino)-12-oxa-2,5,6,7,15,18,19,22,23-nonaazapentacyclo[15.5.2.1\hat{ }(3,10.0\hat{ }(4,8.0\hat{ }(20,24]pentacosa-1(23),3(25),4(8),5,9,17,20(24),21-octaen-16-one (100 mg, 0.25 mmol, 1 equiv) and Cs₂CO₃ (239 mg, 0.74 mmol, 3 equiv) in THF was added Mel (42 mg, 0.3 mmol, 1.2 equiv) dropwise at room temperature under nitrogen atmosphere. The resulting mixture was stirred for 1 h at 70° C. under nitrogen atmosphere. Desired product could be detected by LCMS. The resulting mixture was concentrated under reduced pressure and the crude (50 mg) was purified by Prep-HPLC with the following conditions: (Column: Xselect CSH C18 OBD Column 30*150 mm 5 μm, n; Mobile Phase A: water (0.1% FA), Mobile Phase B: ACN; Flow rate: 60 mL/min mL/min; Gradient: 18% B to 36% B in 8 min; Wave Length: 254 nm/220 nm; RT1(min): 9.00/11.68) to afford (14R)-7,14,19-trimethyl-21-(methylamino)-12-oxa-2,5,6,7,15,18,19,22,23-nonaazapentacyclo[15.5.2.1\hat{ }(3,10.0\hat{ }(4,8.0\hat{ }(20,24]pentacosa-1(23),3(25),4(8),5,9,17,20(24),21-octaen-16-one (3.2 mg, 3%) as a white solid. LCMS: [M+H]′=423.00. NMR: ¹H NMR (400 MHz, DMSO-d₆) δ 9.03 (d, J=7.0 Hz, 2H), 8.58 (d, J=1.2 Hz, 1H), 7.55 (s, 1H), 7.26 (d, J=1.2 Hz, 1H), 4.80-4.67 (m, 2H), 4.26 (d, J=7.9 Hz, 6H), 4.04 (ddt, J=8.7, 6.5, 3.0 Hz, 1H), 3.63 (dd, J=9.6, 2.4 Hz, 1H), 3.52 (dd, J=9.6, 6.5 Hz, 1H), 3.10 (s, 3H), 1.20 (d, J=6.5 Hz, 3H).

### Example 28: Synthesis of Compound 66

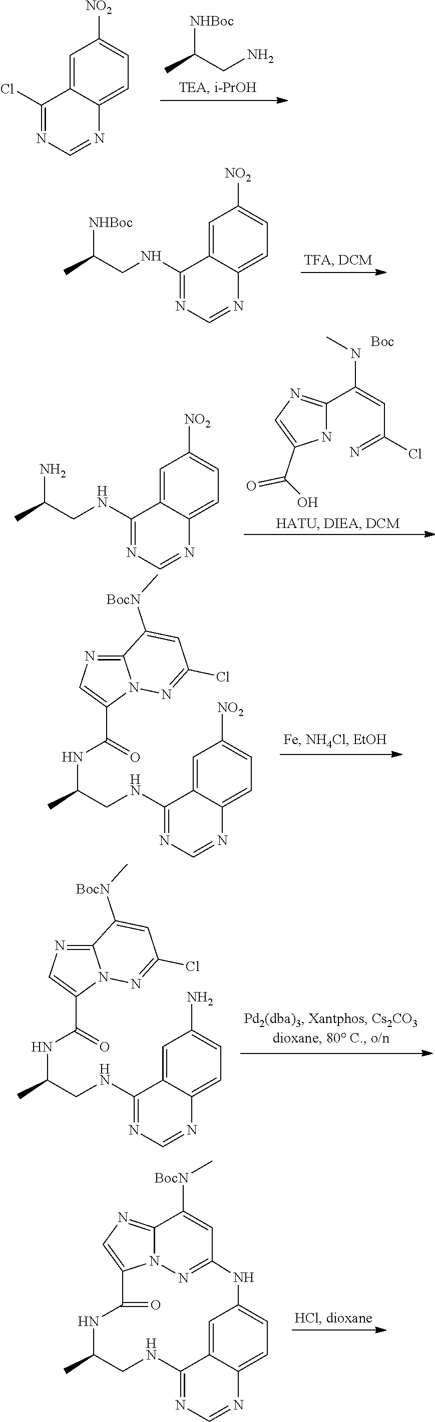

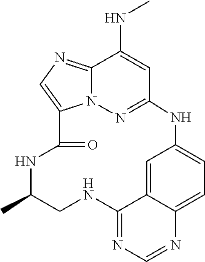 [SMILES: CNc1cc2nn3c(cnc13)C(=O)N[C@H](C)CNc1ncnc3ccc(cc13)N2]

To a stirred solution/mixture of 4-chloro-6-nitroquinazoline (1 g, 4.77 mmol, 1 equiv) and tert-butyl N-[(2R)-1-aminopropan-2-yl]carbamate (831.36 mg, 4.77 mmol, 1 equiv) in 2-propanol (5 mL) were added TEA (1931.26 mg, 19.08 mmol, 4 equiv) dropwise at room temperature. The resulting mixture was stirred for 2 h at 70° C. under argon atmosphere. The reaction was monitored by LCMS. The desired product could be detected by LCMS. The resulting mixture was concentrated under vacuum. The residue was purified by silica gel column chromatography, eluted with PE/EA (1:1) to afford tert-butyl N-[(2R)-1-[(6-nitroquinazolin-4-yl)amino]propan-2-yl]carbamate (1.6 g, 97%) as a colorless oil.

To a stirred solution/mixture of (2R)—N₁-(6-nitroquinazolin-4-yl)propane-1,2-diamine (900 mg, 3.66 mmol, 1 equiv) in DCM (10 mL) was added TFA (2 mL) dropwise/in portions at 0° C. The resulting mixture was stirred for 1 h at room temperature. The reaction was monitored by LCMS. The desired product could be detected by LCMS. The resulting mixture was concentrated under vacuum. The crude product was used in the next step directly without further purification.

To a stirred solution/mixture of (2R)—N₁-(6-nitroquinazolin-4-yl)propane-1,2-diamine (640 mg, 2.588 mmol, 1 equiv) and 8-[(tert-butoxycarbonyl)(methyl)amino]-6-chloroimidazo[1,2-b]pyridazine-3-carboxylic acid (845.73 mg, 2.59 mmol, 1 equiv) in DCM (20 mL) were added HATU (1476.29 mg, 3.88 mmol, 1.5 equiv) and DIEA (1003.62 mg, 7.76 mmol, 3 equiv) dropwise at room temperature. The resulting mixture was stirred for 2 h at room temperature under nitrogen atmosphere. The reaction was monitored by LCMS. The desired product could be detected by LCMS. The resulting mixture was concentrated under vacuum. The residue was purified by silica gel column chromatography, eluted with PE/EA (1:9) to afford tert-butyl N-(6-chloro-3-([(2R)-1-[(6-nitroquinazolin-4-yl)amino]propan-2-yl]carbamoylimidazo[1,2-b]pyridazin-8-yl)-N-methylcarbamate (1.4 g, 97%) as a yellow solid.

To a stirred solution/mixture of tert-butyl N-(6-chloro-3-([(2R)-1-[(6-nitroquinazolin-4-yl)amino]propan-2-yl]carbamoylimidazo[1,2-b]pyridazin-8-yl)-N-methylcarbamate (1.6 g, 2.88 mmol, 1 equiv) and Fe (803.55 mg, 14.39 mmol, 5 equiv) in EtOH (20 mL) and H₂O (4 mL) were added NH₄Cl (769.67 mg, 14.39 mmol, 5 equiv) in portions at room temperature. The resulting mixture was stirred for 1 h at 85° C. under argon atmosphere. The desired product could be detected by LCMS. The precipitated solids were collected by filtration and washed with EtOH (3×10 mL). The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. to afford tert-butyl N-(3-([(2R)-1-[(6-aminoquinazolin-4-yl)amino]propan-2-yl]carbamoyl-6-chloroimidazo[1,2-b]pyridazin-8-yl)-N-methylcarbamate (400 mg, 26%) as a yellow solid.

To a stirred solution/mixture of tert-butyl N-(3-([(2R)-1-[(6-aminoquinazolin-4-yl)amino]propan-2-yl]carbamoyl-6-chloroimidazo[1,2-b]pyridazin-8-yl)-N-methylcarbamate (420 mg, 0.80 mmol, 1 equiv) and Cs₂CO₃ (520.32 mg, 1.60 mmol, 2 equiv) in 1,4-dioxane (8 mL) were added Pd₂(dba)₃ (73.12 mg, 0.08 mmol, 0.1 equiv) and XantPhos (92.41 mg, 0.16 mmol, 0.2 equiv) in portions at room temperature. The resulting mixture was stirred for overnight at 85° C. under argon atmosphere. The reaction was monitored by LCMS. The desired product could be detected by LCMS. The precipitated solids were collected by filtration and washed with 1.4-dioxane (5×3 mL). The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. to afford tert-butyl N-methyl-N-[(12R)-12-methyl-10-oxo-2,4,5,7,11,14,16,18-octaazapentacyclo[13.6.2.2\hat{ }(3,6.0\hat{ }(5,9.0\hat{ }(19,23]pentacosa-1(21),3,6,8,15,17,19,22,24-nonaen-24-yl]carbamate (70 mg, 18%) as a white solid.

To a stirred solution/mixture of tert-butyl N-methyl-N-[(12R)-12-methyl-10-oxo-2,4,5,7,11,14,16,18-octaazapentacyclo[13.6.2.2\hat{ }(3,6.0\hat{ }(5,9.0\hat{ }(19,23]pentacosa-1(21),3,6,8,15,17,19,22,24-nonaen-24-yl]carbamate (67 mg, 0.14 mmol, 1 equiv) was added HCl(gas) in 1,4-dioxane (4 mL, 4N) dropwise at 0° C. under argon atmosphere. The resulting mixture was stirred for 2 h at room temperature under argon atmosphere. The reaction was monitored by LCMS. Desired product could be detected by LCMS. The resulting mixture was concentrated under vacuum. The crude product (58 mg) was purified by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 49% B to 66% B in 10 min; Wave Length: 254 nm/220 nm; RT1(min): 4.83) to afford (12R)-12-methyl-24-(methylamino)-2,4,5,7,11,14,16,18-octaazapentacyclo[13.6.2.2\hat{ }(3,6.0\hat{ }(5,9.0\hat{ }(19,23]pentacosa-1(21),3,6,8,15,17,19,22,24-nonaen-10-one (36.7 mg, 68%) as a white solid. LCMS:(ES,m/z): [M+H]⁺=390.00. NMR: ¹H NMR (400 MHz, Methanol-d4) δ 9.36 (dd, J=2.5, 1.0 Hz, 1H), 8.48 (s, 1H), 7.93 (s, 1H), 7.74 (d, J=9.0 Hz, 1H), 7.54 (dd, J=9.0, 2.5 Hz, 1H), 5.82 (s, 1H), 4.51 (tt, J=7.0, 3.6 Hz, 1H), 4.07 (dd, J=14.3, 3.0 Hz, 1H), 3.56 (dd, J=14.3, 3.7 Hz, 1H), 3.01 (s, 3H), 0.89 (d, J=7.0 Hz, 3H).

### Example 29: Synthesis of Compound 67

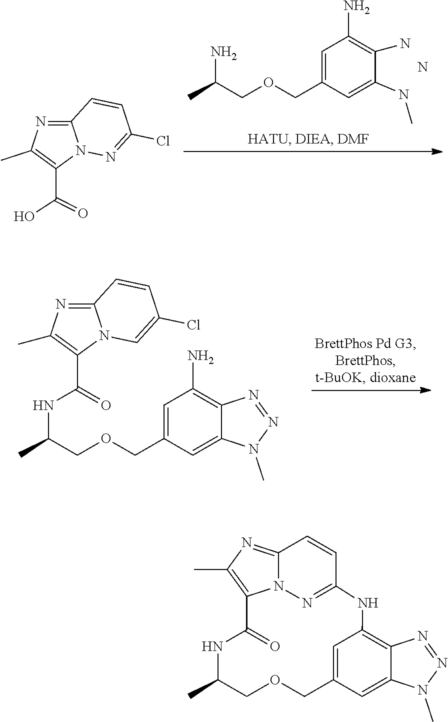

To a stirred solution 6-chloro-2-methylimidazo[1,2-b]-pyridazine-3-carboxylic acid (200 mg, 0.9 mmol, 1 equiv) and 6-{[(2R)-2-aminopropoxy]methyl}-1-methyl-1,2,3-benzotriazol-4-amine (266 mg, 1.1 mmol, 1.2 equiv) in DMF (5 ml) were added HATU (539 mg, 1.4 mmol, 1.5 equiv) and DIEA (244 mg, 1.9 mmol, 2 equiv) in portions at room temperature under nitrogen atmosphere. The resulting mixture was stirred for 2 h at room temperature under nitrogen atmosphere. The desired product could be detected by LCMS. The residue was purified by reversed-phase flash chromatography with the following conditions: column, C18 silica gel; mobile phase, MeCN in water, 30% to 50% gradient in 10 min; detector, UV 254 nm. This resulted in N-[(2R)-1-[(7-amino-3-methyl-1,2,3-benzotriazol-5-yl)methoxy]propan-2-yl]-6-chloro-2-methylimidazo[1,2-b]pyridazine-3-carboxamide (170 mg, 42%) as a yellow solid.

A solution of N-[(2R)-1-[(7-amino-3-methyl-1,2,3-benzotriazol-5-yl)methoxy]propan-2-yl]-6-chloro-2-methylimidazo[1,2-b]pyridazine-3-carboxamide (140 mg, 0.3 mmol, 1 equiv) in 1,4-dioxane (3 ml) was treated with t-BuOK (73.3 mg, 0.7 mmol, 2 equiv) for 5 min at room temperature under nitrogen atmosphere followed by the addition of BrettPhos Pd G3 (29.6 mg, 0.03 mmol, 0.1 equiv) and BrettPhos (17.5 mg, 0.03 mmol, 0.1 equiv) at room temperature. The resulting mixture was stirred for overnight at 90° C. under nitrogen atmosphere. The desired product could be detected by LCMS. The resulting mixture was concentrated under reduced pressure. The residue was purified by silica gel column chromatography, eluted with CH₂Cl₂/MeOH (10:1) to afford (14R)-7,14,18-trimethyl-12-oxa-2,5,6,7,15,19,23,24-octaazapentacyclo[15.5.2.1\hat{ }(3,10.0\hat{ }(4,8.0\hat{ }(20,24]pentacosa-1(23),3,5,8,10(25),17,19,21-octaen-16-one (32.5 mg, 25%) was obtained as a white solid. LCMS: [M+H]⁺=393.10. NMR: 1H NMR (400 MHz, DMSO-d₆) δ 10.52 (s, 1H), 8.69 (d, J=5.9 Hz, 1H), 8.29 (s, 1H), 8.03 (d, J=9.7 Hz, 1H), 7.49 (d, J=9.8 Hz, 1H), 7.37 (s, 1H), 4.87 (d, J=14.2 Hz, 1H), 4.59 (d, J=14.3 Hz, 1H), 4.30 (s, 3H), 3.56 (dd, J=9.7, 2.7 Hz, 1H), 2.61 (s, 3H), 1.10 (d, J=6.5 Hz, 3H).

### Example 30: Synthesis of Compound 68

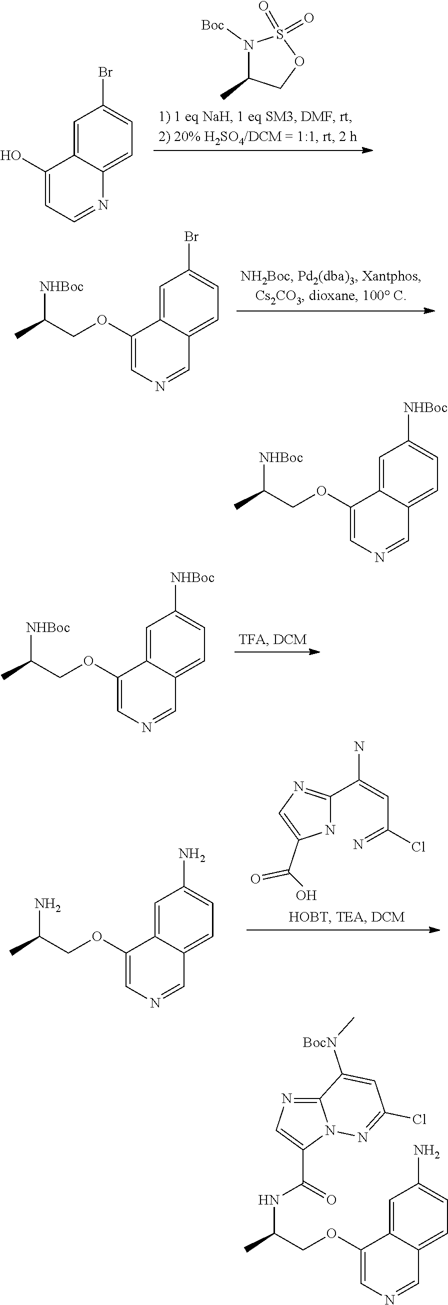

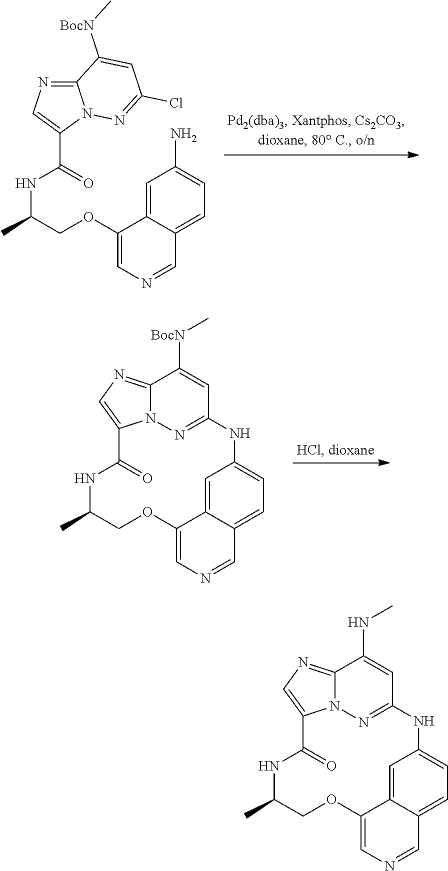 [SMILES: C[C@H](COc1cncc2ccc(N)cc12)NC(=O)c1cnc2c(N(C)C(=O)OC(C)(C)C)cc(Cl)nn12 ; C[C@@H]1COc2cncc3ccc(cc23)Nc2cc(N(C)C(=O)OC(C)(C)C)c3ncc(n3n2)C(=O)N1 ; CNc1cc2nn3c(cnc13)C(=O)N[C@H](C)COc1cncc3ccc(cc13)N2]

To a stirred mixture of 6-bromoisoquinolin-4-ol (1 g, 4.4 mmol, 1 equiv) in DMF (20 mL) was added NaH (214 mg, 5.3 mmol, 1.2 equiv, 60% in oil) dropwise at 0° C. under nitrogen atmosphere. The resulting mixture was stirred for 5 min at 0° C. under nitrogen atmosphere. The stirred solution tert-butyl (4R)-4-methyl-2,2-dioxo-1,2lambda6,3-oxathiazolidine-3-carboxylate (523 mg, 0.22 mmol, 1 equiv) in DMF (5 mL) was dropwise to the above mixture at 0° C. under nitrogen atmosphere. The resulting mixture was stirred for 2 h at room temperature under nitrogen atmosphere. To the above mixture was added 20% H₂SO₄ in water/DCM (1:1, 20 mL) dropwise over 5 min at room temperature. The resulting mixture was stirred for additional 1 h at room temperature. The desired product could be detected by LCMS. The reaction was quenched by the addition of NaHCO₃ (30 mL) at 0° C. The resulting mixture was extracted with EtOAc (3×50 mL). The combined organic layers were washed with brine (1×50 mL), dried over anhydrous Na2SO4. After filtration, the filtrate was concentrated under reduced pressure. The residue was purified by silica gel column chromatography, eluted with PE/EA (1:1) to afford tert-butyl N-[(2R)-1-[(6-bromoisoquinolin-4-yl)oxy]propan-2-yl]carbamate (1 g, 58%) as a white solid.

To a stirred mixture of tert-butyl N-[(2R)-1-[(6-bromoisoquinolin-4-yl)oxy]propan-2-yl]carbamate (960 mg, 2.5 mmol, 1 equiv) and BocNH₂ (589 mg, 5.0 mmol, 2 equiv) in 1,4-dioxane (10 mL) were added Pd₂(dba)₃ (9.6 mg, 0.01 mmol, 0.2 equiv), Xantphos (12.4 mg, 0.02 mmol, 0.4 equiv) and Cs₂CO₃ (1.6 g, 5.0 mmol, 2 equiv) in portions at room temperature under air atmosphere. The resulting mixture was stirred for overnight at 100° C. under nitrogen atmosphere. The desired product could be detected by LCMS. The resulting mixture was concentrated under reduced pressure. The residue was purified by reversed-phase flash chromatography with the following conditions: column, C18 silica gel; mobile phase, MeCN in water, 20% to 60% gradient in 20 mm; detector, UV 254 nm. This resulted in tert-butyl N-[(2R)-1-((6-[(tert-butoxycarbonyl) amino]isoquinolin-4-yloxy)propan-2-yl]carbamate (640 mg, 60%) as a white solid.

To a stirred solution tert-butyl N-[(2R)-1-((6-[(tert-butoxycarbonyl)amino]isoquinolin-4-yloxy)propan-2-yl]carbamate (640 mg, 1.5 mmol, 1 equiv) in DCM (7 mL) was added TFA (1.4 mL) dropwise at 0° C. under nitrogen atmosphere. The resulting mixture was stirred for overnight at room temperature under nitrogen atmosphere. The desired product could be detected by LCMS. The resulting mixture was concentrated under reduced pressure. The crude product was used in the next step directly without further purification.

To a stirred solution 4-[(2R)-2-aminopropoxy]isoquinolin-6-amine (320 mg, 1.4 mmol, 1 equiv) and 8-[(tert-butoxycarbonyl)(methyl)amino]-6-chloroimidazo[1,2-b]pyridazine-3-carboxylic acid (481 mg, 1.4 mmol, 1 equiv) in DMF (5 mL) were added HATU (1.68 g, 4.4 mmol, 3 equiv) and DIEA (1.14 g, 8.8 mmol, 6 equiv) in portions at room temperature under argon atmosphere. The resulting mixture was stirred for 2 h at room temperature under nitrogen atmosphere. The desired product could be detected by LCMS. The residue was purified by reversed-phase flash chromatography with the following conditions: column, C18 silica gel; mobile phase, MeCN in water 10% to 50% gradient in 10 min; detector, UV 254 nm. to afford tert-butyl N-(3-([(2R)-1-[(6-aminoisoquinolin-4-yl)oxy]propan-2-yl]carbamoyl-6-chloroimidazo[1,2-b]pyridazin-8-yl)-N-methylcarbamate (550 mg, 71%) as a white solid.

To a stirred mixture of tert-butyl N-(3-([(2R)-1-[(6-aminoisoquinolin-4-yl)oxy]propan-2-yl]carbamoyl-6-chloroimidazo[1,2-b]pyridazin-8-yl)-N-methylcarbamate (530 mg, 1.0 mmol, 1 equiv) and Cs₂CO₃ (656 mg, 2.0 mmol, 2 equiv) in 1,4-dioxane (5 mL) were added Pd₂(dba)₃ (92 mg, 0.1 mmol, 0.1 equiv) and Xantphos (92 mg, 0.2 mmol, 0.2 equiv) in portions at room temperature under nitrogen atmosphere. The resulting mixture was stirred for overnight at 80° C. under nitrogen atmosphere. The desired product could be detected by LCMS. The resulting mixture was filtered, the filter cake was washed with MeCN (3×5 mL). The filtrate was concentrated under reduced pressure. The residue was purified by reversed-phase flash chromatography with the following conditions: column, C18 silica gel; mobile phase, MeCN in water, 20% to 80% gradient in 10 min; detector, UV 254 nm. This resulted in tert-butyl N-methyl-N-[(12R)-12-methyl-10-oxo-14-oxa-2,4,5,7,11,17-hexaazapentacyclo[13.6.2.2\hat{ }(3,6.0\hat{ }(5,9.0\hat{ }(19,23]pentacosa-1(21),3,6,8,15,17,19,22,24-nonaen-24-yl]carbamate (350 mg, 70%) as a white solid.

To a stirred solution tert-butyl N-methyl-N-[(12R)-12-methyl-10-oxo-14-oxa-2,4,5,7,11,17-hexaazapentacyclo[13.6.2.2\hat{ }(3,6.0\hat{ }(5,9.0\hat{ }(19,23]pentacosa-1(21),3,6,8,15,17,19,22,24-nonaen-24-yl]carbamate (170 mg, 0.34 mmol, 1 equiv) in 1,4-dioxane (5 mL) was added 4M HCl_{(gas)} in 1,4-dioxane (5 mL) dropwise 0° C. under nitrogen atmosphere. The resulting mixture was stirred for 2 h at room temperature under nitrogen atmosphere. The desired product could be detected by LCMS. The resulting mixture was concentrated under vacuum. The residue was purified by reversed-phase flash chromatography with the following conditions: Column: XBridge Shield RP18 OBD Column 30*150 mm, 5 m; Mobile Phase A: water (10 mmol/L NH₄HCO3+0.1% NH₃·H₂O), Mobile Phase B: ACN; Flow rate: 60 m/min mL/min; Gradient: 23% B to 43% B in 8 min; Wave Length: 254 nm/220 nm; RT1(min): 6.12 This resulted in (12R)-12-methyl-24-(methylamino)-14-oxa-2,4,5,7,11,17-hexaazapentacyclo[13.6.2.2\hat{ }(3,6.0\hat{ }(5,9.0\hat{ }(19,23]pentacosa-1(21),3,6,8,15,17,19,22,24-nonaen-10-one (63 mg, 46%) as a white solid. LCMS: [M+H]⁺=390.10. NMR: ¹H NMR (400 MHz, DMSO-d₆) δ 9.97 (s, 1H), 9.24 (d, J=2.2 Hz, 1H), 8.95 (s, 1H), 8.79 (d, J=6.7 Hz, 1H), 8.34 (s, 1H), 8.07 (d, J=8.8 Hz, 1H), 7.91 (s, 1H), 7.63 (q, J=4.8 Hz, 1H), 7.45 (dd, J=8.9, 2.2 Hz, 1H), 5.90 (s, 1H), 4.37 (qd, J=6.6, 2.1 Hz, 1H), 4.19 (dd, J=10.2, 6.5 Hz, 1H), 4.10 (dd, J=10.1, 2.2 Hz, 1H), 2.92 (d, J=4.8 Hz, 3H), 1.33 (d, J=6.7 Hz, 3H).

### Example 31: Synthesis of Compound 69

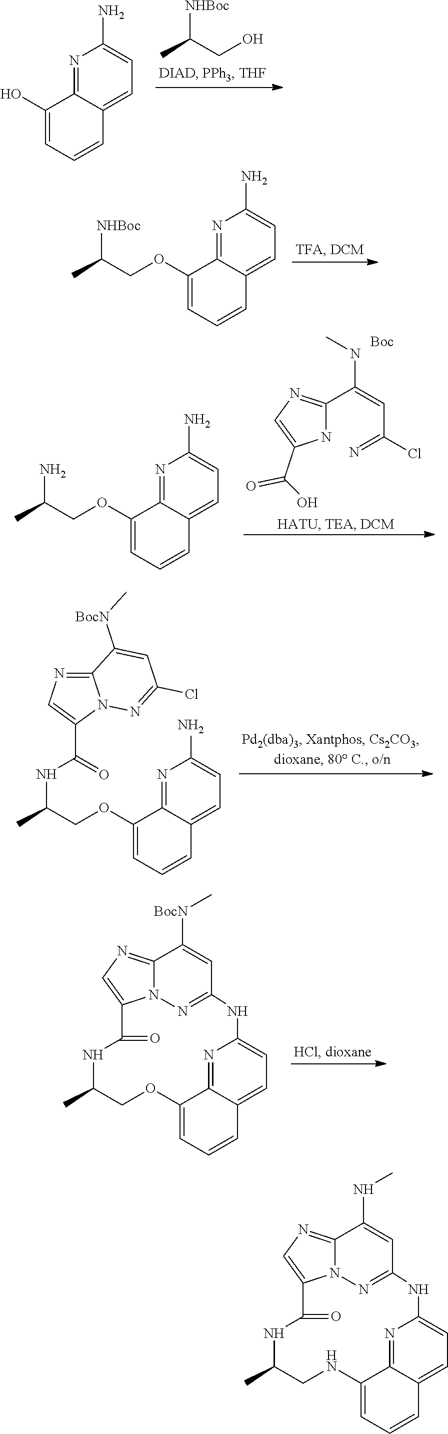

To a stirred solution/mixture of 2-aminoquinolin-8-ol (1 g, 6.243 mmol, 1 equiv) and PPh₃ (3275.05 mg, 12.49 mmol, 2 equiv) in THF (10 mL) was added tert-butyl N-[(2R)-1-hydroxypropan-2-yl]carbamate (1640.96 mg, 9.36 mmol, 1.5 equiv) dropwise at 0° C. under argon atmosphere. To the above mixture was added DIAD (2524.85 mg, 12.49 mmol, 2 equiv) dropwise over 1 min at 0° C. The resulting mixture was stirred for additional overnight at room temperature. The reaction was monitored by LCMS. The desired product could be detected by LCMS. The resulting mixture was concentrated under vacuum. The residue was purified by reversed-phase flash chromatography with the following conditions: column, C18 silica gel; mobile phase, MeCN in Water (0.10% FA), 10% to 90% gradient in 10 min; detector, UV 254 nm. to afford tert-butyl N-[(2R)-1-[(2-aminoquinolin-8-yl)oxy]propan-2-yl]carbamate (400 mg, 20%) as a light yellow solid.

A solution/mixture of tert-butyl N-[(2R)-1-[(2-aminoquinolin-8-yl)oxy]propan-2-yl]carbamate (530 mg, 1.67 mmol, 1 equiv) in DCM (5 mL) was stirred for 5 min at 0° C. under nitrogen atmosphere. To the above mixture was added TFA (1 mL) dropwise over 1 min at 0° C. The resulting mixture was stirred for additional 1 h at room temperature. The reaction was monitored by LCMS. The desired product could be detected by LCMS. The resulting mixture was concentrated under vacuum. The crude product was used in the next step directly without further purification.

To a stirred solution/mixture of 8-[(2R)-2-aminopropoxy]quinolin-2-amine (315 mg, 1.450 mmol, 1 equiv) and 8-[(tert-butoxycarbonyl)(methyl)amino]-6-chloroimidazo[1,2-b]pyridazine-3-carboxylic acid (473.71 mg, 1.45 mmol, 1 equiv) in DCM (4 mL) were added HATU (826.89 mg, 2.18 mmol, 1.5 equiv) and TEA (440.13 mg, 4.35 mmol, 3.0 equiv) dropwise at room temperature under nitrogen atmosphere. The resulting mixture was stirred for 2 h at room temperature under nitrogen atmosphere. The reaction was monitored by LCMS. Desired product could be detected by LCMS. The desired product could be detected by LCMS. The resulting mixture was concentrated under vacuum. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. to afford tert-butyl N-(3-([(2R)-1-[(2-aminoquinolin-8-yl)oxy]propan-2-yl]carbamoyl-6-chloroimidazo[1,2-b]pyridazin-8-yl)-N-methylcarbamate (740 mg, 97%) as a light yellow solid.

To a stirred solution/mixture of tert-butyl N-(3-([(2R)-1-[(2-aminoquinolin-8-yl)oxy]propan-2-yl]carbamoyl-6-chloroimidazo[1,2-b]pyridazin-8-yl)-N-methylcarbamate (300 mg, 0.57 mmol, 1 equiv) and Cs₂CO₃ (371.66 mg, 1.14 mmol, 2 equiv) in 1,4-dioxane (6 mL) were added XantPhos (66.00 mg, 0.11 mmol, 0.2 equiv) and Pd₂(dba)₃ (52.23 mg, 0.06 mmol, 0.1 equiv) in portions at room temperature. The resulting mixture was stirred for overnight at 80° C. under argon atmosphere. The reaction was monitored by LCMS. The resulting mixture was stirred for 3 h at 100° C. under argon atmosphere. The precipitated solids were collected by filtration and washed with MeOH (5×3 mL). The resulting mixture was concentrated under vacuum. The desired product could be detected by LCMS. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. to afford tert-butyl N-methyl-N-[(12R)-12-methyl-10-oxo-14-oxa-2,4,5,7,11,22-hexaazapentacyclo[13.6.2.2\hat{ }(3,6.0\hat{ }(5,9.0\hat{ }(19,23]pentacosa-1(21),3,6,8,15,17,19,22,24-nonaen-24-yl]carbamate (240 mg, 86%) as a light yellow solid.

A solution/mixture of tert-butyl N-methyl-N-[(12R)-12-methyl-10-oxo-14-oxa-2,4,5,7,11,22-hexaazapentacyclo[13.6.2.2\hat{ }(3,6.0\hat{ }(5,9.0\hat{ }(19,23]pentacosa-1(21),3,6,8,15,17,19,22,24-nonaen-24-yl]carbamate (150 mg, 0.31 mmol, 1 equiv) and HCl_{(gas)} in 1,4-dioxane (10 mL, 4 N) was stirred for 4 h at room temperature under nitrogen atmosphere. The reaction was monitored by LCMS. The desired product could be detected by LCMS. The resulting mixture was concentrated under vacuum. The crude product (110 mg) was purified by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 49% B to 66% B in 10 min; Wave Length: 254 nm/220 nm; RT1(min): 4.83) to afford (12R)-12-methyl-24-(methylamino)-14-oxa-2,4,5,7,11,22-hexaazapentacyclo[13.6.2.2\hat{ }(3,6.0\hat{ }(5,9.0\hat{ }(19,23]pentacosa-1(21),3,6,8,15,17,19,22,24-nonaen-10-one (24.7 mg, 21%) as a white solid. LCMS:(ES,m/z): [M+H]⁺=390.00. NMR: ¹H NMR (400 MHz, DMSO-d₆) δ 10.41 (d, J=5.3 Hz, 1H), 10.08 (s, 1H), 8.20 (d, J=8.9 Hz, 1H), 7.91 (s, 1H), 7.58 (d, J=8.1 Hz, 2H), 7.49-7.45 (m, 1H), 7.34 (t, J=7.8 Hz, 1H), 7.19 (d, J=8.9 Hz, 1H), 5.91 (s, 1H), 4.22 (dd, J=15.8, 6.8 Hz, 2H), 4.04 (d, J=8.8 Hz, 1H), 2.92 (d, J=4.8 Hz, 3H), 1.36 (d, J=6.3 Hz, 3H).

### Example 32: Synthesis of Compound 71

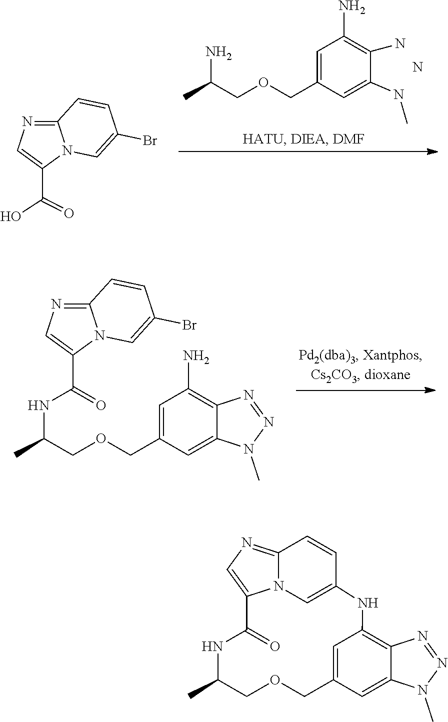

To a stirred mixture of 6-bromoimidazo[1,2-a]pyridine-3-carboxylic acid (300 mg, 1.2 mmol, 1 equiv) and 6-([(2R)-2-aminopropoxy]methyl-1-methyl-1,2,3-benzotriazol-4-amine (351 mg, 1.5 mmol, 1.2 equiv) in DMF (5 mL) was added HATU (946 mg, 2.5 mmol, 2 equiv) and DIEA (804 mg, 6.2 mmol, 5 equiv) in portions at room temperature under nitrogen atmosphere. The resulting mixture was stirred for 2 h at room temperature under nitrogen atmosphere. The desired product could be detected by LCMS. The residue was purified by reversed-phase flash chromatography with the following conditions: column, C18 silica gel; mobile phase, MeCN in water, 30% to 80% gradient in 10 min; detector, UV 254 nm. to afford N-[(2R)-1-[(7-amino-3-methyl-1,2,3-benzotriazol-5-yl)methoxy]propan-2-yl]-6-bromoimidazo[1,2-a]pyridine-3-carboxamide (340 mg, 59%) as a brown solid.

To a stirred mixture of N-[(2R)-1-[(7-amino-3-methyl-1,2,3-benzotriazol-5-yl) methoxy]propan-2-yl]-6-bromoimidazo[1,2-a]pyridine-3-carboxamide (190 mg, 0.4 mmol, 1 equiv) and Pd₂(dba)₃ (37 mg, 0.04 mmol, 0.1 equiv) in 1,4-dioxane (2 mL) were added Xantphos (47 mg, 0.08 mmol, 0.2 equiv) and Cs₂CO₃ (270 mg, 0.8 mmol, 2 equiv) in portions at room temperature under nitrogen atmosphere. The resulting mixture was stirred for overnight at 80° C. under nitrogen atmosphere. The desired product could be detected by LCMS. The resulting mixture was concentrated under reduced pressure. The residue was purified by reversed-phase flash chromatography with the following conditions: column, C18 silica gel; mobile phase, MeCN in water (0.1% FA), 10% to 50% gradient in 10 min; detector, UV 254 nm. This resulted in (14R)-7,14-dimethyl-12-oxa-2,5,6,7,15,19,24-heptaazapentacyclo[15.5.2.1\hat{ }(3,10.0\hat{ }(4,8.0\hat{ }(20,24]pentacosa-1(23),3,5,8,10(25),17,19,21-octaen-16-one (21 mg, 13%) was obtained as a white solid. LCMS: [M+H]⁺=378.10 NMR: ¹H NMR (400 MHz, DMSO-d6) δ 9.17 (s, 1H), 9.09 (d, J=2.2 Hz, 1H), 8.47 (d, J=8.8 Hz, 1H), 7.92 (s, 1H), 7.77 (s, 1H), 7.62 (d, J=9.6 Hz, 1H), 7.47 (dd, J=9.6, 2.1 Hz, 1H), 7.15 (s, 1H), 4.80 (d, J=13.4 Hz, 1H), 4.54 (d, J=13.5 Hz, 1H), 4.26 (s, 3H), 4.17-4.07 (m, 1H), 3.60 (dd, J=9.0, 2.5 Hz, 1H), 3.50 (dd, J=9.0, 4.7 Hz, 1H), 1.22 (d, J=6.7 Hz, 3H).

### Example 33: Synthesis of Compound 72

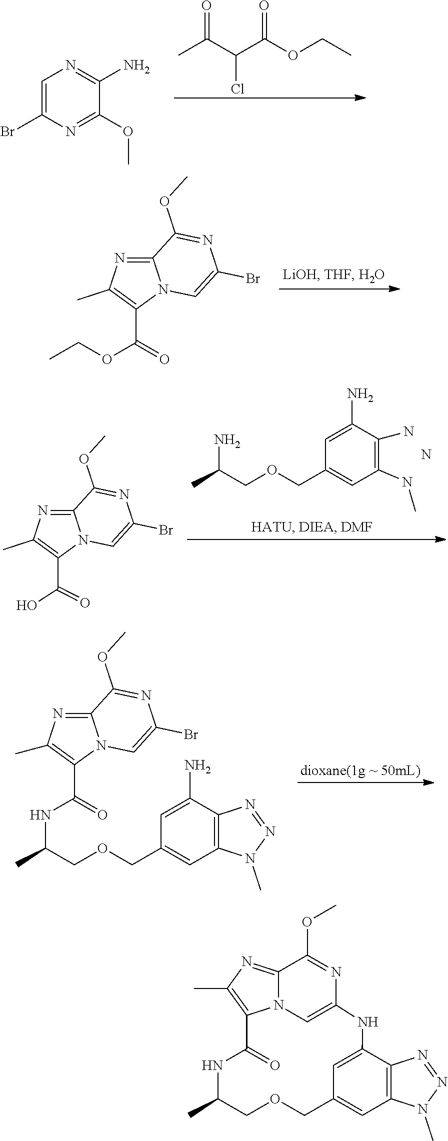

A solution of 5-bromo-3-methoxypyrazin-2-amine (1 g, 4.9 mmol, 1 equiv) and ethyl 2-chloro-3-oxopropanoate (2.4 g, 15.7 mmol, 1.6 equiv) in EtOH (20 mL) was stirred for overnight at 80° C. under argon atmosphere. The desired product could be detected by LCMS. The solvent was removed under reduced pressure. The residue was purified by reversed-phase flash chromatography with the following conditions: column, C18 silica gel; mobile phase, MeCN in Water (0.1% FA), 30% to 80% gradient in 20 min; detector, UV 254 nm. This resulted in ethyl 6-bromo-8-methoxy-2-methylimidazo[1,2-a]pyrazine-3-carboxylate (253 mg, 16%) as a yellow solid.

A solution of ethyl 6-bromo-8-methoxy-2-methylimidazo[1,2-a]pyrazine-3-carboxylate (252 mg, 0.80 mmol, 1 equiv) and LiOH (57.6 mg, 2.4 mmol, 3 equiv) in THF/H₂O (20 mL/5 mL) was stirred for overnight at room temperature under argon atmosphere. The desired product could be detected by LCMS. The mixture was acidified to pH 5 with resin. The mixture was filtered and the filtrate was concentrated under reduced pressure. The resulting mixture was concentrated under vacuum. This resulted in 6-bromo-8-methoxy-2-methylimidazo[1,2-a]pyrazine-3-carboxylic acid (204 mg, 89%) as an off-white solid. The resulting mixture was used in the next step directly without further purification.

To a stirred solution of 6-bromo-8-methoxy-2-methylimidazo[1,2-a]pyrazine-3-carboxylic acid (170 mg, 0.59 mmol, 1 equiv) and 6-([(2R)-2-aminopropoxy]methyl-1-methyl-1,2,3-benzotriazol-4-amine (153 mg, 0.65 mmol, 1.1 equiv) in DMF (10 mL) were added DIEA (614 mg, 4.8 mmol, 8 equiv) and HATU (338 mg, 0.89 mmol, 1.5 equiv) in portions at room temperature under argon atmosphere. The resulting mixture was stirred for 2 h at room temperature under argon atmosphere. The desired product could be detected by LCMS. The residue was purified by reversed-phase flash chromatography with the following conditions: column, C18 silica gel; mobile phase, MeCN in Water, 35% to 80% gradient in 25 min; detector, UV 254 nm. This resulted in N-[(2R)-1-[(7-amino-3-methyl-1,2,3-benzotriazol-5-yl)methoxy]propan-2-yl]-6-bromo-8-methoxy-2-methylimidazo[1,2-a]pyrazine-3-carboxamide (180 mg, 60%) as an off-white solid.

A solution of N-[(2R)-1-[(7-amino-3-methyl-1,2,3-benzotriazol-5-yl)methoxy]propan-2-yl]-6-bromo-8-methoxy-2-methylimidazo[1,2-a]pyrazine-3-carboxamide (180 mg, 0.36 mmol, 1 equiv), RuPhos Palladacycle Gen.3 (29.9 mg, 0.04 mmol, 0.1 equiv) and Cs₂CO₃ (233 mg, 0.72 mmol, 2 equiv) in 1,4-dioxane (5 mL) was stirred for 4 h at 80° C. under argon atmosphere. The desired product could be detected by LCMS. The solvent was removed under reduced pressure, The crude product was purified by Prep-HPLC with the following conditions (Column: YMC-Actus Triart C18 ExRS, 30*150 mm, 5 μm; Mobile Phase A: water (10 mmol/L NH4HCO3), Mobile Phase B: ACN; Flow rate: 60 mL/min; Gradient: 11% B to 31% B in 9 min, 310% B; Wave Length: 254/220 nm; RT1(min): 10.38; Number Of Runs: 0) to afford (14R)-21-methoxy-7,14,18-trimethyl-12-oxa-2,5,6,7,15,19,22,24-octaazapentacyclo[15.5.2.1\hat{ }(3,10.0\hat{ }(4,8.0\hat{ }(20,24]pentacosa-1(23),3(25),4(8),5,9,17,19,21-octaen-16-one (32.3 mg, 21%) as a white solid. LCMS:[M+H]⁺=423.15 HNMR: 1H NMR (400 MHz, DMSO-d6) δ 8.70 (s, 1H), 8.43 (d, J=8.9 Hz, 1H), 8.37 (s, 1H), 7.91 (s, 1H), 7.22 (s, 1H), 4.83 (d, J=13.4 Hz, 1H), 4.52 (d, J=13.4 Hz, 1H), 4.26 (s, 3H). 4.11 (s, 4H), 3.57 (dd, J=9.0, 2.4 Hz, 1H), 3.48 (dd, J=9.0, 4.7 Hz, 1H), 2.39 (s, 3H), 1.26-1.19 (m, 3H).

### Example 34: Synthesis of Compound 73

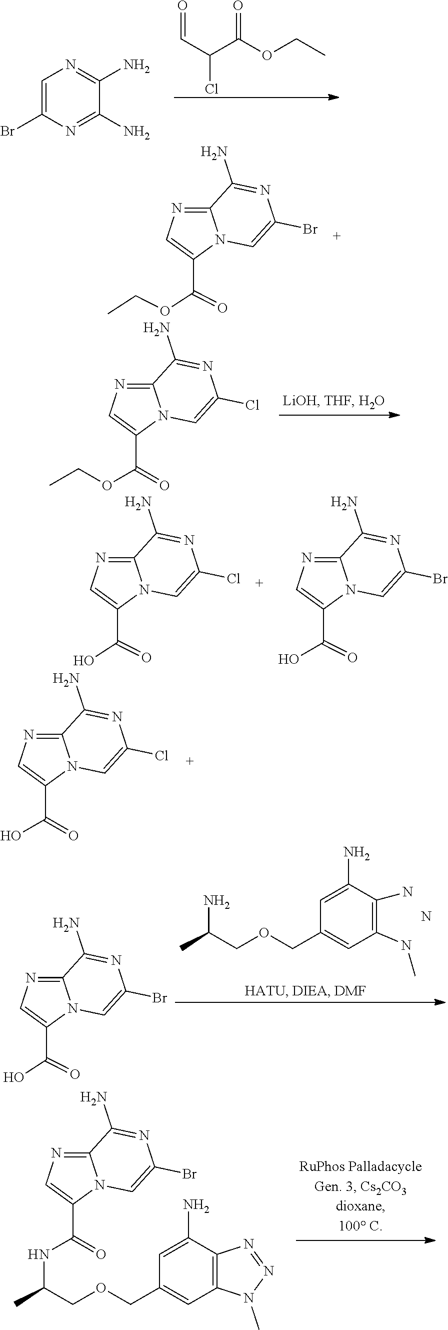

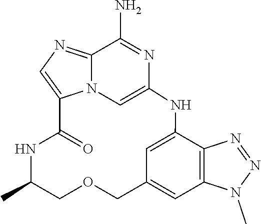 [SMILES: C[C@@H]1COCc2cc(c3nnn(C)c3c2)Nc2cn3c(cnc3c(N)n2)C(=O)N1]

A solution of 5-bromopyrazine-2,3-diamine (1 g, 5.3 mmol, 1 equiv) and ethyl 2-chloro-3-oxopropanoate (1.3 g, 8.5 mmol, 1.6 equiv) in EtOH (20 mL) was stirred for overnight at 80° C. under nitrogen atmosphere. The mixture was allowed to cool down to room temperature. The desired product could be detected by LCMS. The solvent was removed under reduced pressure. The residue was purified by reversed-phase flash chromatography with the following conditions: column, C18 silica gel; mobile phase, MeCN in water (0.1% FA), 60% to 80% gradient in 10 min; detector, UV 254 nm. This resulted in ethyl 8-amino-6-bromoimidazo[1,2-a]pyrazine-3-carboxylate (280 mg, 18%) as a yellow solid.

To a stirred solution of ethyl 8-amino-6-chloroimidazo[1,2-a]pyrazine-3-carboxylate (380 mg, 1.6 mmol, 1 equiv) and H₂O (3 mL) in THF (12 mL) was added LiOH (151 mg, 6.3 mmol, 4 equiv) in portions at room temperature under nitrogen atmosphere. The resulting mixture was stirred for 3 h at room temperature under nitrogen atmosphere. The desired product could be detected by LCMS. The mixture was acidified to pH 4 with resin. The mixture was filtered, and the filtrate was concentrated under reduced pressure. This resulted in 8-amino-6-chloroimidazo[1,2-a]pyrazine-3-carboxylic acid (225 mg, 67%) as a white solid. The resulting mixture was used in the next step directly without further purification.

To a stirred solution of 8-amino-6-bromoimidazo[1,2-a]pyrazine-3-carboxylic acid (205 mg, 0.8 mmol, 1 equiv) and 6-([(2R)-2-aminopropoxy]methyl-1-methyl-1,2,3-benzotriazol-4-amine (281 mg, 1.2 mmol, 1.5 equiv) in DMF (5 mL) were added DIEA (515 mg, 4 mmol, 5 equiv) and HATU (606 mg, 1.6 mmol, 2 equiv) in portions at room temperature under nitrogen atmosphere. The desired product could be detected by LCMS. The residue was purified by reversed-phase flash chromatography with the following conditions: column, C18 silica gel; mobile phase, MeCN in water, 30% to 50% gradient in 10 min; detector, UV 254 nm. This resulted in 8-amino-N-[(2R)-1-[(7-amino-3-methyl-1,2,3-benzotriazol-5-yl)methoxy]propan-2-yl]-6-bromoimidazo[1,2-a]pyrazine-3-carboxamide (321 mg, 85%) as a red solid.

To a stirred solution of 8-amino-N-[(2R)-1-[(7-amino-3-methyl-1,2,3-benzotriazol-5-yl)methoxy]propan-2-yl]-6-bromoimidazo[1,2-a]pyrazine-3-carboxamide (150 mg, 0.32 mmol, 1 equiv) and RuPhos Palladacycle Gen.3 (132 mg, 0.16 mmol, 0.5 equiv) in dioxane (5 mL) was added Cs2CO3 (309 mg, 0.95 mmol, 3 equiv) in portions at room temperature under nitrogen atmosphere. The resulting mixture was stirred for overnight at 100° C. under nitrogen atmosphere. The mixture was allowed to cool down to room temperature. The desired product could be detected by LCMS. The residue was purified by reversed-phase flash chromatography with the following conditions: column, C18; mobile phase, MeCN in water, 60% to 70% gradient in 10 min; detector, UV 254 nm. The crude product was purified by Prep-HPLC with the following conditions (Column: XBridge Prep Phenyl OBD Column, 19*150 mm, 5 μm; Mobile Phase A: water (10 mmol/L NH4HCO3), Mobile Phase B: ACN; Flow rate: 50 mL/min; Gradient: 16% B to 33% B in 8 min, 33% B; Wave Length: 254/220 nm; RT1(min): 7.05; Number Of Runs: 0) to afford (14R)-21-amino-7,14-dimethyl-12-oxa-2,5,6,7,15,19,22,24-octaazapentacyclo[15.5.2.1\hat{ }(3,10.0\hat{ }(4,8.0\hat{ }(20,24]pentacosa-1(23),3(25),4(8),5,9,17,19,21-octaen-16-one (5.7 mg, 4.4%) as a white solid. LCMS: [M+H]′=394.15 NMR: 1H NMR (400 MHz, DMSO-d6) δ 8.39 (d, J=8.7 Hz, 1H), 8.10 (s, 1H), 8.06 (s, 1H), 7.92 (s, 1H), 7.20 (s, 1H), 7.13 (s, 2H), 4.79 (d, J=13.1 Hz, 1H), 4.53 (d, J=13.2 Hz, 1H), 4.35-4.25 (m, 1H), 4.25 (s, 3H), 4.08 (d, J=6.3 Hz, 1H), 3.57 (dd, J=9.1, 2.6 Hz, 1H), 3.50 (dd, J=9.1, 5.1 Hz, 1H), 1.26-1.17 (m, 3H).

### Example 35: Synthesis of Compound 74

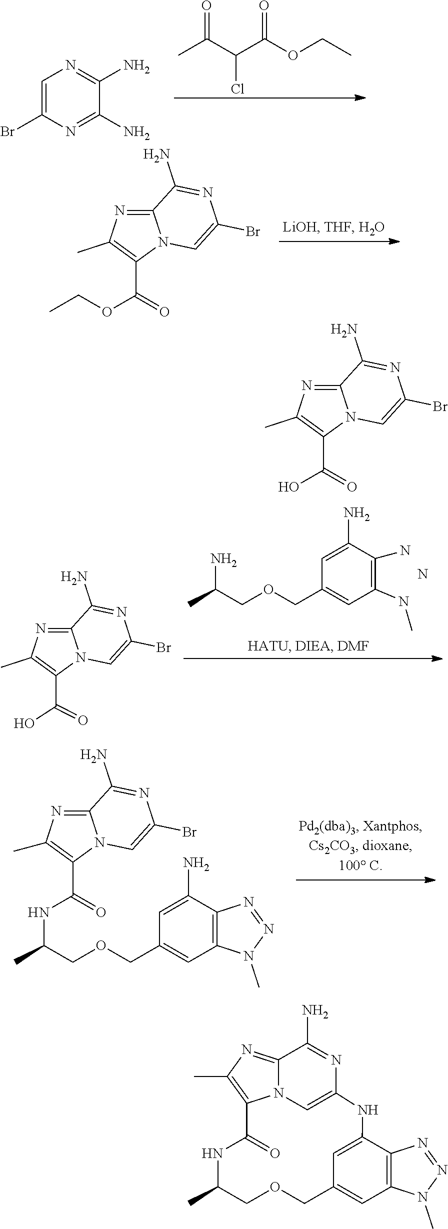

A solution of 3,5-dibromopyrazin-2-amine (2 g, 8 mmol, 1 equiv) and ethyl 2-chloro-3-oxobutanoate (2.1 g, 12.6 mmol, 1.6 equiv) in EtOH (20 mL) was stirred for overnight at 80° C. under nitrogen atmosphere. The mixture was allowed to cool down to room temperature. The desired product could be detected by LCMS. The resulting mixture was concentrated under vacuum. The residue was purified by reversed-phase flash chromatography with the following conditions: column, C18 silica gel; mobile phase, MeCN in water, 50% to 65% gradient in 10 min; detector, UV 254 nm. This resulted in ethyl 6,8-dibromo-2-methylimidazo[1,2-a]pyrazine-3-carboxylate (440 mg, 15%) as a yellow oil.

To a stirred solution of ethyl 8-amino-6-bromo-2-methylimidazo[1,2-a]pyrazine-3-carboxylate (620 mg, 2.1 mmol, 1 equiv) and H2O (3 mL) in THF (6 mL) was added LiOH (435 mg, 10 mmol, 5 equiv) in portions at room temperature under nitrogen atmosphere. The resulting mixture was stirred for 3 h at room temperature under nitrogen atmosphere. The mixture was acidified to pH 7 with resin. The precipitated solids were collected by filtration and washed with MeOH (10×4 mL). The resulting mixture was concentrated under vacuum. This resulted in 8-amino-6-bromo-2-methylimidazo[1,2-a]pyrazine-3-carboxylic acid (500 mg, 89%) as a white solid. The resulting mixture was used in the next step directly without further purification.

To a stirred solution of 8-amino-6-bromo-2-methylimidazo[1,2-a]pyrazine-3-carboxylic acid (200 mg, 0.74 mmol, 1 equiv) and 6-([(2R)-2-aminopropoxy]methyl-1-methyl-1,2,3-benzotriazol-4-amine (208 mg, 0.886 mmol, 1.2 equiv) in DMF (5 mL) were added DIEA (477 mg, 3.7 mmol, 5 equiv) and HATU (561 mg, 1.5 mmol, 2 equiv) in portions at room temperature under nitrogen atmosphere. The desired product could be detected by LCMS. The residue was purified by reversed-phase flash chromatography with the following conditions: column, C18 silica gel; mobile phase, MeCN in water, 40% to 50% gradient in 10 min; detector, UV 254 nm. This resulted in 8-amino-N-[(2R)-1-[(7-amino-3-methyl-1,2,3-benzotriazol-5-yl)methoxy]propan-2-yl]-6-bromo-2-methylimidazo[1,2-a]pyrazine-3-carboxamide (200 mg, 55%) as a white solid.

To a stirred solution of 8-amino-N-[(2R)-1-[(7-amino-3-methyl-1,2,3-benzotriazol-5-yl)methoxy]propan-2-yl]-6-bromo-2-methylimidazo[1,2-a]pyrazine-3-carboxamide (270 mg, 0.55 mmol, 1 equiv) and Pd2(dba)3 (50 mg, 0.055 mmol, 0.1 equiv) in dioxane were added Xantphos (64 mg, 0.11 mmol, 0.2 equiv) and Cs2CO3 (360 mg, 1.1 mmol, 2 equiv) in portions at room temperature under nitrogen atmosphere. The resulting mixture was stirred for overnight at 100° C. under hydrogen atmosphere. The desired product could be detected by LCMS. The mixture was allowed to cool down to room temperature. The resulting mixture was concentrated under vacuum. The crude product was purified by Prep-HPLC with the following conditions (Column: XBridge Prep Phenyl OBD Column, 19*250 mm, 5 μm; Mobile Phase A: water (10 mmol/L NH4HCO3), Mobile Phase B: ACN; Flow rate: 50 mL/min; Gradient: 20% B to 37% B in 8 min, 37% B; Wave Length: 254/220 nm; RT1(min): 6.28; Number Of Runs: 0) to afford (14R)-21-amino-7,14,18-trimethyl-12-oxa-2,5,6,7,15,19,22,24-octaazapentacyclo[15.5.2.1\hat{ }(3,10.0\hat{ }(4,8.0\hat{ }(20,24]pentacosa-1(23),3(25),4(8),5,9,17,19,21-octaen-16-one (14.6 mg, 6.2%) as a light yellow solid. LCMS: [M+H]⁺=408.15 NMR: 1H NMR (400 MHz, DMSO-d6) δ 8.30 (d, J=8.8 Hz, 1H), 8.06 (s, 1H), 7.97 (s, 1H), 7.88 (s, 1H), 7.17 (s, 1H), 7.02 (s, 2H), 4.80 (d, J=13.1 Hz, 1H), 4.52 (d, J=13.3 Hz, 1H), 4.25 (s, 3H), 4.15-4.07 (m, 1H), 3.56 (dd, J=9.1, 2.5 Hz, 1H), 3.49 (dd, J=9.1, 4.8 Hz, 1H), 2.39 (s, 3H), 1.21 (d, J=6.8 Hz, 3H).

### Example 36: Synthesis of Compound 75

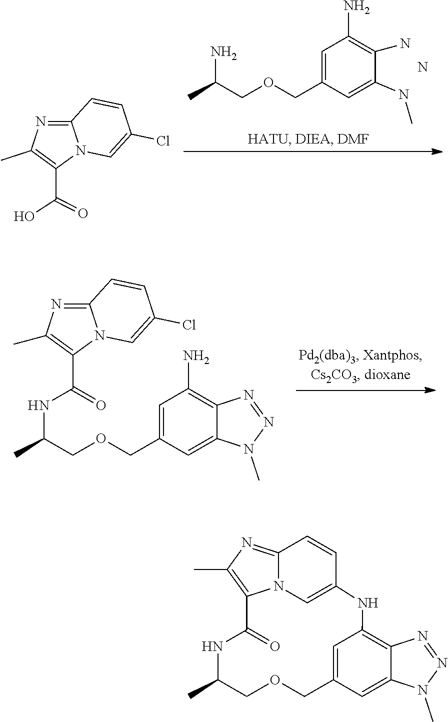

To a stirred solution of 6-chloro-2-methylimidazo[1,2-a]pyridine-3-carboxylic acid (200 mg, 0.9 mmol, 1 equiv) and 6-([(2R)-2-aminopropoxy]methyl-1-methyl-1,2,3-benzotriazol-4-amine (268 mg, 1.1 mmol, 1.2 equiv) in DMF (5 ml) were added DIEA (981 mg, 7.6 mmol, 8 equiv) and HATU (541 mg, 1.4 mmol, 1.5 equiv) in portions at room temperature under argon atmosphere. The resulting mixture was stirred for 2 h at room temperature under argon atmosphere. The desired product could be detected by LCMS. The residue was purified by reversed-phase flash chromatography with the following conditions: column, C18 silica gel; mobile phase, MeCN in water, 10% to 80% gradient in 30 min; detector, UV 254 nm. This resulted in N-[(2R)-1-[(7-amino-3-methyl-1,2,3-benzotriazol-5-yl)methoxy]propan-2-yl]-6-chloro-2-methylimidazo[1,2-a]pyridine-3-carboxamide (300 mg, 73%) as a brown yellow oil.

A solution of N-[(2R)-1-[(7-amino-3-methyl-1,2,3-benzotriazol-5-yl)methoxy]propan-2-yl]-6-chloro-2-methylimidazo[1,2-a]pyridine-3-carboxamide (150 mg, 0.35 mmol, 1 equiv), Pd₂(dba)₃ (32 mg, 0.04 mmol, 0.1 equiv), Xantphos (40 mg, 0.07 mmol, 0.2 equiv) and Cs₂CO₃ (228 mg, 0.7 mmol, 2 equiv) in 1,4-dioxane (3 ml) was stirred for overnight at 110° C. under argon atmosphere. The desired product could be detected by LCMS. The crude product was purified by Prep-HPLC with the following conditions (Column: XBridge Shield RP18 OBD Column 30*150 mm, 5 μm; Mobile Phase A: water (10 mmol/L NH₄HCO₃), Mobile Phase B: ACN; Flow rate: 60 mL/min mL/min; Gradient: isocratic Wave Length: 254 nm/220 nm; RT1(min): 12.07) to afford (14R)-7,14,18-trimethyl-12-oxa-2,5,6,7,15,19,24-heptaazapentacyclo[15.5.2.1\hat{ }(3,10.0\hat{ }(4,8.0\hat{ }(20,24]pentacosa-1(23),3,5,8,10(25),17,19,21 octaen-16-one (43.1 mg, 310%) was obtained as a white solid. LCMS:[M+H]⁺=392.30 HNMR: 1H NMR (400 MHz, DMSO-d6) δ 9.10 (s, 1H), 9.02 (d, J=2.2 Hz, 1H), 8.38 (d, J=9.0 Hz, 1H), 7.87 (s, 1H), 7.48 (d, J=9.5 Hz, 1H), 7.40 (dd, J=9.5, 2.1 Hz, 1H), 7.12 (s, 1H), 4.80 (d, J=13.5 Hz, 1H), 4.52 (d, J=13.6 Hz, 1H), 4.25 (s, 3H), 4.18-4.09 (m, 1H), 3.58 (dd, J=9.1, 2.5 Hz, 1H), 3.49 (dd, J=9.0, 4.6 Hz, 1H), 2.40 (s, 3H), 1.22 (d, J=6.7 Hz, 3H).

### Example 37: Synthesis of Compound 76

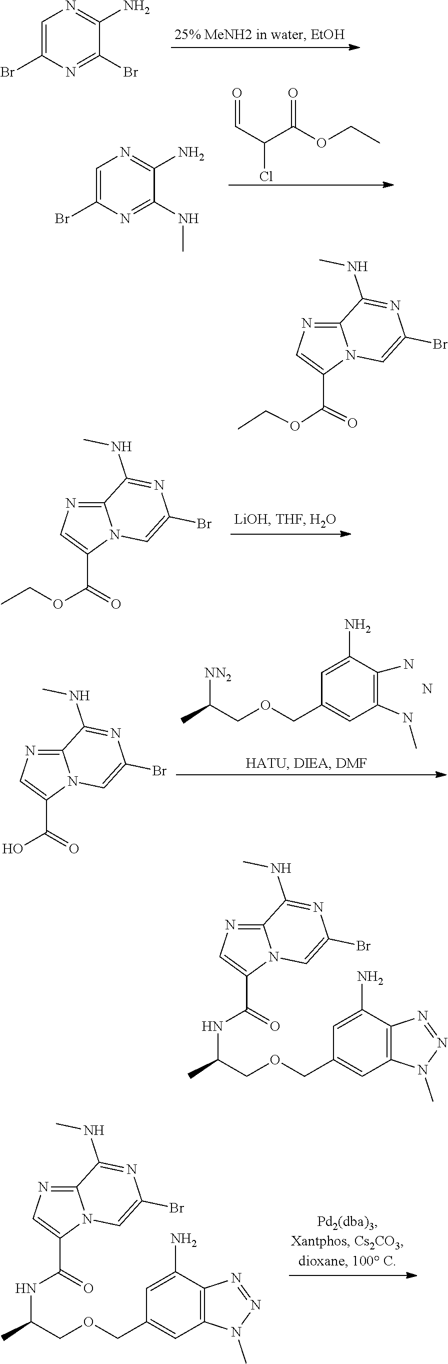

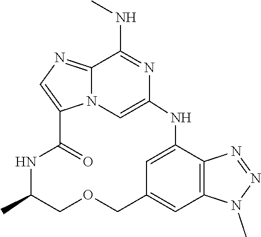 [SMILES: CNc1nc2cn3c(cnc13)C(=O)N[C@H](C)COCc1cc(c3nnn(C)c3c1)N2]

A solution of 3,5-dibromopyrazin-2-amine (10 g, 39.5 mmol, 1 equiv) and methylamine (24.6 g, 198 mmol, 5 equiv, 25% in water) in EtOH (100 mL) was stirred for overnight at 100° C. under nitrogen atmosphere. The mixture was allowed to cool down to room temperature. The resulting mixture was concentrated under vacuum. The residue was purified by silica gel column chromatography, eluted with PE/EA (1:1) to afford 6-bromo-N2-methylpyrazine-2,3-diamine (7.1 g, 88%) as a yellow solid.

A solution of 6-bromo-N2-methylpyrazine-2,3-diamine (2 g, 9.9 mmol, 1 equiv) and ethyl 2-chloro-3-oxopropanoate (2.4 g, 15.8 mmol, 1.6 equiv) in EtOH (50 mL) was stirred for overnight at 80° C. under nitrogen atmosphere. The mixture was allowed to cool down to room temperature. The desired product could be detected by LCMS. The resulting mixture was concentrated under vacuum. The residue was purified by silica gel column chromatography, eluted with PE/EA (2:1) to afford ethyl 6-bromo-8-(methylamino)imidazo[1,2-a]pyrazine-3-carboxylate (1.6 g, 54%) as a yellow solid.

To a stirred solution of ethyl 6-bromo-8-(methylamino)imidazo[1,2-a]pyrazine-3-carboxylate (500 mg, 1.672 mmol, 1 equiv) and THF/H2O (9 mL/3 mL) in water was added LiOH (120 mg, 5 mmol, 3 equiv) in portions at 0° C. under nitrogen atmosphere. The resulting mixture was stirred for 4 h at room temperature under nitrogen atmosphere. The desired product could be detected by LCMS. The mixture was acidified to pH 5 with resin. The mixture was filtered, and the filtrate was concentrated under reduced pressure. The resulting mixture was concentrated under reduced pressure. This resulted in 6-bromo-8-(methylamino)imidazo[1,2-a]pyrazine-3-carboxylic acid (340 mg, 71%) as a white solid.

To a stirred solution of 6-chloro-8-(methylamino)imidazo[1,2-a]pyrazine-3-carboxylic acid (150 mg, 0.66 mmol, 1 equiv) and 6-([(2R)-2-aminopropoxy]methyl-1-methyl-1,2,3-benzotriazol-4-amine (195.30 mg, 0.830 mmol, 1.5 equiv) in DMF (5 mL) were added DIEA (357 mg, 2.8 mmol, 5 equiv) and HATU (420 mg, 1.1 mmol, 2 equiv) in portions at room temperature under nitrogen atmosphere. The resulting mixture was stirred for 3 h at room temperature under nitrogen atmosphere. The desired product could be detected by LCMS. The residue was purified by reversed-phase flash chromatography with the following conditions: column, C18 silica gel; mobile phase. MeCN in water, 50% to 70% gradient in 10 min; detector, UV 254 nm. This resulted in N-[(2R)-1-[(7-amino-3-methyl-1,2,3-benzotriazol-5-yl)methoxy]propan-2-yl]-6-chloro-8-(methylamino)imidazo[1,2-a]pyrazine-3-carboxamide (193 mg, 65%) as a red solid.

To a stirred solution of N-[(2R)-1-[(7-amino-3-methyl-1,2,3-benzotriazol-5-yl)methoxy]propan-2-yl]-6-bromo-2-(methylamino)imidazo[1,2-a]pyrazine-3-carboxamide (150 mg, 0.31 mmol, 1 equiv) and RuPhos Palladacycle Gen.3 (26 mg, 0.03 mmol. 0.1 equiv) in dioxane (5 mL) was added Cs2CO3 (200 mg, 0.6 mmol, 2 equiv) in portions at room temperature under nitrogen atmosphere. The resulting mixture was stirred for overnight at 100° C. under nitrogen atmosphere. The desired product could be detected by LCMS. The solvent was removed under reduced pressure. The residue was purified by reversed-phase flash chromatography with the following conditions: column, C18 silica gel; mobile phase, MeCN in water, 50% to 60% gradient in 10 min; detector, UV 254 nm. The residue was purified by reversed-phase flash chromatography with the following conditions: column, C18 silica gel; mobile phase, MeCN in water, 55% to 66% gradient in 10 min; detector, UV 254 nm. to afford (14R)-7,14-dimethyl-21-(methylamino)-12-oxa-2,5,6,7,15,19,22,24-octaazapentacyclo[15.5.2.1\hat{ }(3,10.0\hat{ }(4,8.0\hat{ }(20,24]pentacosa-1(23),3(25),4(8),5,9,17,19,21-octaen-16-one (12.5 mg, 10%) as a yellow solid. LCMS: [M+H]⁺=408.00 NMR: 1H NMR (400 MHz, DMSO-d6) δ 8.40 (d, J=8.7 Hz, 1H), 8.09 (d, J=7.6 Hz, 2H), 7.92 (s, 1H), 7.69 (d, J=17.0 Hz, 2H), 7.20 (s, 1H), 4.79 (d, J=13.1 Hz, 1H), 4.54 (d, J=13.2 Hz, 1H), 4.25 (s, 3H), 4.09 (s, 1H), 3.58 (dd, J=9.0, 2.5 Hz, 1H), 3.51 (dd, J=9.0, 5.2 Hz, 1H), 3.03 (d, J=4.8 Hz, 3H), 1.26-1.17 (m, 3H).

### Example 38: Synthesis of Compound 77

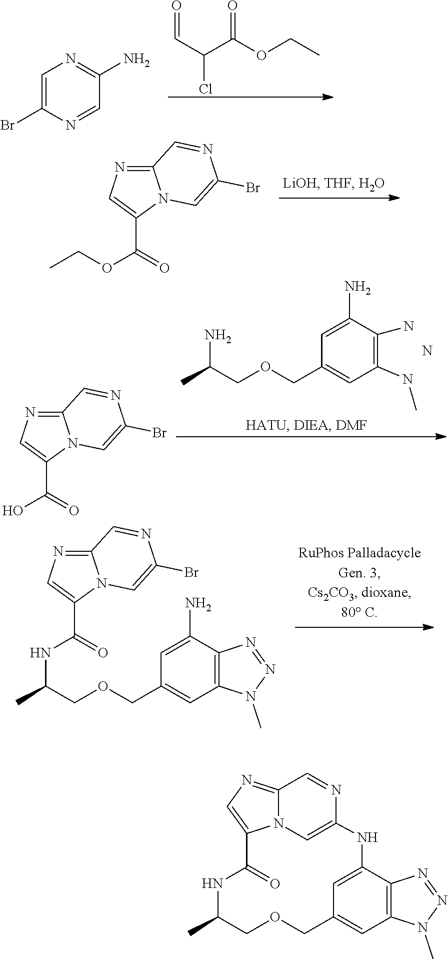

A solution of 5-bromo-3-methoxypyrazin-2-amine (1 g, 4.9 mmol, 1 equiv) and ethyl 2-chloro-3-oxopropanoate (1.2 g, 7.8 mmol, 1.6 equiv) in EtOH (20 mL) was stirred for overnight at 80° C. under argon atmosphere. The mixture was allowed to cool down to room temperature. The desired product could be detected by LCMS. The solvent was removed under reduced pressure. The residue was purified by reversed-phase flash chromatography with the following conditions: column, C18 silica gel; mobile phase, MeCN in Water, 40% to 50% gradient in 10 min; detector, UV 254 nm. This resulted in ethyl 6-bromo-8-methoxyimidazo[1,2-a]pyrazine-3-carboxylate (224 mg, 15%) as a yellow solid.

A solution of ethyl 6-bromoimidazo[1,2-a]pyrazine-3-carboxylate (120 mg, 0.44 mmol, 1 equiv) in THF (5 mL) was treated with H2O (1 mL) for 2 min at 0° C. under nitrogen atmosphere followed by the addition of LiOH (31.9 mg, 1.33 mmol, 3 equiv) in portions at 0° C. The resulting mixture was stirred for 2 h at room temperature under nitrogen atmosphere. The desired product could be detected by LCMS. The mixture was acidified to pH 6 with resin. The resulting mixture was filtered, the filter cake was washed with MeOH (4×10 mL). The filtrate was concentrated under reduced pressure. This resulted in 6-bromoimidazo[1,2-a]pyrazine-3-carboxylic acid (95 mg, 88%) as a yellow solid. The resulting mixture was used in the next step directly without further purification.

To a stirred solution of 6-bromoimidazo[1,2-a]pyrazine-3-carboxylic acid (78 mg, 0.32 mmol, 1 equiv) and 6-([(2R)-2-aminopropoxy]methyl-1-methyl-1,2,3-benzotriazol-4-amine (113 mg, 0.48 mmol, 1.5 equiv) in DMF (5 mL) were added HATU (245 mg, 0.64 mmol, 2 equiv) and DIEA (208 mg, 1.6 mmol, 5 equiv) dropwise at room temperature under nitrogen atmosphere. The resulting mixture was stirred for 2 h at room temperature under nitrogen atmosphere. The desired product could be detected by LCMS. The residue was purified by reversed-phase flash chromatography with the following conditions: column, C18 silica gel; mobile phase, MeCN in Water (0.1% FA), 50% to 65% gradient in 10 min; detector, UV 254 nm. This resulted in N-[(2R)-1-[(7-amino-3-methyl-1,2,3-benzotriazol-5-yl) methoxy]propan-2-yl]-6-bromoimidazo[1,2-a]pyrazine-3-carboxamide (66 mg, 44%) as a yellow solid.

To a stirred solution of N-[(2R)-1-[(7-amino-3-methyl-1,2,3-benzotriazol-5-yl) methoxy]propan-2-yl]-6-bromo-2-methylimidazo[1,2-a]pyrazine-3-carboxamide (66 mg, 0.12 mmol, 1 equiv) and RuPhos Palladacycle Gen.3 (10.2 mg, 0.01 mmol, 0.1 equiv) in dioxane were added Cs2CO3 (79 mg, 0.24 mmol, 2 equiv) in portions at room temperature under nitrogen atmosphere. The resulting mixture was stirred for overnight at 80° C. under nitrogen atmosphere. The mixture was allowed to cool down to room temperature. The desired product could be detected by LCMS. The solvent was removed under reduced pressure, The crude product was purified by Prep-HPLC with the following conditions (Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 25% B to 38% B in 10 min, 38% B; Wave Length: 220/254 nm: RT1(min): 9.8; Number Of Runs: 0) to afford (14R)-7,14-dimethyl-12-oxa-2,5,6,7,15,19,22,24-octaazapentacyclo[15.5.2.1\hat{ }(3,10.0\hat{ }(4,8.0\hat{ }(20,24]pentacosa-1(23),3(25),4(8),5,9,17,19,21-octaen-16-one (10.3 mg, 20%) as a white solid. LCMS:[M+H]⁺=379.00 NMR:1H NMR (400 MHz, DMSO-d6) δ 9.19 (s, 1H), 8.99 (d, J=1.3 Hz, 1H), 8.81 (d, J=1.6 Hz, 1H), 8.54 (d, J=8.8 Hz, 1H), 7.97 (d, J=3.0 Hz, 2H), 7.26 (s, 1H), 4.83 (d, J=13.1 Hz, 1H), 4.59-4.48 (m, 1H), 4.27 (s, 3H), 4.11 (d, J=5.6 Hz, 1H), 3.60 (dd, J=9.1, 2.6 Hz, 1H), 3.53-3.43 (m, 1H), 1.22 (s, 3H).

### Example 39: Synthesis of Compound 78

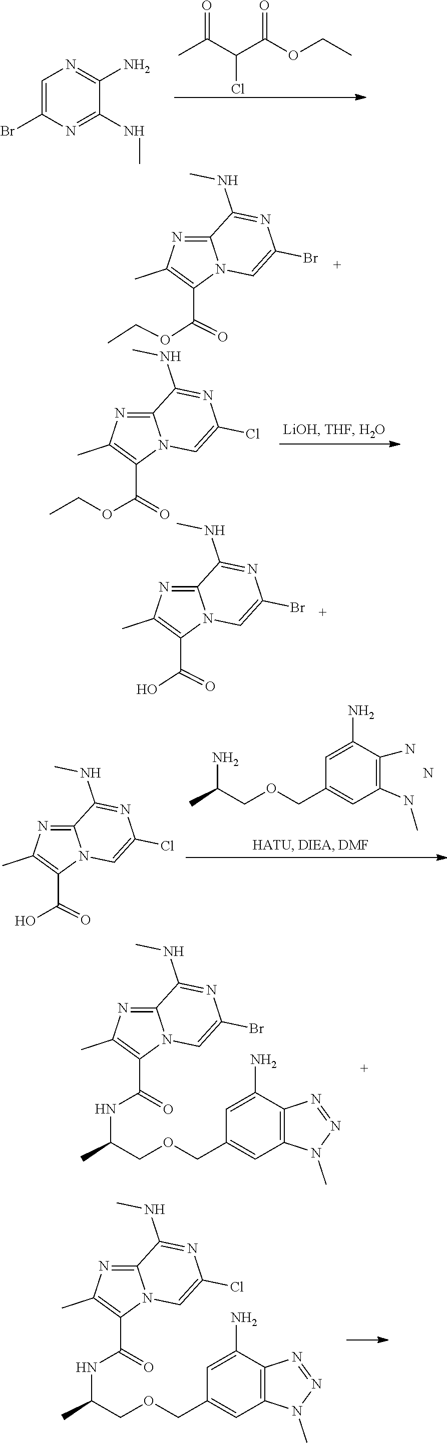

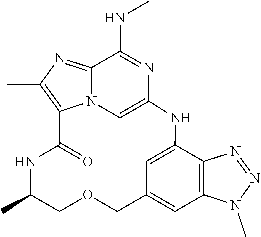 [SMILES: CNc1nc2cn3c(c(C)nc13)C(=O)N[C@H](C)COCc1cc(c3nnn(C)c3c1)N2]

A solution of 6-bromo-N2-methylpyrazine-2,3-diamine (2 g, 9.9 mmol, 1 equiv) and ethyl 2-chloro-3-oxobutanoate (2.6 g, 15.8 mmol, 1.6 equiv) in EtOH (50 mL) was stirred for overnight at 80° C. under argon atmosphere. The desired product could be detected by LCMS. The solvent was removed under reduced pressure. The residue was purified by silica gel column chromatography, eluted with PE/EA (1:1) to afford ethyl 6-bromo-8-(methylamino)imidazo[1,2-a]pyrazine-3-carboxylate (1.3 g, 44%) as an off-white solid.

A solution of ethyl 6-bromo-2-methyl-8-(methylamino)imidazo[1,2-a]pyrazine-3-carboxylate (500 mg, 1.6 mmol, 1 equiv) and LiOH (191 mg, 8.0 mmol, 5 equiv) in THF/H2O (20 mL/5 mL) was stirred for overnight at room temperature under argon atmosphere. The desired product could be detected by LCMS. The mixture was acidified to pH 5 with resin. The mixture was filtered, and the filtrate was concentrated under reduced pressure. The resulting mixture was concentrated under vacuum. This resulted in 6-bromo-2-methyl-8-(methylamino)imidazo[1,2-a]pyrazine-3-carboxylic acid (310 mg, 68%) as an off-white solid. The resulting mixture was used in the next step directly without further purification.

To a stirred solution of 6-bromo-2-methyl-8-(methylamino)imidazo[1,2-a]pyrazine-3-carboxylic acid (150 mg, 0.53 mmol, 1 equiv) and 6-([(2R)-2-aminopropoxy]methyl-1-methyl-1,2,3-benzotriazol-4-amine (185 mg, 0.79 mmol, 1.5 equiv) in DMF (5 mL) were added DIEA (544 mg, 4.2 mmol, 8 equiv) and HATU (300 mg, 0.79 mmol, 1.5 equiv) in portions at room temperature under argon atmosphere. The resulting mixture was stirred for 2 h at room temperature under argon atmosphere. The desired product could be detected by LCMS. The residue was purified by reversed-phase flash chromatography with the following conditions: column, C18 silica gel; mobile phase, MeCN in water, 5% to 70% gradient in 30 min; detector, UV 254 nm. This resulted in N-[(2R)-1-[(7-amino-3-methyl-1,2,3-benzotriazol-5-yl)methoxy]propan-2-yl]-6-bromo-2-methyl-8-(methylamino)imidazo[1,2-a]pyrazine-3-carboxamide (182 mg, 69%) as an orange solid.

A solution of N-[(2R)-1-[(7-amino-3-methyl-1,2,3-benzotriazol-5-yl)methoxy]propan-2-yl]-6-bromo-2-methyl-8-(methylamino)imidazo[1,2-a]pyrazine-3-carboxamide (162 mg, 0.32 mmol, 1 equiv), RuPhos Palladacycle Gen.3 (26.9 mg, 0.03 mmol, 0.1 equiv) and Cs2CO3 (210 mg, 0.64 mmol, 2 equiv) in 1,4-dioxane (5 mL) was stirred for overnight at 80° C. under argon atmosphere. The desired product could be detected by LCMS. The reaction was quenched by the addition of water (5 mL) at room temperature. The aqueous layer was extracted with EtOAc (3×20 mL). The organic layers were combined, dried and concentrated under vacuum. The crude product (140 mg) was purified by Prep-HPLC with the following conditions (Column: XBridge Prep OBD C18 Column, 30*150 mm, 5 μm; Mobile Phase A: water (0.05% NH3·H2O), Mobile Phase B: ACN; Flow rate: 60 mL/min; Gradient: 12% B to 26% B in 10 min, 26% B; Wave Length: 220/254 nm; RT1(min): 14.27; Number Of Runs: 0) to afford (14R)-7,14,18-trimethyl-21-(methylamino)-12-oxa-2,5,6,7,15,19,22,24-octaazapentacyclo[15.5.2.1\hat{ }(3,10.0\hat{ }(4,8.0\hat{ }(20,24]pentacosa-1(23),3(25),4(8),5,9,17,19,21-octaen-16-one (15.4 mg, 11.29%) as a white solid. LCMS:[M+H]⁺=422.20 HNMR: 1H NMR (400 MHz, DMSO-d6) δ 8.31 (d, J=8.8 Hz, 1H), 8.02 (d, J=13.1 Hz, 2H), 7.89 (s, 1H), 7.63 (d, J=5.0 Hz, 1H), 7.17 (s, 1H), 4.80 (d, J=13.3 Hz, 1H), 4.53 (d, J=13.3 Hz, 1H), 4.25 (s, 3H), 4.10 (s, 1H), 3.60-3.54 (m, 1H), 3.50 (dd, J=9.0, 4.9 Hz, 1H), 3.00 (d, J=4.7 Hz, 3H), 2.39 (s, 3H), 1.21 (d, J=6.8 Hz, 3H).

### Example 40: Synthesis of Compound 79

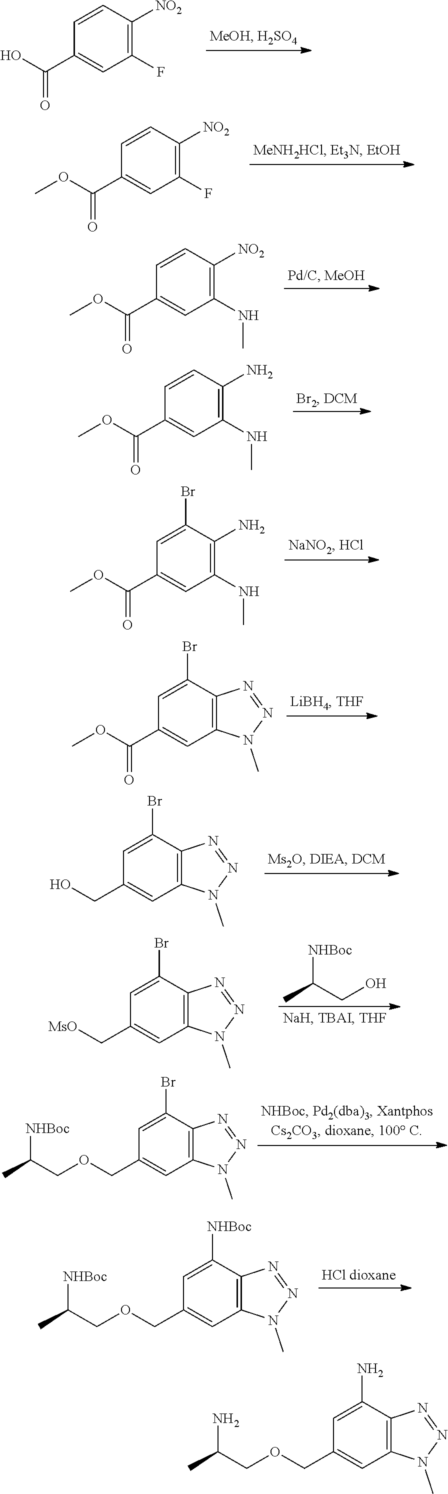

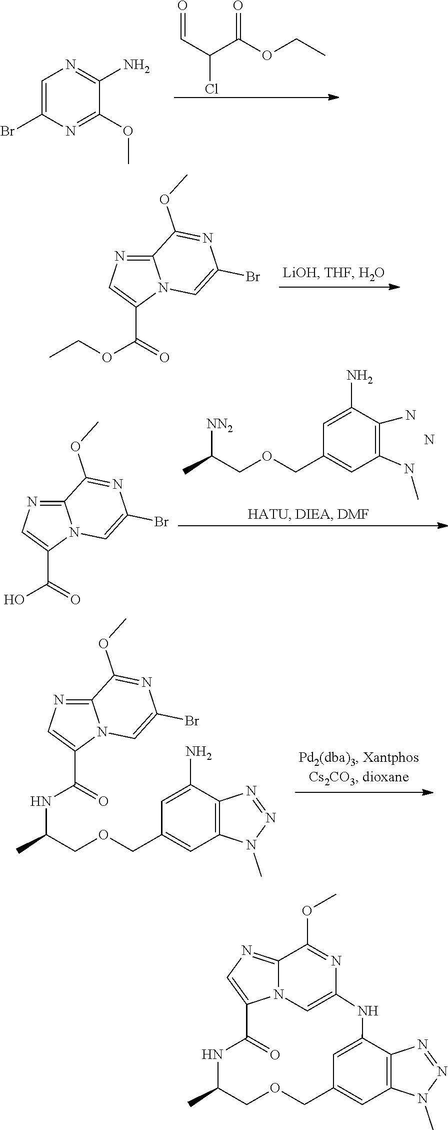

A solution of 3-fluoro-4-nitrobenzoic acid (20 g, 108 mmol, 1 equiv) and H2SO4 (3 mL) in MeOH (200 mL) was stirred for overnight at 70° C. under argon atmosphere. The desired product could be detected by LCMS. The resulting mixture was concentrated under reduced pressure. The mixture was basified to pH 7 with saturated NaHCO_{3(aq.)}. The resulting mixture was extracted with EtOAc (3×300 mL). The combined organic layers were washed with brine (3×100 mL), dried over anhydrous Na2SO4. After filtration, the filtrate was concentrated under reduced pressure. This resulted in methyl 3-fluoro-4-nitrobenzoate (19.7 g, 92%) as a white solid. The resulting mixture was used in the next step directly without further purification.

To a stirred solution of methyl 3-fluoro-4-nitrobenzoate (17.2 g, 86.4 mmol, 1 equiv) and Et3N (26.2 g, 259 mmol, 3 equiv) in EtOH (200 mL) was added methanamine hydrochloride (11.7 g, 173 mmol, 2 equiv) dropwise at 0° C. under argon atmosphere. The resulting mixture was stirred for overnight at room temperature under argon atmosphere. The desired product could be detected by LCMS. The solvent was removed under reduced pressure. The residue was purified by silica gel column chromatography, eluted with PE/EA (2:1) to afford methyl 3-(methylamino)-4-nitrobenzoate (16.3 g, 90%) as an orange solid.

To a solution of methyl 3-(methylamino)-4-nitrobenzoate (16.3 g, 77.5 mmol, 1 equiv) in 100 mL MeOH was added Pd/C (10%, 0.1 g) in a 250 mL round-bottom flask. The mixture was hydrogenated at room temperature for overnight under hydrogen atmosphere using a hydrogen balloon, the resulting mixture was filtered through a Celite pad and concentrated under reduced pressure. This resulted in methyl 4-amino-3-(methylamino)benzoate (12.5 g, 89%) as a grey solid. The resulting mixture was used in the next step directly without further purification.

A solution of methyl 4-amino-3-(methylamino)benzoate (8.4 g, 46.6 mnol, 1 equiv) and Br2 (8.9 g, 55.9 mmol, 1.2 equiv) in DCM (100 mL) was stirred for 2 h at room temperature under argon atmosphere. The desired product could be detected by LCMS. The solvent was removed under reduced pressure. The residue was purified by reversed-phase flash chromatography with the following conditions: column, C18 silica gel; mobile phase, MeCN in water (0.1% FA), 20% to 80% gradient in 30 min; detector, UV 254 nm. This resulted in methyl 4-amino-3-bromo-5-(methylamino)benzoate (3.8 g, 32%) as a brown oil.

To a stirred solution of methyl 4-amino-3-bromo-5-(methylamino) benzoate (3.8 g, 14.8 mmol, 1 equiv) and HCl (6M) (40 mL) was added NaNO2 (1.5 g, 22.2 mmol, 1.5 equiv) in H2O (20 mL) dropwise at 0° C. under argon atmosphere. The resulting mixture was stirred for 2 h at room temperature under argon atmosphere. The desired product could be detected by LCMS. The mixture was neutralized to pH 8 with saturated NaHCO_{3(aq.)}. The resulting mixture was extracted with EtOAc (3×50 mL). The combined organic layers were washed with brine (3×30 mL), dried over anhydrous Na₂SO₄. After filtration, the filtrate was concentrated under reduced pressure. This resulted in methyl 7-bromo-3-methyl-1,2,3-benzotriazole-5-carboxylate (4 g, 99%) as a brown solid. The resulting mixture was used in the next step directly without further purification.

To a stirred solution of methyl 7-bromo-3-methyl-1,2,3-benzotriazole-5-carboxylate (4.5 g, 16.7 mmol, 1 equiv) in THF (50 mL) was added LiAlH4 (10.0 mL, 25.0 mmol, 1.5 equiv) dropwise at −78° C. under argon atmosphere. The resulting mixture was stirred for 1 h at −40° C. under argon atmosphere. The desired product could be detected by LCMS. The reaction was quenched with sat. NH4Cl_{(aq.)} at 0° C. The solvent was removed under reduced pressure. The residue was purified by silica gel column chromatography, eluted with PE/EA (1:3) to afford (7-bromo-3-methyl-1,2,3-benzotriazol-5-yl) methanol (3.3 g, 81%) as a brown solid.

To a stirred solution of (7-bromo-3-methyl-1,2,3-benzotriazol-5-yl) methanol (3 g, 12.4 mmol, 1 equiv) and DIEA (4.8 g, 37.2 mmol, 3 equiv) in DCM (50 mL) was added methanesulfonic anhydride (3.2 g, 18.5 mmol, 1.5 equiv) dropwise at 0° C. under argon atmosphere. The resulting mixture was stirred for 1 h at room temperature under argon atmosphere. The reaction was quenched with water at room temperature. The resulting mixture was extracted with EtOAc (3×100 mL). The combined organic layers were washed with brine (3×50 mL), dried over anhydrous Na2SO4. After filtration, the filtrate was concentrated under reduced pressure. This resulted in (7-bromo-3-methyl-1,2,3-benzotriazol-5-yl) methyl methane sulfonate (4.0 g, 99%) as an off-white solid. The resulting mixture was used in the next step directly without further purification.

To a stirred solution of (7-bromo-3-methyl-1,2,3-benzotriazol-5-yl) methyl methanesulfonate (4.2 g, 13.1 mmol, 1 equiv) and tert-butyl N-[(2R)-1-hydroxypropan-2-yl]carbamate (2.8 g, 15.7 mmol, 1.2 equiv) in THF (50 mL) was added NaH (0.79 g, 19.7 mmol, 1.5 equiv, 60%) in portions at 0° C. under argon atmosphere. To the above mixture was added TBAI (0.48 g, 1.3 mmol, 0.1 equiv) dropwise at 0° C. The resulting mixture was stirred for additional 2 h at room temperature. The desired product could be detected by LCMS. The reaction was quenched with water/ice at 0° C. The resulting mixture was extracted with EtOAc (3×100 mL). The combined organic layers were washed with brine (3×50 mL), dried over anhydrous Na2SO4. After filtration, the filtrate was concentrated under reduced pressure. The residue was purified by silica gel column chromatography, eluted with PE/EA (1:1) to afford tert-butyl N-[(2R)-1-[(7-bromo-3-methyl-1,2,3-benzotriazol-5-yl)methoxy]propan-2-yl]carbamate (4.2 g, 80%) as a pink oil.

To a stirred solution of tert-butyl N-[(2R)-1-[(7-bromo-3-methyl-1,2,3-benzotriazol-5-yl) methoxy]propan-2-yl]carbamate (4.2 g, 10.5 mmol, 1 equiv), tert-butyl carbamate (1.9 g, 15.8 mmol, 1.5 equiv) and Cs2CO3 (6.9 g, 21.0 mmol, 2.0 equiv) in 1,4-dioxane (50 mL) were added Pd2(dba)3 (0.96 g, 1.1 mmol, 0.1 equiv) and Xantphos (1.2 g, 2.1 mmol, 0.2 equiv) in portions at room temperature under argon atmosphere. The resulting mixture was stirred for 2 h at 110° C. under argon atmosphere. The desired product could be detected by LCMS. The solvent was removed under reduced pressure. The residue was purified by silica gel column chromatography, eluted with PE/EA (2:1) to afford tert-butyl N-[(2R)-1-((7-[(tert-butoxycarbonyl)amino]-3-methyl-1,2,3-benzotriazol-5-ylmethoxy)propan-2-yl]carbamate (4.3 g, 93%) as an off-white solid.

A solution of tert-butyl N-[(2R)-1-((7-[(tert-butoxycarbonyl) amino]-3-methyl-1,2,3-benzotriazol-5-ylmethoxy)propan-2-yl]carbamate (2 g, 4.6 mmol, 1 equiv) and 4 N HCl in 1,4-dioxane (50 mL) in 1,4-dioxane was stirred for 2 h at room temperature under argon atmosphere. The desired product could be detected by LCMS. The resulting mixture was concentrated under vacuum. The crude product was used in the next step directly without further purification.

A solution of 5-bromo-3-methoxypyrazin-2-amine (2 g, 9.8 mmol, 1 equiv) and ethyl 2-chloro-3-oxopropanoate (2.4 g, 15.7 mmol, 1.6 equiv) in EtOH (50 mL) was stirred for overnight at 80° C. under argon atmosphere. The desired product could be detected by LCMS. The solvent was removed under reduced pressure. The residue was purified by reversed-phase flash chromatography with the following conditions: column, C18 silica gel; mobile phase, MeCN in water (0.1% FA), 40% to 90% gradient in 30 min; detector, UV 254 nm. This resulted in ethyl 6-bromo-8-methoxyimidazo[1,2-a]pyrazine-3-carboxylate (854 mg, 29%) as an off-white solid.

A solution of ethyl 6-bromo-8-methoxyimidazo[1,2-a]pyrazine-3-carboxylate (854 mg, 2.8 mmol, 1 equiv) and LiOH (204 mg, 8.5 mmol, 3 equiv) in THF/H2O (20 mL/5 mL) was stirred for overnight at room temperature under argon atmosphere. The desired product could be detected by LCMS. The mixture was neutralized with Dowex 50WX8-200 ion-exchange resin, The mixture was filtered and the filtrate was concentrated under reduced pressure. This resulted in 6-bromo-8-methoxyimidazo[1,2-a]pyrazine-3-carboxylic acid (689 mg, 89%) as an off-white solid. The resulting mixture was used in the next step directly without further purification.

To a stirred solution of 6-bromo-8-methoxyimidazo[1,2-a]pyrazine-3-carboxylic acid (500 mg, 1.8 mmol, 1 equiv) and 6-([(2R)-2-aminopropoxy]methyl-1-methyl-1,2,3-benzotriazol-4-amine (475 mg, 2.0 mmol, 1.1 equiv) in DMF (20 mL) were added DIEA (1900 mg, 14.7 mmol, 8 equiv) and HATU (1048 mg, 2.7 mmol, 1.5 equiv) in portions at room temperature under argon atmosphere. The resulting mixture was stirred for 2 h at room temperature under argon atmosphere. The desired product could be detected by LCMS. The residue was purified by reversed-phase flash chromatography with the following conditions: column, C18 silica gel; mobile phase, MeCN in water, 35% to 80% gradient in 25 min; detector, UV 254 nm. This resulted in N-[(2R)-1-[(7-amino-3-methyl-1,2,3-benzotriazol-5-yl)methoxy]propan-2-yl]-6-bromo-8-methoxyimidazo[1,2-a]pyrazine-3-carboxamide (330 mg, 37%) as an off-white oil.

A solution of N-[(2R)-1-[(7-amino-3-methyl-1,2,3-benzotriazol-5-yl)methoxy]propan-2-yl]-6-bromo-8-methoxyimidazo[1,2-a]pyrazine-3-carboxamide (300 mg, 0.61 mmol, 1 equiv), Pd₂(dba)₃ (56.1 mg, 0.06 mmol, 0.1 equiv), Xantphos (70.9 mg, 0.12 mmol, 0.2 equiv) and Cs₂CO₃ (399 mg, 1.2 mmol, 2 equiv) in 1,4-dioxane(l0 mL) was stirred for overnight at 80° C. under argon atmosphere. The desired product could be detected by LCMS. The solvent was removed under reduced pressure. The crude product was purified by Prep-HPLC with the following conditions (Column: YMC-Actus Triart C18 ExRS, 30*150 mm, 5 μm; Mobile Phase A: water (10 mmol/L NH4HCO3), Mobile Phase B: ACN; Flow rate: 60 mL/min; Gradient: 11% B to 31% B in 9 min, 31% B; Wave Length: 254/220 nm; RT1(min): 10.38; Number Of Runs: 0) to afford (14R)-21-methoxy-7,14-dimethyl-12-oxa-2,5,6,7,15,19,22,24-octaazapentacyclo[15.5.2.1\hat{ }(3,10.0\hat{ }(4,8.0\hat{ }(20,24]pentacosa-1(23),3(25),4(8),5,9,17,19,21-octaen-16-one (36.6 mg, 14%) as a white solid. LCMS:[M+H]′=409.05 NMR: 1H NMR (400 MHz, DMSO-d6) δ 8.80 (s, 1H), 8.52 (d, J=8.8 Hz, 1H), 8.41 (s, 1H), 7.95 (s, 1H), 7.79 (s, 1H), 7.24 (s, 1H), 4.82 (d, J=13.2 Hz, 1H), 4.54 (d, J=13.3 Hz, 1H), 4.26 (s, 3H), 4.14 (s, 4H), 3.59 (dd, J=9.0, 2.5 Hz, 1H), 3.49 (dd, J=9.1, 4.8 Hz, 1H), 1.26-1.18 (m, 3H).

### Example 41: Synthesis of Compound 80

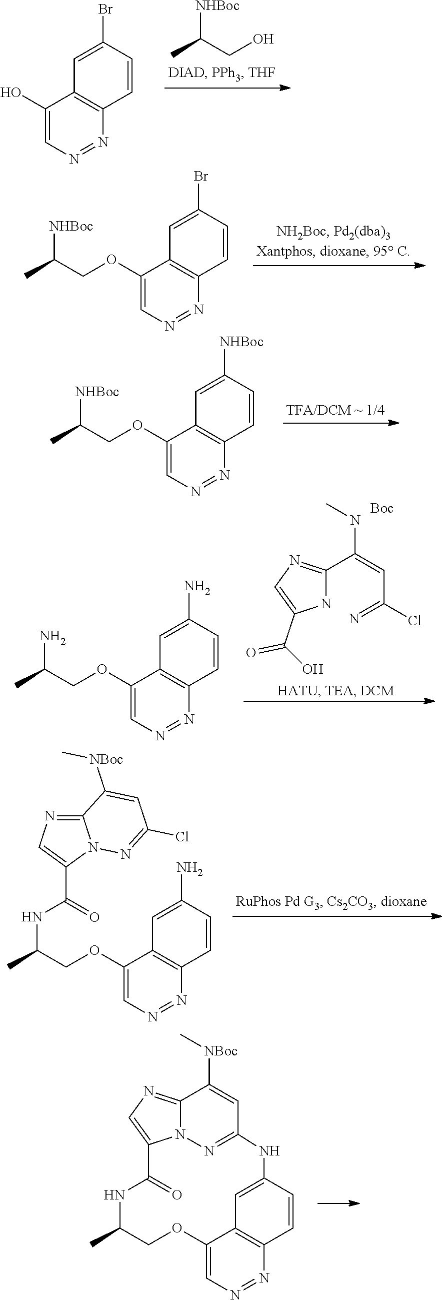

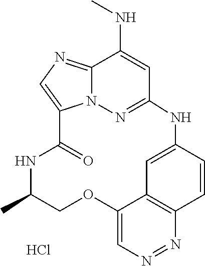 [SMILES: CNc1cc2nn3c(cnc13)C(=O)N[C@H](C)COc1cnnc3ccc(cc13)N2 ; Cl]

To a stirred solution of 6-bromocinnolin-4-ol (5 g, 22.2 mmol, 1 equiv), (tert-butoxycarbonyl)[(2R)-1-hydroxypropan-2-yl]aminyl (5.0 g, 28.9 mmol, 1.3 equiv) and PPh3 (7.6 g, 28.9 mmol, 1.3 equiv) in THF (100 ml) was added DIAD (6.3 g, 31.1 mmol, 1.4 equiv) dropwise at 0° C. under argon atmosphere. The resulting mixture was stirred for 4 h at room temperature under argon atmosphere. The desired product could be detected by LCMS. The solvent was removed under reduced pressure. The residue was purified by silica gel column chromatography, eluted with PE/EA (1:10) to afford [(2R)-1-[(6-bromocinnolin-4-yl)oxy]propan-2-yl](tert-butoxycarbonyl)aminyl (7.3 g, 86%) as an off-white solid.

A solution of tert-butyl N-[(2R)-1-[(6-bromocinnolin-4-yl)oxy]propan-2-yl]carbamate (3 g, 7.8 mmol, 1 equiv), tert-butyl carbamate (1.1 g, 9.4 mmol, 1.2 equiv), Pd2(dba)3 (718 mg, 0.79 mmol, 0.1 equiv), Xantphos (908 mg, 1.6 mmol, 0.2 equiv) and Cs2CO3 (5.1 g, 15.7 mmol, 2 equiv) in 1,4-dioxane (50 ml) was stirred for overnight at 110° C. under argon atmosphere. The desired product could be detected by LCMS. The solvent was removed under reduced pressure. The residue was purified by reversed-phase flash chromatography with the following conditions: column, C18 silica gel; mobile phase, MeCN in water (10 mmol/L NH4HCO3), 5% to 95% gradient in 30 min; detector, UV 254 nm. This resulted in tert-butyl N-[(2R)-1-((6-[(tert-butoxycarbonyl)amino]cinnolin-4-yloxy)propan-2-yl]carbamate (1.4 g, 41%) as a yellow solid.

A solution of tert-butyl N-[(2R)-1-((6-[(tert-butoxycarbonyl)amino]cinnolin-4-yloxy)propan-2-yl]carbamate (1.3 g, 3.0 mmol, 1 equiv) and TFA (10 mL) in DCM (40 mL) was stirred for overnight at room temperature under argon atmosphere. The desired product could be detected by LCMS. The resulting mixture was concentrated under vacuum. The resulting mixture was used in the next step directly without further purification.

To a stirred solution of 4-[(2R)-2-aminopropoxy]cinnolin-6-amine (600 mg, 2.7 mmol, 1 equiv) and 8-[(tert-butoxycarbonyl)(methyl)amino]-6-chloroimidazo[1,2-b]pyridazine-3-carboxylic acid (898 mg, 2.7 mmol, 1 equiv) in DCM (50 mL) were added DIEA (1065 mg, 8.2 mmol, 3 equiv) and HATU (1254 mg, 3.3 mmol, 1.2 equiv) in portions at room temperature under argon atmosphere. The resulting mixture was stirred for 2 h at room temperature under argon atmosphere. The desired product could be detected by LCMS. The residue was purified by reversed-phase flash chromatography with the following conditions: column, C18 silica gel; mobile phase, MeCN in water (0.1% FA), 5% to 95% gradient in 40 min; detector, UV 254 nm. This resulted in tert-butyl N-(3-([(2R)-1-[(6-aminocinnolin-4-yl)oxy]propan-2-yl]carbamoyl-6-chloroimidazo[1,2-b]pyridazin-8-yl)-N-methylcarbamate (504 mg, 35%) as a yellow oil.

To a stirred solution of tert-butyl (R)-(3-((1-((6-aminocinnolin-4-yl)oxy)propan-2-yl)carbamoyl)-6-chloroimidazo[1,2-b]pyridazin-8-yl)(methyl)carbamate (100 mg, 0.19 mmol, 1 equiv) and Cs2CO3 (124 mg, 0.38 mmol, 2 equiv) in dioxane (10 mL) was added RuPhos Palladacycle Gen.3 (15.9 mg, 0.02 mmol, 0.10 equiv) in portions at room temperature under argon atmosphere. The resulting mixture was stirred for o/n at 90° C. under argon atmosphere. The desired product could be detected by LCMS. The reaction was quenched by the addition of water (5 mL) at room temperature. The aqueous layer was extracted with EtOAc (3×20 mL). The organic layers were combined, dried and concentrated under vacuum. The residue was purified by reversed-phase flash chromatography with the following conditions: column, C18 silica gel; mobile phase, MeCN in water (0.1% FA), 30% to 70% gradient in 20 min; detector, UV 254 nm. This resulted in tert-butyl N-methyl-N-[(12R)-12-methyl-10-oxo-14-oxa-2,4,5,7,11,17,18-heptaazapentacyclo[13.6.2.2\hat{ }(3,6.0\hat{ }(5,9.0\hat{ }(19,23]pentacosa-1(22),3,6,8,15,17,19(23),20,24-nonaen-24-yl]carbamate (40 mg, 42%) as a yellow solid. LCMS:[M+H]+=491.40 HNMR: 1H NMR (400 MHz, DMSO-d6) δ 10.05 (s, 1H), 8.82 (s, 1H), 8.14 (s, 1H), 7.78 (d, J=24.9 Hz, 3H), 7.38 (d, J=9.0 Hz, 1H), 7.08 (s, 1H), 4.83 (s, 1H), 4.60 (s, 2H), 3.45 (s, 3H), 1.47 (s, 9H), 1.24 (s, 1H), 1.10 (d, J=7.0 Hz, 3H).

A solution of tert-butyl N-methyl-N-[(12R)-12-methyl-10-oxo-14-oxa-2,4,5,7,11,17,18-heptaazapentacyclo[13.6.2.2\hat{ }(3,6.0\hat{ }(5,9.0\hat{ }(19,23]pentacosa-1(22),3,6,8,15,17,19(23),20,24-nonaen-24-yl]carbamate (30 mg, 0.06 mmol, 1 equiv) and 4N HCl_{(gas)} in 1,4-dioxane (2 mL) was stirred for 4 h at room temperature under argon atmosphere. The desired product could be detected by LCMS. The resulting mixture was concentrated under vacuum. This resulted in (12R)-12-methyl-24-(methylamino)-14-oxa-2,4,5,7,11,17,18-heptaazapentacyclo[13.6.2.2\hat{ }(3,6.0\hat{ }(5,9.0\hat{ }(19,23]pentacosa-1(22),3,6,8,15,17,19(23),20,24-nonaen-10-one (17.6 mg, 70%) as a yellow solid. LCMS:[M+H]⁺=391.15 HNMR: 1H NMR (400 MHz, DMSO-d6) δ 10.38 (s, 1H), 8.83 (s, 1H), 8.31 (s, 1H), 8.11 (s, 1H), 7.83 (t, J=43.3 Hz, 2H), 7.49 (d, J=9.2 Hz, 1H), 6.05 (s, 1H), 4.87 (s, 2H), 4.79-4.63 (m, 1H), 2.96 (s, 3H), 1.21 (d, J=10.6 Hz, 4H).

### Example 42: Synthesis of Compound 96

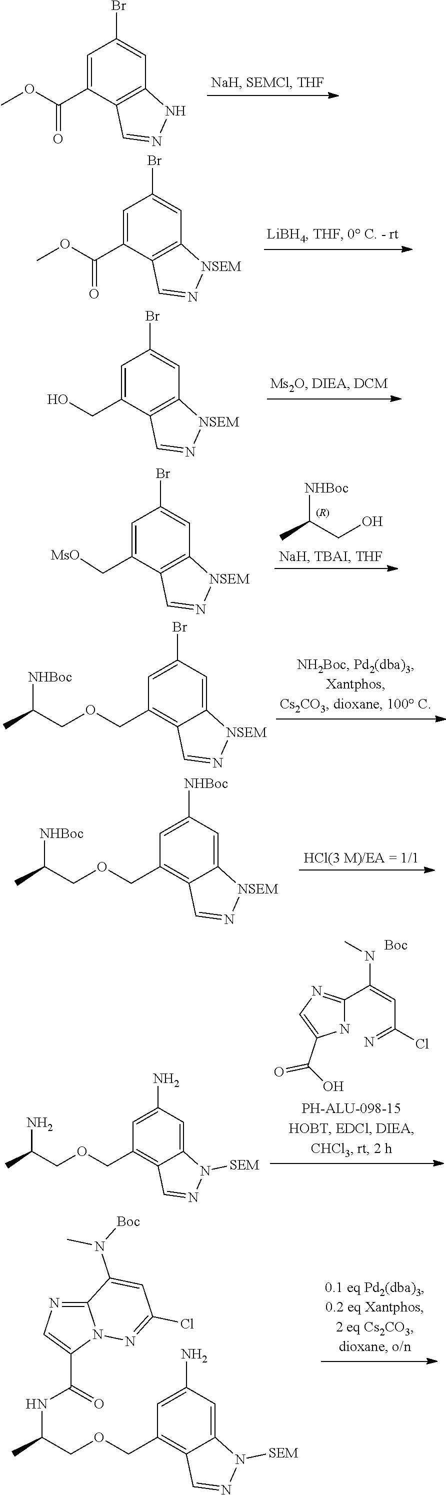

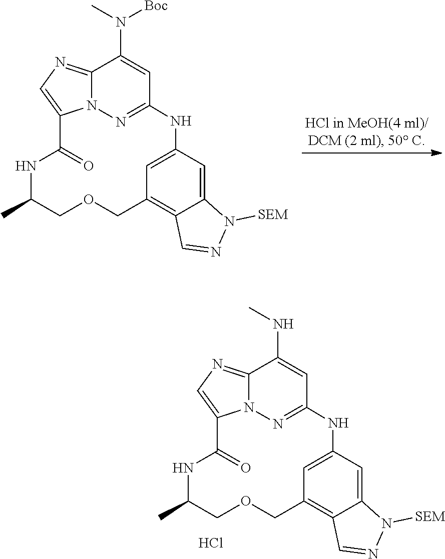 [SMILES: C[C@@H]1COCc2cc(cc3c2cnn3COCC[Si](C)(C)C)Nc2cc(N(C)C(=O)OC(C)(C)C)c3ncc(n3n2)C(=O)N1 ; CNc1cc2nn3c(cnc13)C(=O)N[C@H](C)COCc1cc(cc3c1cnn3COCC[Si](C)(C)C)N2 ; Cl]

To a stirred solution of methyl 6-bromo-1H-indazole-4-carboxylate (5 g, 19.602 mmol, 1 equiv) in THF (100 ml) was added NaH (705.63 mg, 29.403 mmol, 1.5 equiv) in portions at 0° C. under nitrogen atmosphere. The resulting mixture was stirred for 30 min at 0° C. under nitrogen atmosphere. To the stirred mixture was added SEMCl (4.2 g, 25.2 mmol, 1.3 equiv) dropwise at 0° C. under nitrogen atmosphere. The resulting mixture was stirred for 2 h at room temperature under nitrogen atmosphere. The desired product could be detected by LCMS. The reaction was quenched with sat. NH₄Cl (aq.) at 0° C. The resulting mixture was extracted with EtOAc (3×40 mL). The combined organic layers were washed with brine (1×60 mL), dried over anhydrous Na₂SO₄. After filtration, the filtrate was concentrated under reduced pressure. The residue was purified by silica gel column chromatography, eluted with PE/EA (10:1) to afford methyl 6-bromo-1-([2-(trimethylsilyl)ethoxy]methylindazole-4-carboxylate (5.7 g, 75%) as a brown liquid.

To a stirred solution of methyl 6-bromo-1-([2-(trimethylsilyl)ethoxy]methylindazole-4-carboxylate (3.5 g, 9.1 mmol, 1 equiv) in THF (50 ml) was added LiBH₄ (9.08 mL, 18.2 mmol, 2 equiv) in portions at 0^{L}C under nitrogen atmosphere. The resulting mixture was stirred for overnight at room temperature under nitrogen atmosphere. The desired product could be detected by LCMS. The reaction was quenched with sat. NH₄Cl (aq.) at 0° C. The resulting mixture was extracted with EtOAc (3×30 mL). The combined organic layers were washed with brine (1×40 mL), dried over anhydrous Na₂SO₄. After filtration, the filtrate was concentrated under reduced pressure. The resulting mixture was concentrated under reduced pressure. The residue was purified by silica gel column chromatography, eluted with PE/EA (2:1) to afford (6-bromo-1-([2-(trimethylsilyl)ethoxy]methylindazol-4-yl)methanol (3 g, 92%) as a brown solid.

To a stirred mixture of (6-bromo-1-([2-(trimethylsilyl)ethoxy]methylindazol-4-yl)methanol (3.5 g, 9.8 mmol, 1 equiv) and DIEA (5.12 mL, 29.4 mmol, 3 equiv) in DCM (60 ml) was added methanesulfonyl methanesulfonate (3.41 g, 19.6 mmol, 2 equiv) dropwise at 0° C. under nitrogen atmosphere. The resulting mixture was stirred for 3 h at 0° C. under nitrogen atmosphere. The desired product could be detected by LCMS. The reaction was quenched with sat. NH₄Cl (aq.) at 0° C. The resulting mixture was extracted with EtOAc (3×40 mL). The combined organic layers were washed with brine (1×50 mL), dried over anhydrous Na₂SO₄. After filtration, the filtrate was concentrated under reduced pressure. The crude product was used in the next step directly without further purification.

To a stirred mixture of (6-bromo-1-([2-(trimethylsilyl)ethoxy]methylindazol-4-yl)methyl methanesulfonate (3.6 g, 8.268 mmol, 1 equiv) and tert-butyl N-[(2R)-1-hydroxypropan-2-yl]carbamate (2.17 g, 12.4 mmol, 1.5 equiv) in THF (40 ml) were added NaH (397 mg, 9.9 mmol, 1.2 equiv, 60%) and TBAI (305 mg, 0.83 mmol, 0.1 equiv) in portions at 0° C. under nitrogen atmosphere. The resulting mixture was stirred for 2 h at room temperature under nitrogen atmosphere. The desired product could be detected by LCMS. The reaction was quenched with sat. NH₄Cl (aq.) at 0° C. The resulting mixture was extracted with EtOAc (3×40 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (2:1) to afford tert-butyl N-[(2R)-1-[(6-bromo-1-([2-(trimethylsilyl)ethoxy]methylindazol-4-yl)methoxy]propan-2-yl]carbamate (3.9 g, 92%) as a yellow liquid.

To a stirred mixture of tert-butyl N-[(2R)-1-[(6-bromo-1-([2-(trimethylsilyl)ethoxy]methylindazol-4-yl)methoxy]propan-2-yl]carbamate (3.9 g, 7.6 mmol, 1 equiv) and BocNH₂ (1.78 g, 15.2 mmol, 2 equiv) in 1,4-dioxane (40 ml) were added Pd₂(dba)₃ (0.69 g, 0.76 mmol, 0.1 equiv), Xantphos (0.88 g, 15.2 mmol, 0.2 equiv) and Cs₂CO₃ (4.94 g, 15.2 mmol, 2 equiv) in portions at room temperature under nitrogen atmosphere. The resulting mixture was stirred for 3 h at 100° C. under nitrogen atmosphere. The mixture was allowed to cool down to room temperature. The desired product could be detected by LCMS. The resulting mixture was concentrated under reduced pressure. The residue was purified by silica gel column chromatography, eluted with PE/EA (5:1) to afford tert-butyl N-(4-([(2R)-2-[(tert-butoxycarbonyl)amino]propoxy]methyl-1-([2-(trimethylsilyl)ethoxy]methylindazol-6-yl)carbamate (3.3 g, 79%) as a brown liquid.

To a stirred solution of tert-butyl N-(4-([(2R)-2-[(tert-butoxycarbonyl)amino]propoxy]methyl-1-([2-(trimethylsilyl)ethoxy]methylindazol-6-yl)carbamate (500 mg, 0.91 mmol, 1 equiv) in EtOAc (10 ml) was added HCl (10 mL, 30 mmol, 33 equiv) dropwise 0° C. under nitrogen atmosphere. The resulting mixture was stirred for overnight at room temperature under nitrogen atmosphere. The desired product could be detected by LCMS. The reaction was quenched by the addition of sat. NaHCO₃ (aq.) (60 mL) at 0° C. The resulting mixture was extracted with EtOAc (3×40 mL). The combined organic layers were washed with brine (1×60 mL), dried over anhydrous Na2SO4. After filtration, the filtrate was concentrated under reduced pressure. The crude product (220 mg) was used in the next step directly without further purification.

To a stirred mixture of 4-([(2R)-2-aminopropoxy]methyl-1-([2-(trimethylsilyl)ethoxy]methylindazol-6-amine (160 mg, 0.46 mmol, 1 equiv) and 8-[(tert-butoxycarbonyl)(methyl)amino]-6-chloroimidazo[1,2-b]pyridazine-3-carboxylic acid (179 mg, 0.55 mmol, 1.2 equiv) in CHCl₃ (10 ml) were added EDCI (131 mg, 0.68 mmol, 1.5 equiv), HOBt (92.5 mg, 0.68 mmol, 1.5 equiv) and DIEA (88.5 mg, 0.68 mmol, 1.5 equiv) at room temperature under nitrogen atmosphere. The resulting mixture was stirred for overnight at room temperature under nitrogen atmosphere. The desired product (50%) could be detected by LCMS. The resulting mixture was concentrated under reduced pressure. The residue was purified by reversed-phase flash chromatography with the following conditions: column, C18 silica gel; mobile phase, MeCN in water, 40% to 100% gradient in 30 min; detector, UV 254 nm. This resulted in tert-butyl N-(3-([(2R)-1-[(6-amino-1-([2-(trimethylsilyl)ethoxy]methylindazol-4-yl)methoxy]propan-2-yl]carbamoyl-6-chloroimidazo[1,2-b]pyridazin-8-yl)-N-methylcarbamate (170 mg, 56%) as a light brown solid.

To a stirred mixture of tert-butyl N-(3-([(2R)-1-[(6-amino-1-([2-(trimethylsilyl)ethoxy]methylindazol-4-yl)methoxy]propan-2-yl]carbamoyl-6-chloroimidazo[1,2-b]pyridazin-8-yl)-N-methylcarbamate (120 mg, 0.18 mmol, 1 equiv) and Pd₂(dba)₃ (16.7 mg, 0.018 mmol, 0.1 equiv) in 1,4-dioxane (10 ml) were added XantPhos (21.1 mg, 0.036 mmol, 0.2 equiv) and Cs₂CO₃ (119 mg, 0.36 mmol, 2 equiv) at room temperature under argon atmosphere. The resulting mixture was stirred for overnight at 80° C. under argon atmosphere. The desired product (64%) could be detected by LCMS. The resulting mixture was concentrated under reduced pressure. The residue was purified by reversed-phase flash chromatography with the following conditions: column, C18 silica gel; mobile phase, MeCN in water, 20% to 100% gradient in 30 min; detector, UV 254 nm. This resulted in tert-butyl N-methyl-N-[(14R)-14-methyl-16-oxo-6-([2-(trimethylsilyl)ethoxy]methyl-12-oxa-2,6,7,15,19,23,24-heptaazapentacyclo[15.5.2.1\hat{ }(3,10.0\hat{ }(5,9.0\hat{ }(20,24]pentacosa-1(23),3(25),4,7,9,17,19,21-octaen-21-yl]carbamate (75 mg, 66%) as a light yellow solid.

To a stirred solution of tert-butyl N-methyl-N-[(14R)-14-methyl-16-oxo-6-([2-(trimethylsilyl)ethoxy]methyl-12-oxa-2,6,7,15,19,23,24-heptaazapentacyclo[15.5.2.1\hat{ }(3,10.0\hat{ }(5,9.0\hat{ }(20,24]pentacosa-1(23),3(25),4,7,9,17,19,21-octaen-21-yl]carbamate (30 mg, 0.048 mmol, 1 equiv) in DCM (2 ml) was added HCl_{(g)} in MeOH (4 mL, 16 mmol) dropwise at room temperature under nitrogen atmosphere. The resulting mixture was stirred for 2 days at 50° C. under nitrogen atmosphere. The desired product could be detected by LCMS. The resulting mixture was concentrated under reduced pressure. The product was lyophilized directly; This resulted in (14R)-14-methyl-21-(methylamino)-12-oxa-2,6,7,15,19,23,24-heptaazapentacyclo[15.5.2.1\hat{ }(3,10.0\hat{ }(5,9.0\hat{ }(20,24]pentacosa-1(23),3(25),4,7,9,17,19,21-octaen-16-one hydrochloride (11.9 mg, 55.64%) was obtained as a light yellow solid. LCMS :[M+H]=393.00 NMR: ¹H NMR (400 MHz, DMSO-d6) δ 10.00-9.86 (m, 1H), 8.76 (d, J=5.6 Hz, 1H), 8.33-8.28 (m, 1H), 8.21 (s, 1H), 8.04 (d, J=12.2 Hz, 2H), 7.07 (s, 1H), 6.08 (d, J=2.1 Hz, 2H), 4.87-4.72 (m, 2H), 3.98 (q, J=6.6, 6.1 Hz, 1H), 3.51 (dd, J=10.1, 2.6 Hz, 1H), 3.28 (t, J=9.1 Hz, 1H), 2.87 (s, 3H), 1.07 (d, J=6.5 Hz, 3H).

### Example 43: Synthesis of Compound 97

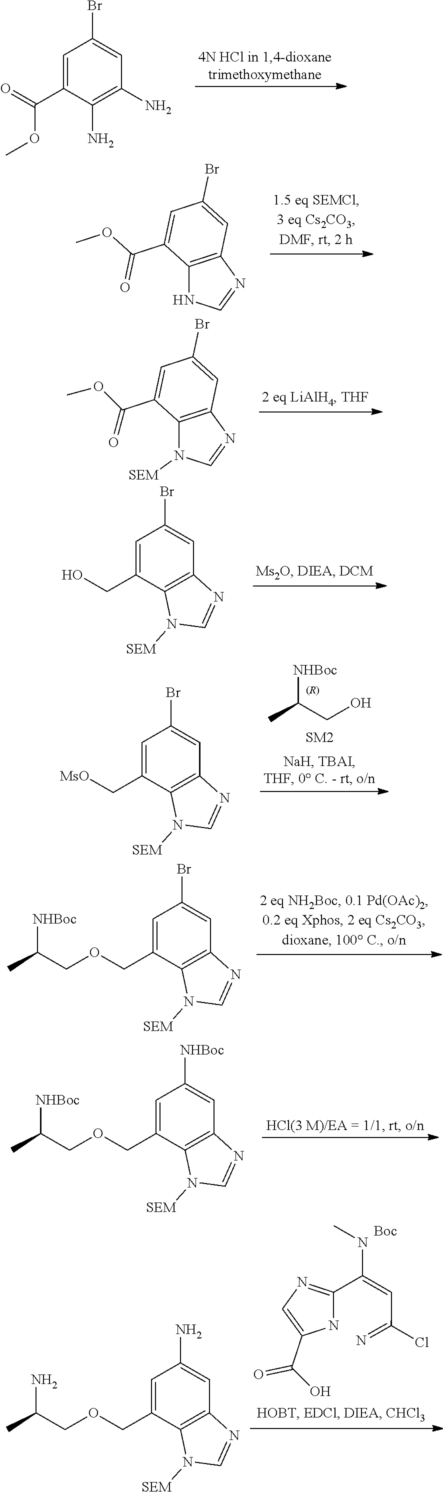

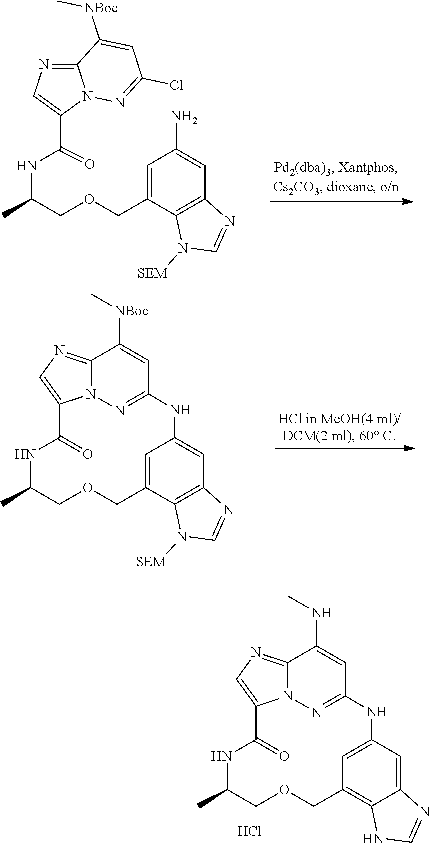

To a stirred mixture of methyl 2,3-diamino-5-bromobenzoate (6 g, 24.482 mmol, 1 equiv) and trimethyl orthoformate (60 mL) was added HCl(gas) in 1,4-dioxane (18 mL, 72.000 mmol, 2.94 equiv) dropwise at 0° C. under nitrogen atmosphere. The resulting mixture was stirred for overnight at room temperature under nitrogen atmosphere. Desired product could be detected by LCMS. The mixture was neutralized to pH 7 with saturated NaHCO₃ (aq.). The resulting mixture was extracted with EtOAc (3×30 mL). The combined organic layers were washed with brine (1×50 mL), dried over anhydrous Na₂SO₄. After filtration, the filtrate was concentrated under reduced pressure. The residue was purified by silica gel column chromatography, eluted with EA/MeOH (4:1) to afford methyl 6-bromo-3H-1,3-benzodiazole-4-carboxylate (5.6 g, 89.68%) as a brown solid.

To a stirred mixture of methyl 6-bromo-3H-1,3-benzodiazole-4-carboxylate (4.4 g, 17.250 mmol, 1 equiv) and Cs₂CO₃ (16.86 g, 51.750 mmol, 3 equiv) in DMF (80 ml) was added SEMCl (4.31 g, 25.875 mmol, 1.5 equiv) at room temperature under nitrogen atmosphere. The resulting mixture was stirred for 2 h at room temperature under nitrogen atmosphere. Desired product could be detected by LCMS. The reaction was quenched with Water at room temperature. The residue was purified by reversed-phase flash chromatography with the following conditions: column, C18 silica gel; mobile phase, MeCN in Water, 20% to 100% gradient in 30 min; detector, UV 254 nm. This resulted in methyl 6-bromo-3-([2-(trimethylsilyl)ethoxy]methyl-1,3-benzodiazole-4-carboxylate (3.9 g, 58.67%) as a brown oil.

To a stirred solution of methyl 6-bromo-3-([2-(trimethylsilyl)ethoxy]methyl-1,3-benzodiazole-4-carboxylate (11.2 g, 29.066 mmol, 1 equiv) in THF (120 ml) was added LiAlH4 (58.13 mL, 58.132 mmol, 2 equiv) dropwise at 0° C. under nitrogen atmosphere. The resulting mixture was stirred for 1 h at 0° C. under nitrogen atmosphere. Desired product could be detected by LCMS. The reaction was quenched with sat. NH₄Cl (aq.) at 0° C. The resulting mixture was extracted with EtOAc (3×100 mL). The combined organic layers were washed with brine (1×150 mL), dried over anhydrous Na₂SO₄. After filtration, the filtrate was concentrated under reduced pressure. The residue was purified by silica gel column chromatography, eluted with PE/EA (1:2) to afford (6-bromo-3-([2-(trimethylsilyl)ethoxy]methyl-1,3-benzodiazol-4-yl)methanol (5 g, 48.14%) as alight yellow oil.

To a stirred mixture of (6-bromo-3-([2-(trimethylsilyl)ethoxy]methyl-1,3-benzodiazol-4-yl)methanol (2.8 g, 7.836 mmol, 1 equiv) and DIEA (3.04 g, 23.508 mmol, 3 equiv) in DCM (40 ml) was added methanesulfonyl methanesulfonate (2.73 g, 15.672 mmol, 2 equiv) n DCM (10 ml) dropwise at 0° C. under nitrogen atmosphere. The resulting mixture was stirred for 1 h at 0° C. under nitrogen atmosphere. Desired product could be detected by LCMS. The reaction was quenched with Water/Ice at 0° C. The resulting mixture was extracted with CH₂Cl₂ (3×15 mL). The combined organic layers were washed with brine (1×20 mL), dried over anhydrous Na₂SO₄. After filtration, the filtrate was concentrated under reduced pressure. The crude product (3 g) was used in the next step directly without further purification.

To a stirred mixture of (6-bromo-3-([2-(trimethylsilyl)ethoxy]methyl-1,3-benzodiazol-4-yl)methyl methanesulfonate (3 g, 6.890 mmol, 1 equiv) and tert-butyl N-[(2R)-1-hydroxypropan-2-yl]carbamate (2.41 g, 13.780 mmol, 2 equiv) in THF (30 nil) were added NaH (496.04 mg, 20.670 mmol, 3 equiv) and TBAI (1272.51 mg, 3.445 mmol, 0.5 equiv) in portions at 0° C. under nitrogen atmosphere. The resulting mixture was stirred for 2 h at room temperature under nitrogen atmosphere. Desired product could be detected by LCMS. The reaction was quenched with Water/Ice at 0° C. The resulting mixture was extracted with EtOAc (3×30 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water, 20% to 100% gradient in 30 min; detector, UV 254 nm. This resulted in tert-butyl N-[(2R)-1-[(6-bromo-3H-1,3-benzodiazol-4-yl)methoxy]propan-2-yl]carbamate (1.6 g, 60.43%) as a light yellow oil.

To a stirred mixture of tert-butyl N-[(2R)-1-[(6-bromo-3-([2-(trimethylsilyl)ethoxy]methyl-1,3-benzodiazol-4-yl)methoxy]propan-2-yl]carbamate (2.1 g, 4.081 mmol, 1 equiv) and tert-butyl carbamate (956.23 mg, 8.162 mmol, 2 equiv) in 1,4-dioxane (25 ml) were added Pd(OAc)₂ (91.63 mg, 0.408 mmol, 0.1 equiv), XPhos (389.14 mg, 0.816 mmol, 0.2 equiv) and Cs₂CO₃ (2.66 g, 8.162 mmol, 2 equiv) at room temperature under nitrogen atmosphere. The resulting mixture was stirred for 2 h at 100° C. under nitrogen atmosphere. Desired product (85%) could be detected by LCMS. The resulting mixture was filtered, the filter cake was washed with EtOAc (3×20 mL). The filtrate was concentrated under reduced pressure. The residue was purified by reversed-phase flash chromatography with the following conditions: column, C18 silica gel; mobile phase, MeCN in Water, 20% to 100% gradient in 30 min; detector, UV 254 nm. This resulted in tert-butyl N-(7-([(2R)-2-[(tert-butoxycarbonyl)amino]propoxy]methyl-1-([2-(trimethylsilyl)ethoxy]methyl-1,3-benzodiazol-5-yl)carbamate (2 g, 88.97%) as a light yellow solid.

To a stirred solution of tert-butyl N-(7-([(2R)-2-[(tert-butoxycarbonyl)amino]propoxy]methyl-1-([2-(trimethylsilyl)ethoxy]methyl-1,3-benzodiazol-5-yl)carbamate (100 mg, 0.182 mmol, 1 equiv) in EtOAc (3 ml) was added HCl (3 mL, 9.000 mmol, 49.57 equiv) dropwise at 0° C. under nitrogen atmosphere. The resulting mixture was stirred for overnight at room temperature under nitrogen atmosphere. Desired product (73%) could be detected by LCMS. The reaction was quenched with sat. NaHCO₃ (aq.) at 0° C. The resulting mixture was extracted with EtOAc (3×20 mL). The combined organic layers were washed with brine (1×30 mL), dried over anhydrous Na₂SO₄. After filtration, the filtrate was concentrated under reduced pressure. The crude product (350 mg) was used in the next step directly without further purification.

To a stirred mixture of 7-([(2R)-2-aminopropoxy]methyl-1-([2-(trimethylsilyl)ethoxy]methyl-1,3-benzodiazol-5-amine (300 mg, 0.856 mmol, 1 equiv) and 8-[(tert-butoxycarbonyl)(methyl)amino]-6-chloroimidazo[1,2-b]pyridazine-3-carboxylic acid (335.56 mg, 1.027 mmol, 1.2 equiv) in CHCl₃(15 ml) were added EDCI (246.09 mg, 1.284 mmol, 1.5 equiv), HOBT (173.47 mg, 1.284 mmol, 1.5 equiv) and DIEA (331.84 mg, 2.568 mmol, 3 equiv) at room temperature under nitrogen atmosphere. The resulting mixture was stirred for overnight at room temperature under nitrogen atmosphere. Desired product (50%) could be detected by LCMS. The resulting mixture was concentrated under reduced pressure. The residue was purified by reversed-phase flash chromatography with the following conditions: column, C18 silica gel; mobile phase, MeCN in Water, 40% to 100% gradient in 30 min; detector, UV 254 nm. This resulted in tert-butyl N-(3-([(2R)-1-[(6-amino-3-([2-(trimethylsilyl)ethoxy]methyl-1,3-benzodiazol-4-yl)methoxy]propan-2-yl]carbamoyl-6-chloroimidazo[1,2-b]pyridazin-8-yl)-N-methylcarbamate (210 mg, 37.22%) as a light brown solid.

To a stirred mixture of tert-butyl N-(3-([(2R)-1-[(6-amino-3-([2-(trimethylsilyl)ethoxy]methyl-1,3-benzodiazol-4-yl)methoxy]propan-2-yl]carbamoyl-6-chloroimidazo[1,2-b]pyridazin-8-yl)-N-methylcarbamate (210 mg, 0.319 mmol, 1 equiv) and Pd₂(dba)₃ (29.17 mg, 0.032 mmol, 0.1 equiv) in 1,4-dioxane (10 ml) were added Xantphos (36.86 mg, 0.064 mmol, 0.2 equiv) and Cs₂CO₃ (207.57 mg, 0.638 mmol, 2 equiv) at room temperature under argon atmosphere. The resulting mixture was stirred for overnight at 80° C. under argon atmosphere. Desired product (85%) could be detected by LCMS. The residue was purified by reversed-phase flash chromatography with the following conditions: column, C18 silica gel; mobile phase, MeCN in Water, 10% to 80% gradient in 30 min; detector, UV 254 nm. This resulted in tert-butyl N-methyl-N-[(14R)-14-methyl-16-oxo-8-([2-(trimethylsilyl)ethoxy]methyl-12-oxa-2,6,8,15,19,23,24-heptaazapentacyclo[15.5.2.1\hat{ }(3,10.0\hat{ }(5,9.0\hat{ }(20,24]pentacosa-1(23),3(25),4,6,9,17,19,21-octaen-21-yl]carbamate (90 mg, 45.37%) as a light yellow solid.

To a stirred solution of tert-butyl N-methyl-N-[(14R)-14-methyl-16-oxo-8-([2-(trimethylsilyl)ethoxy]methyl-12-oxa-2,6,8,15,19,23,24-heptaazapentacyclo[15.5.2.1\hat{ }(3,10.0\hat{ }(5,9.0\hat{ }(20,24]pentacosa-1(23),3,5(9),6,10(25),17,19,21-octaen-21-yl]carbamate (80 mg, 0.128 mmol, 1 equiv) in CH₂Cl₂(3 ml) was added HCl(g) in MeOH (6 mL, 24.000 mmol, 186.84 equiv) dropwise at room temperature under nitrogen atmosphere. The resulting mixture was stirred for 3 days at 60° C. under nitrogen atmosphere. Desired product could be detected by LCMS. The resulting mixture was directly concentrated under reduced pressure and directly lyophilized. This resulted in (14R)-14-methyl-21-(methylamino)-12-oxa-2,6,8,15,19,23,24-heptaazapentacyclo[15.5.2.1\hat{ }(3,10.0\hat{ }(5,9.0\hat{ }(20,24]pentacosa-1(23),3(25),4,6,9,17,19,21-octaen-16-one hydrochloride (16.1 mg, 28.32%) as a white solid. LCMS: [M+H]=392.95 NMR: ¹H NMR (400 MHz, DMSO-d6) (15.15 (s, 1H), 10.18 (s, 1H), 9.60 (s, 1H), 8.75 (d, J=5.7 Hz, 1H), 8.54 (d, J=2.0 Hz, 1H), 8.16 (s, 1H), 8.01 (s, 1H), 7.49 (d, J=1.9 Hz, 1H), 6.05 (s, 1H), 5.00 (q, J=15.4 Hz, 2H), 4.13-4.03 (m, 1H), 3.67-3.55 (m, 1H), 3.40 (t, J=9.2 Hz, 1H), 2.93 (s, 3H), 1.16 (d, J=6.5 Hz, 3H).

### Example 44: Synthesis of Compound 98

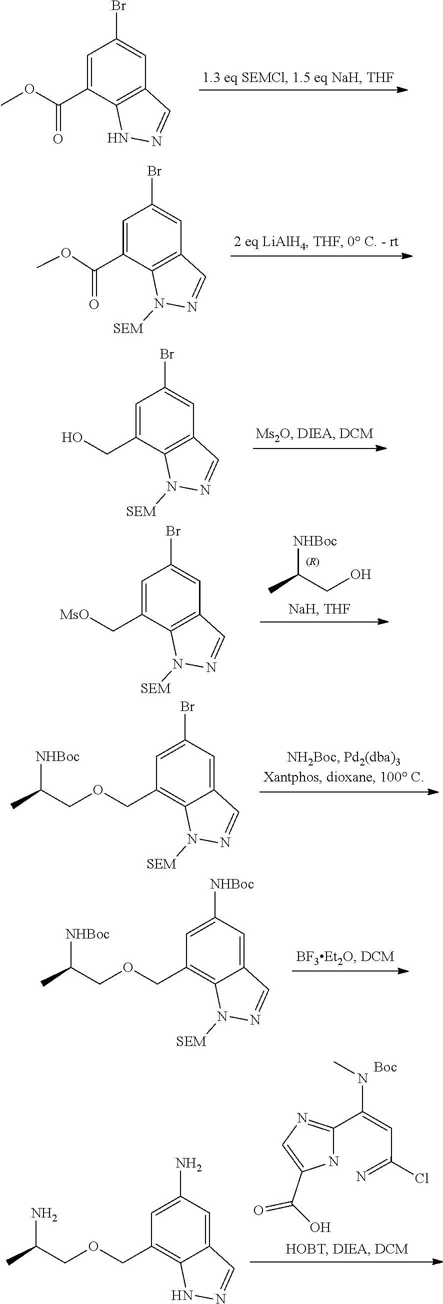

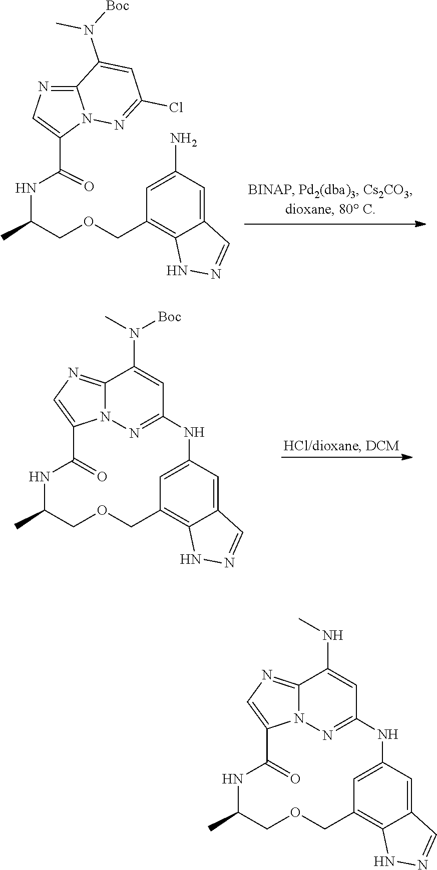

A solution of methyl 5-bromo-TH-indazole-7-carboxylate (7.8 g, 30.6 mmol, 1 equiv) in THF was treated with NaH (60% in oil, 1.1 g, 45.9 mmol, 1.5 equiv) for 30 min at 0° C. under nitrogen atmosphere followed by the addition of SEMCl (6.63 g, 39.8 mmol, 1.3 equiv) dropwise at room temperature. The resulting mixture was stirred for 1 h at room temperature under nitrogen atmosphere. The reaction was quenched with sat. NH₄Cl at room temperature. The aqueous layer was extracted with EtOAc (3×200 mL). The organic layers were combined, dried and concentrated under vacuum. The residue was purified by reversed-phase flash chromatography with the following conditions: column, C18 silica gel; mobile phase, MeCN in Water, 10% to 50% gradient in 10 min; detector, UV 254 nm. to afford methyl 5-bromo-1-([2-(trimethylsilyl)ethoxy]methylindazole-7-carboxylate (7 g, 59%) as a yellow liquid.

A solution of methyl 5-bromo-1-([2-(trimethylsilyl)ethoxy]methylindazole-7-carboxylate (7.8 g, 20.2 mmol, 1 equiv) in THF (100 ml) at room temperature under nitrogen atmosphere followed by the addition of LiBH₄ (0.88 g, 40.5 mmol, 2.00 equiv) dropwise at 0° C. The resulting mixture was stirred for 2 h at room temperature under nitrogen atmosphere. The desired product could be detected by LCMS. The reaction was quenched with water at 0° C. The resulting mixture was concentrated under reduced pressure. The residue was purified by reversed-phase flash chromatography with the following conditions: column, C18 silica gel; mobile phase, MeCN in Water, 10% to 50% gradient in 10 min; detector, UV 254 nm. to afford (5-bromo-1-([2-(trimethylsilyl)ethoxy]methylindazol-7-yl)methanol (5 g, 69%) as a yellow liquid.

To a stirred mixture of (5-bromo-1-([2-(trimethylsilyl)ethoxy]methylindazol-7-yl)methanol (5 g, 14.0 mmol, 1 equiv) and DIEA (7.23 g, 55.9 mmol, 4 equiv) in DCM (100 ml) was added methanesulfonyl methanesulfonate (6.09 g, 35 mmol, 2.5 equiv) dropwise at 0° C. under nitrogen atmosphere. The resulting mixture was stirred for 3 h at 0° C. under nitrogen atmosphere. The desired product could be detected by LCMS. The resulting mixture was concentrated under vacuum. to afford (5-bromo-1-([2-(trimethylsilyl)ethoxy]methylindazol-7-yl)methyl methanesulfonate (5.2 g, 85.35%) as a yellow liquid. The crude product was used in the next step directly without further purification.

To a stirred solution of (5-bromo-1-([2-(trimethylsilyl)ethoxy]methylindazol-7-yl)methyl methanesulfonate (6.7 g, 15.4 mmol, 1 equiv) and tert-butyl N-[(2R)-1-hydroxypropan-2-yl]carbamate (4.04 g, 23.1 mmol, 1.5 equiv) in THF (100 ml) were added NaH (60% in oil, 0.44 g, 18.5 mmol, 1.2 equiv) and TBAI (0.57 g, 1.54 mmol, 0.1 equiv) in portions at 0° C. under nitrogen atmosphere. The resulting mixture was stirred for 2 h at room temperature under nitrogen atmosphere. The desired product could be detected by LCMS. The reaction was quenched with sat. NH₄Cl_{(aq.)} at 0° C. The aqueous layer was extracted with EtOAc (3×100 mL). The organic layers were combined, dried and concentrated under vacuum. The residue was purified by reversed-phase flash chromatography with the following conditions: column, C18 silica gel; mobile phase, MeCN in Water, 10% to 50% gradient in 10 min; detector, UV 254 nm. This resulted in tert-butyl N-[(2R)-1-[(5-bromo-1-([2-(trimethylsilyl)ethoxy]methylindazol-7-yl)methoxy]propan-2-yl]carbamate (5 g, 63%) as a yellow liquid.

To a stirred mixture of tert-butyl N-[(2R)-1-[(5-bromo-1-([2-(trimethylsilyl)ethoxy]methylindazol-7-yl)methoxy]propan-2-yl]carbamate (3.7 g, 7.2 mmol, 1 equiv) and tert-butyl carbamate (1.7 g, 14.4 mmol, 2 equiv) in 1,4-dioxane (50 ml) were added Pd₂(dba)₃ (0.66 g, 0.72 mmol, 0.1 equiv) Xantphos (0.83 g, 1.4 mmol, 0.2 equiv) and Cs₂CO₃ (4.7 g, 14.4 mmol, 2 equiv) in portions at room temperature under nitrogen atmosphere. The resulting mixture was stirred for 2 h at 100° C. under nitrogen atmosphere. The reaction was quenched with water at room temperature. The aqueous layer was extracted with EtOAc (3×100 mL). The organic layers were combined, dried and concentrated under vacuum. The residue was purified by silica gel column chromatography, eluted with PE/EA (1:1) to afford tert-butyl N-(7-([(2R)-2-[(tert-butoxycarbonyl)amino]propoxy]methyl-1-([2-(trimethylsilyl)ethoxy]methylindazol-5-yl)carbamate (1.2 g, 30%) as a yellow solid.

A solution of tert-butyl N-(7-([(2R)-2-[(tert-butoxycarbonyl)amino]propoxy]methyl-1-([2-(trimethylsilyl)ethoxy]methylindazol-5-yl)carbamate (1 g, 1.80 mmol, 1 equiv) in DCM (20 ml) was treated with BF₃·Et₂O (5 mL) for 5 min at 0° C. under nitrogen atmosphere. The resulting mixture was stirred for 2 h at room temperature under nitrogen atmosphere. The desired product could be detected by LCMS. The resulting mixture was concentrated under reduced pressure. to afford 7-([(2R)-2-aminopropoxy]methyl-1H-indazol-5-amine (380 mg, 95%) as a yellow solid. The crude product was used in the next step directly without further purification.

A solution of 8-[(tert-butoxycarbonyl)(methyl)amino]-6-chloroimidazo[1,2-b]pyridazine-3-carboxylic acid (439 mg, 1.34 mmol, 0.8 equiv) in DMF (10 ml) was treated with DIEA (1736 mg, 13.4 mmol, 8 equiv), HATU (957 mg, 2.5 mmol, 1.5 equiv) for 20 min at room temperature under nitrogen atmosphere followed by the addition of 7-([(2R)-2-aminopropoxy]methyl-1H-1,3-benzodiazol-5-amine (370 mg, 1.68 mmol, 1 equiv) in portions at room temperature. The resulting mixture was stirred for 2 h at room temperature under nitrogen atmosphere. The desired product could be detected by LCMS. The residue was purified by reversed-phase flash chromatography with the following conditions: column, C18 silica gel; mobile phase, MeCN in Water, 10% to 50% gradient in 10 min; detector, UV 254 nm. to afford tert-butyl N-(3-([(2R)-1-[(6-amino-3H-1,3-benzodiazol-4-yl)methoxy]propan-2-yl]carbamoyl-6-chloroimidazo[1,2-b]pyridazin-8-yl)-N-methylcarbamate (300 mg, 34%) as a yellow solid.

To a stirred mixture of tert-butyl N-(3-([(2R)-1-[(5-amino-1H-indazol-7-yl)methoxy]propan-2-yl]carbamoyl-6-chloroimidazo[1,2-b]pyridazin-8-yl)-N-methylcarbamate (120 mg, 0.23 mmol, 1 equiv) and BINAP (28.3 mg, 0.045 mmol, 0.2 equiv) in 1,4-dioxane (10 ml) were added Cs₂CO₃ (148 mg, 0.45 mmol, 2 equiv) and Pd₂(dba)₃ (20.77 mg, 0.023 mmol, 0.1 equiv) in portions at room temperature under nitrogen atmosphere. The resulting mixture was stirred for overnight at 80° C. under nitrogen atmosphere. The desired product could be detected by LCMS. The reaction was quenched by the addition of water (5 mL) at room temperature. The aqueous layer was extracted with EtOAc (3×20 mL). The organic layers were combined, dried and concentrated under vacuum. The residue was purified by reversed-phase flash chromatography with the following conditions: column, C18 silica gel; mobile phase, MeCN in Water, 10% to 50% gradient in 10 min; detector, UV 254 nm. to afford tert-butyl N-methyl-N-[(14R)-14-methyl-16-oxo-12-oxa-2,7,8,15,19,23,24-heptaazapentacyclo[15.5.2.1\hat{ }(3,10.0\hat{ }(5,9.0\hat{ }(20,24]pentacosa-1(23),3(25),4,6,9,17,19,21-octaen-21-yl]carbamate (80 mg, 71%) as a yellow solid.

A solution of tert-butyl N-methyl-N-[(14R)-14-methyl-16-oxo-12-oxa-2,7,8,15,19,23,24-heptaazapentacyclo[15.5.2.1\hat{ }(3,10.0\hat{ }(5,9.0\hat{ }(20,24]pentacosa-1(23),3(25),4,6,9,17,19,21-octaen-21-yl]carbamate (40 mg, 0.08 mmol, 1 equiv) in DCM (1 ml) was stirred for 5 min at 0° C. under air atmosphere. To the above mixture was added 4 N HCl_{(g)}in 1,4-dioxane (2 mL) dropwise over 5 min at 0° C. The resulting mixture was stirred for additional 2 h at room temperature. The desired product could be detected by LCMS. The resulting mixture was concentrated under reduced pressure. The crude product was purified by Prep-HPLC with the following conditions (Column: YMC-Actus Triart C18 ExRS, 30*150 mm, 5 μm; Mobile Phase A: Water (10 mmol/L NH₄HCO3), Mobile Phase B: ACN; Flow rate: 60 m/min; Gradient: 10% B to 25% B in 9 min, 25% B; Wave Length: 254/220 nm; RT1(min): 15.05; Number Of Runs: 0) to afford (14R)-14-methyl-21-(methylamino)-12-oxa-2,7,8,15,19,23,24-heptaazapentacyclo[15.5.2.1\hat{ }(3,10.0\hat{ }(5,9.0\hat{ }(20,24]pentacosa-1(23),3(25),4,6,9,17,19,21-octaen-16-one (11.2 mg, 35%) as a white solid. LCMS:[M+H]⁺=393.00 NMR: ¹H NMR (400 MHz, DMSO-d₆) δ 13.15-13.03 (m, 1H), 9.23 (s, 1H), 8.83 (d, J=5.3 Hz, 1H), 8.32 (s, 1H), 8.06 (s, 1H), 7.80 (s. 1H), 7.43 (d, J=5.1 Hz, 1H), 7.29 (d, J=1.8 Hz, 1H), 5.76 (s, 1H), 4.88 (s, 2H), 4.02 (d, J=4.2 Hz, 1H), 3.54 (d, J=7.8 Hz, 1H), 2.89 (d, J=4.8 Hz, 3H), 1.24 (s, 2H), 1.14 (d, J=6.4 Hz, 3H), 0.85 (d, J=7.0 Hz, 1H).

### Example 45: Synthesis of Compound 99

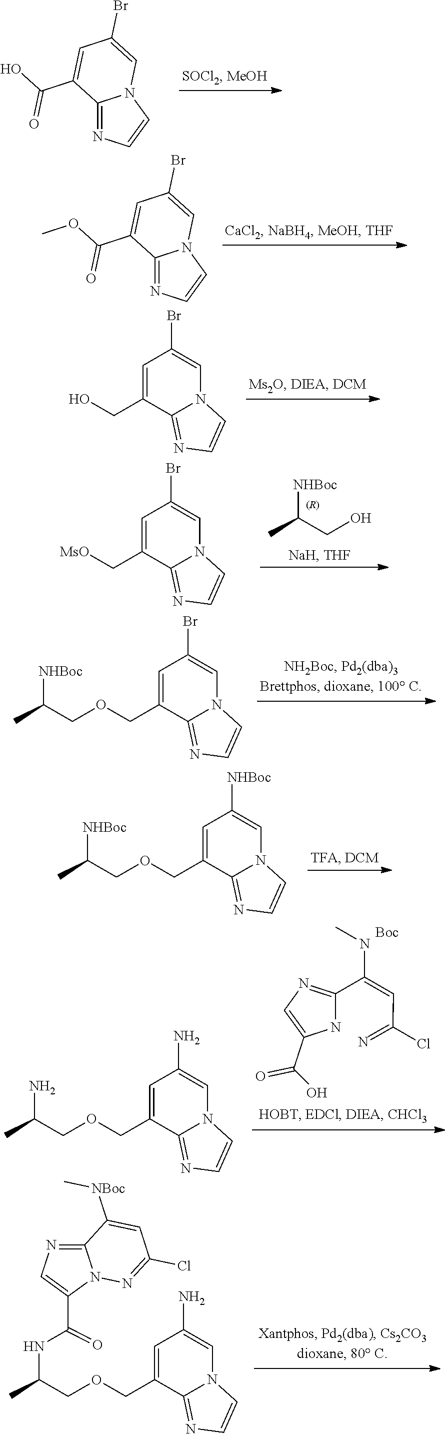

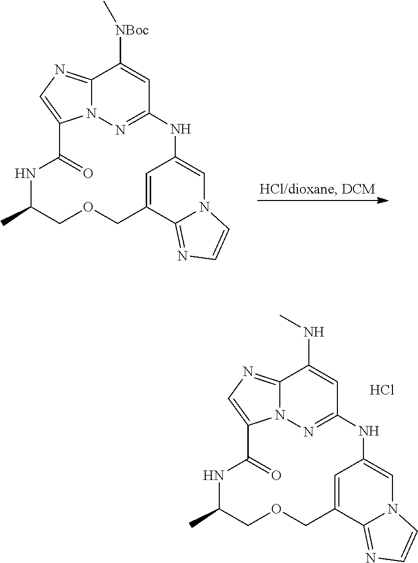 [SMILES: C[C@@H]1COCc2cc(cn3ccnc23)Nc2cc(N(C)C(=O)OC(C)(C)C)c3ncc(n3n2)C(=O)N1 ; CNc1cc2nn3c(cnc13)C(=O)N[C@H](C)COCc1cc(cn3ccnc13)N2 ; Cl]

To a stirred mixture of 6-bromoimidazo[1,2-a]pyridine-8-carboxylic acid (6 g, 24.9 mmol, 1 equiv) in MeOH (100 ml), was added SOCl₂ (27.1 mL, 373 mmol, 15 equiv) dropwise at room temperature under nitrogen atmosphere. The resulting mixture was stirred for 2 days at 65° C. under nitrogen atmosphere. The desired product could be detected by LCMS. The reaction was quenched with sat. NaHCO₃ (aq.) at room temperature. The aqueous layer was extracted with EtOAc (3×100 mL). The organic layers were combined, dried and concentrated under vacuum. The residue was purified by silica gel column chromatography, eluted with CH₂Cl₂/MeOH (10:1) to afford methyl 6-bromoimidazo[1,2-a]pyridine-8-carboxylate (5 g, 79%) as a white solid.

To a stirred mixture of methyl 6-bromoimidazo[1,2-a]pyridine-8-carboxylate (5 g, 19.6 mmol, 1 equiv) and CaCl₂) (3.26 g, 29.4 mmol, 1.5 equiv) in THF (100 mL) and MeOH (20 mL) were added NaBH₄ (1.5 g, 39.2 mmol, 2 equiv) in portions at −5° C. under nitrogen atmosphere. The resulting mixture was stirred for 2 h at 0° C. under nitrogen atmosphere. The reaction was quenched with water at 0° C. The aqueous layer was extracted with EtOAc (3×200 mL). The organic layers were combined, dried and concentrated under vacuum. The residue was purified by silica gel column chromatography, eluted with CH₂Cl₂/MeOH (10:1) to afford (6-bromoimidazo[1,2-a]pyridin-8-ylmethanol (3.8 g, 85%) as a white solid.

A solution of (6-bromoimidazo[1,2-a]pyridin-8-ylmethanol (3 g, 13.2 mmol, 1 equiv) in DCM (50 ml) was treated with DIEA (6.8 g, 52.8 mmol, 4 equiv) for 5 mm at room temperature under nitrogen atmosphere followed by the addition of methanesulfonyl methanesulfonate (6.9 g, 39.6 mmol, 3 equiv) in portions at 0° C. The resulting mixture was stirred for 3 h at 0° C. under nitrogen atmosphere. The desired product could be detected by LCMS. The aqueous layer was extracted with CH₂Cl₂ (3×50 mL). The organic layers were combined, dried and concentrated under vacuum. The resulting mixture was concentrated under reduced pressure to afford (6-bromoimidazo[1,2-a]pyridin-8-ylmethyl methanesulfonate (3.1 g, 76%) as a yellow solid. The crude product was used in the next step directly without further purification.

To a stirred mixture of (6-bromoimidazo[1,2-a]pyridin-8-ylmethyl methanesulfonate (4 g, 13.1 mmol, 1 equiv) and tert-butyl N-[(2R)-1-hydroxypropan-2-yl]carbamate (3.45 g, 19.6 mmol, 1.5 equiv) in THF (100 ml) were added NaH (60% in oil, 1.3 g, 52.4 mmol, 4 equiv) and TBAI (0.48 g, 1.3 mmol, 0.10 equiv) in portions at 0° C. under nitrogen atmosphere. The resulting mixture was stirred for 5 h at room temperature under nitrogen atmosphere. The desired product could be detected by LCMS. The reaction was quenched with water at room temperature. The aqueous layer was extracted with EtOAc (3×200 mL). The organic layers were combined, dried and concentrated under vacuum. The residue was purified by reversed-phase flash chromatography with the following conditions: column, C18 silica gel; mobile phase, MeCN in water, 10% to 50% gradient in 10 min; detector, UV 254 nm. to afford tert-butyl N-[(2R)-1-((6-bromoimidazo[1,2-a]pyridin-8-ylmethoxy)propan-2-yl]carbamate (2.1 g, 42%) as a yellow solid.

To a stirred mixture of tert-butyl N-[(2R)-1-((6-bromoimidazo[1,2-a]pyridin-8-ylmethoxy)propan-2-yl]carbamate (1.2 g, 3.1 mmol, 1 equiv) and tert-butyl carbamate (0.73 g, 6.2 mmol, 2 equiv) in 1,4-dioxane (20 ml) were added Pd₂(dba)₃ (0.29 g, 0.3 mmol, 0.1 equiv), Brettphos (0.34 g, 0.63 mmol, 0.2 equiv) and Cs₂CO₃ (2.03 g, 6.25 mmol, 2 equiv) in portions at room temperature under air atmosphere. The resulting mixture was stirred for overnight at 100° C. under nitrogen atmosphere. The desired product could be detected by LCMS. The reaction was quenched with water at room temperature. The aqueous layer was extracted with EtOAc (3×100 mL). The organic layers were combined, dried and concentrated under vacuum. The residue was purified by reversed-phase flash chromatography with the following conditions: column, C18 silica gel; mobile phase, MeCN in water, 10% to 50% gradient in 10 min; detector, UV 254 nm. to afford tert-butyl N-(8-([(2R)-2-[(tert-butoxycarbonyl)amino]propoxy]methylimidazo[1,2-a]pyridin-6-yl)carbamate (800 mg, 61%) as a yellow solid.

To a stirred solution of tert-butyl N-(8-([(2R)-2-[(tert-butoxycarbonyl)amino]propoxy]methylimidazo[1,2-a]pyridin-6-yl)carbamate (800 mg, 1.9 mmol, 1 equiv) in DCM (16 ml) was added TFA (4 mL) dropwise at 0° C. under nitrogen atmosphere. The resulting mixture was stirred for 1 h at room temperature under nitrogen atmosphere. The desired product could be detected by LCMS. The resulting mixture was concentrated under reduced pressure. The crude product was used in the next step directly without further purification.

To a stirred mixture of 8-([(2R)-2-aminopropoxy]methylimidazo[1,2-a]pyridin-6-amine (400 mg, 1.8 mmol, 1 equiv) and 8-[(tert-butoxycarbonyl)(methyl)amino]-6-chloroimidazo[1,2-b]pyridazine-3-carboxylic acid (593 mg, 1.82 mmol, 1 equiv) in were added HOBt (270 mg, 2 mmol, 1.1 equiv) EDCI (383 mg, 2 mmol, 1.1 equiv) and DIEA (1.2 g, 9.1 mmol, 5 equiv) in portions at room temperature under air atmosphere. The resulting mixture was stirred for 2 h at room temperature under nitrogen atmosphere. The desired product could be detected by LCMS. The resulting mixture was concentrated under reduced pressure. The residue was purified by reversed-phase flash chromatography with the following conditions: column, C18 silica gel; mobile phase, MeCN in water, 10% to 50% gradient in 10 min; detector, UV 254 nm. to afford tert-butyl N-(3-([(2R)-1-((6-aminoimidazo[1,2-a]pyridin-8-ylmethoxy)propan-2-yl]carbamoyl-6-chloroimidazo[1,2-b]pyridazin-8-yl)-N-methylcarbamate (400 mg, 42%) as a yellow solid.

To a stirred mixture of tert-butyl N-(3-{[(2R)-1-({6-aminoimidazo[1,2-a]pyridin-8-yl}methoxy)propan-2-yl]carbamoyl}-6-chloroimidazo[1,2-b]pyridazin-8-yl)-N-methylcarbamate (280 mg, 0.53 mmol, 1 equiv) and Cs₂CO₃ (345 mg, 1.06 mmol, 2 equiv) in 1,4-dioxane were added Xantphos (61 mg, 0.11 mmol, 0.2 equiv) and Pd₂(dba)₃ (48 mg, 0.05 mmol, 0.1 equiv) in portions at room temperature under air atmosphere. The resulting mixture was stirred for overnight at 80° C. under nitrogen atmosphere. The desired product could be detected by LCMS. The resulting mixture was concentrated under vacuum. The residue was purified by reversed-phase flash chromatography with the following conditions: column, C18 silica gel; mobile phase, MeCN in water, 10% to 50% gradient in 10 min; detector, UV 254 nm. to afford tert-butyl N-methyl-N-[(14R)-14-methyl-16-oxo-12-oxa-2,5,8,15,19,23,24-heptaazapentacyclo[15.5.2.1\hat{ }{3,10}0.\hat{ }{5,9}0.\hat{ }{20,24}]pentacosa-1(23),3,6,8,10(25),17,19,21-octaen-21-yl]carbamate (90 mg, 34%) as a yellow solid..

To a stirred solution of tert-butyl N-methyl-N-[(14R)-14-methyl-16-oxo-12-oxa-2,5,8,15,19,23,24-heptaazapentacyclo[15.5.2.1\hat{ }(3,10.0\hat{ }(5,9.0\hat{ }(20,24]pentacosa-1(23),3,6,8,10(25),17,19,21-octaen-21-yl]carbamate (70 mg, 0.14 mmol, 1 equiv) in DCM (1 ml) was added 4 N HCl_{(gas)} in 1,4-dioxane (2 mL) dropwise at 0° C. under nitrogen atmosphere. The resulting mixture was stirred for 2 h at room temperature under nitrogen atmosphere. The desired product could be detected by LCMS. The resulting mixture was concentrated under reduced pressure. to afford (14R)-14-methyl-21-(methylamino)-12-oxa-2,5,8,15,19,23,24-heptaazapentacyclo[15.5.2.1\hat{ }(3,10.0\hat{ }(5,9.0\hat{ }(20,24]pentacosa-1(23),3,6,8,10(25),17,19,21-octaen-16-one hydrochloride (45.4 mg, 74%) as a white solid. LCMS: [M+H]f=393.00 NMR: ¹H NMR (400 MHz, DMSO-d₆) δ 14.68 (s, 1H), 10.12 (s, 1H), 8.95 (d, J=1.9 Hz, 1H), 8.58 (d, J=1.9 Hz, 1H), 8.53-8.42 (m, 2H), 8.22 (d, J=2.1 Hz, 1H), 7.89 (s, 1H), 7.71 (d, J=5.2 Hz, 1H), 5.89 (s, 1H), 5.12-4.78 (m, 2H), 4.19-3.96 (m, 1H), 3.62 (dd, J=9.9, 2.6 Hz, 1H), 3.49 (s, 1H), 2.92 (d, J=4.1 Hz, 3H), 1.15 (d, J=6.5 Hz, 3H).

### Example 46: TYK2 JH2 Domain Binding Assay

Binding constants for the compounds described herein against the JH2 domain were determined by the following protocol for a KINOMEscan® assay (DiscoveRx). A fusion protein of a partial length construct of human TYK2 (JH2domain-pseudokinase) (amino acids G556 to D888 based on reference sequence NP_003322.3) and the DNA binding domain of NFkB was expressed in transiently transfected HEK293 cells. From these HEK 293 cells, extracts were prepared in M-PER extraction buffer (Pierce) in the presence of Protease Inhibitor Cocktail Complete (Roche) and Phosphatase Inhibitor Cocktail Set II (Merck) per manufacturers' instructions. The TYK2 (JH2domain-pseudokinase) fusion protein was labeled with a chimeric double-stranded DNA tag containing the NFkB binding site fused to an amplicon for qPCR readout, which was added directly to the expression extract (the final concentration of DNA-tag in the binding reaction is 0.1 nM).

Streptavidin-coated magnetic beads (Dynal M280) were treated with a biotinylated small molecule ligand for 30 minutes at room temperature to generate affinity resins the binding assays. The liganded beads were blocked with excess biotin and washed with blocking buffer (SeaBlock (Pierce), 1% BSA, 0.05% Tween 20, 1 mM DTT) to remove unbound ligand and to reduce nonspecific binding.

The binding reaction was assembled by combining 16 μl of DNA-tagged kinase extract, 3.8 μl liganded affinity beads, and 0.18 μl test compound (PBS/0.05% Tween 20/10 mM DTT/0.1% BSA/2 μg/ml sonicated salmon sperm DNA)]. Extracts were used directly in binding assays without any enzyme purification steps at a ≥10,000-fold overall stock dilution (final DNA-tagged enzyme concentration <0.1 nM). Extracts were loaded with DNA-tag and diluted into the binding reaction in a two-step process. First extracts were diluted 1:100 in 1×binding buffer (PBS/0.05% Tween 20/10 mM DTT/0.1% BSA/2 g/ml sonicated salmon sperm DNA) containing 10 nM DNA-tag. This dilution was allowed to equilibrate at room temperature for 15 minutes and then subsequently diluted 1:100 in 1× binding buffer. Test compounds were prepared as 111× stocks in 100% DMSO. Kds were determined using an 11-point 3-fold compound dilution series with three DMSO control points. All compounds for Kd measurements were distributed by acoustic transfer (non-contact dispensing) in 100% DMSO. The compounds were then diluted directly into the assays such that the final concentration of DMSO was 0.9%. All reactions were performed in polypropylene 384-well plates. Each was a final volume of 0.02 mL. Assays were incubated with shaking for 1 hour at room temperature. Then the beads were pelleted and washed with wash buffer (1×PBS, 0.05% Tween 20) to remove displaced kinase and test compound. The washed based were resuspended in elution buffer (1 λPBS, 0.05% Tween 20, 0.5 M non-biotinylated affinity ligand) and incubated at room temperature with shaking for 30 minutes. The kinase concentration in the eluates was measured by qPCR. qPCR reactions were assembled by adding 2.5 μL of kinase eluate to 7.5 μL of qPCR master mix containing 0.15 M amplicon primers and 0.15 μM amplicon probe. The qPCR protocol consisted of a 10 minute hot start at 95° C., followed by 35 cycles of 95° C. for 15 seconds, 60° C. for 1 minute.

Test compounds were prepared as 111× stocks in 100% DMSO. Kds were determined using an 11-point 3-fold compound dilution series with three DMSO control points. All compounds for Kd measurements were distributed by acoustic transfer (non-contact dispensing) in 100% DMSO. The compounds were then diluted directly into the assays such that the final concentration of DMSO was 0.9%. The Kds were determined using a compound top concentration of 30,000 nM. Kd measurements were performed in duplicate.

Binding constants (Kds) were calculated with a standard dose-response curve using the Hill equation:

$$Response = Background + \frac{( Signal - Background )}{( 1 + ( \frac{{Kd}^{Hill Slope}}{{Dose}^{Hill Slope}} )}$$

The Hill Slope was set to −1. Curves are fitted using a non-linear least square fit with the Levenberg-Marquardt algorithm (Levenberg, K., A method for the solution of certain non-linear problems in least squares, Q. Appl. Math. 2, 164-168 (1944)).

The results are shown in Table 2.

### Example 47: INFα Induced pSTAT5 in Human PBMC

Fresh Human PBMCs were resuspended in RPMI 1640 medium with 10% FBS. Cells were seeded in a round bottom 96-well plate at the concentration of 200,000 cells/well. A 10-point dilution series of test compound (top dose 10 uM, 1:5 dilution) was added to the well using the liquid dispenser (Tecan D300e) and incubated for 1 hour at 37 C. Then human INFα recombinant protein (R&D Systems) was added to the well at the final concentration of 5000 units/ml and incubated for 15 minutes at 37 C. Cell lysates were prepared and analyzed by Phospho STAT5 (Tyr693) Kit (Meso Scale Discovery) following manufacturer's protocol.

For calculation of the inhibition rate, the relative pSTAT5 signal of each well=pSTAT5 signal of each well−the average pSTAT5 signal of baseline.

The inhibition %=(the average pSTAT5 signal of INFα treatment wells−the relative of pSTAT5 signal in each compound containing well)/the average pSTAT5 signal of INFα treatment wells*100%

The curve was plotted as the inhibition % (y-axis) vs. compounds concentration (x-axis) and is fitted with log(inhibitor) vs. normalized response—Variable slope by GraphPad Prism7.0.

The results are shown in Table 3.

TABLE 2

| | TYK2 JH2 | TYK2 JH1 | JAK3 JH1 | JAK2 JH2 | JAK2 JH1 | JAK1 JH2 | JAK1 JH1 |
|---|---|---|---|---|---|---|---|
| Cmpd | (Kd, nM) | (Kd, nM) | (Kd, nM) | (Kd, nM) | (Kd, nM) | (Kd, nM) | (Kd, nM) |
| 1 | E | C | A | C | A | D | A |
| 2 | E | A | | | | D | |
| 3 | E | A | | | | C | |
| 4 | E | A | | | | C | |
| 5 | E | A | A | B | A | C | A |
| 6 | E | C | A | C | A | D | A |
| 7 | E | C | B | C | A | D | A |
| 8 | E | C | B | C | A | D | A |
| 9 | E | A | | | | C | |
| 10 | E | C | | | | C | |
| 11 | E | B | | | | C | |
| 12 | E | C | | | | D | |
| 13 | E | C | | | | B | |
| 14 | E | B | | | | B | |
| 15 | E | A | | | | C | |
| 16 | E | A | | | | A | |
| 33 | E | A | A | A | A | C | A |
| 34 | E | A | A | B | A | D | A |
| 35 | E | A | | | | D | |
| 36 | E | C | | | | D | |
| 37 | E | A | A | A | A | B | A |
| 38 | E | A | | | | C | |
| 52 | E | B | | | | C | |
| 53 | E | B | | | | D | |
| 54 | E | | | | | | |
| 55 | E | | | | | | |
| 56 | E | | | | | | |
| 57 | E | | | | | | |
| 58 | E | | | | | | |
| 59 | E | | | | | | |
| 60 | E | B | | | | D | |
| 61 | E | | | | | | |
| 62 | E | | | | | | |
| 63 | E | | | | | | |
| 64 | E | | | | | | |
| 65 | E | | | | | | |
| 66 | E | | | | | | |
| 67 | E | | | | | | |
| 68 | E | | | | | | |
| 69 | E | | | | | | |
| 70 | E | | | | | | |
| 71 | D | | | | | | |
| 72 | D | | | | | | |
| 73 | D | | | | | | |
| 74 | D | | | | | | |
| 75 | D | | | | | | |
| 76 | C | | | | | | |
| 77 | C | | | | | | |
| 78 | C | | | | | | |
| 79 | C | A | | | | A | |
| 80 | A | A | | | | A | |
| 81 | C | A | | | | A | |
| 90 | E | A | | | | D | |
| 91 | E | D | | | | C | |
| 92 | E | A | | | | B | |
| 93 | E | A | | | | C | |
| 94 | E | A | | | | B | |
| 95 | E | A | | | | A | |
| 96 | E | C | | | | D | |
| 97 | E | C | | | | D | |
| 98 | E | D | | | | D | |
| 99 | E | | | | | | |

A ≥ 0000 nM; B ≥ 10000 nM and <30000 nM; C ≥ 1000 nM and <10000 nM; D ≥ 10 nM and <1000 nM; E < 10 nM.

TABLE 3

| | IFNa, PBMCs, p-STAT3 | IFNa, PBMCs, p-STAT5 |
|---|---|---|
| Compound | (IC50, nM) | (IC50, nM) |
| 1 | C | C |
| 2 | A | B |
| 5 | C | C |
| 6 | C | C |

A ≥ 100 nM; B ≥ nM and < 50 nM; C < 50 nM.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, joumal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound represented by Formula I: 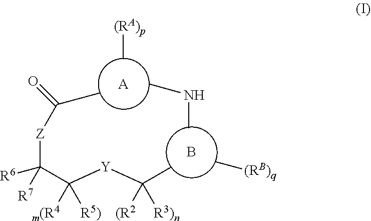
or a pharmaceutically acceptable salt and/or a stereoisomer thereof, wherein
Ring A is a 6,5 or 6,6 fused bicyclic heteroaryl containing two, three, or four ring nitrogens;
R^{A} is independently selected for each occurrence from the group consisting of hydrogen, halogen, —NR^{a}R^{b}, C₁-C₆alkyl, and C₁-C₆alkoxy; wherein C₁-C₆alkyl and C₁-C₆alkoxy may optionally be substituted by one or more halogens or deuteriums;
Ring B is a 6,5 or 6,6 fused bicyclic heteroaryl containing one, two or three ring nitrogens;
R^{B} is independently selected for each occurrence from the group consisting of hydrogen, halogen, —NR^{a}R^{b}, C₁-C₆alkyl, and C₁-C₆alkoxy; wherein C₁-C₆alkyl and C₁-C₆alkoxy may optionally be substituted by one or more halogens or deuteriums;
Y is selected from the group consisting of O, N(R^{a}), S(O)_{w}, CH₂ and a bond;
Z is selected from the group consisting of N(R^{a}) and O;
R² and R³ are each independently selected from the group consisting of hydrogen, deuterium, and C₁-C₆alkyl optionally substituted by one or more substituents each independently selected from R^{P}; or
R² and R³, together with the carbon to which they are attached, may be joined together to form a C₃-C₆cycloalkyl optionally substituted by one or more substituents each independently selected from R^{P};
R⁴ and R⁵ are each independently selected for each occurrence from the group consisting of hydrogen, deuterium, and C₁-C₆alkyl optionally substituted by one or more substituents each independently selected from R^{P}; or
R⁴ and R⁵, together with the carbon to which they are attached, may be joined together to form a C₃-C₆cycloalkyl optionally substituted by one or more substituents each independently selected from R^{P};
R⁶ and R⁷ are independently selected from the group consisting of hydrogen, deuterium, halogen, hydroxyl, —NR^{a}R^{b}, cyano, oxo, C₁-C₆alkyl, C₂-C₆alkenyl, C₂-C₆alkynyl, C₁-C₆heteroalkyl, and C₁-C₆alkoxy; wherein R⁴ may be substituted on an available carbon by hydroxyl or one or more substituents each independently selected from R; or
R⁶ and R⁷, together with the carbon to which they are attached, may be joined together to form a C₃-C₆cycloalkyl optionally substituted by one or more substituents each independently selected from R^{P};
R is independently selected for each occurrence from the group consisting of deuterium, halogen, hydroxyl, —NR^{a}R^{b}, cyano, oxo, C₁-C₆alkyl, and C₁-C₆alkoxy;
R^{a} and R^{b} are independently selected for each occurrence from the group consisting of hydrogen and C₁-C₆alkyl, wherein C₁-C₆alkyl may optionally substituted by one or more halogens or deuteriums;
m is 1 or 2;
n is 0 or 1;
p is 0, 1, or 2;
q is 0, 1 or 2; and
w is 0, 1 or 2.

2. The compound of claim 1, wherein ring A is represented by: 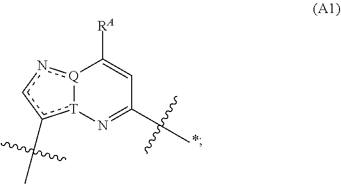
wherein:
Q is N and T is C; or
Q is C and T is N; and
R^{A} is selected from the group consisting of —N(H)CH₃, —NH₂, hydrogen, and —OCH₃;
wherein * represents the point of attachment to the —NH— group in Formula I.

3. The compound of claim 1 or 2, wherein ring A is selected from the group consisting of:

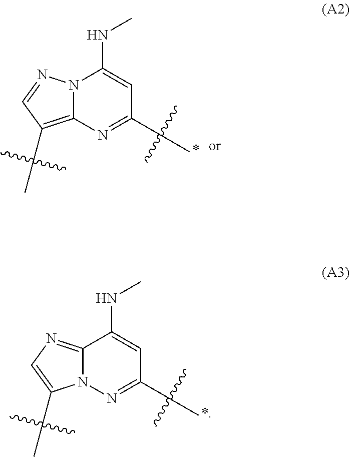 [SMILES: *c1cc(NC)n2ncc(*)c2n1 ; *c1cc(NC)c2ncc(*)n2n1]

4. The compound of claim 1, wherein ring A is represented by: 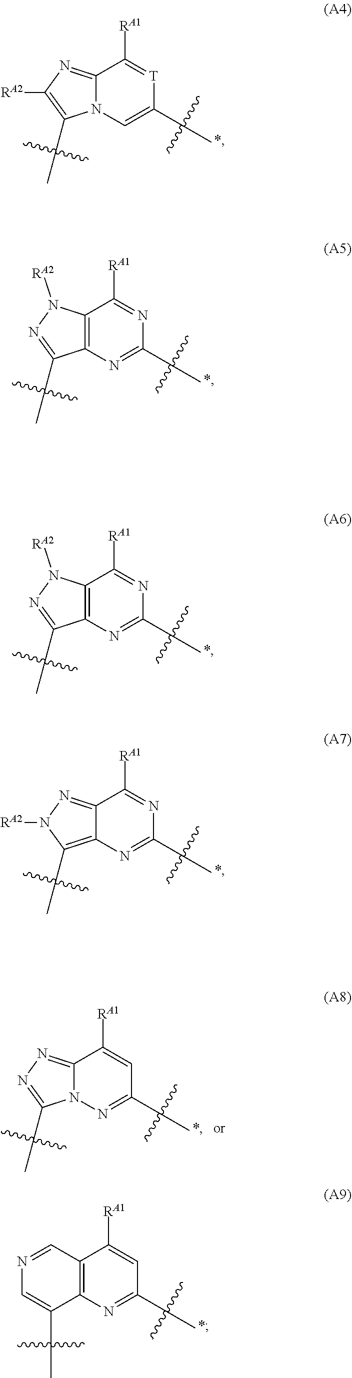
wherein:
T is N or CH;
R^{A1} is selected from the group consisting of —N(H)CH₃, —NH₂, hydrogen, and —OCH₃; and
R^{A2} is selected from the group consisting of hydrogen and —CH₃;
wherein * represents the point of attachment to the —NH— group in Formula I.

5. The compound of claim 1 or 4, wherein ring A is selected from the group consisting of:

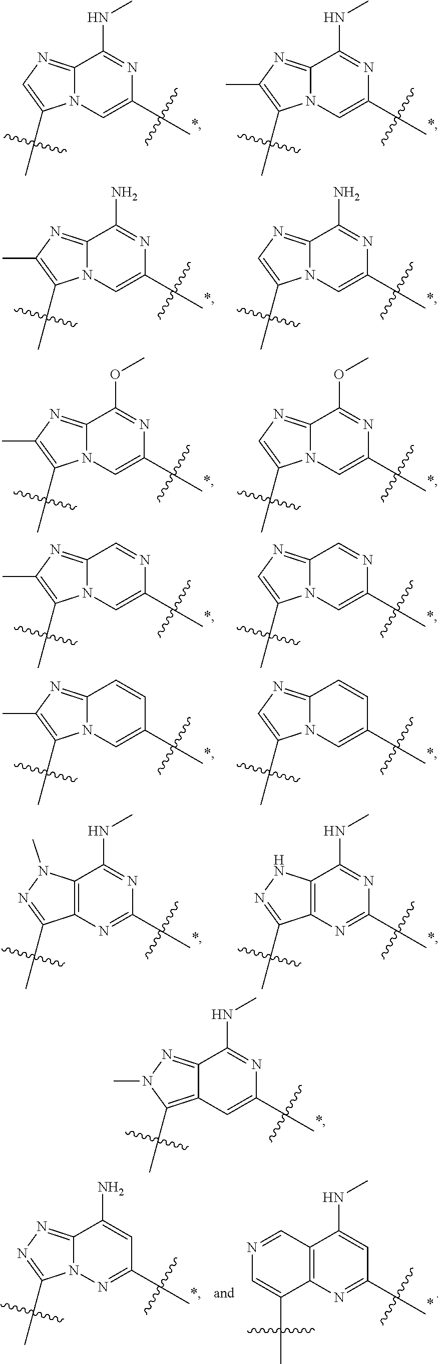

6. The compound of any one of claims 1-5, wherein ring B is represented by: 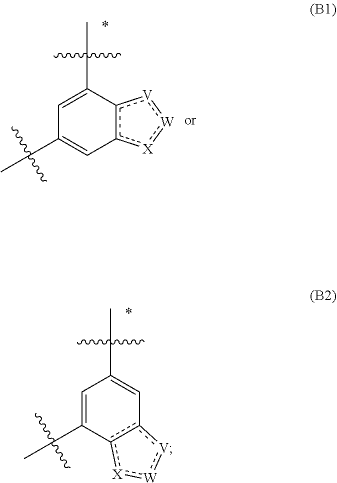
wherein:
V is N(R^{V}), W is N, and X is N; or
V is N, W is N(R^{V}), and X is N; or
V is N, W is N, and X is N(R^{X}); or
V is N, W is C(R^{W}), and X is N(R^{X}); or
V is N(R^{V}), W is C(R^{W}), and X is N; or
V is N(R^{V}), W is N, and X is C(R^{X}); or
V is N, W is N(R^{W}), and C is C(R^{X}); or
V is C(R^{V}), W is N(R^{W}), and X is N; or
V is C(R^{V}), W is N, and X is N(R^{X}); or
V is N, W is C(R^{W}), and X is C(R^{X}); or
V is C(R^{V}), W is C(R^{W}), and X is N; or
V is O, W is N, and X is N(R^{X}); or
V is C(R′), W is N, and X is O; or
V is O, W is C(R^{W}), and X is N; or
V is N, W is C(R^{W}), and X is O; and
R^{Y}, R^{W} and R^{X} are each independently selected from the group consisting of hydrogen and C₁-C₆alkyl; wherein C₁-C₆alkyl may optionally be substituted by one or more substituents each independently selected from the group consisting of deuterium, halogen, and hydroxyl;
wherein * represents the point of attachment to the —NH— group in Formula I.

7. The compound of any one of claims 1-6, wherein ring B is represented by: 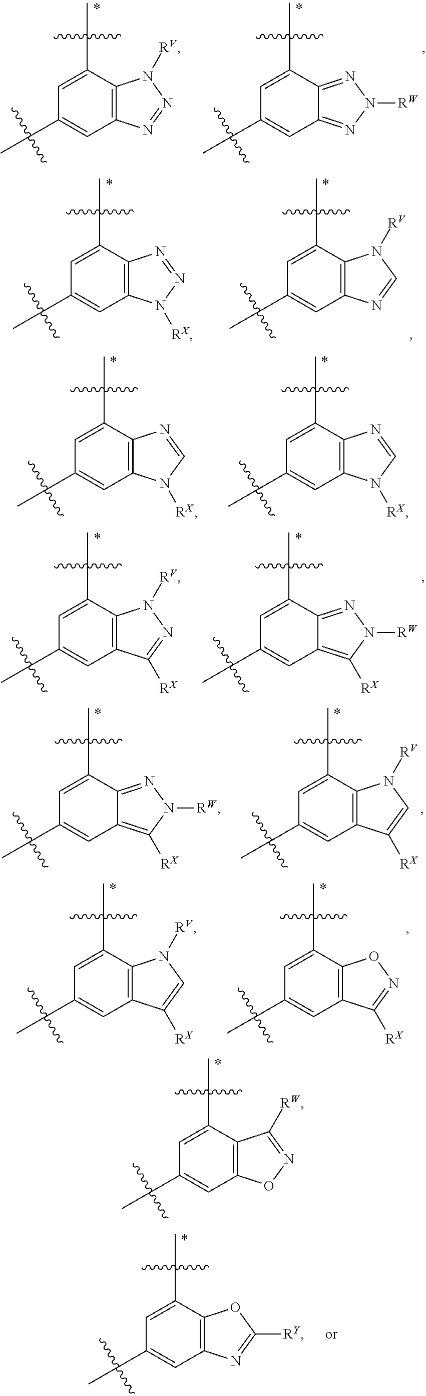 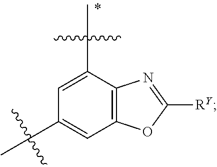
wherein R^{V}, R^{W} and R^{X} are each independently selected from the group consisting of hydrogen, —CH₃, and —CD₃.

8. The compound of any one of claims 1-6, wherein ring B is represented by: 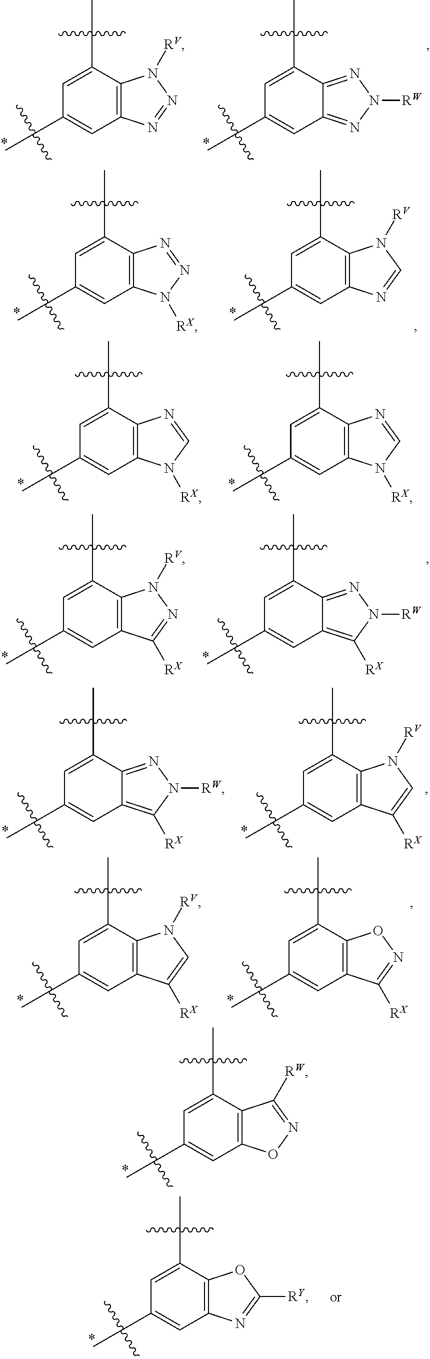 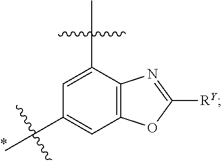
wherein R^{V}, R^{W} and R^{X} are each independently selected from the group consisting of hydrogen, —CH₃, and —CD₃.

9. The compound of any one of claims 1-5, wherein ring B is represented by: 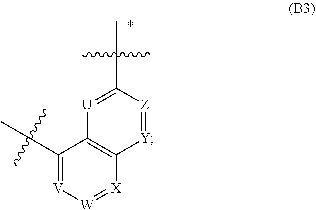
wherein:
U, V, W, X, Y, and Z are each independently selected from N and C(R^{B}); wherein one or two of U, V, W, X, Y, and Z are N; and
R^{B} is independently selected for each occurrence from the group consisting of hydrogen, halogen, C₁-C₃alkyl, and C₁-C₃alkoxy;
wherein * represents the point of attachment to the —NH— group in Formula I.

10. The compound of any one of claims 1-5 and 9, wherein ring B is selected from the group consisting of: 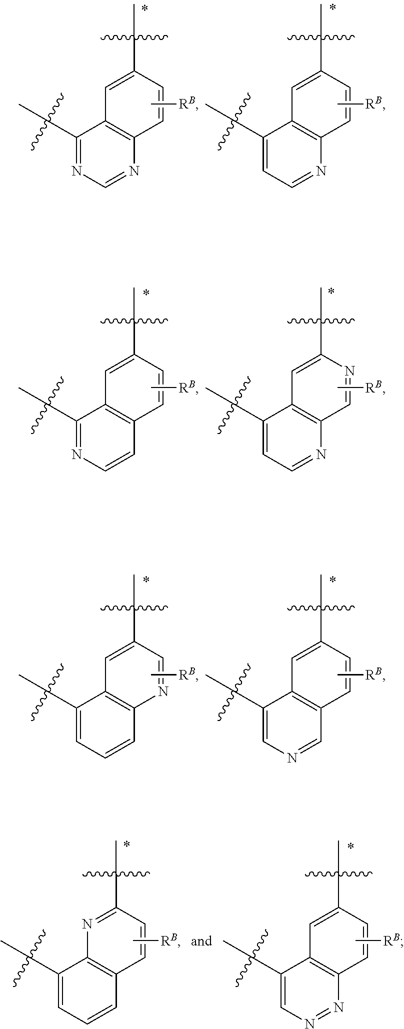
wherein R^{B} is selected from the group consisting of hydrogen, —CH₃, and —OCH₃.

11. The compound of any one of claims 1-5, wherein ring B is selected from the group consisting of: 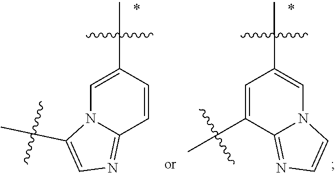 [SMILES: *c1ccc2ncc(*)n2c1 ; *c1cc(*)c2nccn2c1]
wherein * represents the point of attachment to the —NH— group in Formula I.

12. The compound of any one of claims 1, 2 and 6, wherein the compound is represented by Formula II:

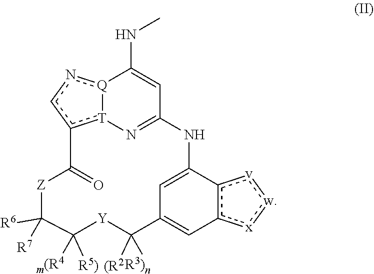

13. The compound of claim 12, wherein the compound is represented by:

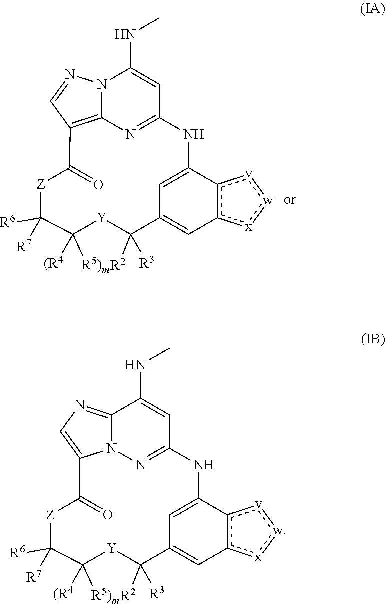

14. The compound of claim 12, wherein the compound is represented by:

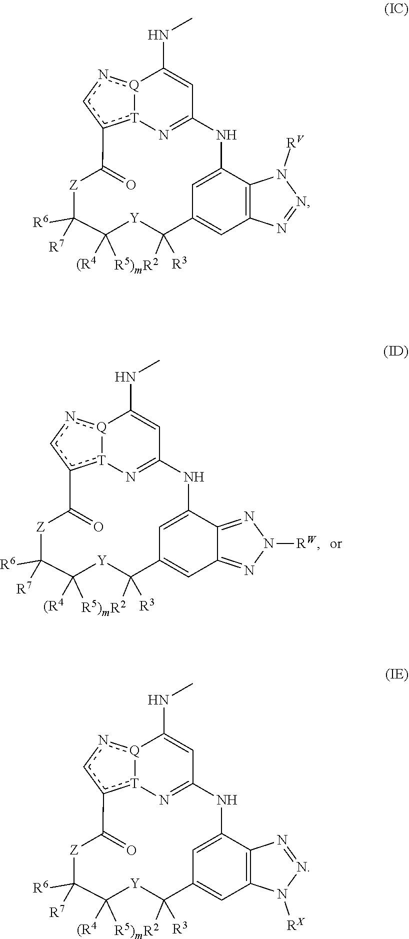

15. The compound of claim 12, wherein the compound is represented by:

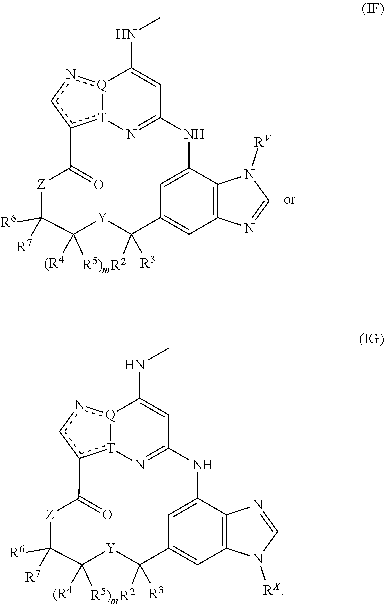

16. The compound of claim 12, wherein the compound is represented by:

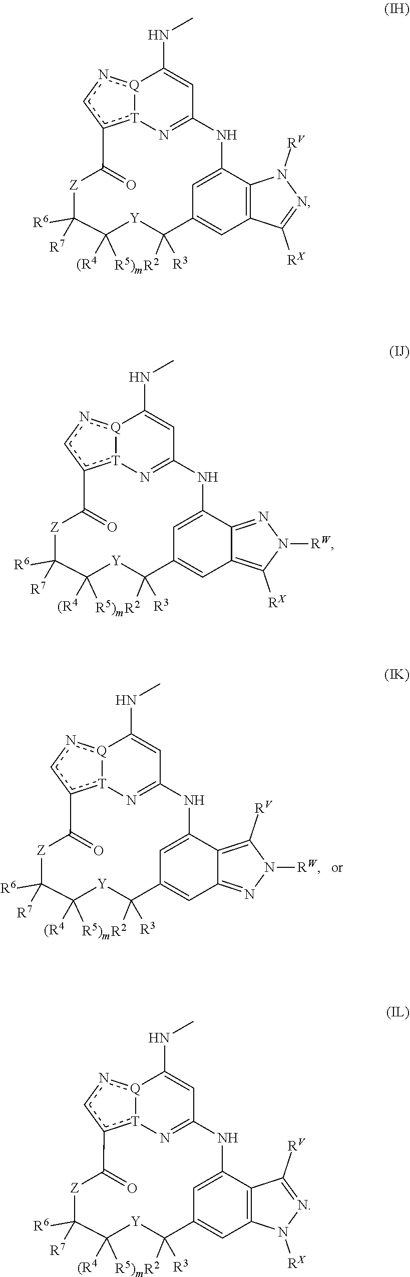

17. The compound of claim 12, wherein the compound is represented by:

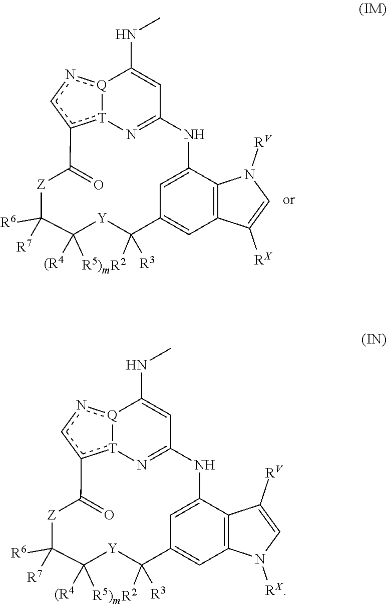

18. The compound of claim 12, wherein the compound is represented by:

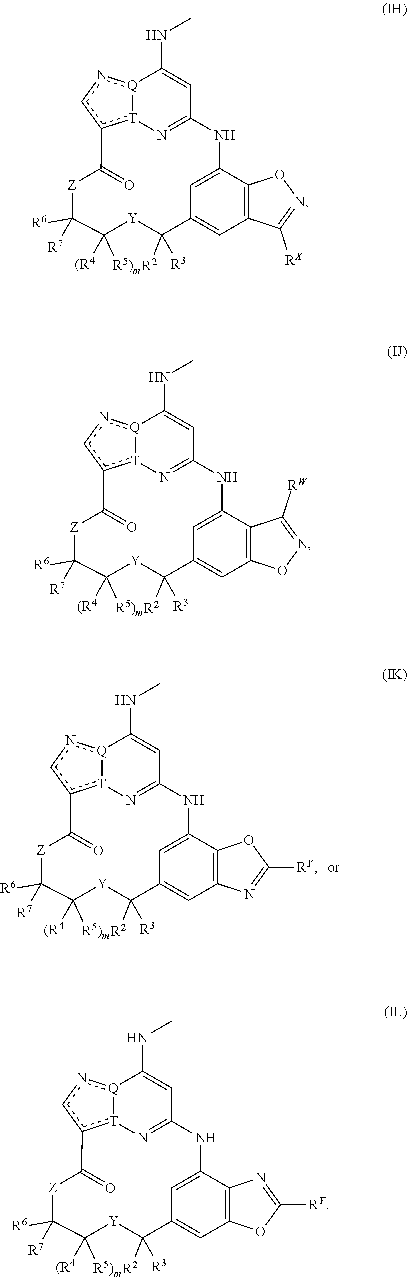

19. The compound of any one of claims 12-18, wherein R^{X}, R^{W} and R^{V} are each hydrogen or C₁-C₆alkyl optionally substituted by one or more deuteriums.

20. The compound of any one of claims 12-19, wherein R^{X}, R^{W} and R^{V} are each independently selected from the group consisting of hydrogen, —CH₃, and —CD₃.

21. The compound of any one of claims 1-20, wherein Y is selected from the group consisting of O, NH, NCH₃, S, S(O), and S(O)₂.

22. The compound of any one of claims 1-21, wherein Y is O.

23. The compound of any one of claims 1-22, wherein Z is selected from O, NH, and NCH₃.

24. The compound of any one of claims 1-23, wherein Z in NH.

25. The compound of any one of claims 1-24, wherein R² and R³ are each independently selected from the group consisting of hydrogen, deuterium, —CH₃, and —CF₃; or R² and R³, together with the carbon to which they are attached, join to form cyclopropyl.

26. The compound of any one of claims 1-25, wherein m is 1.

27. The compound of any one of claims 1-25, wherein m is 2.

28. The compound of any one of claims 1-27, wherein n is 0.

29. The compound of any one of claims 1-27, wherein n is 1.

30. The compound of any one of claims 1-29, wherein R⁴ and R⁵ are independently selected for each occurrence from the group consisting of hydrogen, deuterium, —CH₃, and —CF₃; or R⁴ and R⁵, together with the carbon to which they are attached, join to form cyclopropyl.

31. The compound of any one of claims 1-30, wherein R⁶ and R⁷ are each independently selected from the group consisting of hydrogen, deuterium, and C₁-C₆alkyl, wherein C₁-C₆alkyl may optionally be substituted by one or more halogen or hydroxyl groups.

32. The compound of any one of claims 1-31, wherein R⁶ and R⁷ are each independently selected from the group consisting of hydrogen, deuterium, —CH₃, —CF₃, —CH₂OH, —CH(CH₃)₂, and —C(CH₃)₂OH.

33. The compound of any one of claim 1-32, wherein R⁶ and R⁷, together with the carbon to which they are attached, join to form cyclopropyl.

34. A compound represented by: 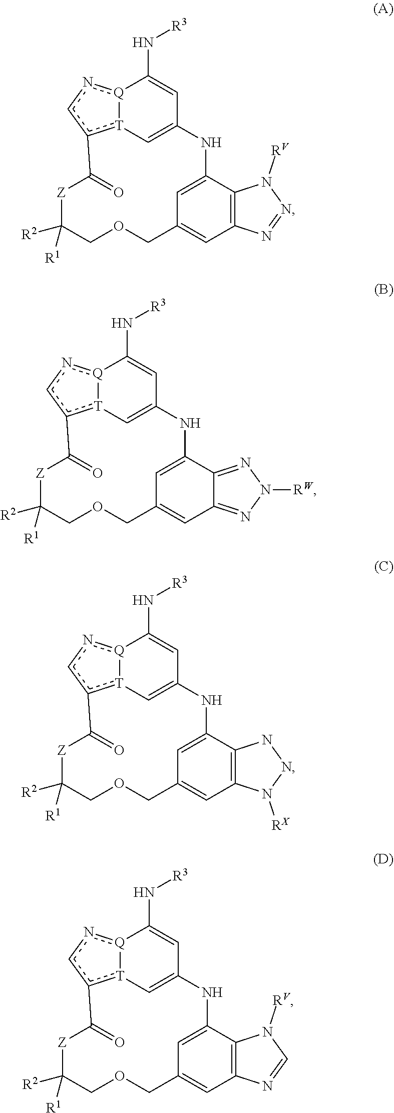 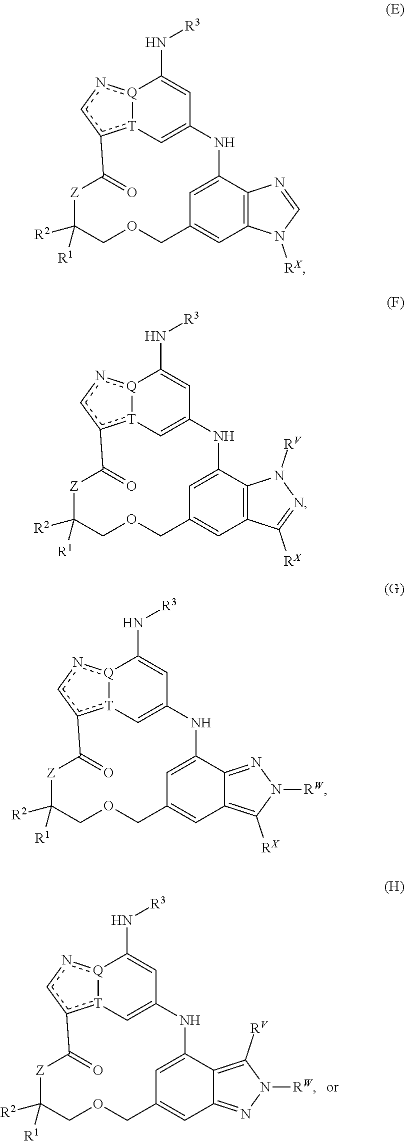 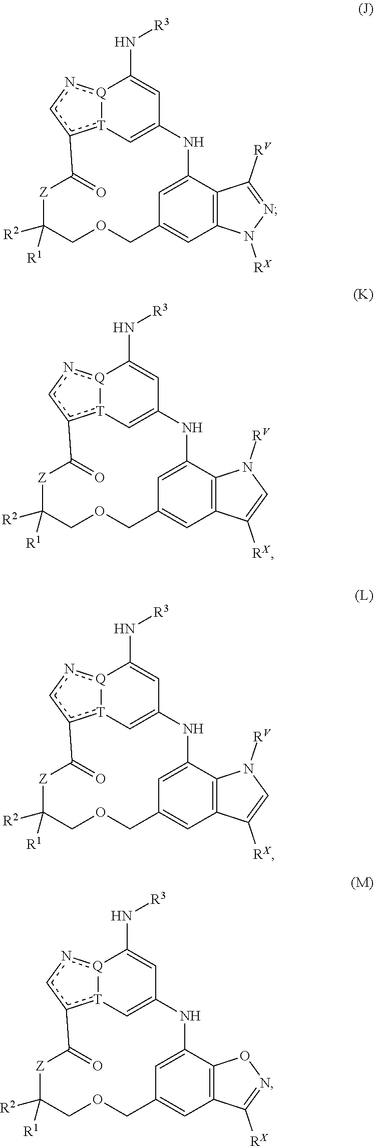 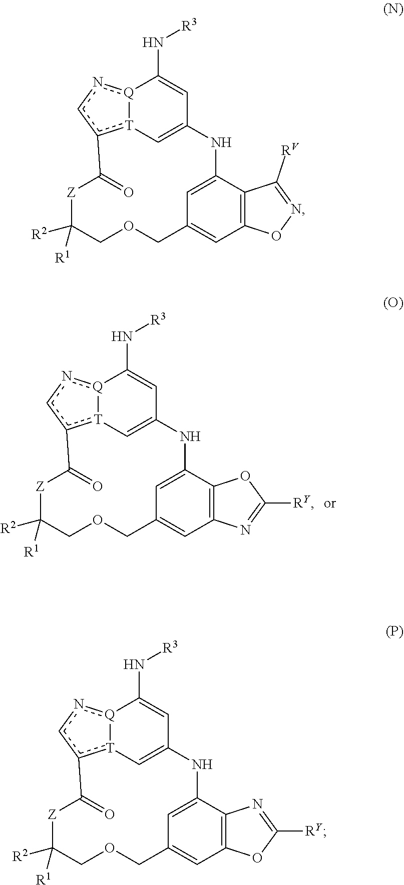
or a pharmaceutically acceptable salt and/or a stereoisomer thereof, wherein:
Q is N and T is C; or
Q is C and T is N;
wherein Q and T are each members of a heteroaromatic ring system;
Z is NH or O;
R^{V}, R^{W} and R^{X} are each independently selected from the group consisting of hydrogen and C₁-C₄alkyl optionally substituted by one or more deuteriums;
R¹ and R² are independently selected from the group consisting of hydrogen, deuterium, and C₁-C₄alkyl, wherein C₁-C₄alkyl may be substituted on an available carbon by one or more halogen or hydroxyl; or
R¹ and R², together with the carbon to which they are attached, may be joined together to form cyclopropyl; and
R³ is hydrogen or C₁-C₄alkyl optionally substituted by one or more deuteriums.

35. The compound of claim 34, wherein R^{X}, R^{W} and R^{V} are each independently selected from hydrogen, —CH₃ and —CD₃.

36. The compound of claim 34 or 35, wherein Z is NH.

37. The compound of any one of claims 34-36, wherein R¹ and R² are each independently selected from the group consisting of hydrogen, deuterium, —CH₃, —CF₃, —CH₂OH, —CH(CH₃)₂, and —C(CH₃)₂OH.

38. The compound of any one of claim 34-37, wherein R¹ and R², together with the carbon to which they are attached, join to form cyclopropyl.

39. The compound of any one of claim 34-38, wherein R³ is —CH₃.

40. The compound of any one of claims 34-39, wherein Q is N and T is C.

41. The compound of any one of claims 34-39, wherein Q is C and T is N.

42. A compound selected from the group consisting of: 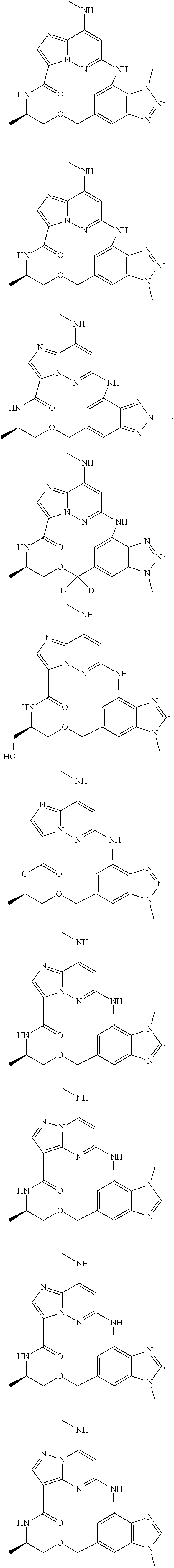 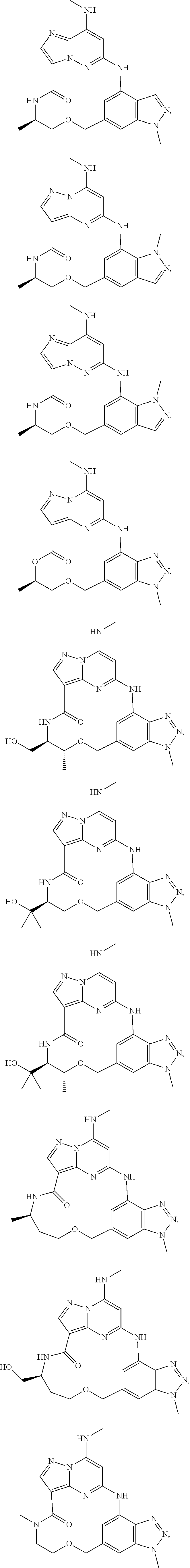 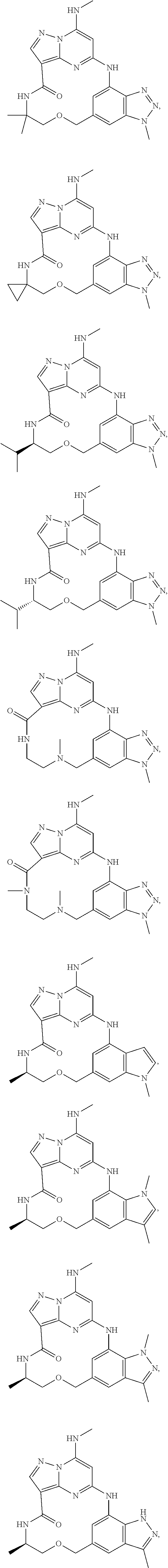 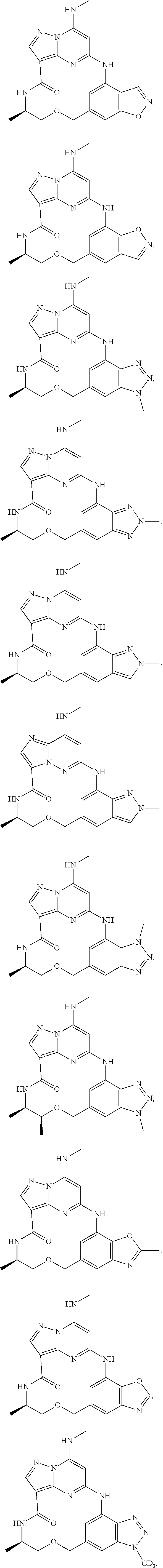 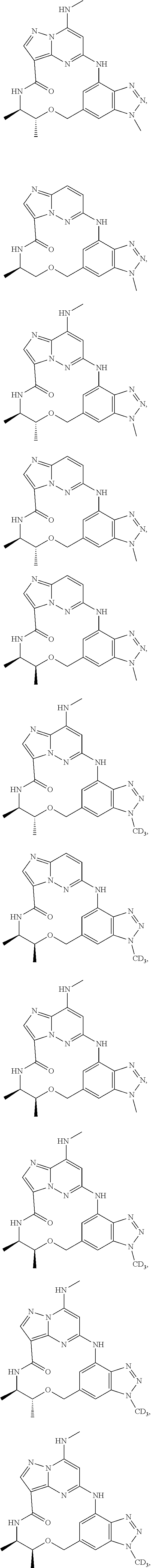 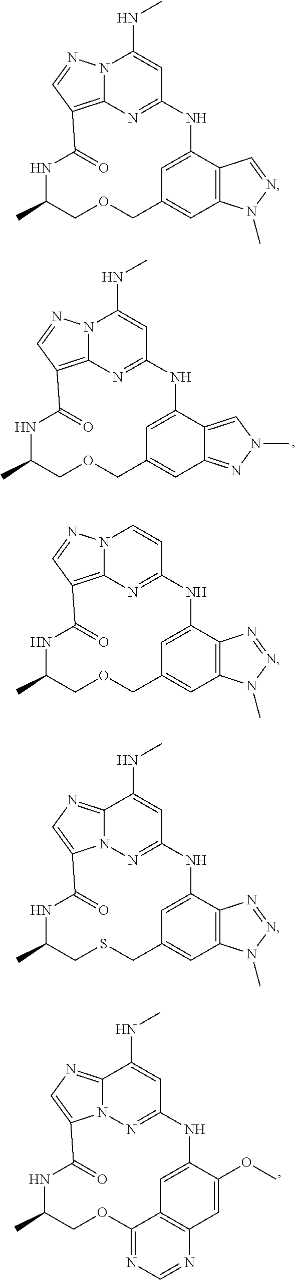 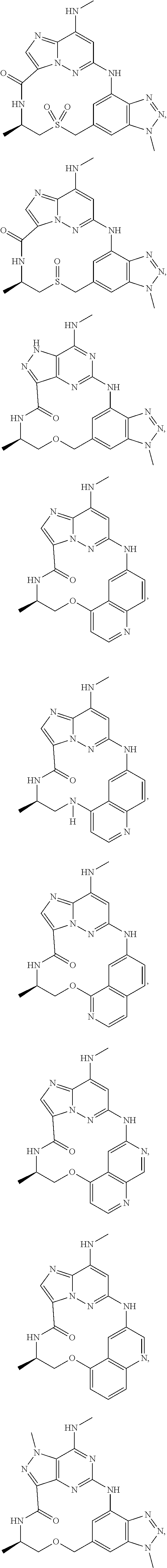 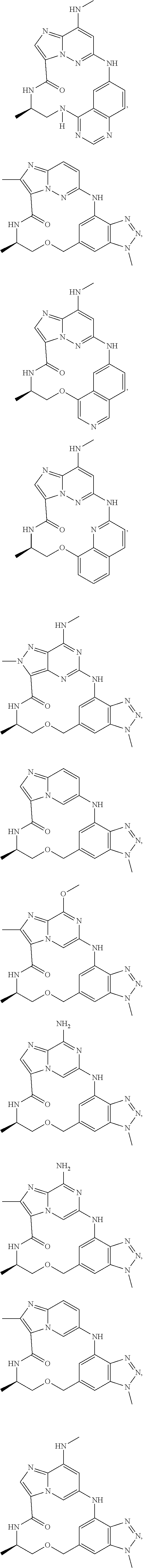 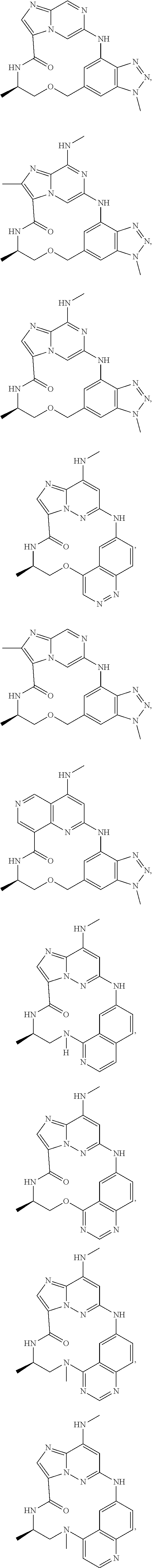 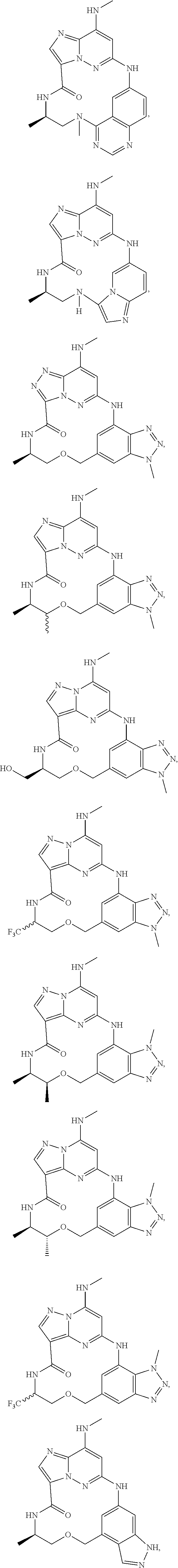 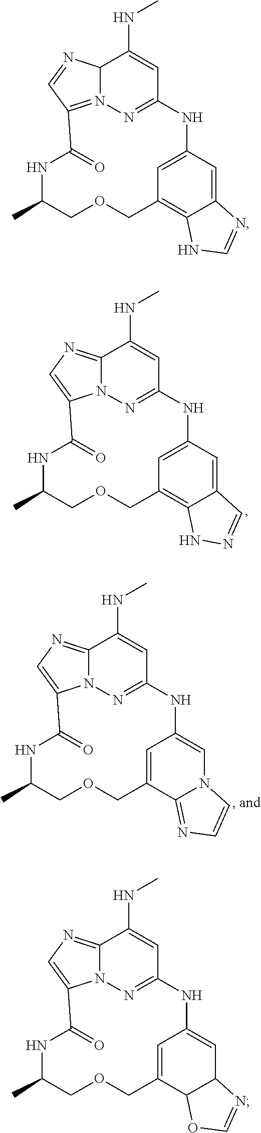
or a pharmaceutically acceptable salt and/or a stereoisomer thereof.

43. A pharmaceutical composition comprising a compound of any one of claims 1-42, or a pharmaceutically acceptable salt and/or a stereoisomer thereof, and a pharmaceutically acceptable excipient.

44. A method of inhibiting a TYK2 enzyme in a patient or biological sample, comprising contacting said patient or biological sample with a therapeutically effective amount of a compound of any one of claims 1-42, or the pharmaceutical composition of claim 43.

45. A method of inhibiting TYK2 activity in a patient in need thereof, comprising administering to the patient a therapeutically effective amount of a compound of any one of claims 1-42, or the pharmaceutical composition of claim 43.

46. The method of claim 45, wherein inhibiting TYK2 activity is associated with treating a disease or disorder selected from the group consisting of Crohn's disease, rheumatoid arthritis, psoriasis, systemic lupus erythematosus, ulcerative colitis, psoriatic arthritis, and systemic sclerosis.

47. A method of treating a TYK2-mediated disorder in a patient in need thereof, comprising administering to the patient a therapeutically effective amount of a compound of any one of claims 1-42, or the pharmaceutical composition of claim 44.

48. The method of claim 47, wherein the TYK2-mediated disorder is an autoimmune disorder, an inflammatory disorder, a proliferative disorder, an endocrine disorder, a neurological disorder, or a disorder associated with transplantation.

49. The method of claim 48, wherein the disorder is associated with type I interferon, IL-10, IL-12, or IL-23 signalling.

50. A method of treating one or more of: Crohn's disease, rheumatoid arthritis, psoriasis, systemic lupus erythematosus, ulcerative colitis, psoriatic arthritis, and systemic sclerosis in a patient in need thereof, comprising administering to the patient an effective amount of any one of the compounds of claims 1-42, or the pharmaceutical composition of claim 43.
